(19) United States
(12) Patent Application Publication
(10) Pub. No.: US 2003/0215803 A1
(43) Pub. Date: Nov. 20, 2003

(54) Human genes and gene expression products isolated from human prostate

(76) Inventors: Pablo Dominguez Garcia, San Francisco, CA (US); Jaime Escobedo, Alamo, CA (US); Altaf Kassam, Oakland, CA (US); George Lamson, Moraga, CA (US); Elizabeth Scott, Petaluma, CA (US); Radoje Drmanac, Palo Alto, CA (US); Radomir Crkvenjakov, Sunnyvale, CA (US); Mark Dickson, Hollister, CA (US); Snezana Drmanac, Palo Alto, CA (US); Ivan Labat, Mountain View, CA (US); Dena Leshkowitz, Kiryat Hasavionim (IL); David Kita, Milpitas, CA (US); Veronica Garcia, Hayward, CA (US); Lee William Jones, Hayward, CA (US); Birgit Stache-Crain, Sunnyvale, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/012,697

(22) Filed: Dec. 7, 2001

Related U.S. Application Data
(60) Provisional application No. 60/275,688, filed on Mar. 13, 2001.
(60) Provisional application No. 60/254,648, filed on Dec. 7, 2000.

(51) Int. Cl. C12Q 1/68; C07H 21/04; C12N 9/00; C12P 21/02; C12N 5/06; C07K 14/47; C07K 16/40

(52) U.S. Cl. 435/6; 435/69.1; 435/183; 435/320.1; 435/325; 530/350; 536/23.2; 530/388.1

(57) ABSTRACT

This invention relates to novel human polynucleotides and variants thereof, their encoded polypeptides and variants thereof, to genes corresponding to these polynucleotides and to proteins expressed by the genes. The invention also relates to diagnostics and therapeutics comprising such novel human polynucleotides, their corresponding genes or gene products, including probes, antisense nucleotides, and antibodies. The polynucleotides of the invention correspond to a polynucleotide comprising the sequence information of at least one of SEQ ID NOS: 1-1477. The polypeptides of the invention correspond to a polypeptide comprising the amino acid sequence information of at least one of SEQ ID NOS: 1478-1568.

### CROSS-REFERENCE TO RELATED APPLICATION

Ths application claims the benefit of earlier-filed U.S. provisional application serial No. 60/254,648 filed Dec. 11, 2000, and of ealier-filed U.S. provisional application serial No. 60/275,688 filed Mar. 13, 2001, which applications are incorporated herein by reference in their entirety.

### FIELD OF THE INVENTION

The present invention relates to polynucleotides of human origin, particularly in human prostate, and the encoded gene products.

### BACKGROUND OF THE INVENTION

Identification of novel polynucleotides, particularly those that encode an expressed gene product, is important in the advancement of drug discovery, diagnostic technologies, and the understanding of the progression and nature of complex diseases such as cancer. Identification of genes expressed in different cell types isolated from sources that differ in disease state or stage, developmental stage, exposure to various environmental factors, the tissue of origin, the species from which the tissue was isolated, and the like is key to identifying the genetic factors that are responsible for the phenotypes associated with these various differences.

This invention provides novel human polynucleotides, the polypeptides encoded by these polynucleotides, and the genes and proteins corresponding to these novel polynucleotides.

### SUMMARY OF THE INVENTION

This invention relates to novel human polynucleotides and variants thereof, their encoded polypeptides and variants thereof, to genes corresponding to these polynucleotides and to proteins expressed by the genes. The invention also relates to diagnostics and therapeutics comprising such novel human polynucleotides, their corresponding genes or gene products, including probes, antisense nucleotides, and antibodies. The polynucleotides of the invention correspond to a polynucleotide comprising the sequence information of at least one of SEQ ID NOS: 1-1477. The polypeptides of the invention correspond to a polypeptide comprising the amino acid sequence information of at least one of SEQ ID NOS: 1478-1568.

Various aspects and embodiments of the invention will be readily apparent to the ordinarily skilled artisan upon reading the description provided herein.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It must be noted that as used herein and in the appended claims, the singular forms “a,” “and,” and “the” include plural referents unless the context clearly dictates otherwise. Thus, for example, reference to “a polynucleotide” includes a plurality of such polynucleotides and reference to “the colon cancer cell” includes reference to one or more cells and equivalents thereof known to those skilled in the art, and so forth.

The publications and applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “polynucleotide” and “nucleic acid,” used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, branched nucleic acid (see, e.g., U.S. Pat. Nos. 5,124,246; 5,710,264; and 5,849,481), or a polymer comprising purine and pyrimidine bases or other natural, chemically or biochemically modified, non-natural, or derivatized nucleotide bases. These terms furhter include, but are not limited to, mRNA or cDNA that comprise intronic sequences (see, e.g., Niwa et al. (1999) Cell 99(7):691-702). The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynuclotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The terms “polypeptide” and “protein,” used interchangebly herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Sample” or “biological sample” as used herein encompasses a variety of sample types, and are generally meant to refer to samples of biological fluids or tissues, particularly samples obtained from tissues, especially from cells of the type associated with a disease or condition for which a diagnostic application is designed (e.g., ductal adenocarcinoma), and the like. “Sample” or “biological sample” are meant to encompass blood and other liquid samples of biological origin, solid tissue samples, such as a biopsy specimen or tissue cultures or cells derived therefrom and the progeny thereof. These terms encompass samples that have been manipulated in any way after their procurement as well as derivatives and fractions of samples, where the samples may be maniuplated by, for example, treatment with reagents, solubilization, or enrichment for certain components. The terms also encompass clinical samples, and also includes cells in cell culture, cell supernatants, cell lysates, serum, plasma, biological fluids, and tissue samples. Where the sample is solid tissue, the cells of the tissue can be dissociated or tissue sections can be analyzed.

The terms “treatment,” “treating,” “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and so on.

As used herein the term “isolated” refers to a polynucleotide, a polypeptide, an antibody, or a host cell that is in an environment different from that in which the polynucleotide, the polypeptide, the antibody, or the host cell naturally occurs. A polynucleotide, a polypeptide, an antibody, or a host cell which is isolated is generally substantially purified. As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight.

A “host cell,” as used herein; refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer,” “neoplasm,” “tumor,” and “carcinoma,” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, metastatic, and non-metastatic cells. Detection of cancerous cell is of particular interest.

The use of “e”, as in 10e−3, indicates that the number to the left of “e” is raised to the power of the number to the right of “e” (thus, 10e−3 is 10⁻³).

The term “heterologous” as used herein in the context of, for example, heterologous nucleic acid or amino acid sequences, heterologous polypeptides, or heterologous nucleic acid, is meant to refer to material that originates from a source different from that with which it is joined or associated. For example, two DNA sequences are heterologous to one another if the sequences are from different genes or from different species. A recombinant host cell containing a sequence that is heterologous to the host cell can be, for example, a bacterial cell containing a sequence encoding a human polypeptide.

The invention relates to polynucleotides comprising the disclosed nucleotide sequences, to full length cDNA, mRNA, genomic sequences, and genes corresponding to these sequences and degenerate variants thereof, and to polypeptides encoded by the polynucleotides of the invention and polypeptide variants. The following detailed description describes the polynucleotide compositions encompassed by the invention, methods for obtaining cDNA or genomic DNA encoding a full-length gene product, expression of these polynucleotides and genes, identification of structural motifs of the polynucleotides and genes, identification of the function of a gene product encoded by a gene corresponding to a polynucleotide of the invention, use of the provided polynucleotides as probes and in mapping and in tissue profiling, use of the corresponding polypeptides and other gene products to raise antibodies, and use of the polynucleotides and their encoded gene products for therapeutic and diagnostic purposes.

Polynucleotide Compositions

The scope of the invention with respect to polynucleotide compositions includes, but is not necessarily limited to, polynucleotides having a sequence set forth in any one of SEQ ID NOS: 1-1477; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicated.

The invention features polynucleotides that are expressed in human tissue, especially human colon, prostate, breast, lung and/or endothelial tissue. Novel nucleic acid compositions of the invention of particular interest comprise a sequence set forth in any one of SEQ ID NOS: 1-1477 or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from any one of SEQ ID NOS: 1-1477.

The polynucleotides of the invention also include polynucleotides having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences (SEQ ID NOS: 1-1477) under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

Preferably, hybridization is performed using at least 15 contiguous nucleotides (nt) of at least one of SEQ ID NOS: 1-1477. That is, when at least 15 contiguous nt of one of the disclosed SEQ ID NOS. is used as a probe, the probe will preferentially hybridize with a nucleic acid comprising the complementary sequence, allowing the identification and retrieval of the nucleic acids that uniquely hybridize to the selected probe. Probes from more than one SEQ ID NO. can hybridize with the same nucleic acid if the cDNA from which they were derived corresponds to one mRNA. Probes of more than 15 nt can be used, e.g., probes of from about 18 nt to about 100 nt, but 15 nt represents sufficient sequence for unique identification.

The polynucleotides of the invention also include naturally occurring variants of the nucleotide sequences (e.g., degenerate variants, allelic variants, etc.). Variants of the polynucleotides of the invention are identified by hybridization of putative variants with nucleotide sequences disclosed herein, preferably by hybridization under stringent conditions. For example, by using appropriate wash conditions, variants of the polynucleotides of the invention can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to the polynucleotides of SEQ ID NOS: 1-1477, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST, described in Altschul, et al. Nucleic Acids Res. (1997) 25:3389-3402, or TeraBLAST available from TimeLogic Corp. (Crystal Bay, Nev.).

In general, variants of the invention have a sequence identity greater than at least about 65%, preferably at least about 75%, more preferably at least about 85%, and can be greater than at least about 90% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject polypeptides. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments of the invention comprise at least about 10, about 15, about 20, about 35, about 50, about 100, about 150 to about 200, about 250 to about 300, or about 350 contiguous nt selected from the polynucleotide sequences as shown in SEQ ID NOS: 1-1477. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from the group consisting of the polynucleotides shown in SEQ ID NOS: 1-1477.

Probes specific to the polynucleotides of the invention can be generated using the polynucleotide sequences disclosed in SEQ ID NOS: 1-1477. The probes are preferably at least about 12, 15, 16, 18, 20, 22, 24, or 25 nt fragment of a corresponding contiguous sequence of SEQ ID NOS: 1-1477, and can be less than 10, 5, 2, 1, 0.5, 0.1, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of a polynucleotide of one of SEQ ID NOS: 1-1477. More preferably, probes are designed based on a contiguous sequence of one of the subject polynucleotides that remain unmasked following application of a masking program for masking low complexity (e.g., XBLAST, RepeatMasker, etc.) to the sequence., i.e., one would select an unmasked region, as indicated by the polynucleotides outside the poly-n stretches of the masked sequence produced by the masking program.

The polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The polynucleotides of the invention can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used, for example, to produce polypeptides, as probes for the detection of mRNA of the invention in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of the polynucleotide sequences as shown in SEQ ID NOS: 1-1477 or variants thereof in a sample. These and other uses are described in more detail below.

Use of Polynucleotides to Obtain Full-Length cDNA, Gene, and Promoter Region

In one embodiment, the polynucleotides are useful as starting materials to construct larger molecules. In one example, the polynucleotides of the invention are used to construct polynucleotides that encode a larger polypeptide (e.g., up to the full-length native polypeptide as well as fusion proteins comprising all or a portion of the native polypeptide) or may be used to produce haptens of the polypeptide (e.g., polypeptides useful to generate antibodies).

In one particular example, the polynucleotides of the invention are used to make or isolate to cDNA molecules encoding all or portion of a naturally-occuring polypeptide. Full-length cDNA molecules comprising the disclosed polynucleotides are obtained as follows. A polynucleotide having a sequence of one of SEQ ID NOS: 1-1477, or a portion thereof comprising at least 12, 15, 18, or 20 nt, is used as a hybridization probe to detect hybridizing members of a cDNA library using probe design methods, cloning methods, and clone selection techniques such as those described in U.S. Pat. No. 5,654,173. Libraries of cDNA are made from selected tissues, such as normal or tumor tissue, or from tissues of a mammal treated with, for example, a pharmaceutical agent. Preferably, the tissue is the same as the tissue from which the polynucleotides of the invention were isolated, as both the polynucleotides described herein and the cDNA represent expressed genes. Most preferably, the cDNA library is made from the biological material described herein in the Examples. The choice of cell type for library construction can be made after the identity of the protein encoded by the gene corresponding to the polynucleotide of the invention is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human prostate cells, more preferably, human prostate cancer cells

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA can be prepared by using primers based on polynucleotides comprising a sequence of SEQ ID NOS: 1-1477. In one embodiment, the cDNA library can be made from only poly-adenylated mRNA. Thus, poly-T primers can be used to prepare cDNA from the mRNA.

Members of the library that are larger than the provided polynucleotides, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2nd Ed., (1989) Cold Spring Harbor Press, Cold Spring Harbor, N.Y. In order to obtain additional sequences 5′ to the end of a partial cDNA, 5′ RACE (PCR Protocols: A Guide to Methods and Applications, (1990) Academic Press, Inc.) can be performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the invention, but this is not essential. Most preferably, the genomic DNA is obtained from the biological material described herein in the Examples. Such libraries can be in vectors suitable for carrying large segments of a genome, such as P1 or YAC, as described in detail in Sambrook et al., supra, 9.4-9.30. In addition, genomic sequences can be isolated from human BAC libraries, which are commercially available from Research Genetics, Inc., Huntsville, Ala., USA, for example. In order to obtain additional 5′ or 3′ sequences, chromosome walking is performed, as described in Sambrook et al., such that adjacent and overlapping fragments of genomic DNA are isolated. These are mapped and pieced together, as is known in the art, using restriction digestion enzymes and DNA ligase.

Using the polynucleotide sequences of the invention, corresponding full-length genes can be isolated using both classical and PCR methods to construct and probe cDNA libraries. Using either method, Northern blots, preferably, are performed on a number of cell types to determine which cell lines express the gene of interest at the highest level. Classical methods of constructing cDNA libraries are taught in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods. Gene trapping entails inserting a member of a cDNA library into a vector. The vector then is denatured to produce single stranded molecules. Next, a substrate-bound probe, such as a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the polynucleotide sequences of the invention. Random primers or primers specific to the library vector can be used to amplify the trapped cDNA. Such gene trapping techniques are described in Gruber et al., WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs are ligated to an oligonucleotide linker, and amplified by PCR using two primers. One primer is based on sequence from the instant polynucleotides, for which full length sequence is desired, and a second primer comprises sequence that hybridizes to the oligonucleotide linker to amplify the cDNA. A description of this method is reported in WO 97/19110. In preferred embodiments of RACE, a common primer is designed to anneal to an arbitrary adaptor sequence ligated to cDNA ends (Apte and Siebert, Biotechniques (1993) 15:890-893; Edwards et al., Nuc. Acids Res. (1991) 19:5227-5232). When a single gene-specific RACE primer is paired with the common primer, preferential amplification of sequences between the single gene specific primer and the common primer occurs. Commercial cDNA pools modified for use in RACE are available.

Another PCR-based method generates full-length cDNA library with anchored ends without needing specific Knowledge of the cDNA sequence. The method uses lock-docking primers (I-VI), where one primer, poly TV (I-III) locks over the polyA tail of eukaryotic mRNA producing first strand synthesis and a second primer, polyGH (IV-VI) locks onto the polyC tail added by terminal deoxynucleotidyl transferase (TdT)(see, e.g., WO 96/40998).

The promoter region of a gene generally is located 5′ to the initiation site for RNA polymerase II. Hundreds of promoter regions contain the “TATA” box, a sequence such as TATTA or TATAA, which is sensitive to mutations. The promoter region can be obtained by performing 5′ RACE using a primer from the coding region of the gene. Alternatively, the cDNA can be used as a probe for the genomic sequence, and the region 5′ to the coding region is identified by “walking up.” If the gene is highly expressed or differentially expressed, the promoter from the gene can be of use in a regulatory construct for a heterologous gene.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the invention can be synthesized. Thus, the invention encompasses nucleic acid molecules ranging in length from 15 nt (corresponding to at least 15 contiguous nt of one of SEQ ID NOS: 1-1477) up to a maximum length suitable for one or more biological manipulations, including replication and expression, of the nucleic acid molecule. The invention includes but is not limited to (a) nucleic acid having the size of a full gene, and comprising at least one of SEQ ID NOS: 1-1477; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b). Once provided with the polynucleotides disclosed herein, construction or preparation of (a)-(e) are well within the skill in the art.

The sequence of a nucleic acid comprising at least 15 contiguous nt of at least any one of SEQ ID NOS: 1-1477, preferably the entire sequence of at least any one of SEQ ID NOS: 1-1477, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of any one of SEQ ID NOS: 1-1477 is within the nucleic acid, the nucleic acid obtained is referred to herein as a polynucleotide comprising the sequence of any one of SEQ ID NOS: 1-1477.

Expression of Polypeptide Encoded by Full-Length cDNA or Full-Length Gene

The provided polynucleotides (e.g., a polynucleotide having a sequence of one of SEQ ID NOS: 1-1477), the corresponding cDNA, or the full-length gene is used to express a partial or complete gene product. Constructs of polynucleotides having sequences of SEQ ID NOS: 1-1477 can also be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process.

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research. The gene product encoded by a polynucleotide of the invention is expressed in any expression system, including, for example, bacterial, yeast, insect, amphibian and mammalian systems. Vectors, host cells and methods for obtaining expression in same are well known in the art. Suitable vectors and host cells are described in U.S. Pat. No. 5,654,173.

Polynucleotide molecules comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in SEQ ID NOS: 1-1477 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670.

Identification of Functional and Structural Motifs

Translations of the nucleotide sequence of the provided polynucleotides, cDNAs or full genes can be aligned with individual known sequences. Similarity with individual sequences can be used to determine the activity of the polypeptides encoded by the polynucleotides of the invention. Also, sequences exhibiting similarity with more than one individual sequence can exhibit activities that are characteristic of either or both individual sequences.

The full length sequences and fragments of the polynucleotide sequences of the nearest neighbors as identified through, for example, BLAST-based searching,can be used as probes and primers to identify and isolate the full length sequence corresponding to provided polynucleotides. The nearest neighbors can indicate a tissue or cell type to be used to construct a library for the full-length sequences corresponding to the provided polynucleotides.

Typically, a selected polynucleotide is translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences. Databases with individual sequences are described in “Computer Methods for Macromolecular Sequence Analysis” Methods in Enzymology (1996) 266, Doolittle, Academic Press, Inc., a division of Harcourt Brace & Co., San Diego, Calif., USA. Databases include GenBank, EMBL, and DNA Database of Japan (DDBJ).

Query and individual sequences can be aligned using the methods and computer programs described above, and include BLAST 2.0, available over the world wide web at a site supported by the National Center for Biotechnology Information, which is supported by the National Library of Medicine and the National Institutes of Health, or TeraBLAST available from TimeLogic Corp. (Crystal Bay, Nev.). See also Altschul, et al. Nucleic Acids Res. (1997) 25:3389-3402. Another alignment algorithm is Fasta, available in the Genetics Computing Group (GCG) package, Madison, Wis., USA, a wholly owned subsidiary of Oxford Molecular Group, Inc. Other techniques for alignment are described in Doolittle, supra. Preferably, an alignment program that permits gaps in the sequence is utilized to align the sequences. The Smith-Waterman is one type of algorithm that permits gaps in sequence alignments. See Meth. Mol. Biol. (1997) 70: 173-187. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to identify sequences that are distantly related matches, and is especially tolerant of small gaps and nucleotide sequence errors. Amino acid sequences encoded by the provided polynucleotides can be used to search both protein and DNA databases. Incorporated herein by reference are all sequences that have been made public as of the filing date of this application by any of the DNA or protein sequence databases, including the patent databases (e.g., GeneSeq). Also incorporated by reference are those sequences that have been submitted to these databases as of the filing date of the present application but not made public until after the filing date of the present application.

Results of individual and query sequence alignments can be divided into three categories: high similarity, weak similarity, and no similarity. Individual alignment results ranging from high similarity to weak similarity provide a basis for determining polypeptide activity and/or structure. Parameters for categorizing individual results include: percentage of the alignment region length where the strongest alignment is found, percent sequence identity, and p value. The percentage of the alignment region length is calculated by counting the number of residues of the individual sequence found in the region of strongest alignment, e.g., contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is: 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%

P value is the probability that the alignment was produced by chance. For a single alignment, the p value can be calculated according to Karlin et al., Proc. Natl. Acad. Sci. (1990) 87:2264 and Karlin et al., Proc. Natl. Acad. Sci. (1993) 90. The p value of multiple alignments using the same query sequence can be calculated using an heuristic approach described in Altschul et al., Nat. Genet. (1994) 6:119. Alignment programs, such as BLAST or TeraBLAST, can calculate the p value. See also Altschul et al., Nucleic Acids Res. (1997) 25:3389-3402.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences. The boundaries of the region where the sequences align can be determined according to Doolittle, supra; BLAST 2.0 (see, e.g., Altschul, et al. Nucleic Acids Res. (1997) 25:3389-3402), TeraBLAST (available from TimeLogic Corp., Crystal Bay, Nev.), or FAST programs; or by determining the area where sequence identity is highest.

High Similarity. In general, in alignment results considered to be of high similarity, the percent of the alignment region length is typically at least about 55% of total length query sequence; more typically, at least about 58%; even more typically; at least about 60% of the total residue length of the query sequence. Usually, percent length of the alignment region can be as much as about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. If high similarity is found, the query sequence is considered to have high similarity with a profile sequence when the p value is less than or equal to about 10e−2; more usually; less than or equal to about 10e−3; even more usually; less than or equal to about 10e−4. More typically, the p value is no more than about 10e−5; more typically; no more than or equal to about 10e−10; even more typically, no more than or equal to about 10e−15 for the query sequence to be considered high similarity.

Weak Similarity. In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically, at least about 25 amino acid residues in length. Usually, length of the alignment region can be as much as about 30 amino acid residues; more usually, as much as about 40; even more usually, as much as about 60 amino acid residues. Further, for weak similarity, the region of alignment, typically, exhibits at least about 35% of sequence identity; more typically, at least about 40%; even more typically, at least about 45% sequence identity. Usually, percent sequence identity can be as much as about 50%; more usually, as much as about 55%; even more usually, as much as about 60%.

If low similarity is found, the query sequence is considered to have weak similarity with a profile sequence when the p value is usually less than or equal to about 10e−2; more usually, less than or equal to about 10e−3; even more usually; less than or equal to about 10e4. More typically, the p value is no more than about 10e−5; more usually; no more than or equal to about 10e−10; even more usually, no more than or equal to about 10e−15 for the query sequence to be considered weak similarity.

Similarity Determined by Sequence Identity Alone. Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, at least 90 residues in length; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

Alignments with Profile and Multiple Aligned Sequences. Translations of the provided polynucleotides can be aligned with amino acid profiles that define either protein families or common motifs. Also, translations of the provided polynucleotides can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided polynucleotides or corresponding cDNA or genes. For example, sequences that show an identity or similarity with a chemokine profile or MSA can exhibit chemokine activities.

Profiles can be designed manually by (1) creating an MSA, which is an alignment of the amino acid sequence of members that belong to the family and (2) constructing a statistical representation of the alignment. Such methods are described, for example, in Birney et al., Nucl. Acid Res. (1996) 24(14): 2730-2739. MSAs of some protein families and motifs are publicly available. For example, the Genome Sequencing Center at thw Washington University School of Medicine provides a web set (Pfam) which provides MSAs of 547 different families and motifs. These MSAs are described also in Sonnhammer et al., Proteins (1997) 28: 405-420. Other sources over the world wide web include the site supported by the European Molecular Biology Laboratories in Heidelberg, Germany. A brief description of these MSAs is reported in Pascarella et al., Prot. Eng. (1996) 9(3):249-251. Techniques for building profiles from MSAs are described in Sonnhammer et al., supra; Birney et al., supra; and “Computer Methods for Macromolecular Sequence Analysis,” Methods in Enzymology (1996) 266, Doolittle, Academic Press, Inc., San Diego, Calif., USA.

Similarity between a query sequence and a protein family or motif can be determined by (a) comparing the query sequence against the profile and/or (b) aligning the query sequence with the members of the family or motif. Typically, a program such as Searchwise is used to compare the query sequence to the statistical representation of the multiple alignment, also known as a profile (see Birney et al., supra). Other techniques to compare the sequence and profile are described in Sonnhammer et al., supra and Doolittle, supra.

Next, methods described by Feng et al., J. Mol. Evol. (1987) 25:351 and Higgins et al., CABIOS (1989) 5:151 can be used align the query sequence with the members of a family or motif, also known as a MSA. Sequence alignments can be generated using any of a variety of software tools. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:443. GAP is best suited for global alignment of sequences. A third method, BestFit, functions by inserting gaps to maximize the number of matches using the local homology algorithm of Smith et al., Adv. Appl. Math. (1981) 2:482. In general, the following factors are used to determine if a similarity between a query sequence and a profile or MSA exists: (1) number of conserved residues found in the query sequence, (2) percentage of conserved residues found in the query sequence, (3) number of frameshifts, and (4) spacing between conserved residues.

Some alignment programs that both translate and align sequences can make any number of frameshifts when translating the nucleotide sequence to produce the best alignment. The fewer frameshifts needed to produce an alignment, the stronger the similarity or identity between the query and profile or MSAs. For example, a weak similarity resulting from no frameshifts can be a better indication of activity or structure of a query sequence, than a strong similarity resulting from two frameshifts. Preferably, three or fewer frameshifts are found in an alignment; more preferably two or fewer frameshifts; even more preferably, one or fewer frameshifts; even more preferably, no frameshifts are found in an alignment of query and profile or MSAs.

Conserved residues are those amino acids found at a particular position in all or some of the family or motif members. Alternatively, a position is considered conserved if only a certain class of amino acids is found in a particular position in all or some of the family members. For example, the N-terminal position can contain a positively charged amino acid, such as lysine, arginine, or histidine.

Typically, a residue of a polypeptide is conserved when a class of amino acids or a single amino acid is found at a particular position in at least about 40% of all class members; more typically, at least about 50%; even more typically, at least about 60% of the members. Usually, a residue is conserved when a class or single amino acid is found in at least about 70% of the members of a family or motif; more usually, at least about 80%; even more usually, at least about 90%; even more usually, at least about 95%.

A residue is considered conserved when three unrelated amino acids are found at a particular position in some or all of the members; more usually, two unrelated amino acids. These residues are conserved when the unrelated amino acids are found at particular positions in at least about 40% of all class member; more typically, at least about 50%; even more typically, at least about 60% of the members. Usually, a residue is conserved when a class or single amino acid is found in at least about 70% of the members of a family or motif; more usually, at least about 80%; even more usually, at least about 90%; even more usually, at least about 95%.

A query sequence has similarity to a profile or MSA when the query sequence comprises at least about 25% of the conserved residues of the profile or MSA; more usually, at least about 30%; even more usually; at least about 40%. Typically, the query sequence has a stronger similarity to a profile sequence or MSA when the query sequence comprises at least about 45% of the conserved residues of the profile or MSA; more typically, at least about 50%; even more typically, at least about 55%.

Identification of Secreted & Membrane-Bound Polypeptides. Both secreted and membrane-bound polypeptides of the present invention are of particular interest. For example, levels of secreted polypeptides can be assayed in body fluids that are convenient, such as blood, plasma, serum, and other body fluids such as urine, prostatic fluid and semen. Membrane-bound polypeptides are useful for constructing vaccine antigens or inducing an immune response. Such antigens would comprise all or part of the extracellular region of the membrane-bound polypeptides. Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 190: 207-219.

Another method of identifying secreted and membrane-bound polypeptides is to translate the polynucleotides of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine

### Identification of the Function of an Expression Product of a Full-Length Gene

Ribozymes, antisense constructs, and dominant negative mutants can be used to determine function of the expression product of a gene corresponding to a polynucleotide provided herein. These methods and compositions are particularly useful where the provided novel polynucleotide exhibits no significant or substantial homology to a sequence encoding a gene of known function. Antisense molecules and ribozymes can be constructed from synthetic polynucleotides. Typically, the phosphoramidite method of oligonucleotide synthesis is used. See Beaucage et al., Tet. Lett. (1981) 22:1859 and U.S. Pat. No. 4,668,777. Automated devices for synthesis are available to create oligonucleotides using this chemistry. Examples of such devices include Biosearch 8600, Models 392 and 394 by Applied Biosystems, a division of Perkin-Elmer Corp., Foster City, Calif., USA; and Expedite by Perceptive Biosystems, Framingham, Mass., USA. Synthetic RNA, phosphate analog oligonucleotides, and chemically derivatized oligonucleotides can also be produced, and can be covalently attached to other molecules. RNA oligonucleotides can be synthesized, for example, using RNA phosphoramidites. This method can be performed on an automated synthesizer, such as Applied Biosystems, Models 392 and 394, Foster City, Calif., USA.

Phosphorothioate oligonucleotides can also be synthesized for antisense construction. A sulfurizing reagent, such as tetraethylthiruam disulfide (TETD) in acetonitrile can be used to convert the internucleotide cyanoethyl phosphite to the phosphorothioate triester within 15 minutes at room temperature. TETD replaces the iodine reagent, while all other reagents used for standard phosphoramidite chemistry remain the same. Such a synthesis method can be automated using Models 392 and 394 by Applied Biosystems, for example.

Oligonucleotides of up to 200 nt can be synthesized, more typically, 100 nt; more typically 50 nt; even more typically, 30 to 40 nt. These synthetic fragments can be annealed and ligated together to construct larger fragments. See, for example, Sambrook et al., supra. Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect. One commonly used ribozyme motif is the hammerhead, for which the substrate sequence requirements are minimal. Design of the hammerhead ribozyme, as well as therapeutic uses of ribozymes, are disclosed in Usman et al., Current Opin. Struct. Biol. (1996) 6:527. Methods for production of ribozymes, including hairpin structure ribozyme fragments, methods of increasing ribozyme specificity, and the like are known in the art.

The hybridizing region of the ribozyme can be modified or can be prepared as a branched structure as described in Horn and Urdea, Nucleic Acids Res. (1989) 17:6959. The basic structure of the ribozymes can also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1.

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence can interfere with expression of the corresponding gene. Antisense polynucleotides are typically generated within the cell by expression from antisense constructs that contain the antisense strand as the transcribed strand. Antisense polynucleotides based on the disclosed polynucleotides will bind and/or interfere with the translation of mRNA comprising a sequence complementary to the antisense polynucleotide. The expression products of control cells and cells treated with the antisense construct are compared to detect the protein product of the gene corresponding to the polynucleotide upon which the antisense construct is based. The protein is isolated and identified using routine biochemical methods.

Given the extensive background literature and clinical experience in antisense therapy, one skilled in the art can use selected polynucleotides of the invention as additional potential therapeutics. The choice of polynucleotide can be narrowed by first testing them for binding to “hot spot” regions of the genome of cancerous cells. If a polynucleotide is identified as binding to a “hot spot,” testing the polynucleotide as an antisense compound in the corresponding cancer cells is warranted.

As an alternative method for identifying function of the gene corresponding to a polynucleotide disclosed herein, dominant negative mutations are readily generated for corresponding proteins that are active as homomultimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants (see, e.g., Herskowitz, Nature (1987) 329:219). Such techniques can be used to create loss of function mutations, which are useful for determining protein function.

Polypeptides and Variants Thereof

The polypeptides of the invention include those encoded by the disclosed polynucleotides, as well as nucleic acids that, by virtue of the degeneracy of the genetic code, are not identical in sequence to the disclosed polynucleotides. Thus, the invention includes within its scope a polypeptide encoded by a polynucleotide having the sequence of any one of SEQ ID NOS: 1-1477 or a variant thereof. Also included in the invention are the polypeptides comprising the amino acid sequences of SEQ ID NOS: 1478-1568.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the invention, as measured by BLAST 2.0 or TeraBLAST using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 or TeraBLAST algorithm, with the parameters described supra.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions within proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly haptens, biologically active fragments, and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any SEQ ID NOS: 1-1477, a polypeptide comrpsing a sequence of at least one of SEQ ID NOS: 1478-1568, or a homolog thereof. The protein variants described herein are encoded by polynucleotides that are within the scope of the invention. The genetic code can be used to select the appropriate codons to construct the corresponding variants.

Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a breast ductal cell affected by cancer relative to a normal (i.e., substantially disease-free) breast cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of SEQ ID NOS: 1-1477. By plurality is meant at least 2, usually at least 3 and can include up to all of SEQ ID NOS: 1-1477. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of SEQ ID NOS: 1-1477, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information is publicly available. For example, the gapped BLAST (Altschul et al. Nucleic Acids Res. (1997) 25:3389-3402) and BLAZE (Brutlag et al. Comp. Chem. (1993) 17:203) search algorithms on a Sybase system, or the TeraBLAST (TimeLogic, Crystal Bay, Nev.) program optionally running on a specialized computer platform available from TimeLogic, can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information. Search means can be used to identify fragments or regions of the genome that match a particular target sequence or target motif. A variety of known algorithms are publicly known and commercially available, e.g. MacPattern (EMBL), BLASTN and BLASTX (NCBI), TeraBLAST (TimeLogic, Crystal Bay, Nev.). A “target sequence” can be any polynucleotide or amino acid sequence of six or more contiguous nucleotides or two or more amino acids, preferably from about 10 to 100 amino acids or from about 30 to 300 nt A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but arc not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile.

As discussed above, the “library” of the invention also encompasses biochemical libraries of the polynucleotides of SEQ ID NOS: 1-1477, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of SEQ ID NOS: 1-1477 is represented on the array. By array is meant a an article of manufacture that has at least a substrate with at least two distinct nucleic acid targets on one of its surfaces, where the number of distinct nucleic acids can be considerably higher, typically being at least 10, usually at least 20, and often at least 25 distinct nucleic acid molecules. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the polypeptides of the library will represent at least a portion of the polypeptides encoded by a gene corresponding to one or more of SEQ ID NOS: 1-1477.

Utilities

The polynucleotides of the invention are useful in a variety of applications. Exemplary utilies of the polynucleotides of the invention are described below.

Construction of Larger Molecules: Recombinant DNAs and Nucleic Acid Multimers. In one embodiment of particular interest, the polynucleotides described herein as useful as the building blocks for larger molecules. In one example, the polynucleotide is a component of a larger cDNA molecule which in turn can be adapted for expression in a host cell (e.g., a bacterial or eukaryotic (e.g., yeast or mammalian) host cell). The cDNA can include, in addition to the polypeptide encoded by the starting material polynucleotide (i.e., a polynucleotide described herein), an amino acid sequence that is heterologous to the polypeptide encoded by the polynucleotide described herein (e.g., as in a sequence encoding a fusion protein). In some embodiments, the polynucleotides described herein is used as starting material polynucleotide for synthesizing all or a portion of the gene to which the described polynucleotide corresponds. For example, a DNA molecule encoding a full-length human polypeptide can be constructed using a polynucleotide described herein as starting material.

In another embodiment, the polynucleotides of the invention are used in nucleic acid multimers. Nucleic acid multimers can be linear or branched polymers of the same repeating single-stranded oligonucleotide unit or different single-stranded oligonucleotide units. Where the molecules are branched, the multimers are generally described as either “fork” or “comb” structures. The oligonucleotide units of the multimer may be composed of RNA, DNA, modified nucleotides or combinations thereof. At least one of the units has a sequence, length, and composition that permits it to bind specifically to a first single-stranded nucleotide sequence of interest, typically analyte or an oligonucleotide bound to the analyte. In order to achieve such specificity and stability, this unit will normally be 15 to 50 nt, preferably 15 to 30 nt, in length and have a GC content in the range of 40% to 60%. In addition to such unit(s), the multimer includes a multiplicity of units that are capable of hybridizing specifically and stably to a second single-stranded nucleotide of interest, typically a labeled oligonucleotide or another multimer. These units will also normally be 15 to 50 nt, preferably 15 to 30 nt, in length and have a GC content in the range of 40% to 60%. When a multimer is designed to be hybridized to another multimer, the first and second oligonucleotide units are heterogeneous (different). One or more of the polynucleotides described herein, or a portion of a polynucleotide described herein, can be used as a repeating unit of such nucleic acid multimers.

The total number of oligonucleotide units in the multimer will usually be in the range of 3 to 50, more usually 10 to 20. In multimers in which the unit that hybridizes to the nucleotide sequence of interest is different from the unit that hybridizes to the labeled oligonucleotide, the number ratio of the latter to the former will usually be 2:1 to 30:1, more usually 5:1 to 20:1, and-preferably 10:1 to 15:1.

The oligonucleotide units of the multimer may be covalently linked directly to each other through phosphodiester bonds or through interposed linking agents such as nucleic acid, amino acid, carbohydrate or polyol bridges, or through other cross-linking agents that are capable of cross-linking nucleic acid or modified nucleic acid strands. The site(s) of linkage may be at the ends of the unit (in either normal 3,-5′ orientation or randomly oriented) and/or at one or more internal nucleotides in the strand. In linear multimers the individual units are linked end-to-end to form a linear polymer. In one type of branched multimer three or more oligonucleotide units emanate from a point of origin to form a branched structure. The point of origin may be another oligonucleotide unit or a multifunctional molecule to which at least three units can be covalently bound. In another type, there is an oligonucleotide unit backbone with one or more pendant oligonucleotide units. These latter-type multimers are “fork-like”, “comb-like” or combination “fork-” and “comb-like” in structure. The pendant units will normally depend from a modified nucleotide or other organic moiety having appropriate functional groups to which oligonucleotides may be conjugated or otherwise attached. The multimer may be totally linear, totally branched, or a combination of linear and branched portions. Preferably there will be at least two branch points in the multimer, more preferably at least 3, preferably 5 to 10. The multimer may include one or more segments of double-stranded sequences.

Multimeric nucleic acid molecules are useful in amplifying the signal that results from hybridization of one the first sequence of the multimeric molecule to a target sequence. The amplification is theoretically proportional to the number of iterations of the second segment.

Without being held to theory, forked structures of greater than about eight branches exhibited steric hindrance which inhibited binding of labeled probes to the multimer. On the other hand, comb structures exhibit little or no steric problems and are thus a preferred type of branched multimer. For a description of branched nucleic acid multimers of both the fork and comb types, as well as methods of use and synthesis, see, e.g., U.S. Pat. Nos. 5,124,246 (fork-type structures); 5,710,264 (synthesis of comb structures); and 5,849,481.

Use of Polynucleotide Probes in Mapping, and in Tissue Profiling. Polynucleotide probes, generally comprising at least 12 contiguous nt of a polynucleotide as shown in the Sequence Listing, are used for a variety of purposes, such as chromosome mapping of the polynucleotide and detection of transcription levels. Additional disclosure about preferred regions of the disclosed polynucleotide sequences is found in the Examples. A probe that hybridizes specifically to a polynucleotide disclosed herein should provide a detection signal at least 5-, 10-, or 20-fold higher than the background hybridization provided with other unrelated sequences.

Detection of Expression Levels. Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluors, and enzymes. Other examples of nucleotide hybridization assays are described in WO92/02526 and U.S. Pat. No. 5,124,246.

Alternatively, the Polymerase Chain Reaction (PCR) is another means for detecting small amounts of target nucleic acids (see, e.g., Mullis et al., Meth. Enzymol. (1987) 155:335; U.S. Pat. Nos. 4,683,195; and 4,683,202). Two primer polynucleotides nucleotides that hybridize with the target nucleic acids are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Mapping. Polynucleotides of the present invention can be used to identify a chromosome on which the corresponding gene resides. Such mapping can be useful in identifying the function of the polynucleotide-related gene by its proximity to other genes with known function. Function can also be assigned to the polynucleotide-related gene when particular syndromes or diseases map to the same chromosome. For example, use of polynucleotide probes in identification and quantification of nucleic acid sequence aberrations is described in U.S. Pat. No. 5,783,387. An exemplary mapping method is fluorescence in situ hybridization (FISH), which facilitates comparative genomic hybridization to allow total genome assessment of changes in relative copy number of DNA sequences (see, e.g., Valdes et al., Methods in Molecular Biology (1997) 68:1). Polynucleotides can also be mapped to particular chromosomes using, for example, radiation hybrids or chromosome-specific hybrid panels. See Leach et al., Advances in Genetics, (1995) 33:63-99; Walter et al., Nature Genetics (1994) 7:22; Walter and Goodfellow, Trends in Genetics (1992) 9:352. Panels for radiation hybrid mapping are available from Research Genetics, Inc., Huntsville, Ala., USA. Databases for markers using various panels are available via the world wide web at sites supported by the Stanford Human Genome Center (Stanford University) and the Whitehead Institute for Biomedical Research/MIT Center for Genome Research. The statistical program RHMAP can be used to construct a map based on the data from radiation hybridization with a measure of the relative likelihood of one order versus another. RHMAP is available via the world wide web at a site supported by the University of Michigan. In addition, commercial programs are available for identifying regions of chromosomes commonly associated with disease, such as cancer.

Tissue Typing or Profiling. Expression of specific mRNA corresponding to the provided polynucleotides can vary in different cell types and can be tissue-specific. This variation of mRNA levels in different cell types can be exploited with nucleic acid probe assays to determine tissue types. For example, PCR, branched DNA probe assays, or blotting techniques utilizing nucleic acid probes substantially identical or complementary to polynucleotides listed in the Sequence Listing can determine the presence or absence of the corresponding cDNA or mRNA.

Tissue typing can be used to identify the developmental organ or tissue source of a metastatic lesion by identifying the expression of a particular marker of that organ or tissue. If a polynucleotide is expressed only in a specific tissue type, and a metastatic lesion is found to express that polynucleotide, then the developmental source of the lesion has been identified. Expression of a particular polynucleotide can be assayed by detection of either the corresponding mRNA or the protein product. As would be readily apparent to any forensic scientist, the sequences disclosed herein are useful in differentiating human tissue from non-human tissue. In particular, these sequences are useful to differentiate human tissue from bird, reptile, and amphibian tissue, for example.

Use of Polymorphisms. A polynucleotide of the invention can be used in forensics, genetic analysis, mapping, and diagnostic applications where the corresponding region of a gene is polymorphic in the human population. Any means for detecting a polymorphism in a gene can be used, including, but not limited to electrophoresis of protein polymorphic variants, differential sensitivity to restriction enzyme cleavage, and hybridization to allele-specific probes.

Antibody Production. The present invention further provides antibodies, which may be isolated antibodies, that are specific for a polypeptide encoded by a polynucleotide described herein (e.g., a polypeptide encoded by a sequence corresponding to SEQ ID NOS: 1-1477, a polypeptide comprising an amino acid sequence of SEQ ID NOS: 1478-1568). Antibodies can be provided in a composition comprising the antibody and a buffer and/or a pharmaceutically acceptable excipient. Antibodies specific for a polypeptide associated with prostate cancer are useful in a variety of diagnostic and therapeutic methods, as discussed in detail herein.

Expression products of a polynucleotide of the invention, as well as the corresponding mRNA, cDNA, or complete gene, can be prepared and used for raising antibodies for experimental, diagnostic, and therapeutic purposes. For polynucleotides to which a corresponding gene has not been assigned, this provides an additional method of identifying the corresponding gene. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Methods for production of antibodies that specifically bind a selected antigen are well known in the art. Immunogens for raising antibodies can be prepared by mixing a polypeptide encoded by a polynucleotide of the invention with an adjuvant, and/or by making fusion proteins with larger immunogenic proteins. Polypeptides can also be covalently linked to other larger immunogenic proteins, such as keyhole limpet hemocyanin. Immunogens are typically administered intradermally, subcutaneously, or intramuscularly to experimental animals such as rabbits, sheep, and mice, to generate antibodies. Monoclonal antibodies can be generated by isolating spleen cells and fusing myeloma cells to form hybridomas. Alternatively, the selected polynucleotide is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for polypeptides encoded by a selected polynucleotide are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. Epitopes that involve non-contiguous amino acids may require a longer polypeptide, e.g., at least 15, 25, or 50 amino acids. Antibodies that specifically bind to human polypeptides encoded by the provided polypeptides should provide a detection signal at least 5-, 10-, or 20-fold higher than a detection signal provided with other proteins when used in Western blots or other immunochemical assays. Preferably, antibodies that specifically bind polypeptides contemplated by the invention do not bind to other proteins in immunochemical assays at detectable levels and can immunoprecipitate the specific polypeptide from solution.

The invention also contemplates naturally occurring antibodies specific for a polypeptide of the invention. For example, serum antibodies to a polypeptide of the invention in a human population can be purified by methods well known in the art, e.g., by passing antiserum over a column to which the corresponding selected polypeptide or fusion protein is bound. The bound antibodies can then be eluted from the column, for example, using a buffer with a high salt concentration.

In addition to the antibodies discussed above, the invention also contemplates genetically engineered antibodies antibodies (e.g., chimeric antibodies, humanized antibodies, human antibodies produced by a transgenic animal (e.g., a transgenic mouse such as the XenomousTM), antibody derivatives (e.g., single chain antibodies, antibody fragments (e.g., Fab, etc.)), according to methods well known in the art.

The invention also contemplates other molecules that can specifically bind a polynucleotide or polypeptide of the invention. Examples of such molecules include, but are not necessarily limited to, single-chain binding proteins (e.g., mono- and multi-valent single chain antigen binding proteins (see, e.g., U.S. Pat. Nos. 4,704,692; 4,946,778; 4,946,778; 6,027,725; 6,121,424)), oligonucleotide-based synthetic antibodies (e.g., oligobodies (see, e.g., Radrizzani et al., Medicina (B Aires) (1999) 59:753-8; Radrizzani et al., Medicina (B Aires) (2000) 60(Suppl 2):55-60)), aptamers (see, e.g., Gening et al., Biotechniques (2001) 3:828, 830, 832, 834; Cox and Ellington, Bioorg. Med. Chem. (2001) 9:2525-31), and the like.

Polynucleotides or Arrays for Diagnostics.

Polynucleotide arrays provide a high throughput technique that can assay a large number of polynucleotides in a sample. This technology can be used as a diagnostic and as tool to test for differential expression expression, e.g., to determine function of an encoded protein. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci U S A. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. Nos. 5,593,839; 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of a gene corresponding to a polynucleotide of the invention, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Differential Expression in Diagnosis

The polynucleotides of the invention can also be used to detect differences in expression levels between two cells, e.g., as a method to identify abnormal or diseased tissue in a human. For polynucleotides corresponding to profiles of protein families, the choice of tissue can be selected according to the putative biological function. In general, the expression of a gene corresponding to a specific polynucleotide is compared between a first tissue that is suspected of being diseased and a second, normal tissue of the human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon). A difference between the polynucleotide-related gene, mRNA, or protein in the two tissues which are compared, for example, in molecular weight, amino acid or nucleotide sequence, or relative abundance, indicates a change in the gene, or a gene which regulates it, in the tissue of the human that was suspected of being diseased. Examples of detection of differential expression and its use in diagnosis of cancer are described in U.S. Pat. Nos. 5,688,641 and 5,677,125.

A genetic predisposition to disease in a human can also be detected by comparing expression levels of an mRNA or protein corresponding to a polynucleotide of the invention in a fetal tissue with levels associated in normal fetal tissue. Fetal tissues that are used for this purpose include, but are not limited to, amniotic fluid, chorionic villi, blood, and the blastomere of an in vitro-fertilized embryo. The comparable normal polynucleotide-related gene is obtained from any tissue. The mRNA or protein is obtained from a normal tissue of a human in which the polynucleotide-related gene is expressed. Differences such as alterations in the nucleotide sequence or size of the same product of the fetal polynucleotide-related gene or mRNA, or alterations in the molecular weight, amino acid sequence, or relative abundance of fetal protein, can indicate a germline mutation in the polynucleotide-related gene of the fetus, which indicates a genetic predisposition to disease. In general, diagnostic, prognostic, and other methods of the invention based on differential expression involve detection of a level or amount of a gene product, particularly a differentially expressed gene product, in a test sample obtained from a patient suspected of having or being susceptible to a disease (e.g., breast cancer, lung cancer, colon cancer and/or metastatic forms thereof), and comparing the detected levels to those levels found in normal cells (e.g., cells substantially unaffected by cancer) and/or other control cells (e.g., to differentiate a cancerous cell from a cell affected by dysplasia). Furthermore, the severity of the disease can be assessed by comparing the detected levels of a differentially expressed gene product with those levels detected in samples representing the levels of differentially expressed gene product associated with varying degrees of severity of disease. It should be noted that use of the term “diagnostic” herein is not necessarily meant to exclude “prognostic” or “prognosis,” but rather is used as a matter of convenience.

The term “differentially expressed gene” is generally intended to encompass a polynucleotide that can, for example, include an open reading frame encoding a gene product (e.g., a polypeptide), and/or introns of such genes and adjacent 5′ and 3′ non-coding nucleotide sequences involved in the regulation of expression, up to about 20 kb beyond the coding region, but possibly further in either direction. The gene can be introduced into an appropriate vector for extrachromosomal maintenance or for integration into a host genome. In general, a difference in expression level associated with a decrease in expression level of at least about 25%, usually at least about 50% to 75%, more usually at least about 90% or more is indicative of a differentially expressed gene of interest, i.e., a gene that is underexpressed or down-regulated in the test sample relative to a control sample. Furthermore, a difference in expression level associated with an increase in expression of at least about 25%, usually at least about 50% to 75%, more usually at least about 90% and can be at least about 1½-fold, usually at least about 2-fold to about 10-fold, and can be about 100-fold to about 1,000-fold increase relative to a control sample is indicative of a differentially expressed gene of interest, i.e., an overexpressed or up-regulated gene.

“Differentially expressed polynucleotide” as used herein means a nucleic acid molecule (RNA or DNA) comprising a sequence that represents a differentially expressed gene, e.g., the differentially expressed polynucleotide comprises a sequence (e.g., an open reading frame encoding a gene product) that uniquely identifies a differentially expressed gene so that detection of the differentially expressed polynucleotide in a sample is correlated with the presence of a differentially expressed gene in a sample. “Differentially expressed polynucleotide” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

Methods of the subject invention useful in diagnosis or prognosis typically involve comparison of the abundance of a selected differentially expressed gene product in a sample of interest with that of a control to determine any relative differences in the expression of the gene product, where the difference can be measured qualitatively and/or quantitatively. Quantitation can be accomplished, for example, by comparing the level of expression product detected in the sample with the amounts of product present in a standard curve. A comparison can be made visually; by using a technique such as densitometry, with or without computerized assistance; by preparing a representative library of cDNA clones of mRNA isolated from a test sample, sequencing the clones in the library to determine that number of cDNA clones corresponding to the same gene product, and analyzing the number of clones corresponding to that same gene product relative to the number of clones of the same gene product in a control sample; or by using an array to detect relative levels of hybridization to a selected sequence or set of sequences, and comparing the hybridization pattern to that of a control. The differences in expression are then correlated with the presence or absence of an abnormal expression pattern. A variety of different methods for determining the nucleic acid abundance in a sample are known to those of skill in the art (see, e.g., WO 97/27317).

In general, diagnostic assays of the invention involve detection of a gene product of a polynucleotide sequence (e.g., mRNA or polypeptide) that corresponds to a sequence of SEQ ID NOS: 1-1477. The patient from whom the sample is obtained can be apparently healthy, susceptible to disease (e.g., as determined by family history or exposure to certain environmental factors), or can already be identified as having a condition in which altered expression of a gene product of the invention is implicated.

Diagnosis can be determined based on detected gene product expression levels of a gene product encoded by at least one, preferably at least two or more, at least 3 or more, or at least 4 or more of the polynucleotides having a sequence set forth in SEQ ID NOS: 1-1477, and can involve detection of expression of genes corresponding to all of SEQ ID NOS: 1-1477 and/or additional sequences that can serve as additional diagnostic markers and/or reference sequences. Where the diagnostic method is designed to detect the presence or susceptibility of a patient to cancer, the assay preferably involves detection of a gene product encoded by a gene corresponding to a polynucleotide that is differentially expressed in cancer. Examples of such differentially expressed polynucleotides are described in the Examples below. Given the provided polynucleotides and information regarding their relative expression levels provided herein, assays using such polynucleotides and detection of their expression levels in diagnosis and prognosis will be readily apparent to the ordinarily skilled artisan.

Any of a variety of detectable labels can be used in connection with the various embodiments of the diagnostic methods of the invention. Suitable detectable labels include fluorochromes, (e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. 32P, 35S, 3H, etc.), and the like. The detectable label can involve a two stage systems (e.g., biotin-avidin, hapten-anti-hapten antibody, etc.).

Reagents specific for the polynucleotides and polypeptides of the invention, such as antibodies and nucleotide probes, can be supplied in a kit for detecting the presence of an expression product in a biological sample. The kit can also contain buffers or labeling components, as well as instructions for using the reagents to detect and quantify expression products in the biological sample. Exemplary embodiments of the diagnostic methods of the invention are described below in more detail.

Polypeptide detection in diagnosis. In one embodiment, the test sample is assayed for the level of a differentially expressed polypeptide, such as a polypeptide of a gene corresponding to SEQ ID NOS: 1-1477 and/or a polypeptide comprising a sequence of SEQ ID NO: 1478-1568. Diagnosis can be accomplished using any of a number of methods to determine the absence or presence or altered amounts of the differentially expressed polypeptide in the test sample. For example, detection can utilize staining of cells or histological sections with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example, ELISA, western blot, immunoprecipitation, radioimmunoassay, etc.

mRNA detection. The diagnostic methods of the invention can also or alternatively involve detection of mRNA encoded by a gene corresponding to a differentially expressed polynucleotide of the invention. Any suitable qualitative or quantitative methods known in the art for detecting specific mRNAs can be used. mRNA can be detected by, for example, in situ hybridization in tissue sections, by reverse transcriptase-PCR, or in Northern blots containing poly A+ mRNA. One of skill in the art can readily use these methods to determine differences in the size or amount of mRNA transcripts between two samples. mRNA expression levels in a sample can also be determined by generation of a library of expressed sequence tags (ESTs) from the sample, where the EST library is representative of sequences present in the sample (Adams, et al., (1991) Science 252:1651). Enumeration of the relative representation of ESTs within the library can be used to approximate the relative representation of the gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein. Alternatively, gene expression in a test sample can be performed using serial analysis of gene expression (SAGE) methodology (e.g., Velculescu et al., Science (1995) 270:484) or differential display (DD) methodology (see, e.g., U.S. Pat. Nos. 5,776,683 and 5,807,680).

Alternatively, gene expression can be analyzed using hybridization analysis. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Use of a single gene in diagnostic applications. The diagnostic methods of the invention can focus on the expression of a single differentially expressed gene. For example, the diagnostic method can involve detecting a differentially expressed gene, or a polymorphism of such a gene (e.g., a polymorphism in a coding region or control region), that is associated with disease. Disease-associated polymorphisms can include deletion or truncation of the gene, mutations that alter expression level and/or affect activity of the encoded protein, etc.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express a differentially expressed gene can be used as a source of mRNA, which can be assayed directly or reverse transcribed into cDNA for analysis. The nucleic acid can be amplified by conventional techniques, such as the polymerase chain reaction (PCR), to provide sufficient amounts for analysis, and a detectable label can be included in the amplification reaction (e.g., using a detectably labeled primer or detectably labeled oligonucleotides) to facilitate detection. Alternatively, various methods are also known in the art that utilize oligonucleotide ligation as a means of detecting polymorphisms, see, e.g., Riley et al., Nucl. Acids Res. (1990) 18:2887; and Delahunty et al., Am. J. Hum. Genet. (1996) 58:1239.

The amplified or cloned sample nucleic acid can be analyzed by one of a number of methods known in the art. The nucleic acid can be sequenced by dideoxy or other methods, and the sequence of bases compared to a selected sequence, e.g., to a wild-type sequence. Hybridization with the polymorphic or variant sequence can also be used to determine its presence in a sample (e.g., by Southern blot, dot blot, etc.). The hybridization pattern of a polymorphic or variant sequence and a control sequence to an array of oligonucleotide probes immobilized on a solid support, as described in U.S. Pat. No. 5,445,934, or in WO 95/35505, can also be used as a means of identifying polymorphic or variant sequences associated with disease. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in a gene can be based on the functional or antigenic characteristics of the protein. Protein truncation assays are useful in detecting deletions that can affect the biological activity of the protein. Various immunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

Diagnosis, Prognosis Assessment of Therapy (Therametrics), and Management of Cancer

The polynucleotides of the invention, as well as their gene products, are of particular interest as genetic or biochemical markers (e.g., in blood or tissues) that will detect the earliest changes along the carcinogenesis pathway and/or to monitor the efficacy of various therapies and preventive interventions. For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy. Determining expression of certain polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides of the invention are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides of the invention, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for metastatic colon cancer can also have clinical implications for stomach cancer or endometrial cancer.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides of the invention can facilitate fine-tuning of the staging process by identifying markers for the aggresivity of a cancer, e.g., the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors being more aggressive than well-differentiated or low-grade tumors. The following guidelines are generally used for grading tumors: 1) GX Grade cannot be assessed; 2) G1 Well differentiated; 3) G2 Moderately well differentiated; 4) G3 Poorly differentiated; 5) G4 Undifferentiated. The polynucleotides of the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

For prostate cancer, the Gleason Grading/Scoring system is most commonly used. A prostate biopsy tissue sample is examined under a microscope and a grade is assigned to the tissue based on: 1) the appearance of the cells, and 2) the arrangement of the cells. Each parameter is assessed on a scale of one (cells are almost normal) to five (abnormal), and the individual Gleason Grades are presented separated by a “+” sign. Alternatively, the two grades are combined to give a Gleason Score of 2-10. Thus, for a tissue sample that received a grade of 3 for each parameter, the Gleason Grade would be 3+3 and the Gleason Score would be 6. A lower Gleason Score indicates a well-differentiated tumor, while a higher Gleason Score indicates a poorly differentiated cancer that is more likely to spread. The majority of biopsies in general are Gleason Scores 5, 6 and 7.

| Gleason Score | Gleason Score | Gleason Score |
|---|---|---|
| 2, 3, 4 | 5, 6, 7 | 8, 9, 10 |
| Low-grade tumor | Medium-grade tumor | High-grade tumor |
| Slow Growth | Unpredictable Growth | Aggressive Growth |
| Least dangerous. | Intermediate cancers may | High-grade cancers are usually |
| Cells look most like normal | behave like low-grade or high- | very aggressive and quick to |
| prostate cells and are described | grade cancers. | spread to the tissue |
| as being “well-differentiated”. | The cells' behavior may | surrounding the prostate. |
| Tends to be slow growing. | depend on the volume of the | These cancer cells look least |
| | cancer and the PSA level. | like normal prostate cells and |
| | This is the most common | are usually described as |
| | grade of prostate cancer. | “poorly-differentiated”. |

The polynucleotides of the Sequence Listing, and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential. Detection of colon cancer. The polynucleotides corresponding to genes that exhibit the appropriate expression pattern can be used to detect colon cancer in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews. The expression of appropriate polynucleotides of the invention can be used in the diagnosis, prognosis and management of colorectal cancer. Detection of colon cancer can be determined using expression levels of any of these sequences alone or in combination with the levels of expression. Determination of the aggressive nature and/or the metastatic potential of a colon cancer can be determined by comparing levels of one or more polynucleotides of the invention and comparing total levels of another sequence known to vary in cancerous tissue, e.g., expression of p53, DCC ras, lor FAP (see, e.g., Fearon E R, et al., Cell (1990) 61(5):759; Hamilton S R et al., Cancer (1993) 72:957; Bodmer W, et al., Nat Genet. (1994) 4(3):217; Fearon E R, Ann N Y Acad Sci. (1995) 768:101). For example, development of colon cancer can be detected by examining the ratio of any of the polynucleotides of the invention to the levels of oncogenes (e.g., ras) or tumor suppressor genes (e.g., FAP or p53). Thus, expression of specific marker polynucleotides can be used to discriminate between normal and cancerous colon tissue, to discriminate between colon cancers with different cells of origin, to discriminate between colon cancers with different potential metastatic rates, etc. For a review of markers of cancer, see, e.g., Hanahan et al. (2000) Cell 100:57-70.

Detection of prostate cancer. The polynucleotides and their corresponding genes and gene products exhibiting the appropriate differential expression pattern can be used to detect prostate cancer in a subject. Prostate cancer is quite common in humans, with one out of every six men at a lifetime risk for prostate cancer, and can be relatively harmless or extremely aggressive. Some prostate tumors are slow growing, causing few clinical symptoms, while aggressive tumors spread rapidly to the lymph nodes, other organs and especially bone. Over 95% of primary prostate cancers are adenocarcinomas. Signs and symptoms may include: frequent urination, especially at night; inability to urinate; trouble starting or holding back urination; a weak or interrupted urine flow; and frequent pain or stiffness in the lower back, hips or upper thighs.

The prostate is divided into three areas-the peripheral zone, the transition zone, and the central zone-with a layer of tissue surrounding all three. Most prostate tumors form in the peripheral zone; the larger, glandular portion of the organ. Prostate cancer can also form in the tissue of the central zone. Surrounding the prostate is the prostate capsule, a tissue that separates the prostate from the rest of the body. When prostate cancer remains inside the prostate capsule, it is considered localized and treatable with surgery. Once the cancer punctures the capsule and spreads outside, treatment options are more limited. Prevention and early detection are key factors in controlling and curing prostate cancer.

While the Gleason Grade or Score of a prostate cancer can provide information useful in determining the appropriate treatment of a prostate cancer, the majority of prostate cancers are Gleason Scores 5, 6, and 7, which exhibit unpredictable behavior. These cancers may behave like less dangerous low-grade cancers or like extremely dangerous high-grade cancers. As a result, a patient living with a medium-grade prostate cancer is at constant risk of developing high-grade cancer.

The expression of appropriate polynucleotides can be used in the diagnosis, prognosis and management of prostate cancer. Detection of prostate cancer can be determined using expression levels of any of these sequences alone or in combination with the levels of expression of any other nucleotide sequences. Determination of the aggressive nature and/or the metastatic potential of a prostate cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon E R, et al., Cell (1990) 61(5):759; Hamilton S R et al., Cancer (1993) 72:957; Bodmer W, et al., Nat Genet. (1994) 4(3):217; Fearon E R, Ann N Y Acad Sci. (1995) 768:101).

For example, development of prostate cancer can be detected by examining the level of expression of a gene corresponding to a polynucleotides described herein to the levels of oncogenes (e.g. ras) or tumor suppressor genes (e.g. FAP or p53). Thus expression of specific marker polynucleotides can be used to discriminate between normal and cancerous prostate tissue, to discriminate between prostate cancers with different cells of origin, to discriminate between prostate cancers with different potential metastatic rates, etc. For a review of markers of cancer, see, e.g., Hanahan et al. (2000) Cell 100:57-70.

In addition, many of the signs and symptoms of prostate cancer can be caused by a variety of other non-cancerous conditions. For example, one common cause of many of these signs and symptoms is a condition called benign prostatic hypertrophy, or BPH. In BPH, the prostate gets bigger and may block the flow of urine or interfere with sexual function. The methods and compositions of the invention can be used to distinguish between prostate cancer and such non-cancerous conditions. The methods of the invention can be used in conjunction with conventional methods of diagnosis, e.g., digital rectal exam and/or detection of the level of prostate specific antigen (PSA), a substance produced and secreted by the prostate.

Detection of breast cancer. The majority of breast cancers are adenocarcinoma subtypes, which can be summarized as follows: 1) ductal carcinoma in situ (DCIS), including comedocarcinoma; 2) infiltrating (or invasive) ductal carcinoma (IDC); 3) lobular carcinoma in situ (LCIS); 4) infiltrating (or invasive) lobular carcinoma (ILC); 5) inflammatory breast cancer; 6) medullary carcinoma; 7) mucinous carcinoma; 8) Paget's disease of the nipple; 9) Phyllodes tumor; and 10) tubular carcinoma;

The expression of polynucleotides of the invention can be used in the diagnosis and management of breast cancer, as well as to distinguish between types of breast cancer. Detection of breast cancer can be determined using expression levels of any of the appropriate polynucleotides of the invention, either alone or in combination. Determination of the aggressive nature and/or the metastatic potential of a breast cancer can also be determined by comparing levels of one or more polynucleotides of the invention and comparing levels of another sequence known to vary in cancerous tissue, e.g., ER expression. In addition, development of breast cancer can be detected by examining the ratio of expression of a differentially expressed polynucleotide to the levels of steroid hormones (e.g., testosterone or estrogen) or to other hormones (e.g., growth hormone, insulin). Thus, expression of specific marker polynucleotides can be used to discriminate between normal and cancerous breast tissue, to discriminate between breast cancers with different cells of origin, to discriminate between breast cancers with different potential metastatic rates, etc.

Detection of lung cancer. The polynucleotides of the invention can be used to detect lung cancer in a subject. Although there are more than a dozen different kinds of lung cancer, the two main types of lung cancer are small cell and nonsmall cell, which encompass about 90% of all lung cancer cases. Small cell carcinoma (also called oat cell carcinoma) usually starts in one of the larger bronchial tubes, grows fairly rapidly, and is likely to be large by the time of diagnosis. Nonsmall cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and can vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

The polynucleotides of the invention, e.g., polynucleotides differentially expressed in normal cells versus cancerous lung cells (e.g., tumor cells of high or low metastatic potential) or between types of cancerous lung cells (e.g., high metastatic versus low metastatic), can be used to distinguish types of lung cancer as well as identifying traits specific to a certain patient's cancer and selecting an appropriate therapy. For example, if the patient's biopsy expresses a polynucleotide that is associated with a low metastatic potential, it may justify leaving a larger portion of the patient's lung in surgery to remove the lesion. Alternatively, a smaller lesion with expression of a polynucleotide that is associated with high metastatic potential may justify a more radical removal of lung tissue and/or the surrounding lymph nodes, even if no metastasis can be identified through pathological examination.

Identification of Therapeutic Targets and Anti-Cancer Therapeutic Agents

The present invention also encompasses methods for identification of agents having the ability to modulate activity of a differentially expressed gene product, as well as methods for identifying a differentially expressed gene product as a therapeutic target for treatment of cancer, especially prostate cancer.

Candidate Agents

Identification of compounds that modulate activity of a differentially expressed gene product can be accomplished using any of a variety of drug screening techniques. Such agents are candidates for development of cancer therapies. Of particular interest are screening assays for agents that have tolerable toxicity for normal, non-cancerous human cells. The screening assays of the invention are generally based upon the ability of the agent to modulate an activity of a differentially expressed gene product and/or to inhibit or suppress phenomenon associated with cancer (e.g., cell proliferation, colony formation, cell cycle arrest, metastasis, and the like).

The term “agent” as used herein describes any molecule, e.g. protein or pharmaceutical, with the capability Of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

Screening of Candidate Agents

Screening assays can be based upon any of a variety of techniques readily available and known to one of ordinary skill in the art. In general, the screening assays involve contacting a cancerous cell (preferably a cancerous prostate cell) with a candidate agent, and assessing the effect upon biological activity of a differentially expressed gene product. The effect upon a biological activity can be detected by, for example, detection of expression of a gene product of a differentially expressed gene (e.g., a decrease in mRNA or polypeptide levels, would in turn cause a decrease in biological activity of the gene product). Alternatively or in addition, the effect of the candidate agent can be assessed by examining the effect of the candidate agent in a functional assay. For example, where the differentially expressed gene product is an enzyme, then the effect upon biological activity can be assessed by detecting a level of enzymatic activity associated with the differentially expressed gene product. The functional assay will be selected according to the differentially expressed gene product. In general, where the differentially expressed gene is increased in expression in a cancerous cell, agents of interest are those that decrease activity of the differentially expressed gene product.

Assays described infra can be readily adapted in the screening assay embodiments of the invention. Exemplary assays useful in screening candidate agents include, but are not limited to, hybridization-based assays (e.g., use of nucleic acid probes or primers to assess expression levels), antibody-based assays (e.g., to assess levels of polypeptide gene products), binding assays (e.g., to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

Identification of Therapeutic Targets

In another embodiment, the invention contemplates identification of differentially expressed genes and gene products as therapeutic targets. In some respects, this is the converse of the assays described above for identification of agents having activity in modulating (e.g., decreasing or increasing) activity of a differentially expressed gene product.

In this embodiment, therapeutic targets are identified by examining the effect(s) of an agent that can be demonstrated or has been demonstrated to modulate a cancerous phenotype (e.g., inhibit or suppress or prevent development of a cancerous phenotype). Such agents are generally referred to herein as an “anti-cancer agent”, which agents encompass chemotherapeutic agents. For example, the agent can be an antisense oligonucleotide that is specific for a selected gene transcript. For example, the antisense oligonucleotide may have a sequence corresponding to a sequence of a differentially expressed gene described herein, e.g., a sequence of one of SEQ ID NOS: 1-2164.

Assays for identification of therapeutic targets can be conducted in a variety of ways using methods that are well known to one of ordinary skill in the art. For example, a test cancerous cell that expresses or overexpresses a differentially expressed gene is contacted with an anti-cancer agent, the effect upon a cancerous phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product. The cancerous phenotype can be, for example, cellular proliferation, loss of contact inhibition of growth (e.g., colony formation), tumor growth (in vitro or in vivo), and the like. Alternatively or in addition, the effect of modulation of a biological activity of the candidate target gene upon cell death/apoptosis or cell cycle regulation can be assessed.

Inhibition or suppression of a cancerous phenotype, or an increase in cell/death apoptosis as a result of modulation of biological activity of a candidate gene product indicates that the candidate gene product is a suitable target for cancer therapy. Assays described infra can be readily adapted in for assays for identification of therapeutic targets. Generally such assays are conducted in vitro, but many assays can be adapted for in vivo analyses, e.g., in an appropriate, art-accepted animal model of the cancer.

Use of Polynucleotides to Screen for Peptide Analogs and Antagonists

Polypeptides encoded by the instant polynucleotides and corresponding full-length genes can be used to screen peptide libraries to identify binding partners, such as receptors, from among the encoded polypeptides. Peptide libraries can be synthesized according to methods known in the art (see, e.g., U.S. Pat. No. 5,010,175, and WO 91/17823).

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a novel polypeptide binding partner, such as a receptor, encoded by a gene or a cDNA corresponding to a polynucleotide of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding can facilitate development of improved agonists/antagonists of the known receptor.

Vaccines and Uses

The differentially expressed nucleic acids and polypeptides produced by the nucleic acids of the invention can also be used to modulate primary immune response to prevent or treat cancer. Every immune response is a complex and intricately regulated sequence of events involving several cell types. It is triggered when an antigen enters the body and encounters a specialized class of cells called antigen-presenting cells (APCs). These APCs capture a minute amount of the antigen and display it in a form that can be recognized by antigen-specific helper T lymphocytes. The helper (Th) cells become activated and, in turn, promote the activation of other classes of lymphocytes, such as B cells or cytotoxic T cells. The activated lymphocytes then proliferate and carry out their specific effector functions, which in many cases successfully activate or eliminate the antigen. Thus, activating the immune response to a particular antigen associated with a cancer cell can protect the patient from developing cancer or result in lymphocytes eliminating cancer cells expressing the antigen.

Gene products, including polypeptides, mRNA (particularly mRNAs having distinct secondary and/or tertiary structures), cDNA, or complete gene, can be prepared and used in vaccines for the treatment or prevention of hyperproliferative disorders and cancers. The nucleic acids and polypeptides can be utilized to enhance the immune response, prevent tumor progression, prevent hyperproliferative cell growth, and the like. Methods for selecting nucleic acids and polypeptides that are capable of enhancing the immune response are known in the art. Preferably, the gene products for use in a vaccine are gene products which are present on the surface of a cell and are recognizable by lymphocytes and antibodies.

The gene products may be formulated with pharmaceutically acceptable carriers into pharmaceutical compositions by methods known in the art. The composition is useful as a vaccine to prevent or treat cancer. The composition may further comprise at least one co-immunostimulatory molecule, including but not limited to one or more major histocompatibility complex (MHC) molecules, such as a class I or class II molecule, preferably a class I molecule. The composition may further comprise other stimulator molecules including B7.1, B7.2, ICAM-1, ICAM-2, LFA-1, LFA-3, CD72 and the like, immunostimulatory polynucleotides (which comprise an 5′-CG-3′ wherein the cytosine is unmethylated), and cytokines which include but are not limited to IL-1 through IL-15, TNF-α, IFN-γ, RANTES, G-CSF, M-CSF, IFN-α, CTAP III, ENA-78, GRO, I-309, PF-4, IP-10, LD-78, MGSA, MIP-1α, MIP-1β, or combination thereof, and the like for immunopotentiation. In one embodiment, the immunopotentiators of particular interest are those which facilitate a Th1 immune response.

The gene products may also be prepared with a carrier that will protect the gene products against rapid elimination from the body, such as a controlled release formulation, including implants and microencapsulated delivery systems. Biodegradable polymers can be used, such as ethylene vinyl acetate, polyanhydrides, polyglycolic acid, collagen, polyorthoesters, polylactic acid, and the like. Methods for preparation of such formulations are known in the art.

In the methods of preventing or treating cancer, the gene products may be administered via one of several routes including but not limited to transdermal, transmucosal, intravenous, intramuscular, subcutaneous, intradermal, intraperitoneal, intrathecal, intrapleural, intrauterine, rectal, vaginal, topical, intratumor, and the like. For transmucosal or transdermal administration, penetrants appropriate to the barrier to be permeated are used in the formulation. Such penetrants are generally known in the art, and include, for example, administration bile salts and fusidic acid derivatives. In addition, detergents may be used to facilitate permeation. Transmucosal administration may be by nasal sprays or suppositories. For oral administration, the gene products are formulated into conventional oral administration form such as capsules, tablets and toxics.

The gene product is administered to a patient in an amount effective to prevent or treat cancer. In general, it is desirable to provide the patient with a dosage of gene product of at least about 1 pg per Kg body weight, preferably at least about 1 ng per Kg body weight, more preferably at least about 1 μg or greater per Kg body weight of the recipient. A range of from about 1 ng per Kg body weight to about 100 mg per Kg body weight is preferred although a lower or higher dose may be administered. The dose is effective to prime, stimulate and/or cause the clonal expansion of antigen-specific T lymphocytes, preferably cytotoxic T lymphocytes, which in turn are capable of preventing or treating cancer in the recipient. The dose is administered at least once and may be provided as a bolus or a continuous administration. Multiple administrations of the dose over a period of several weeks to months may be preferable. Subsequent doses may be administered as indicated.

In another method of treatment, autologous cytotoxic lymphocytes or tumor infiltrating lymphocytes may be obtained from a patient with cancer. The lymphocytes are grown in culture, and antigen-specific lymphocytes are expanded by culturing in the presence of the specific gene products alone or in combination with at least one co-immunostimulatory molecule with cytokines. The antigen-specific lymphocytes are then infused back into the patient in an amount effective to reduce or eliminate the tumors in the patient. Cancer vaccines and their uses are further described in U.S. Pat. Nos. 5,961,978; 5,993,829; 6,132,980; and WO 00/38706.

Pharmaceutical Compositions and Uses

Pharmaceutical compositions can comprise polypeptides, receptors that specifically bind a polypeptide produced by a differentially expressed gene (e.g., antibodies, or polynucleotides (including antisense nucleotides and ribozymes) of the claimed invention in a therapeutically effective amount. The compositions can be used to treat primary tumors as well as metastases of primary tumors. In addition, the pharmaceutical compositions can be used in conjunction with conventional methods of cancer treatment, e.g., to sensitize tumors to radiation or conventional chemotherapy.

Where the pharmaceutical composition comprises a receptor (such as an antibody) that specifically binds to a gene product encoded by a differentially expressed gene, the receptor can be coupled to a drug for delivery to a treatment site or coupled to a detectable label to facilitate imaging of a site comprising colon cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Delivery Methods

Once formulated, the compositions of the invention can be (1) administered directly to the subject (e.g., as polynucleotide or polypeptides); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumorally or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, e.g., WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once differential expression of a gene corresponding to a polynucleotide of the invention has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents of the invention includes local or systemic administration, including injection, oral administration, particle gun or catheterized administration, and topical administration. Preferably, the therapeutic polynucleotide composition contains an expression construct comprising a promoter operably linked to a polynucleotide of at least 12, 22, 25, 30, or 35 contiguous nt of the polynucleotide of the invention. Various methods can be used to administer the therapeutic composition directly to a specific site in the body. For example, a small metastatic lesion is located and the therapeutic composition injected several times in several different locations within the body of tumor. Alternatively, arteries that serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 micrograms to about 2 mg, about 5 micrograms to about 500 micrograms, and about 20 micrograms to about 100 micrograms of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the antisense subgenomic polynucleotides.

Where greater expression is desired over a larger area of tissue, larger amounts of antisense subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. For polynucleotide related genes encoding polypeptides or proteins with anti-inflammatory activity, suitable use, doses, and administration are described in U.S. Pat. No. 5,654,173.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus, as described in Curiel, Hum. Gene Ther. (1992) 3:147, can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581

Further non-viral delivery suitable for use includes mechanical delivery systems such as the approach described in Woffendin et al., Proc. Natl. Acad. Sci. USA (1994) 91(24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous embodiments. However, it should be noted that these embodiments are illustrative and are not to be construed as restricting the invention in any way.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. It will be readily apparent to those skilled in the art that the formulations, dosages, methods of administration, and other parameters of this invention may be further modified or substituted in various ways without departing from the spirit and scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Source of Biological Materials and Overview of Novel Polynucleotides Expressed by the Biological Materials

Candidate polynucleotides that may represent novel polynucleotides were obtained from cDNA libraries generated from selected cell lines and patient tissues. In order to obtain the candidate polynucleotides, mRNA was isolated from several selected cell lines and patient tissues, and used to construct cDNA libraries. The cells and tissues that served as sources for these cDNA libraries are summarized in Table 1 below.

Human colon cancer cell line Km12L4-A (Morikawa, et al., Cancer Research (1988) 48:6863) is derived from the KM12C cell line. The KM12C cell line (Morikawa et al. Cancer Res. (1988) 48:1943-1948), which is poorly metastatic (low metastatic) was established in culture from a Dukes' stage B2 surgical specimen (Morikawa et al. Cancer Res. (1988) 48:6863). The KM12L4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246).

The MDA-MB-231 cell line (Brinkley et al. Cancer Res. (1980) 40:3118-3129) was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. The MV-522 cell line is derived from a human lung carcinoma and is of high metastatic potential. The UCP-3 cell line is a low metastatic human lung carcinoma cell line; the MV-522 is a high metastatic variant of UCP-3. These cell lines are well-recognized in the art as models for the study of human breast and lung cancer (see, e.g., Chandrasekaran et al., Cancer Res. (1979) 39:870 (MDA-MB-231 and MCF-7); Gastpar et al., J Med Chem (1998) 41:4965 (MDA-MB-231 and MCF-7); Ranson et al., Br J Cancer (1998) 77:1586 (MDA-MB-231 and MCF-7); Kuang et al., Nucleic Acids Res (1998) 26:1116 (MDA-MB-231 and MCF-7); Varki et al., Int J Cancer (1987) 40:46 (UCP-3); Varki et al., Tumour Biol. (1990) 11:327; (MV-522 and UCP-3); Varki et al., Anticancer Res. (1990) 10:637; (MV-522); Kelner et al., Anticancer Res (1995) 15:867 (MV-522); and Zhang et al., Anticancer Drugs (1997) 8:696 (MV522)).

The samples of libraries 15-20 are derived from two different patients (UC#2, and UC#3). The bFGF-treated HMVEC were prepared by incubation with bFGF at 10 ng/ml for 2 hrs; the VEGF-treated HMVEC were prepared by incubation with 20 ng/ml VEGF for 2 hrs. Following incubation with the respective growth factor, the cells were washed and lysis buffer added for RNA preparation.

GRRpz was derived from normal prostate epithelium. The WOca cell line is a Gleason Grade 4 cell line.

The source materials for generating the normalized prostate libraries of libraries 25 and 26 were cryopreserved prostate tumor tissue from a patient with Gleason grade 3+3 adenocarcinoma and matched normal prostate biopsies from a pool of at-risk subjects under medical surveillance. The source materials for generating the normalized prostate libraries of libraries 30 and 31 were cryopreserved prostate tumor tissue from a patient with Gleason grade 4+4 adenocarcinoma and matched normal prostate biopsies from a pool of at-risk subjects under medical surveillance.

The source materials for generating the normalized breast libraries of libraries 27, 28 and 29 were cryopreserved breast tissue from a primary breast tumor (infiltrating ductal carcinoma)(library 28), from a lymph node metastasis (library 29), or matched normal breast biopsies from a pool of at-risk subjects under medical surveillance. In each case, prostate or breast epithelia were harvested directly from frozen sections of tissue by laser capture microdissection (LCM, Arcturus Enginering Inc., Mountain View, Calif.), carried out according to methods well known in the art (see Simone et al. Am J Pathol. 156(2):445-52 (2000)) to provide substantially homogenous cell samples.

TABLE 1
Description of cDNA Libraries

| | | Number |
|---|---|---|
| Library | | of Clones |
| (lib#) | Description | in Library |
| 0 | Artificial library composed of deselected clones (clones with no | 673 |
| | associated variant or cluster) | |
| 1 | Human Colon Cell Line Km12 L4: High Metastatic Potential | 308731 |
| | (derived from Km12C) | |
| 2 | Human Colon Cell Line Km12C: Low Metastatic Potential | 284771 |
| 3 | Human Breast Cancer Cell Line MDA-MB-231: High Metastatic | 326937 |
| | Potential; micro-mets in lung | |
| 4 | Human Breast Cancer Cell Line MCF7: Non Metastatic | 318979 |
| 8 | Human Lung Cancer Cell Line MV-522: High Metastatic Potential | 223620 |
| 9 | Human Lung Cancer Cell Line UCP-3: Low Metastatic Potential | 312503 |
| 12 | Human microvascular endothelial cells (HMEC) - UNTREATED | 41938 |
| | (PCR (OligodT) cDNA library) | |
| 13 | Human microvascular endothelial cells (HMEC) - bFGF TREATED | 42100 |
| | (PCR (OligodT) cDNA library) | |
| 14 | Human microvascular endothelial cells (HMEC) - VEGF TREATED | 42825 |
| | (PCR (OligodT) cDNA library) | |
| 15 | Normal Colon - UC#2 Patient (MICRODISSECTED PCR (OligodT) | 282722 |
| | cDNA library) | |
| 16 | Colon Tumor - UC#2 Patient (MICRODISSECTED PCR (OligodT) | 298831 |
| | cDNA library) | |
| 17 | Liver Metastasis from Colon Tumor of UC#2 Patient | 303467 |
| | (MICRODISSECTED PCR (OligodT) cDNA library) | |
| 18 | Normal Colon - UC#3 Patient (MICRODISSECTED PCR (OligodT) | 36216 |
| | cDNA library) | |
| 19 | Colon Tumor - UC#3 Patient (MICRODISSECTED PCR (OligodT) | 41388 |
| | cDNA library) | |
| 20 | Liver Metastasis from Colon Tumor of UC#3 Patient | 30956 |
| | (MICRODISSECTED PCR (OligodT) cDNA library) | |
| 21 | GRRpz Cells derived from normal prostate epithelium | 164801 |
| 22 | WOca Cells derived from Gleason Grade 4 prostate cancer | 162088 |
| | epithelium | |
| 23 | Normal Lung Epithelium of Patient #1006 (MICRODISSECTED | 306198 |
| | PCR (OligodT) cDNA library) | |
| 24 | Primary tumor, Large Cell Carcinoma of Patient #1006 | 309349 |
| | (MICRODISSECTED PCR (OligodT) cDNA library) | |
| 25 | Normal Prostate Epithelium from Patient IF97-26811 | 279444 |
| 26 | Prostate Cancer Epithelium Gleason 3 + 3 Patient IF97-26811 | 269406 |
| 27 | Normal Breast Epithelium from Patient 515 | 239494 |
| 28 | Primary Breast tumor from Patient 515 | 259960 |
| 29 | Lymph node metastasis from Patient 515 | 326786 |
| 30 | Normal Prostate Epithelium from Chiron Patient ID 884 | 298431 |
| 31 | Prostate Cancer Epithelium (Gleason 4 + 4) from Chiron Patient ID | 331941 |
| | 884 | |

Characterization of Sequences in the Libraries

After using the software program Phred (ver 0.000925.c, Green and Weing, ©993-2000) to select those polynucleotides having the best quality sequence, the polynucleotides were compared against the public databases to identify any homologous sequences. The sequences of the isolated polynucleotides were first masked to eliminate low complexity sequences using the RepeatMasker masking program, publicly available through a web site supported by the University of Washington (See also Smit, A. F. A. and Green, P., unpublished results). Generally, masking does not influence the final search results, except to eliminate sequences of relatively little interest due to their low complexity, and to eliminate multiple “hits” based on similarity to repetitive regions common to multiple sequences, e.g., Alu repeats.

The remaining sequences were then used in a homology search of the GenBank database using the TeraBLAST program (TimeLogic, Crystal Bay, Nev.). TeraBLAST is a version of the publicly available BLAST search algorithm developed by the National Center for Biotechnology, modified to operate at an accelerated speed with increased sensitivity on a specialized computer hardware platform. The program was run with the default parameters recommended by TimeLogic to provide the best sensitivity and speed for searching DNA and protein sequences. Sequences that exhibited greater than 70% overlap, 99% identity, and a p value of less than 1×10e−40 were discarded. Sequences from this search also were discarded if the inclusive parameters were met, but the sequence was ribosomal or vector-derived.

The resulting sequences from the previous search were classified into three groups (1, 2 and 3 below) and searched in a TeraBLASTX vs. NRP (non-redundant proteins) database search: (1) unknown (no hits in the GenBank search), (2) weak similarity (greater than 45% identity and p value 1×10e−5), and (3) high similarity (greater than 60% overlap, greater than 80% identity, and p value less than 1×10e−5). Sequences having greater than 70% overlap, greater than 99% identity, and p value of less than 1×10e−40 were discarded.

The remaining sequences were classified as unknown (no hits), weak similarity, and high similarity (parameters as above). Two searches were performed on these sequences. First, a TeraBLAST vs. EST database search was performed and sequences with greater than 99% overlap, greater than 99% similarity and a p value of less than 1×10e−40 were discarded. Sequences with a p value of less than 1×10e−65 when compared to a database sequence of human origin were also excluded. Second, a TeraBLASTN vs. Patent GeneSeq database was performed and sequences having greater than 99% identity, p value less than 1×10e−40, and greater than 99% overlap were discarded.

The remaining sequences were subjected to screening using other rules and redundancies in the dataset. Sequences with a p value of less than 1×10e−111 in relation to a database sequence of human origin were specifically excluded. The final result provided the sequences listed as SEQ ID NOS: 1-1267 in the accompanying Sequence Listing and summarized in Table 2 (inserted prior to claims). Each identified polynucleotide represents sequence from at least a partial mRNA transcript.

Summary of polynucleotides of the invention

Table 2 (inserted prior to claims) provides a summary of polynucleotides isolated as described. Specifically, Table 2 provides: 1) the SEQ ID NO (“SEQ ID”) assigned to each sequence for use in the present specification; 2) theCluster Identification No. (“CLUSTER”); 3) the Sequence Name assigned to each sequence; 3) the sequence name (“SEQ NAME”) used as an internal identifier of the sequence; 4) the orientation of the sequence (“ORIENT”) (either forward (F) or reverse (R)); 5) the name assigned to the clone from which the sequence was isolated (“CLONE ID”); and 6) the name of the library from which the sequence was isolated (“LIBRARY”). Because at least some of the provided polynucleotides represent partial mRNA transcripts, two or more polynucleotides may represent different regions of the same mRNA transcript and the same gene and/or may be contained within the same clone. Thus, for example, if two or more SEQ ID NOS: are identified as belonging to the same clone, then either sequence can be used to obtain the full-length mRNA or gene. Clones which comprise the sequences described herein were deposited as set out in the tables indicated below (see Example entitled “Deposit Information”).

### Example 2

Contig Assembly

The sequences of the polynucleotides provided in the present invention can be used to extend the sequence information of the gene to which the polynucleotides correspond (e.g., a gene, or mRNA encoded by the gene, having a sequence of the polynucleotide described herein). This expanded sequence information can in turn be used to further characterize the corresponding gene, which in turn provides additional information about the nature of the gene product (e.g., the normal function of the gene product). The additional information can serve to provide additional evidence of the gene product's use as a therapeutic target, and provide further guidance as to the types of agents that can modulate its activity.

For example, a contig was assembled using the sequence of a polynucleotide described herein. A “contig” is a contiguous sequence of nucleotides that is assembled from nucleic acid sequences having overlapping (e.g., shared or substantially similar) sequence information. The sequences of publicly-available ESTs (Expressed Sequence Tags) and the sequences of various of the above-described polynucleotides were used in the contig assembly. The contig was assembled using the software program Sequencher, version 4.05, according to the manufacturer's instructions. The sequence information obtained in the contig assembly was then used to obtain a consensus sequence derived from the contig using the Sequencher program. The resulting consensus sequence was used to search both the public databases as well as databases internal to the applicants to match the consensus polynucleotide with homology data and/or differential gene expressed data.

The final result provided the sequences listed as SEQ ID NOS: 1268-1385 in the accompanying Sequence Listing and summarized in Table 3 (inserted prior to claims). Table 3 provides a summary of the consensus sequences assembled as described. Specifically, Table 3 provides: 1) the SEQ ID NO (“SEQ ID”) assigned to each sequence for use in the present specification; 2) the consensus sequence name (“CONSENSUS SEQ NAME”) used as an internal identifier of the sequence; and 3) the sequence name (“POLYNTD SEQ NAME”) of a polynucleotide of SEQ ID NOS: 1-1267 used in assembly of the consensus sequence.

### Example 3

Additional Gene Characterization

Sequences of the polynucleotides of SEQ ID NOS: 1-1267 were used as a query sequence in a TeraBLASTN search of the DoubleTwist Human Genome Sequence Database (DoubleTwist, Inc., Oakland, Calif.), which contains all the human genomic sequences that have been assembled into a contiguous model of the human genome. Predicted cDNA and protein sequences were obtained where a polynucleotide of the invention was homologous to a predicted full-length gene sequence. Alternatively, a sequence of a contig or consensus sequence described herein could be used directly as a query sequence in a TeraBLASTN search of the DoubleTwist Human Genome Sequence Database.

The final results of the search provided the predicted cDNA sequences listed as SEQ ID NOS: 1386-1477 in the accompanying Sequence Listing and summarized in Table 4 (inserted prior to claims), and the predicted protein sequences listed as SEQ ID NOS: 1478-1568 in the accompanying Sequence Listing and summarized in Table 5 (inserted prior to claims). Specifically, Table 4 provides: 1) the SEQ ID NO (“SEQ ID”) assigned to each cDNA sequence for use in the present specification; 2) the cDNA sequence name (“cDNA SEQ NAME”) used as an internal identifier of the sequence; 3) the sequence name (“POLYNTD SEQ NAME”) of the polynucleotide of SEQ ID NOS: 1-1267 that maps to the cDNA; 4)The gene id number (GENE) of the DoubleTwist predicted gene; 5) the chromosome (“CHROM”) containing the gene corresponding to the cDNA sequence; Table 5 provides: 1) the SEQ ID NO (“SEQ ID”) assigned to each protein sequence for use in the present specification; 2) the protein sequence name (“PROTEIN SEQ NAME”) used as an internal identifier of the sequence; 3) the sequence name (“POLYNTD SEQ NAME”) of the polynucleotide of SEQ ID NOS: 1-1267 that maps to the protein sequence; 4)The gene id number (GENE) of the DoubleTwist predicted gene; 5) the chromosome (“CHROM”) containing the gene corresponding to the cDNA sequence.

A correlation between the polynucleotide used as a query sequence as described above and the corresponding predicted cDNA and protein sequences is contained in Table 6. Specifically Table 6 provides: 1) the SEQ ID NO of the cDNA (“cDNA SEQ ID”); 2) the cDNA sequence name (“cDNA SEQ NAME”) used as an internal identifier of the sequence; 3) the SEQ ID NO of the protein (“PROTEIN SEQ ID”) encoded by the cDNA sequence 4) the sequence name of the protein (“PROTEIN SEQ NAME”) encoded by the cDNA sequence; 5) the SEQ ID NO of the polynucleotide (“POLYNTD SEQ ID”) of SEQ ID NOS: 1-1267 that maps to the cDNA and protein; and 6) the sequence name (“POLYNTD SEQ NAME”) of the polynucleotide of SEQ ID NOS: 1-1267 that maps to the cDNA and protein.

Through contig and consensus sequence assembly and the use of homology searching software programs, the sequence information provided herein can be readily extended to confirm, or confirm a predicted, gene having the sequence of the polynucleotides described in the present invention. Further the information obtained can be used to identify the function of the gene product of the gene corresponding to the polynucleotides described herein. While not necessary to the practice of the invention, identification of the function of the corresponding gene, can provide guidance in the design of therapeutics that target the gene to modulate its activity and modulate the cancerous phenotype (e.g., inhibit metastasis, proliferation, and the like).

### Example 4

Results of Public Database Search to Identify Function of Gene Products

SEQ ID NOS: 1-1477 were translated in all three reading frames, and the nucleotide sequences and translated amino acid sequences used as query sequences to search for homologous sequences in the GenBank (nucleotide sequences) database. Query and individual sequences were aligned using the TeraBLAST program available from TimeLogic, Crystal Bay, Nev. The sequences were masked to various extents to prevent searching of repetitive sequences or poly-A sequences, using the RepeatMasker masking program for masking low complexity as described above.

Table 7 (inserted prior to claims) provides the alignment summaries having a p value of 1×10e−2 or less indicating substantial homology between the sequences of the present invention and those of the indicated public databases. Specifically, Table 7 provides: 1) the SEQ ID NO (“SEQ ID”) of the query sequence; 2) the sequence name (“SEQ NAME”) used as an internal identifier of the query sequence; 3) the accession number (“ACCESSION”) of the GenBank database entry of the homologous sequence; 4) a description of the GenBank sequences (“GENBANK DESCRIPTION”); and 5) the score of the similarity of the polynucleotide sequence and the GenBank sequence (“GENBANK SCORE”). The alignments provided in Table 7 are the best available alignment to a DNA sequence at a time just prior to filing of the present specification. Also incorporated by reference is all publicly available information regarding the sequence listed in Table 6 and their related sequences. The search program and database used for the alignment, as well as the calculation of the p value are also indicated. Full length sequences or fragments of the polynucleotide sequences can be used as probes and primers to identify and isolate the full length sequence of the corresponding polynucleotide.

### Example 5

Members of Protein Families

SEQ ID NOS: 1-1477 were used to conduct a profile search as described in the specification above. Several of the polynucleotides of the invention were found to encode polypeptides having characteristics of a polypeptide belonging to a known protein family (and thus represent members of these protein families) and/or comprising a known functional domain. Table 8 (inserted prior to claims) provides: 1) the SEQ ID NO (“SEQ ID”) of the query polynucleotide sequence; 2) the sequence name (“SEQ NAME”) used as an internal identifier of the query sequence; 3) the accession number (“PFAM ID”) of the the protein family profile hit; 4) a brief description of the profile hit (“PFAM DESCRIPTION”); 5) the score (“SCORE”) of the profile hit; 6) the starting nucleotide of the profile hit (“START”); and 7) the ending nucleotide of the profile hit (“END”).

In addition, SEQ ID NOS: 1478-1568 were also used to conduct a profile search as described above. Several of the polypeptides of the invention were found to have characteristics of a polypeptide belonging to a known protein family (and thus represent members of these protein families) and/or comprising a known functional domain. Table 9 (inserted prior to claims) provides: 1) the SEQ ID NO (“SEQ ID”) of the query protein sequence; 2) the sequence name (“PROTEIN SEQ NAME”) used as an internal identifier of the query sequence; 3) the accession number (“PFAM ID”) of the the protein family profile hit; 4) a brief description of the profile hit (“PFAM DESCRIPTION”); 5) the score (“SCORE”) of the profile hit; 6) the starting residue of the profile hit (“START”); and 7) the ending residue of the profile hit (“END”).

Some SEQ ID NOS exhibited multiple profile hits where the query sequence contains overlapping profile regions, and/or where the sequence contains two different functional domains. Each of the profile hits of Tables 8 and 9 is described in more detail below. The acronyms for the profiles (provided in parentheses) are those used to identify the profile in the Pfam, Prosite, and InterPro databases. The Pfam database can be accessed through web sites supported by Genome Sequencing Center at the Washington University School of Medicine or by the European Molecular Biology Laboratories in Heidelberg, Germany. The Prosite database can be accessed at the ExPASy Molecular Biology Server on the internet. The InterPro database can be accessed at a web site supported by the EMBL European Bioinformatics Institute. The public information available on the Pfam, Prosite, and InterPro databases regarding the various profiles, including but not limited to the activities, function, and consensus sequences of various proteins families and protein domains, is incorporated herein by reference.

Ank Repeats (ANK; Pfam Accession No. PF0023). SEQ ID NOS: 482, 818, 914, 1216, 1484, 1537, and 1564 represent Ank repeat-containing proteins. The ankyrin motif is a 33 amino acid sequence named after the protein ankyrin which has 24 tandem 33-amino-acid motifs. Ank repeats were originally identified in the cell-cycle-control protein cdc10 (Breeden et al., Nature (1987) 329:651). Proteins containing ankyrin repeats include ankyrin, myotropin, I-kappaB proteins, cell cycle protein cdc10, the Notch receptor (Matsuno et al., Development (1997) 124(21):4265); G9a (or BAT8) of the class III region of the major histocompatibility complex (Biochem J. (1993) 290:811-818); FABP, GABP, 53BP2, Lin12, glp-1, SW14, and SW16. The functions of the ankyrin repeats are compatible with a role in protein-protein interactions (Bork, Proteins (1993) 17(4):363; Lambert and Bennet, Eur. J. Biochem. (1993) 211:1; Kerr et al., Current Op. Cell Biol. (1992) 4:496; Bennet et al., J. Biol. Chem. (1980) 255:6424).

Epidermal Growth Factor (EGF; Pfam Accession No. PF00008). SEQ ID NO: 967 represents a polynucleotide encoding a member of the EGF family of proteins. The distinguishing characteristic of this family is the presence of a sequence of about thirty to forty amino acid residues found in epidermal growth factor (EGF) which has been shown to be present, in a more or less conserved form, in a large number of other proteins (Davis, New Biol. (1990) 2:410-419; Blomquist et al., Proc. Natl. Acad. Sci U.S.A. (1984) 81:7363-7367; Barkert et al., Protein Nuc. Acid Enz. (1986) 29:54-86; Doolittle et al., Nature. (1984) 307:558-560; Appella et al., FEBS Lett. (1988) 231:1-4; Campbell and Bork, Curr. Opin. Struct. Biol. (1993) 3:385-392). A common feature of the domain is that the conserved pattern is generally found in the extracellular domain of membrane-bound proteins or in proteins known to be secreted. The EGF domain includes six cysteine residues which have been shown to be involved in disulfide bonds. The main structure is a two-stranded beta-sheet followed by a loop to a C-terminal short two-stranded sheet. Subdomains between the conserved cysteines strongly vary in length. These consensus patterns are used to identify members of this family: C-x-C-x(5)-G-x(2)-C and C-x-C-x(s)-[GP]-[FYW]-x(4,8)-C.

Zinc Finger, C2H2 Type (Zincfing C2H2; Pfam Accession No. PF00096). SEQ ID NO: 521 corresponds to polynucleotides encoding members of the C2H2 type zinc finger protein family, which contain zinc finger domains that facilitate nucleic acid binding (Klug et al., Trends Biochem. Sci. (1987) 12:464; Evans et al., Cell (1988) 52:1; Payre et al., FEBS Lett. (1988) 234:245; Miller et al., EMBO J. (1985) 4:1609; and Berg, Proc. Natl. Acad. Sci. USA (1988) 85:99). In addition to the conserved zinc ligand residues, a number of other positions are also important for the structural integrity of the C2H2 zinc fingers (Rosenfeld et al., J. Biomol. Struct. Dyn. (1993) 11:557). The best conserved position, which is generally an aromatic or aliphatic residue, is located four residues after the second cysteine. The consensus pattern for C2H2 zinc fingers is: C-x(2,4)-C-x(3)-[LIVMFYWC]-x(8)-H-x(3,5)-H. The two C's and two H's are zinc ligands.

PDZ Domain (PDZ; Pfam Accession No. PF00595.) SEQ ID NOS: 527, 1523, and 1551 correspond to genes comprising a PDZ domain (also known as DHR or GLGF domain). PDZ domains comprise 80-100 residue repeats, several of which interact with the C-terminal tetrapeptide motifs X-Ser/Thr-X-Val-COO— of ion channels and/or receptors, and are found in mammalian proteins as well as in bacteria, yeast, and plants (Pontig et al. Protein Sci (1997) 6(2):464-8). Proteins comprising one or more PDZ domains are found in diverse membrane-associated proteins, including members of the MAGUK family of guanylate kinase homologues, several protein phosphatases and kinases, neuronal nitric oxide synthase, and several dystrophin-associated proteins, collectively known as syntrophins (Ponting et al. Bioessays (1997) 19(6):469-79). Many PDZ domain-containing proteins are localised to highly specialised submembranous sites, suggesting their participation in cellular junction formation, receptor or channel clustering, and intracellular signalling events. For example, PDZ domains of several MAGUKs interact with the C-terminal polypeptides of a subset of NMDA receptor subunits and/or with Shaker-type K+ channels. Other PDZ domains have been shown to bind similar ligands of other transmembrane receptors. In cell junction-associated proteins,the PDZ mediates the clustering of membrane ion channels by binding to their C-terminus. The X-ray crystallographic structure of some proteins comrpising PDZ domains have been solved (see, e.g., Doyle et al. Cell (1996) 85(7):1067-76).

Zinc knuckle, CCHC type (Zf-CCHC; Pfam Accession No. PF00098). SEQ ID NOS: 543 and 1069 correspond to a gene encoding a member of the family of CCHC zinc fingers. Because the prototype CCHC type zinc finger structure is from an HIV protein, this domain is also referred to as a retrovrial-type zinc finger domain. The family also contains proteins involved in eukaryotic gene regulation, such as C. elegans GLH-1. The structure is an 18-residue zinc finger; no examples of indels in the alignment. The motif that defines a CCHC type zinc finger domain is: C-X2-C-X4-H-X4-C (Summers J Cell Biochem 1991 January;45(1):41-8). The domain is found in, for example, HIV-1 nucleocapsid protein, Moloney murine leukemia virus nucleocapsid protine NCp10 (De Rocquigny et al. Nucleic Acids Res. (1993) 21:823-9), and myelin transcription factor 1 (Myt1) (Kim et al. J. Neurosci. Res. (1997) 50:272-90).

RNA Recognition Motif (rrm; Pfam Accession No. PF00076). SEQ ID NOS: 514 and 910 correspond to sequence encoding an RNA recognition motif, also known as an RRM, RBD, or RNP domain. This domain, which is about 90 amino acids long, is contained in eukaryotic proteins that bind single-stranded RNA (Bandziulis et al. Genes Dev. (1989) 3:431-437; Dreyfuss et al. Trends Biochem. Sci. (1988) 13:86-91). Two regions within the RNA-binding domain are highly conserved: the first is a hydrophobic segment of six residues (which is called the RNP-2 motif), the second is an octapeptide motif (which is called RNP-1 or RNP-CS). The consensus pattern is: [RK]-G-{EDRKHPCG}-[AGSCI]-[FY]-[LIVA]-x-[FYLM].

Metallothioneins (metalthio; Pfam Accession No. PF00131). SEQ ID NO: 335 corresponds to a polynucleotide encoding a member of the metallothionein (MT) protein family (Hamer Annu. Rev. Biochem. (1986) 55:913-951; and Kagi et al. Biochemistry (1988) 27:8509-8515), small proteins which bind heavy metals such as zinc, copper, cadmium, nickel, etc., through clusters of thiolate bonds. MT's occur throughout the animal kingdom and are also found in higher plants, fungi and some prokaryotes. On the basis of structural relationships MT's have been subdivided into three classes. Class I includes mammalian MT's as well as MT's from crustacean and molluscs, but with clearly related primary structure. Class II groups together MT's from various species such as sea urchins, fungi, insects and cyanobacteria which display none or only very distant correspondence to class I MT's. Class III MT's are atypical polypeptides containing gamma-glutamylcysteinyl units. The consensus pattern for this protein family is: C-x-C-[GSTAP]-x(2)-C-x-C-x(2)-C-x-C-x(2)-C-x-K.

Trypsin (trypsin; Pfam Accession No. PF00089). SEQ ID NOS: 422 and 1558 correspond to a novel serine protease of the trypsin family. The catalytic activity of the serine proteases from the trypsin family is provided by a charge relay system involving an aspartic acid residue hydrogen-bonded to a histidine, which itself is hydrogen-bonded to a serine. The sequences in the vicinity of the active site serine and histidine residues are well conserved in this family of proteases (Brenner S., Nature (1988) 334:528). The consensus patterns for this trypsin protein family are: 1) [LIVM]-[ST]-A-[STAG]-H-C, where H is the active site residue; and 2) [DNSTAGC]-[GSTAPIMVQH]-x(2)-G-[DE]-S-G-[GS]-[SAPHV]-[LPVMFYWH]-[LIVMFYSTANQH], where S is the active site residue. All sequences known to belong to this family are detected by the above consensus sequences, except for 18 different proteases which have lost the first conserved glycine. If a protein includes both the serine and the histidine active site signatures, the probability of it being a trypsin family serine protease is 100%.

HSP70 protein (HSP70; Pfam Accession No. PF00012) SEQ ID NOS: 952 and 1482 correspond to members of the family of ATP-binding heat shock proteins having an average molecular weight of 70 kD (Pelham, Cell (1986) 46:959-961; Pelham, Nature (1988) 332:776-77; Craig, BioEssays (1989) 11:48-52). In most species, there are many proteins that belong to the hsp70 family, some of which are expressed under unstressed conditions. Hsp70 proteins can be found in different cellular compartments, including nuclear, cytosolic, mitochondrial, endoplasmic reticulum, etc. A variety of functions have been postulated for hsp70 proteins. Some play an important role in the transport of proteins across membranes (Deshaies et al., Trends Biochem. Sci. (1988) 13:384-388), while others are involved in protein folding and in the assembly/disassembly of protein complexes (Craig and Gross, Trends Biochem. Sci. (1991) 16:135-140).

There are three signature patterns for the hsp70 family of proteins. The first is centered on a conserved pentapeptide found in the N-terminal section of these proteins and the two others on conserved regions located in the central part of the sequence. The consensus patterns are: 1) [IV]-D-L-G-T-[ST]-x-[SC]; 2) [LIVMF]-[LIVMFY]-[DN]-[LIVMFS]-G-[GSH]-[GS]-[AST]-x(3)-[ST]-[LIVM]-[LIVMFC]; and 3) [LIVMY]-x-[LIVMF]-x-G-G-x-[ST]-x-[LIVM]-P-x-[LIVM]-x-[DEQKRSTA].

WD Domain (WD40), G-Beta Repeats (WD domain; Pfam Accession No. PF00400). SEQ ID NOS: 1510 and 1536 represent members of the WD domain/G-beta repeat family. Beta-transducin (G-beta) is one of the three subunits (alpha, beta, and gamma) of the guanine nucleotide-binding proteins (G proteins) which act as intermediaries in the transduction of signals generated by transmembrane receptors (Gilman, Annu. Rev. Biochem. (1987) 56:615). The alpha subunit binds to and hydrolyzes GTP; the beta and gamma subunits are required for the replacement of GDP by GTP as well as for membrane anchoring and receptor recognition. In higher eukaryotes, G-beta exists as a small multigene family of highly conserved proteins of about 340 amino acid residues. Structurally, G-beta has eight tandem repeats of about 40 residues, each containing a central Trp-Asp motif (this type of repeat is sometimes called a WD-40 repeat). The consensus pattern for the WD domain/G-Beta repeat family is: [LIVMSTAC]-[LIVMFYWSTAGC]-[LIMSTAG]-[LIVMSTAGC]-x(2)-[DN]-x(2)-[LIVMWSTAC]-x-[LIVMFSTAG]-W-[DEN]-[LIVMFSTAGCN].

Protein Kinase (protkinase; Pfam Accession No. PF00069). SEQ ID NO: 1540 represents a protein kinase. Protein kinases catalyze phosphorylation of proteins in a variety of pathways, and are implicated in cancer. Eukaryotic protein kinases (Hanks S. K., et al., FASEB J. (1995) 9:576; Hunter T., Meth. Enzymol. (1991) 200:3; Hanks S. K., et al., Meth. Enzymol. (1991) 200:38; Hanks S. K., Curr. Opin. Struct. Biol. (1991) 1:369; Hanks S. K., et al, Science (1988) 241:42) are enzymes that belong to a very extensive family of proteins which share a conserved catalytic core common to both serine/threonine and tyrosine protein kinases. There are a number of conserved regions in the catalytic domain of protein kinases. The first region, which is located in the N-terminal extremity of the catalytic domain, is a glycine-rich stretch of residues in the vicinity of a lysine residue, which has been shown to be involved in ATP binding. The second region, which is located in the central part of the catalytic domain, contains a conserved aspartic acid residue which is important for the catalytic activity of the enzyme (Knighton D. R., et al., Science (1991) 253:407). The protein kinase profile includes two signature patterns for this second region: one specific for serine/threonine kinases and the other for tyrosine kinases. A third profile is based on the alignment in (Hanks S. K., et al., FASEB J. (1995) 9:576) and covers the entire catalytic domain.

The consensus patterns are as follows: 1) [LIV]-G-{P}-G-{P}-[FYWMGSTNH]-[SGA]-{PW}-[LIVCAT]-{PD}-x-[GSTACLIVMFY]-x(5,18)-[LIVMFYWCSTAR]-[AIVP]-[LIVMFAGCKR]-K, where K binds ATP; 2) [LIVMFYC]-x-[HY]-x-D-[LIVMFY]-K-x(2)-N-[LIVMFYCT](3), where D is an active site residue; and 3) [LIVMFYC]-x-[HY]-x-D-[LIVMFY]-[RSTAC]-x(2)-N-[LIVMFYC], where D is an active site residue.

If a protein analyzed includes the two of the above protein kinase signatures, the probability of it being a protein kinase is close to 100%. Eukaryotic-type protein kinases have also been found in prokaryotes such as Myxococcus xanthus (Munoz-Dorado J., et al., Cell (1991) 67:995) and Yersinia pseudotuberculosis. The patterns shown above has been updated since their publication in (Bairoch A., et al., Nature (1988) 331:22).

C2 domain (C2; Pfam Accession No. PF00168). SEQ ID NO: 1550 corresponds to a C2 domain, which is involved in calcium-dependent phospholipid binding (Davletov J. Biol. Chem. (1993) 268:26386-26390) or, in proteins that do not bind calcium, the domain may facilitate binding to inositol-1,3,4,5-tetraphosphate (Fukuda et al. J. Biol. Chem. (1994) 269:29206-29211; Sutton et al. Cell (1995) 80:929-938). The consensus sequence is: [ACG]-x(2)-L-x(2,3)-D-x(1,2)-[NGSTLIF]-[GTMR]-x-[STAP]-D-[PA]-[FY].

Myosin head (motor domain) (myosin head; Pfam Accession No. PF00063). SEQ ID NOS: 189, 1548, and 1557 correspond to a myosin head domain, a glycine-rich region that typically forms a flexible loop between a beta-strand and an alpha-helix. This loop interacts with one of the phosphate groups of ATP or GTP in binding of a protein to the nucleotide. The myosin head sequence motif is generally referred to as the “A” consensus sequence (Walker et al., EMBO J. (1982) 1:945-951) or the “P-loop” (Saraste et al., Trends Biochem. Sci. (1990) 15:430-434). The consensus sequence is: [AG]-x(4)-G-K-[ST].

Sugar (and other) transporter (sugar tr; Pfam Accession No. PF00083). SEQ ID NOS: 334, 1244, and 1512 represent members of the sugar (and other) transporter family. In mammalian cells the uptake of glucose is mediated by a family of closely related transport proteins which are called the glucose transporters (Silverman, Annu. Rev. Biochem. (1991) 60:757-794; Gould and Bell, Trends Biochem. Sci. (1990) 15:18-23; Baldwin, Biochim. Biophys. Acta (1993) 1154:17-49). At least seven of these transporters are currently known to exist and in Humans are encoded by the GLUT1 to GLUT7 genes. These integral membrane proteins are predicted to comprise twelve membrane spanning domains and show sequence similarities with a number of other sugar or metabolite transport proteins (Maiden et al., Nature (1987) 325:641-643; Henderson, Curr. Opin. Struct. Biol. (1991) 1:590-601).

Two patterns have been developed to detect this family of proteins. The first pattern is based on the G-R-[KR] motif; but because this motif is too short to be specific to this family of proteins, a second pattern has been derived from a larger region centered on the second copy of this motif. The second pattern is based on a number of conserved residues which are located at the end of the fourth transmembrane segment and in the short loop region between the fourth and fifth segments. The two consensus sequences are: 1) [LIVMSTAG]-[LIVMFSAG]-x(2)-[LIVMSA]-[DE]-x-[LIVMFYWA]-G-R-[RK]-x(4,6)-[GSTA]; and 2) [LIVMF]-x-G-[LIVMFA]-x(2)-G-x(8)-[LIFY]-x(2)-[EQ]-x(6)-[RK].

HSP 90 protein (Pfam Accession No. PF00183). SEQ ID NO: 1538 represents a polypeptide having a consensus sequence of a Hsp90 protein family member. Hsp90 proteins are proteins of an average molecular weight of approximately 90 kDa that respond to heat shock or other environmental stress by the induction of the synthesis of proteins collectively known as heat-shock proteins (hsp) (Lindquist et al. Annu. Rev. Genet. 22:631-677 (1988). Proteins known to belong to this family include vertebrate hsp 90-alpha (hsp 86) and hsp 90-beta (hsp 84); Drosophila hsp 82 (hsp 83); and the endoplasmic reticulum protein ‘endoplasmin’ (also known as Erp99 in mouse, GRP94 in hamster, and hsp 108 in chicken). Hsp90 proteins have been found associated with steroid hormone receptors, with tyrosine kinase oncogene products of several retroviruses, with eIF2alpha kinase, and with actin and tubulin. Without being held to theory, Hsp90 proteins are probable chaperonins that possess ATPase activity (Nadeau et al. J. Biol. Chem. 268:1479-1487 (1993); Jakob et al. Trends Biochem Sci 19:205-211 (1994). Hsp90 family proteins have the following signature pattern, which represents a highly conserved region found in the N-terminal part of these proteins: Y-x-[NQH]-K-[DE]-[IVA]-F-[LM]-R-[ED]

KOW motif (Ribosomal protein L24 signature; Pfam Accession No. PF00467). SEQ ID NO: 1553 represents a polypeptide having a KOW motif such as that found in the ribosomal protein L24, one of the proteins from the large ribosomal subunit. L24 belongs to a family of ribosomal proteins. In their mature form, these proteins have 103 to 150 amino-acid residues. As a signature pattern, The consensus sequence is based on a conserved stretch of 20 residues in the N-terminal section: [GDEN]-D-x-[IV]-x-[IV]-[LIVMA]-x-G-x(2)-[KRA]-[GNQ]-x(2,3)-[GA]-x-[IV].

TPR Domain (Pfam Accession No. PF00515). SEQ ID NO: 1532 represents a polypeptide having at least one or more tetratricopeptide repeat (TPR) domains. The TPR is a degenerate 34 amino acid sequence identified in a wide variety of proteins, present in tandem arrays of 3-16 motifs, which form scaffolds to mediate protein-protein interactions and often the assembly of multiprotein complexes. TPR-containing proteins include the anaphase promoting complex (APC) subunits cdc16, cdc23 and cdc27, the NADPH oxidase subunit p67 phox, hsp90-binding immunophilins, transcription factors, the PKR protein kinase inhibitor, and peroxisomal and mitochondrial import proteins (see, e.g., Das et al. EMBO J;17(5):1192-9 (1998); and Lamb Trends Biochem Sci 20:257-259 (1995).

tRNA synthetase class II core domain (G, H, P, S and T) (Pfam Accession No. PF00587). SEQ ID NO: 1481 represents a polypeptide having a tRNA synthetase class II core domain. Aminoacyl-tRNA synthetases (EC 6.1.1.-) (Schimmel Annu. Rev. Biochem. 56:125-158(1987)) are a group of enzymes which activate amino acids and transfer them to specific tRNA molecules as the first step in protein biosynthesis. In prokaryotic organisms there are at least twenty different types of aminoacyl-tRNA synthetases, one for each different amino acid. In eukaryotes there are generally two aminoacyl-tRNA synthetases for each different amino acid: one cytosolic form and a mitochondrial form. While all these enzymes have a common function, they are widely diverse in terms of subunit size and of quaternary structure.

The synthetases specific for alanine, asparagine, aspartic acid, glycine, histidine, lysine, phenylalanine, proline, serine, and threonine are referred to as class-II synthetases and probably have a common folding pattern in their catalytic domain for the binding of ATP and amino acid which is different to the Rossmann fold observed for the class I synthetases. Class-II tRNA synthetases do not share a high degree of similarity, however at least three conserved regions are present (Delarue et al. BioEssays 15:675-687(1993); Cusack et al. Nucleic Acids Res. 19:3489-3498(1991); Leveque et al. Nucleic Acids Res. 18:305-312(1990)]. The consensus sequences are derived from these regions: [FYH]-R-x-[DE]-x(4,12)-[RH]-x(3)-F-x(3)-[DE] (found in the majority of class-II tRNA synthetases with the exception of those specific for alanine, glycine as well as bacterial histidine); and [GSTALVF]-{DENQHRKP}-[GSTA]-[LIVMF]-[DE]-R-[LIVMF]-x-[LIVMSTAG]-[LIVMFY] (found in the majority of class-II tRNA synthetases with the exception of those specific for serine and proline).

IQ calmodulin-binding motif (Pfam Accession No. PF00612). SEQ ID NOS: 189 and 1548 represent polypeptides having an IQ calmodulin-binding motif. The IQ motif is an extremely basic unit of about 23 amino acids, whose conserved core usually fits the consensus A-x(3)-I-Q-x(2)-F-R-x(4)-K-K. The IQ motif, which can be present in one or more copies, serves as a binding site for different EF-hand proteins including the essential and regulatory myosin light chains, calmodulin (CaM), and CaM-like proteins (see, e.g., Cheney et al. Curr. Opin. Cell Biol. 4:27-35(1992); and Rhoads et al. FASEB J. 11:331-340(1997)). Many IQ motis are protein kinase C (PKC) phosphorylation sites (Baudier et al. J. Biol. Chem. 266:229-237(1991); and Chen et al. Biochemistry 32:1032-1039(1993)). Resolution of the 3D structure of scallop myosin has shown that the IQ motif forms a basic amphipathic helix (Xie et al. Nature 368:306-312(1994)). Exemplary proteins containing an IQ motif include neuromodulin (GAP-43), neurogranin (NG/p 17), sperm surface protein Sp17, and Ras GTPase-activating-like protein IQGAP1. IQGAP1 contains 4 IQ motifs.

Phophotyrosine interaction domain (PTB/PID) (Pfam Accession No. PF00640). SEQ ID NO: 1523 represents a polypeptide having a phosphotyrosine interaction domain (PID or PI domain). PID is the second phosphotyrosine-binding domain found in the transforming protein Shc (Kavanaugh et al. Science 266:1862-1865(1994); Blaikie et al. J. Biol. Chem. 269:32031-32034(1994); and Bork et al. Cell 80:693-694(1995)). Shc couples activated growth factor receptors to a signaling pathway that regulates the proliferation of mammalian cells and it might participate in the transforming activity of oncogenic tyrosine kinases. The PID of Shc specifically binds to the Asn-Pro-Xaa-Tyr(P) motif found in many tyrosine-phosphorylated proteins including growth factor receptors. PID has also been found in, for example, human Shc-related protein Sck, mammalian protein X11 which is expressed prominently in the nervous system, rat FE65, a transcription-factor activator expressed preferentially in liver, mammalian regulator of G-protein signalling 12 (RGS12), and N-terminal insulinase-type domain. PID has an average length of about 160 amino acids. It is probably a globular domain with an antiparallel beta sheet. The function of this domain might be phosphotyrosine-binding. It is at least expected to be involved in regulatory protein/protein-binding (Bork et al. Cell 80:693-694(1995)).

Syntaxin (Pfam Accession No. PF00804). SEQ ID NOS: 1039 and 1496 represent polypeptides having sequence similarity to syntaxin protein family. Members of the syntaxin family of proteins include, for example, epimorphin (or syntaxin 2), a mammalian mesenchymal protein which plays an essential role in epithelial morphogenesis; syntaxin 1A, syntaxin 1B, and syntaxin 4, which are synaptic proteins involved in docking of synaptic vesicles at presynaptic active zones; syntaxin 3; syntaxin 5, which mediates endoplasmic reticulum to golgi transport; and syntaxin 6, which is involved in intracellular vesicle trafficking (Bennett et al. Cell 74:863-873(1993); Spring et al. Trends Biochem. Sci. 18:124-125(1993); Pelham et al. Cell 73:425-426(1993)). The syntaxin family of proteins each range in size from 30 Kd to 40 Kd; have a C-terminal extremity which is highly hydrophobic and is involved in anchoring the protein to the membrane; a central, well conserved region, which may be present in a coiled-coil conformation. The pattern specific for this family is based on the most conserved region of the coiled coil domain: [RQ]-x(3)-[LIVMA]-x(2)-[LIVM]-[ESH]-x(2)-[LIVMT]-x-[DEVM]-[LIVM]-x(2)-[LIVM]-[FS]-x(2)-[LIVM]-x(3)-[LIVT]-x(2)-Q-[GADEQ]-x(2)-[LFVM]-[DNQT]-x-[LIVMF]-[DESV]-x(2)-[LIVM].

Ribosomal L10 (Pfam Accession No. PF00826). SEQ ID NOS: 759, 1207, and 1566 represents a polypeptide having sequence similarity to the ribosomal L10 protein family (see, e.g., Chan et al. Biochem. Biophys. Res. Commun. 225:952-956(1996)). The members of this family generally have 174 to 232 amino-acid residues and contain the following signature pattern (based on a conserved region located in the central section of the proten): A-D-R-x(3)-G-M-R-x-[SAP]-[FYW]-G-[KRVT]-[PA]-x-[GS]-x(2)-A-[KRLV]-[LIV]

GTP1/OBG Family (Pfam Accession No. PF01018). SEQ ID NO: 126, 721, and 1518 represent polypeptides that have similarities to the members of the GTP1/OBG family, a widespread family of GTP-binding proteins (Sazuka et al. Biochem. Biophys. Res. Commun. 189:363-370(1992); Hudson et al. Gene 125:191-193(1993)). This family includes, for example, protein DRG (found in mouse, human, and xenopus), fission yeast protein gtp1, and Bacillus subtilis protein obg (which binds GTP). Family members are generally about 40 to 48 Kd and contain the five small sequence elements characteristic of GTP-binding proteins (Bourne et al. Nature 349:117-127(1991)). The signature pattern corresponds to the ATP/GTP B motif (also called G-3 in GTP-binding proteins): D-[LIVM]-P-G-[LIVM](2)-[DEY]-[GN]-A-x(2)-G-x-G

KRAB box (Pfam Accession No. PF01352). SEQ ID NOS: 1556 and 349 represent polypeptides having a Krueppel-associated box (KRAB). A KRAB box is a domain of around 75 amino acids that is found in the N-terminal part of about one third of eukaryotic Krueppel-type C2H2 zinc finger proteins (ZFPs). It is enriched in charged amino acids and can be divided into subregions A and B, which are predicted to fold into two amphipathic alpha-helices. The KRAB A and B boxes can be separated by variable spacer segments and many KRAB proteins contain only the A box.

The KRAB domain functions as a transcriptional repressor when tethered to the template DNA by a DNA-binding domain. A sequence of 45 amino acids in the KRAB A subdomain has been shown to be necessary and sufficient for transcriptional repression. The B box does not repress by itself but does potentiate the repression exerted by the KRAB A subdomain. Gene silencing requires the binding of the KRAB domain to the RING-B box-coiled coil (RBCC) domain of the KAP-1/TIF1-beta corepressor. As KAP-1 binds to the heterochromatin proteins HP1, it has been proposed that the KRAB-ZFP-bound target gene could be silenced following recruitment to heterochromatin.

KRAB-ZFPs constitute one of the single largest class of transcription factors within the human genome, and appear to play important roles during cell differentiation and development. The KRAB domain is generally encoded by two exons. The regions coded by the two exons are known as KRAB-A and KRAB-B.

Small ribonucleoprotein (Sm protein; Pfam Accession No. PF01423). SEQ ID NO: 1495 represents a polypeptide having sequence similarity to small ribonucleoprotein (Sm protein). The U1, U2, U4/U6, and U5 small nuclear ribonucleoprotein particles (snRNPs) involved in pre-mRNA splicing contain seven Sm proteins (B/B′, D1, D2, D3, E, F and G) in common, which assemble around the Sm site present in four of the major spliceosomal small nuclear RNAs (Hermann et al. EMBO J. 14: 2076-2088(1995)). The Sm proteins are essential for pre-mRNA splicing and are implicated in the formation of stable, biologically active snRNP structures.

Cation efflux family (Pfam Accession No. PF01545. SEQ ID NO: 563, 766, and 1545 represent polypeptides having sequence similarity to members of the cation efflux family. Members of this family are integral membrane proteins which increase tolerance to divalent metal ions such as cadmium, zinc, and cobalt. These proteins are efflux pumps that remove these ions from cells (Xiong et al. J. Bacteriol. 180: 4024-4029(1998); Kunito et al. Biosci. Biotechnol. Biochem. 60: 699-704(1996)).

FG-GAP repeat (Pfam Accession No. PF01839). SEQ ID NO: 1486 represents a polypeptide having an FG-GAP repeat. This family contains the extracellular repeat that is found in up to seven copies in alpha integrins. This repeat has been predicted to fold into a beta propeller structure (Springer et al. Proc Natl Acad Sci U S A 1997;94:65-72). The repeat is called the FG-GAP repeat after two conserved motifs in the repeat (Spring, ibid). The FG-GAP repeats are found in the N terminus of integrin alpha chains, a region that has been shown to be important for ligand binding (Loftus et al. J Biol Chem 1994;269:25235-25238). A putative Ca2+ binding motif is found in some of the repeats.

Dilute (DIL) domain (Pfam Accession No. PF01843). SEQ ID NO: 1548 represents a polypeptide having a DIL domain. Dilute encodes a type of myosin heavy chain, with a tail, or C-terminal, region that has elements of both type II (alpha-helical coiled-coil) and type I (non-coiled-coil) myosin heavy chains. The DIL non alpha-helical domain is found in dilute myosin heavy chain proteins and other myosins. In mouse the dilute protein plays a role in the elaboration, maintenance, or function of cellular processes of melanocytes and neurons (Mercer et al. Nature 349(6311): 709-713(1991)). The DIL-containing MYO2 protein of Saccharomyces cerevisiae is implicated in vectorial vesicle transport and is homologous to the dilute protein over practically its entire length (Johnston et al. J. Cell Biol. 113(3): 539-551(1991).

Ubiquinol-cytochrome C reductase complex 14 kD subunit (Pfam Accession No. PF022771). SEQ ID NOS: 419 and 1519 represent a polypeptide having sequence similarity to Ubiquinol-cytochrome C reductase complex 14 kD subunit. The cytochrome bd type terminal oxidases catalyse quinol dependent, Na+ independent oxygen uptake. Members of this family are integral membrane proteins and contain a protoheame IX center B558. Cytochrome bd plays a role in microaerobic nitrogen fixation in the enteric bacterium Klebsiella pneumoniae, where it is expressed under all conditions that permit diazotrophy. The 14 kD (or VI) subunit of the complex is not directly involved in electron transfer, but has a role in assembly of the complex (Braun et al Plant Physiol. 107(4): 1217-1223(1995)).

Cytidylytransferase (Pfam Accession No. PF02348). SEQ ID NOS: 109, 394, 569, 1128, and 1535 represent polypeptides having sequence similarity to the cytidylytransferase family of proteins, which are involved in lipopolysaccharide biosynthesis. This family consists of two main cytidylyltransferase activities: 1) 3-deoxy-manno-octulosonate cytidylyltransferase (Strohmaier et al. J Bacteriol 1995;177:4488-4500.) EC:2.7.7.38 catalysing the reaction:- CTP+3-deoxy-D-manno-octulosonate<=>diphosphate+CMP-3-deoxy-D-manno-octulosonate; and 2) acylneuraminate cytidylyltransferase EC:2.7.7.43 (Munster et al. Proc Natl Acad Sci U S A 1998;95:9140-9145; Tullius et al. J Biol Chem 1996;271:15373-15380) catalysing the reaction:- CTP+N-acylneuraminate<=>diphosphate+CMP-N-acylneuraminate N-acetylneuraminic acid cytidylyltransferase (EC 2.7.7.43) (CMP-NeuAc synthetase) catalyzes the reaction of CTP and NeuAc to form CMP-NeuAc, which is the nucleotide sugar donor used by sialyltransferases. The outer membrane lipooligosaccharides of some microorganisms contain terminal sialic acid attached to N-acetyllactosamine; thus this modification may be important in pathogenesis.

Laminin G domain (Pfam Accession No. PF00054). SEQ ID NO: 1521 represents a polypeptide having a laminin G domain, a homology domain first described in the long arm globular domain of laminin (Vuolteenaho et al. J. Biol. Chem. 265: 15611-15616(1990)). Similar sequences also occurs in a large number of extracellular proteins. Laminin binds to heparin (Yurchenco et al. J. Biol. Chem. 268(11): 8356-8365(1993); Sung et al. Eur. J. Biochem. 250(1): 138-143(1997)). The structure of the laminin-G domain has been predicted to resemble that of pentraxin (Beckmann et al. J. Mol. Biol. 275: 725-730(1998)). Exemplary proteins having laminin-G domains include laminin, merosin, agrin, neurexins, vitamin K dependent protein S, and sex steroid binding protein SBP/SHBG.

4Fe-4S iron sulfur cluster binding proteins, NifH/frxC family (Pfam Accession No. PF00142). SEQ ID NO: 1100 represents a polypeptide having sequence similarity to the 4Fe-4S iron sulfur cluster binding proteins, NifH/frxC family. Nitrogen fixing bacteria possess a nitrogenase enzyme complex (EC 1.18.6.1) that comprises 2 components, which catalyse the reduction of molecular nitrogen to ammonia: component I (nitrogenase MoFe protein or dinitrogenase) contains 2 molecules each of 2 non-identical subunits; component II (nitrogenase Fe protein or dinitrogenase reductase) is a homodimer, the monomer being coded for by the nifH gene. Component II has 2 ATP-binding domains and one 4Fe-4S cluster per homodimer: it supplies energy by ATP hydrolysis, and transfers electrons from reduced ferredoxin or flavodoxin to component I for the reduction of molecular nitrogen to ammonia. There are a number of conserved regions in the sequence of these proteins: in the N-terminal section there is an ATP-binding site motif ‘A’ (P-loop) and in the central section there are two conserved cysteines which have been shown, in nifH, to be the ligands of the 4Fe-4S cluster.

Cyclophilin-type peptidyl-prolyl cis-trans isomerase (Pfam Accession No. PF00160). SEQ ID NOS: 134, 259, 363, 1101, and 1267 represent polypeptides having sqeuence simlarity to the cyclophilin-type peptidyl-prolyl cis-trans isomerase protein family. Cyclophilin (Stamnes et al. Trends Cell Biol. 2: 272-276(1992)) is the major high-affinity binding protein in vertebrates for the immunosuppressive drug cyclosporin A (CSA), but is also found in other organisms. It exhibits a peptidyl-prolyl cis-trans isomerase activity (EC 5.2.1.8) (PPIase or rotamase). PPIase is an enzyme that accelerates protein folding by catalyzing the cis-trans isomerization of proline imidic peptide bonds in oligopeptides (Fischer et al. Biochemistry 29: 2205-2212(1990)). It is probable that CSA mediates some of its effects via an inhibitory action on PPIase. Cyclophilin A is a cytosolic and highly abundant protein. The protein belongs to a family of isozymes, including cyclophilins B and C, and natural killer cell cyclophilin-related protein (Trandinh et al. FASEB J. 6: 3410-3420(1992); Galat Eur. J. Biochem. 216: 689-707(1993); Hacker et al. Mol. Microbiol. 10: 445-456(1993)). Major isoforms have been found throughout the cell, including the ER, and some are even secreted. The sequences of the different forms of cyciophilin-type PPlases are well conserved.

Ubiquitin-conjugating enyme (Pfam Accession No. PF00179). SEQ ID NO: 7 represents a polypeptide having sequence similarity to ubiquitin-conjugating enyme. Ubiquitin-conjugating enzymes (EC 6.3.2.19) (UBC or E2 enzymes) (Jentsch et al. Biochim. Biophys. Acta 1089: 127-139(1991); Jentsch et al. Trends Biochem. Sci. 15: 195-198(1990); Hershko et al. Trends Biochem. Sci. 16: 265-268(1991)). catalyze the covalent attachment of ubiquitin to target proteins. An activated ubiquitin moiety is transferred from an ubiquitin-activating enzyme (E1) to E2 which later ligates ubiquitin directly to substrate proteins with or without the assistance of ‘N-end’ recognizing proteins (E3). A cysteine residue is required for ubiquitin-thiolester formation. There is a single conserved cysteine in UBC's and the region around that residue is conserved in the sequence of known UBC isozymes. There are, however, exceptions, the breast cancer gene product TSG101 is one of several UBC homologues that lacks this active site cysteine (Ponting et al. J. Mol. Med. 75: 467-469(1997); Koonin et al. Nat. Genet. 16: 330-331(1997)). In most species there are many forms of UBC which are implicated in diverse cellular functions.

NADH-ubiquinone/plastoquinone oxidoreductase chain 6 (Pfam Accession No. PF00499). SEQ ID NOS: 507 and 1002 represent polypeptides having sequence similarity with NADH-ubiquinone/plastoquinone oxidoreductase chain 6 protein family. In bacteria, the proton-translocating NADH-quinone oxidoreductase (NDH-1) is composed of 14 different subunits. The chain belonging to this family is a subunit that constitutes the membrane sector of the complex. It reduces ubiquinone to ubiquinol utilising NADH. In plants, chloroplastic NADH-plastoquinone oxidoreductase reduces plastoquinone to plastoquinol. Mitochondrial NADH-ubiquinone oxidoreductase from a variety of sources reduces ubiquinone to ubiquinol.

AP endonucleases family 1 (Pfam Accession No. PF00895). SEQ ID NO: 10 and 1107 represent polypeptides having sequence similarity to members of the AP endonucleases family 1. DNA damaging agents such as the antitumor drugs bleomycin and neocarzinostatin or those that generate oxygen radicals produce a variety of lesions in DNA. Amongst these is base-loss which forms apurinic/apyrimidinic (AP) sites or strand breaks with atypical 3′termini. DNA repair at the AP sites is initiated by specific endonuclease cleavage of the phosphodiester backbone. Such endonucleases are also generally capable of removing blocking groups from the 3′terminus of DNA strand breaks.

AP endonucleases can be classified into two families on the basis of sequence similarity. This family contains members of AP endonuclease family 1. Except for Rrp1 and arp, these enzymes are proteins of about 300 amino-acid residues. Rrp1 and arp both contain additional and unrelated sequences in their N-terminal section (about 400 residues for Rrp1 and 270 for arp). The proteins contain glutamate which has been shown (Mol et al. Nature 374: 381-386(1995), in the Escherichia coli enzyme to bind a divalent met al ion such as magnesium or manganese.

Late Expression Factor 2 (lef-2; Pfam Accession No. PF03041). SEQ ID NO: 405 represents a polynucleotide encoding a member of the late expression factor 2 family of polypeptides. The lef-2 gene from baculovirus is required for expression of late genes and has been shown to be specifically required for expression from the vp39 and polh promoters (Passarelli and Miller, J. Virol. (1993) April;67(4):2149-58). Lef-2 has been found in both Lymantria dispar multicapsid nuclear polyhedrosis virus (LdMNPV) and Orgyia pseudotsugata multicapsid polyhedrosis virus (OpMNPV).

Papillomavirus E5 (Papilloma E5; Pfam Accession No. PF03025). SEQ ID NO: 1051 corresponds to a polynucleotide encoding a member of the papillomavirus E5 family of polypeptides. The E5 protein from papillomaviruses is about 80 amino acids long and contains three regions that have been predicted to be transmembrane alpha helices.

Male sterility protein (Sterile; Pfam Accession No. PF03015). SEQ ID NO: 391 encodes a member of the male sterility protein family. This family represents the C-terminal region of the male sterility protein in a number of organisms. One member of this family, the Arabidopsis thaliana male sterility 2 (MS2) protein, is involved in male gametogenesis. The MS2 protein shows sequence similarity to reductases in elongation/condensation complexes, such as jojoba protein (also a member of this group), an acyl CoA reductase that converts wax fatty acids to fatty alcohols. The MS2 protein may be a fatty acyl reductase involved in the formation of pollen wall substances (Aarts et al., Plant. J. (1997) September;12(3):615-23).

Cytochrome C oxidase subunit II, transmembrane domain (COX2_TM; Pfam Accession No. PF02790). SEQ ID NO: 1183 corresponds to a gene comprising a cytochlrome C oxidase subunit II transmembrane domain (COX2_TM). Cytochrome C oxidase is an oligomeric enzymatic complex which is a component of the respiratory chain and is involved in the transfer of electrons from cytochrome C to oxygen (Capaldi et al., Biochim. Biophys. Acta (1983) 726:135-148; Garcia-Horsman et al., J. Bacteriol. (1994) 176:5587-5600). In eukaryotes this enzyme complex is located in the mitochondrial inner membrane; in aerobic prokaryotes it is found in the plasma membrane. The enzyme complex consists of 3-4 subunits (prokaryotes) to up to 13 polypeptides (mammals).

Subunit 2 of cytochrome C oxidase (COX2_TM) transfers the electrons from cytochrome C to the catalytic subunit 1. It contains two adjacent transmembrane regions in its N-terminus and the major part of the protein is exposed to the periplasmic or to the mitochondrial intermembrane space, respectively. COX2_TM provides the substrate-binding site and contains a copper center called Cu(A), probably the primary acceptor in cytochrome C oxidase. Several bacterial COX2_TM have a C-terminal extension that contains a covalently bound heme c. The consensus pattern is: V-x-H-x(33,40)-C-x(3)-C-x(3)-H-x(2)-M, where the two C's and two H's are copper ligands.

Uncharacterized ACR, YggU family COG1872 (DUF167; Pfam Accession No. PF02594). SEQ ID NOS: 46, 813, 935, and 1225 correspond to a polynucleotide encoding a member of the uncharacterized ACR, YggU family COG1872 of proteins of E. coli. This protein in E. coli is a hypothetical 10.5 kDa protein in the GSHB-ANSB intergenic region.

Phosducin (Phosducin; Pfam Accession No. PF02114). SEQ ID NOS: 267 and 771 correspond to sequence encoding a Phosducin motif The outer and inner segments of vertebrate rod photoreceptor cells contain phosducin, a soluble phosphoprotein that complexes with the beta/gamma-subunits of the GTP-binding protein, transducin (Lee et al., J. Biol. Chem. (1990) 265:15867-15873). Light-induced changes in cyclic nucleotide levels modulate the phosphorylation of phosducin by protein kinase A (Lee et al., J. Biol. Chem. (1990) 265:15867-15873). The protein is thought to participate in the regulation of visual phototransduction or in the integration of photo-receptor metabolism. Similar proteins have been isolated from the pineal gland (Abe et al., Gene (1990) 91:209-215): the 33 kDa proteins have the same sequences and the same phosphorylation site, suggesting that the functional role of the protein is the same in both retina and pineal gland.

The Phosducin motif is an 8-element fingerprint that provides a signature for phosducins. The fingerprint was derived from an initial alignment of 7 sequences where the motifs were drawn from conserved regions spanning virtually the full alignment length. The sequences of the 8 elements are as follows: (1) EEDFEGQASHTGPKGVINDW; (2) DSVAHSKKEILRQMSSPQSR; (3) SRKMSVQEYELIHKDKEDE; (4) CLRKYRRQCMQDMHQKLSF; (5) GPRYGFVYELESGEQFLETIEKE; (6) YEDGIKGCDALNSSLICLAAEY; (7) DRFSSDVLPTLLVYKGGELLSNF; and (8) EQLAEEFFTGDVESFLNEYG.

### Example 6

Detection of Differential Expression Using Arrays and Source of Patient Tissue Samples

mRNA isolated from samples of cancerous and normal breast, colon, and prostate tissue obtained from patients were analyzed to identify genes differentially expressed in cancerous and normal cells. Normal and cancerous tissues were collected from patients using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al (1997) J. Clin. Lab. Anal. 11:28-38; Emmert-Buck et al. (1996) Science 274:998-1001).

Table 10 (inserted prior to claims) provides information about each patient from which colon tissue samples were isolated, including: the Patient ID (“PT ID”) and Path ReportID (“Path ID”), which are numbers assigned to the patient and the pathology reports for identification purposes; the group (“Grp”) to which the patients have been assigned; the anatomical location of the tumor (“Anatom Loc”); the primary tumor size (“Size”); the primary tumor grade (“Grade”); the identification of the histopathological grade (“Histo Grade”); a description of local sites to which the tumor had invaded (“Local Invasion”); the presence of lymph node metastases (“Lymph Met”); the incidence of lymph node metastases (provided as a number of lymph nodes positive for metastasis over the number of lymph nodes examined) (“Lymph Met Incid”); the regional lymphnode grade (“Reg Lymph Grade”); the identification or detection of metastases to sites distant to the tumor and their location (“Dist Met & Loc”); the grade of distant metastasis (“Dist Met Grade”); and general comments about the patient or the tumor (“Comments”). Histophatology of all primary tumors incidated the tumor was adenocarcinmoa except for Patient ID Nos. 130 (for which no information was provided), 392 (in which greater than 50% of the cells were mucinous carcinoma), and 784 (adenosquamous carcinoma). Extranodal extensions were described in three patients, Patient ID Nos. 784, 789, and 791. Lymphovascular invasion was described in Patient ID Nos. 128, 278, 517, 534, 784, 786, 789, 791, 890, and 892. Crohn's-like infiltrates were described in seven patients, Patient ID Nos. 52, 264, 268, 392, 393, 784, and 791.

Table 11 below provides information about each patient from which the prostate tissue samples were isolated, including: 1) the “Patient ID”, which is a number assigned to the patient for identification purposes; 2) the “Tissue Type”; and 3) the “Gleason Grade” of the tumor. Histopathology of all primary tumors indicated the tumor was adenocarcinoma.

TABLE 11
Prostate patient data.

| | | Gleason |
|---|---|---|
| Patient ID | Tissue Type | Grade |
| 93 | Prostate Cancer | 3 + 4 |
| 94 | Prostate Cancer | 3 + 3 |
| 95 | Prostate Cancer | 3 + 3 |
| 96 | Prostate Cancer | 3 + 3 |
| 97 | Prostate Cancer | 3 + 2 |
| 100 | Prostate Cancer | 3 + 3 |
| 101 | Prostate Cancer | 3 + 3 |
| 104 | Prostate Cancer | 3 + 3 |
| 105 | Prostate Cancer | 3 + 4 |
| 106 | Prostate Cancer | 3 + 3 |
| 138 | Prostate Cancer | 3 + 3 |
| 151 | Prostate Cancer | 3 + 3 |
| 153 | Prostate Cancer | 3 + 3 |
| 155 | Prostate Cancer | 4 + 3 |
| 171 | Prostate Cancer | 3 + 4 |
| 173 | Prostate Cancer | 3 + 4 |
| 231 | Prostate Cancer | 3 + 4 |
| 232 | Prostate Cancer | 3 + 3 |
| 251 | Prostate Cancer | 3 + 4 |
| 282 | Prostate Cancer | 4 + 3 |
| 286 | Prostate Cancer | 3 + 3 |
| 294 | Prostate Cancer | 3 + 4 |
| 351 | Prostate Cancer | 5 + 4 |
| 361 | Prostate Cancer | 3 + 3 |
| 362 | Prostate Cancer | 3 + 3 |
| 365 | Prostate Cancer | 3 + 2 |
| 368 | Prostate Cancer | 3 + 3 |
| 379 | Prostate Cancer | 3 + 4 |
| 388 | Prostate Cancer | 5 + 3 |
| 391 | Prostate Cancer | 3 + 3 |
| 420 | Prostate Cancer | 3 + 3 |
| 425 | Prostate Cancer | 3 + 3 |
| 428 | Prostate Cancer | 4 + 3 |
| 431 | Prostate Cancer | 3 + 4 |
| 492 | Prostate Cancer | 3 + 3 |
| 493 | Prostate Cancer | 3 + 4 |
| 496 | Prostate Cancer | 3 + 3 |
| 510 | Prostate Cancer | 3 + 3 |
| 511 | Prostate Cancer | 4 + 3 |
| 514 | Prostate Cancer | 3 + 3 |
| 549 | Prostate Cancer | 3 + 3 |
| 552 | Prostate Cancer | 3 + 3 |
| 858 | Prostate Cancer | 3 + 4 |
| 859 | Prostate Cancer | 3 + 4 |
| 864 | Prostate Cancer | 3 + 4 |
| 883 | Prostate Cancer | 4 + 4 |
| 895 | Prostate Cancer | 3 + 3 |
| 901 | Prostate Cancer | 3 + 3 |
| 909 | Prostate Cancer | 3 + 3 |
| 921 | Prostate Cancer | 3 + 3 |
| 923 | Prostate Cancer | 4 + 3 |
| 934 | Prostate Cancer | 3 + 3 |
| 1134 | Prostate Cancer | 3 + 4 |
| 1135 | Prostate Cancer | 3 + 3 |
| 1136 | Prostate Cancer | 3 + 4 |
| 1137 | Prostate Cancer | 3 + 3 |
| 1138 | Prostate Cancer | 4 + 3 |

Table 12 provides information about each patient from which the breast tissue samples were isolated, including: 1) the “Pat Num”, a number assigned to the patient for identification purposes; 2) the “Histology”, which indicates whether the tumor was characterized as an intraductal carcinoma (IDC) or ductal carcinoma in situ (DCIS); 3) the incidence of lymph node metastases (LMF), represented as the number of lymph nodes positive to metastases out of the total number examined in the patient; 4) the “Tumor Size”; 5) “TNM Stage”, which provides the tumor grade (T#), where the number indicates the grade and “p” indicates that the tumor grade is a pathological classification; regional lymph node metastasis (N#), where “0” indicates no lymph node metastases were found, “1” indicates lymph node metastases were found, and “X” means information not available and; the identification or detection of metastases to sites distant to the tumor and their location (M#), with “X” indicating that no distant mesatses were reported; and the stage of the tumor (“Stage Grouping”). “nr” indicates “reported”.

TABLE 12
Breast cancer patient data

| Pat | | | Tumor | | |
|---|---|---|---|---|---|
| Num | Histology | LMF | Size | TNM Stage | Stage Grouping |
| 280 | IDC, DCIS + | nr | 2 cm | T2NXMX | probable Stage II |
| | D2 | | | | |
| 284 | IDC, DCIS | 0/16 | 2 cm | T2pN0MX | Stage II |
| 285 | IDC, DCIS | nr | 4.5 cm | T2NXMX | probable Stage II |
| 291 | IDC, DCIS | 0/24 | 4.5 cm | T2pN0MX | Stage II |
| 302 | IDC, DCIS | nr | 2.2 cm | T2NXMX | probable Stage II |
| 375 | IDC, DCIS | nr | 1.5 cm | T1NXMX | probable Stage I |
| 408 | IDC | 0/23 | 3.0 cm | T2pN0MX | Stage II |
| 416 | IDC | 0/6 | 3.3 cm | T2pN0MX | Stage II |
| 421 | IDC, DCIS | nr | 3.5 cm | T2NXMX | probable Stage II |
| 459 | IDC | 2/5 | 4.9 cm | T2pN1MX | Stage II |
| 465 | IDC | 0/10 | 6.5 cm | T3pN0MX | Stage II |
| 470 | IDC, DCIS | 0/6 | 2.5 cm | T2pN0MX | Stage II |
| 472 | IDC, DCIS | 6/45 | 5.0+ cm | T3pN1MX | Stage III |
| 474 | IDC | 0/18 | 6.0 cm | T3pN0MX | Stage II |
| 476 | IDC | 0/16 | 3.4 cm | T2pN0MX | Stage II |
| 605 | IDC, DCIS | 1/25 | 5.0 cm | T2pN1MX | Stage II |
| 649 | IDC, DCIS | 1/29 | 4.5 cm | T2pN1MX | Stage II |

Identification of Differentially Expressed Genes

cDNA probes were prepared from total RNA isolated from the patient cells described above. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red), and vice versa.

Each array used had an identical spatial layout and control spot set. Each microarray was divided into two areas, each area having an array with, on each half, twelve groupings of 32×12 spots, for a total of about 9,216 spots on each array. The two areas are spotted identically which provide for at least two duplicates of each clone per array.

Polynucleotides for use on the arrays were obtained from both publicly available sources and from cDNA libraries generated from selected cell lines and patient tissues. PCR products of from about 0.5 kb to 2.0 kb amplified from these sources were spotted onto the array using a Molecular Dynamics Gen III spotter according to the manufacturer's recommendations. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about four duplicate measurements for each clone, two of one color and two of the other, for each sample.

The differential expression assay was performed by mixing equal amounts of probes from tumor cells and normal cells of the same patient. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

Table 13 (inserted prior to claims) provides the results for gene products expressed by at least 2-fold or greater in cancerous prostate, colon, or breast tissue samples relative to normal tissue samples in at least 20% of the patients tested. Table 12 includes: 1) the SEQ ID NO (“SEQ ID”) assigned to each sequence for use in the present specification; 2) the Cluster Identification No. (“CLUSTER”); 3) the percentage of patients tested in which expression levels (e.g., as message level) of the gene was at least 2-fold greater in cancerous breast tissue than in matched normal tissue (“BREAST PATIENTS>=2x”); 4) the percentage of patients tested in which expression levels (e.g., as message level) of the gene was less than or equal to ½ of the expression level in matched normal breast cells (“BREAST PATIENTS<=halfx”); 5) the percentage of patients tested in which expression levels (e.g., as message level) of the gene was at least 2-fold greater in cancerous colon tissue than in matched normal tissue (“COLON PATIENTS>=2x”); 6) the percentage of patients tested in which expression levels (e.g., as message level) of the gene was less than or equal to ½ of the expression level in matched normal colon cells (“COLON PATIENTS<=halfx”); 7) the percentage of patients tested in which expression levels (e.g., as message level) of the gene was at least 2-fold greater in cancerous prostate tissue than in matched normal tissue (“PROSTATE PATIENTS>=2x”); and 8) the percentage of patients tested in which expression levels (e.g., as message level) of the gene was less than or equal to ½ of the expression level in matched normal prostate cells (“PROSTATE PATIENTS<=halfx”).

These data provide evidence that the genes represented by the polynucleotides having the indicated sequences are differentially expressed in breast cancer as compared to normal non-cancerous breast tissue, are differentially expressed in colon cancer as compared to normal non-cancerous colon tissue, and are differentially expressed in prostate cancer as compared to normal non-cancerous prostate tissue.

### Example 7

Antisense Regulation of Gene Expression

The expression of the differentially expressed genes represented by the polynucleotides in the cancerous cells can be further analyzed using antisense knockout technology to confirm the role and function of the gene product in tumorigenesis, e.g., in promoting a metastatic phenotype.

Methods for analysis using antisense technology are well known in the art. For example, a number of different oligonucleotides complementary to the mRNA generated by the differentially expressed genes identified herein can be designed as antisense oligonucleotides, and tested for their ability to suppress expression of the genes. Sets of antisense oligomers specific to each candidate target are designed using the sequences of the polynucleotides corresponding to a differentially expressed gene and the software program HYBsimulator Version 4 (available for Windows 95/Windows NT or for Power Macintosh, RNAture, Inc. 1003 Health Sciences Road, West, Irvine, Calif. 92612 USA). Factors considered when designing antisense oligonucleotides include: 1) the The expression of the differentially expressed genes represented by the polynucleotides in the cancerous cells can be analyzed using antisense knockout technology to confirm the role and function of the gene product in tumorigenesis, e.g., in promoting a metastatic phenotype.

A number of different oligonucleotides complementary to the mRNA generated by the differentially expressed genes identified herein can be designed as potential antisense oligonucleotides, and tested for their ability to suppress expression of the genes. Sets of antisense oligomers specific to each candidate target are designed using the sequences of the polynucleotides corresponding to a differentially expressed gene and the software program H-YBsimulator Version 4 (available for Windows 95/Windows NT or for Power Macintosh, RNAture, Inc. 1003 Health Sciences Road, West, Irvine, Calif. 92612 USA). Factors that are considered when designing antisense oligonucleotides include: 1) the secondary structure of oligonucleotides; 2) the secondary structure of the target gene; 3) the specificity with no or minimum cross-hybridization to other expressed genes; 4) stability; 5) length and 6) terminal GC content. The antisense oligonucleotide is designed so that it will hybridize to its target sequence under conditions of high stringency at physiological temperatures (e.g., an optimal temperature for the cells in culture to provide for hybridization in the cell, e.g., about 37° C.), but with minimal formation of homodimers.

Using the sets of oligomers and the HYBsimulator program, three to ten antisense oligonucleotides and their reverse controls are designed and synthesized for each candidate mRNA transcript, which transcript is obtained from the gene corresponding to the target polynucleotide sequence of interest. Once synthesized and quantitated, the oligomers are screened for efficiency of a transcript knock-out in a panel of cancer cell lines. The efficiency of the knock-out is determined by analyzing mRNA levels using lightcycler quantification. The oligomers that resulted in the highest level of transcript knock-out, wherein the level was at least about 50%, preferably about 80-90%, up to 95% or more up to undetectable message, are selected for use in a cell-based proliferation assay, an anchorage independent growth assay, and an apoptosis assay.

The ability of each designed antisense oligonucleotide to inhibit gene expression is tested through transfection into LNCaP, PC3, 22Rv1, MDA-PCA-2b, or DU145 prostate carcinoma cells. For each transfection mixture, a carrier molecule (such as a lipid, lipid derivative, lipid-like molecule, cholesterol, cholesterol derivative, or cholesterol-like molecule) is prepared to a working concentration of 0.5 mM in water, sonicated to yield a uniform solution, and filtered through a 0.45 μm PVDF membrane. The antisense or control oligonucleotide is then prepared to a working concentration of 100 μM in sterile Millipore water. The oligonucleotide is further diluted in OptiMEM™ (Gibco/BRL), in a microfuge tube, to 2 μM, or approximately 20 μg oligo/ml of OptiMEM™. In a separate microfuge tube, the carrier molecule, typically in the amount of about 1.5-2 nmol carrier/μg antisense oligonucleotide, is diluted into the same volume of OptiMEM™ used to dilute the oligonucleotide. The diluted antisense oligonucleotide is immediately added to the diluted carrier and mixed by pipetting up and down. Oligonucleotide is added to the cells to a final concentration of 30 nM.

The level of target mRNA that corresponds to a target gene of interest in the transfected cells is quantitated in the cancer cell lines using the Roche LightCycler™ real-time PCR machine. Values for the target mRNA are normalized versus an internal control (e.g., beta-actin). For each 20 μl reaction, extracted RNA (generally 0.2-1 μg total) is placed into a sterile 0.5 or 1.5 ml microcentrifuge tube, and water is added to a total volume of 12.5 μl. To each tube is added 7.5 μl of a buffer/enzyme mixture, prepared by mixing (in the order listed) 2.5 μl H₂O, 2.0 μl 10×reaction buffer, 10 μl oligo dT (20 pmol), 1.0 μl dNTP mix (10 mM each), 0.5 μl RNAsin® (20 u) (Ambion, Inc., Hialeah, Fla.), and 0.5 μl MMLV reverse transcriptase (50 u) (Ambion, Inc.). The contents are mixed by pipetting up and down, and the reaction mixture is incubated at 42° C. for 1 hour. The contents of each tube are centrifuged prior to amplification.

An amplification mixture is prepared by mixing in the following order: 1×PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10×concentration from Perkin-Elmer, Norwalk, Conn.). In 1×concentration it contains 10 mM Tris pH 8.3 and 50 mM KCl. SYBR® Green (Molecular Probes, Eugene, Oreg.) is a dye which fluoresces when bound to double stranded DNA. As double stranded PCR product is produced during amplification, the fluorescence from SYBR® Green increases. To each 20 μl aliquot of amplification mixture, 2 μl of template RT is added, and amplification is carried out according to standard protocols. The results are expressed as the percent decrease in expression of the corresponding gene product relative to non-transfected cells, vehicle-only transfected (mock-transfected) cells, or cells transfected with reverse control oligonucleotides.

### Example 8

Effect of Expression on Proliferation

The effect of gene expression on the inhibition of cell proliferation can be assessed in metastatic breast cancer cell lines (MDA-MB-231 (“231 ”)); SW620 colon colorectal carcinoma cells; SKOV3 cells (a human ovarian carcinoma cell line); or LNCaP, PC3, 22Rv1, MDA-PCA-2b, or DU145 prostate cancer cells.

Cells are plated to approximately 60-80% confluency in 96-well dishes. Antisense or reverse control oligonucleotide is diluted to 2 μM in OptiMEM™. The oligonucleotide-OptiMEM™ can then be added to a delivery vehicle, which delivery vehicle can be selected so as to be optimized for the particular cell type to be used in the assay. The oligo/delivery vehicle mixture is then further diluted into medium with serum on the cells. The final concentration of oligonucleotide for all experiments can be about 300 nM.

Antisense oligonucleotides are prepared as described above (see Example 3). Cells are transfected overnight at 37° C. and the transfection mixture is replaced with fresh medium the next morning. Transfection is carried out as described above in Example 8.

Those antisense oligonucleotides that result in inhibition of proliferation of SW620 cells indicate that the corresponding gene plays a role in production or maintenance of the cancerous phenotype in cancerous colon cells. Those antisense oligonucleotides that inhibit proliferation in SKOV3 cells represent genes that play a role in production or maintenance of the cancerous phenotype in cancerous breast cells. Those antisense oligonucleotides that result in inhibition of proliferation of MDA-MB-231 cells indicate that the corresponding gene plays a role in production or maintenance of the cancerous phenotype in cancerous ovarian cells. Those antisense oligonucleotides that inhibit proliferation in LNCaP, PC3, 22Rv1, MDA-PCA-2b, or DU145 cells represent genes that play a role in production or maintenance of the cancerous phenotype in cancerous prostate cells.

### Example 9

Effect of Gene Expression on Cell Migration

The effect of gene expression on the inhibition of cell migration can be assessed in LNCaP, PC3, 22Rv1, MDA-PCA-2b, or DU145 prostate cancer cells using static endothelial cell binding assays, non-static endothelial cell binding assays, and transmigration assays.

For the static endothelial cell binding assay, antisense oligonucleotides are prepared as described above (see Example 8). Two days prior to use, prostate cancer cells (CaP) are plated and transfected with antisense oligonucleotide as described above (see Examples 3 and 4). On the day before use, the medium is replaced with fresh medium, and on the day of use, the medium is replaced with fresh medium containing 2 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in DMEM/1% BSA/10 mM HEPES pH 7.0. Finally, CaP cells are counted and resuspended at a concentration of 1×10⁶ cells/ml.

Endothelial cells (EC) are plated onto 96-well plates at 40-50% confluence 3 days prior to use. On the day of use, EC are washed 1× with PBS and 50λ DMDM/1% BSA/10 mM HEPES pH 7 is added to each well. To each well is then added 50λ (50λ) CaP cells in DMEM/1% BSA/10 mM HEPES pH 7. The plates are incubated for an additional 30 min and washed 5× with PBS containing Ca⁺⁺ and Mg⁺⁺. After the final wash, 100 μL PBS is added to each well and fluorescence is read on a fluorescent plate reader (Ab492/Em 516 nm).

For the non-static endothelial cell binding assay, CaP are prepared as described above. EC are plated onto 24-well plates at 30-40% confluence 3 days prior to use. On the day of use, a subset of EC are treated with cytokine for 6 hours then washed 2× with PBS. To each well is then added 150-200K CaP cells in DMEM/1% BSA/10 mM HEPES pH 7. Plates are placed on a rotating shaker (70 RPM) for 30 min and then washed 3× with PBS containing Ca⁺⁺ and Mg⁺⁺. After the final wash, 500 μL PBS is added to each well and fluorescence is read on a fluorescent plate reader (Ab492/Em 516 nm).

For the transmigration assay, CaP are prepared as described above with the following changes. On the day of use, CaP medium is replaced with fresh medium containing 5 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in EGM-2-MV medium. Finally, CaP cells are counted and resuspended at a concentration of 1×10⁶ cells/ml.

EC are plated onto FluorBlok transwells (BD Biosciences) at 30-40% confluence 5-7 days before use. Medium is replaced with fresh medium 3 days before use and on the day of use. To each transwell is then added 50K labeled CaP. 30 min prior to the first fluorescence reading, 10 μg of FITC-dextran (10K MW) is added to the EC plated filter. Fluorescence is then read at multiple time points on a fluorescent plate reader (Ab492/Em 516 nm).

Those antisense oligonucleotides that result in inhibition of binding of LNCaP, PC3, 22Rv1, MDA-PCA-2b, or DU145 prostate cancer cells to endothelial cells indicate that the corresponding gene plays a role in the production or maintenance of the cancerous phenotype in cancerous prostate cells. Those antisense oligonucleotides that result in inhibition of endothelial cell transmigration by LNCaP, PC3, 22Rv1, MDA-PCA-2b, or DU145 prostate cancer cells indicate that the corresponding gene plays a role in the production or maintenance of the cancerous phenotype in cancerous prostate cells.

### Example 10

Effect of Gene Expression on Colony Formation

The effect of gene expression upon colony formation of SW620 cells, SKOV3 cells, MD-MBA-231 cells, LNCaP cells, PC3 cells, 22Rv1 cells, MDA-PCA-2b cells, and DU145 cells can be tested in a soft agar assay. Soft agar assays are conducted by first establishing a bottom layer of 2 ml of 0.6% agar in media plated fresh within a few hours of layering on the cells. The cell layer is formed on the bottom layer by removing cells transfected as described above from plates using 0.05% trypsin and washing twice in media. The cells are counted in a Coulter counter, and resuspended to 10⁶ per ml in media. 10 μl aliquots are placed with media in 96-well plates (to check counting with WST1), or diluted further for the soft agar assay. 2000 cells are plated in 800 μl 0.4% agar in duplicate wells above 0.6% agar bottom layer. After the cell layer agar solidifies, 2 ml of media is dribbled on top and antisense or reverse control oligo (produced as described in Example 8) is added without delivery vehicles. Fresh media and oligos are added every 3-4 days. Colonies form in 10 days to 3 weeks. Fields of colonies are counted by eye. Wst-1 metabolism values can be used to compensate for small differences in starting cell number. Larger fields can be scanned for visual record of differences.

Those antisense oligonucleotides that result in inhibition of colony formation of SW620 cells indicate that the corresponding gene plays a role in production or maintenance of the cancerous phenotype in cancerous colon cells. Those antisense oligonucleotides that inhibit colony formation in SKOV3 cells represent genes that play a role in production or maintenance of the cancerous phenotype in cancerous breast cells. Those antisense oligonucleotides that result in inhibition of colony formation of MDA-MB-231 cells indicate that the corresponding gene plays a role in production or maintenance of the cancerous phenotype in cancerous ovarian cells. Those antisense oligonucleotides that inhibit colony formation in LNCaP, PC3, 22Rv1, MDA-PCA-2b, or DU145 cells represent genes that play a role in production or maintenance of the cancerous phenotype in cancerous prostate cells.

### Example 11

Induction of Cell Death upon Depletion of Polypeptides by Depletion of mRNA (“Antisense Knockout”)

In order to assess the effect of depletion of a target message upon cell death, LNCaP, PC3, 22Rv1, MDA-PCA-2b, or DU145 cells, or other cells derived from a cancer of interest, can be transfected for proliferation assays. For cytotoxic effect in the presence of cisplatin (cis), the same protocol is followed but cells are left in the presence of 2 μM drug. Each day, cytotoxicity is monitored by measuring the amount of LDH enzyme released in the medium due to membrane damage. The activity of LDH is measured using the Cytotoxicity Detection Kit from Roche Molecular Biochemicals. The data is provided as a ratio of LDH released in the medium vs. the total LDH present in the well at the same time point and treatment (rLDH/tLDH). A positive control using antisense and reverse control oligonucleotides for BCL2 (a known anti-apoptotic gene) is included; loss of message for BCL2 leads to an increase in cell death compared with treatment with the control oligonucleotide (background cytotoxicity due to transfection).

### Example 12

Functional Analysis of Gene Products Differentially Expressed in Cancer

The gene products of sequences of a gene differentially expressed in cancerous cells can be further analyzed to confirm the role and function of the gene product in tumorigenesis, e.g., in promoting or inhibiting development of a metastatic phenotype. For example, the function of gene products corresponding to genes identified herein can be assessed by blocking function of the gene products in the cell. For example, where the gene product is secreted or associated with a cell surface membrane, blocking antibodies can be generated and added to cells to examine the effect upon the cell phenotype in the context of, for example, the transformation of the cell to a cancerous, particularly a metastatic, phenotype. In order to generate antibodies, a clone corresponding to a selected gene product is selected, and a sequence that represents a partial or complete coding sequence is obtained. The resulting clone is expressed, the polypeptide produced isolated, and antibodies generated. The antibodies are then combined with cells and the effect upon tumorigenesis assessed.

Where the gene product of the differentially expressed genes identified herein exhibits sequence homology to a protein of known function (e.g., to a specific kinase or protease) and/or to a protein family of known function (e.g., contains a domain or other consensus sequence present in a protease family or in a kinase family), then the role of the gene product in tumorigenesis, as well as the activity of the gene product, can be examined using small molecules that inhibit or enhance function of the corresponding protein or protein family.

Additional functional assays include, but are not necessarily limited to, those that analyze the effect of expression of the corresponding gene upon cell cycle and cell migration. Methods for performing such assays are well known in the art.

### Example 13

Deposit Information.

A deposit of the biological materials in the tables referenced below was made with the American Type Culture Collection, 10801 University Blvd., Manasas, Va. 20110-2209, under the provisions of the Budapest Treaty, on or before the filing date of the present application. The accession number indicated is assigned after successful viability testing, and the requisite fees were paid. Access to said cultures will be available during pendency of the patent application to one determined by the Commissioner to be entitled to such under 37 C.F.R. §1.14 and 35 U.S.C. §122. All restriction on availability of said cultures to the public will be irrevocably removed upon the granting of a patent based upon the application. Moreover, the designated deposits will be maintained for a period of thirty (30) years from the date of deposit, or for five (5) years after the last request for the deposit; or for the enforceable life of the U.S. patent, whichever is longer. Should a culture become nonviable or be inadvertently destroyed, or, in the case of plasmid-containing strains, lose its plasmid, it will be replaced with a viable culture(s) of the same taxonomic description.

These deposits are provided merely as a convenience to those of skill in the art, and are not an admission that a deposit is required. A license may be required to make, use, or sell the deposited materials, and no such license is hereby granted. The deposit below was received by the ATCC on or before the filing date of the present application.

TABLE 14A
Cell Lines Deposited with ATCC

| | | ATCC | |
|---|---|---|---|
| Cell Line | Deposit Date | Accession No. | CMCC Accession No. |
| KM12L4-A | Mar. 19, 1998 | CRL-12496 | 11606 |
| Km12C | May 15, 1998 | CRL-12533 | 11611 |
| MDA-MB- | May 15, 1998 | CRL-12532 | 10583 |
| 231 | | | |
| MCF-7 | Oct. 9, 1998 | CRL-12584 | 10377 |

In addition, pools of selected clones, as well as libraries containing specific clones, were assigned an “ES” number (internal reference) and deposited with the ATCC. Table 14 below provides the ATCC Accession Nos. of the clones deposited as a library named ES217. The deposit was made on Jan. 18, 2001. Table 15 (inserted before the claims) provides the ATCC Accession Nos. of the clones deposited as libraries named ES210-ES216 on Jul. 25, 2000.

TABLE 14B
Clones Deposited as Library No. ES217 with ATCC on or before Jan. 18, 2001.

| CloneID | CMCC# | ATCC# | CloneID | CMCC# | ATCC# |
|---|---|---|---|---|---|
| M00073094B:A01 | 5418 | PTA-2918 | M00073425A:H12 | 5418 | PTA-2918 |
| M00073096B:A12 | 5418 | PTA-2918 | M00073427B:E04 | 5418 | PTA-2918 |
| M00073412C:E07 | 5418 | PTA-2918 | M00073408A:D06 | 5418 | PTA-2918 |
| M00073408C:F06 | 5418 | PTA-2918 | M00073428D:H03 | 5418 | PTA-2918 |
| M00073435C:E06 | 5418 | PTA-2918 | M00073435B:E11 | 5418 | PTA-2918 |
| M00073403B:F06 | 5418 | PTA-2918 | M00074323D:F09 | 5418 | PTA-2918 |
| M00073412D:B07 | 5418 | PTA-2918 | M00074333D:A11 | 5418 | PTA-2918 |
| M00073421C:B07 | 5418 | PTA-2918 | M00074335A:H08 | 5418 | PTA-2918 |
| M00073429B:H10 | 5418 | PTA-2918 | M00074337A:G08 | 5418 | PTA-2918 |
| M00073412D:E02 | 5418 | PTA-2918 | M00074340B:D06 | 5418 | PTA-2918 |
| M00073097C:A03 | 5418 | PTA-2918 | M00074343C:A03 | 5418 | PTA-2918 |
| M00073403C:C10 | 5418 | PTA-2918 | M00074346A:H09 | 5418 | PTA-2918 |
| M00073425D:F08 | 5418 | PTA-2918 | M00074347B:F11 | 5418 | PTA-2918 |
| M00073403C:E11 | 5418 | PTA-2918 | M00074349A:E08 | 5418 | PTA-2918 |
| M00073431A:G02 | 5418 | PTA-2918 | M00074355D:H06 | 5418 | PTA-2918 |
| M00073412A:C03 | 5418 | PTA-2918 | M00074361C:B01 | 5418 | PTA-2918 |
| M00073424D:C03 | 5418 | PTA-2918 | M00074365A:E09 | 5418 | PTA-2918 |
| M00073430C:A01 | 5418 | PTA-2918 | M00074366A:D07 | 5418 | PTA-2918 |
| M00073407A:E12 | 5418 | PTA-2918 | M00074366A:H07 | 5418 | PTA-2918 |
| M00073412A:H09 | 5418 | PTA-2918 | M00074370D:G09 | 5418 | PTA-2918 |
| M00073418B:B09 | 5418 | PTA-2918 | M00074375D:E05 | 5418 | PTA-2918 |
| M00073403C:H09 | 5418 | PTA-2918 | M00074382D:F04 | 5418 | PTA-2918 |
| M00073416B:F01 | 5418 | PTA-2918 | M00074384D:G07 | 5418 | PTA-2918 |
| M00073425A:G10 | 5418 | PTA-2918 | M00074388B:E07 | 5418 | PTA-2918 |
| M00073427B:C08 | 5418 | PTA-2918 | M00074392C:D02 | 5418 | PTA-2918 |
| M00073430C:B02 | 5418 | PTA-2918 | M00074405B:A04 | 5418 | PTA-2918 |
| M00073418B:H09 | 5418 | PTA-2918 | M00074417D:F07 | 5418 | PTA-2918 |
| M00073423C:E01 | 5418 | PTA-2918 | M00074392D:D01 | 5418 | PTA-2918 |
| M00074391B:D02 | 5418 | PTA-2918 | M00074406B:F10 | 5418 | PTA-2918 |
| M00074390C:E04 | 5418 | PTA-2918 | M00074430D:G09 | 5418 | PTA-2918 |
| M00074411B:G07 | 5418 | PTA-2918 | M00074395A:B11 | 5418 | PTA-2918 |
| M00074415B:A01 | 5418 | PTA-2918 | M00074404B:H01 | 5418 | PTA-2918 |

Retrieval of Individual Clones from Deposit of Pooled Clones. Where the ATCC deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Those skilled in the art will recognize, or be able to ascertain, using not more than routine experimentation, many equivalents to the specific embodiments of the invention described herein. Such specific embodiments and equivalents are intended to be encompassed by the following claims.

TABLE 2

| SEQ | | | | | |
|---|---|---|---|---|---|
| ID | CLUSTER | SEQNAME | ORIENT | CLONE ID | LIBRARY |
| 1 | 38838 | 2504.A17.GZ43_365806 | F | M00072942B:E02 | IF97-26811-NormBPHProstate |
| 2 | 558959 | 2504.B06.GZ43_365819 | F | M00072942D:F07 | IF97-26811-NormBPHProstate |
| 3 | 19061 | 2504.B11.GZ43_365824 | F | M00072943B:E04 | IF97-26811-NormBPHProstate |
| 4 | 139979 | 2504.B21.GZ43_365834 | F | M00072944A:C07 | IF97-26811-NormBPHProstate |
| 5 | 24540 | 2504.B23.GZ43_365836 | F | M00072944A:E06 | IF97-26811-NormBPHProstate |
| 6 | 40164 | 2504.C08.GZ43_365845 | F | M00072944C:C02 | IF97-26811-NormBPHProstate |
| 7 | 53675 | 2504.C11.GZ43_365848 | F | M00072944D:C08 | IF97-26811-NormBPHProstate |
| 8 | 119614 | 2504.D09.GZ43_365870 | F | M00072947B:G04 | IF97-26811-NormBPHProstate |
| 9 | 918867 | 2504.D16.GZ43_365877 | F | M00072947D:G05 | IF97-26811-NormBPHProstate |
| 10 | 823 | 2504.E23.GZ43_365908 | F | M00072950A:A06 | IF97-26811-NormBPHProstate |
| 11 | 604822 | 2504.F20.GZ43_365929 | F | M00072961A:G04 | IF97-26811-NormBPHProstate |
| 12 | 343686 | 2504.G01.GZ43_365934 | F | M00072961B:G10 | IF97-26811-NormBPHProstate |
| 13 | 21554 | 2504.G04.GZ43_365937 | F | M00072961C:B06 | IF97-26811-NormBPHProstate |
| 14 | 204211 | 2504.G07.GZ43_365940 | F | M0072962A:B05 | IF97-26811-NormBPHProstate |
| 15 | 21567 | 2504.H02.GZ43_365959 | F | M00072963B:G11 | IF97-26811-NormBPHProstate |
| 16 | 956537 | 2504.I11.GZ43_365992 | F | M00072967A:G07 | IF97-26811-NormBPHProstate |
| 17 | 44238 | 2504.I13.GZ43_365994 | F | M00072967B:G06 | IF97-26811-NormBPHProstate |
| 18 | 56663 | 2504.I19.GZ43_366000 | F | M00072968A:F08 | IF97-26811-NormBPHProstate |
| 19 | 49884 | 2504.I23.GZ43_366004 | F | M00072968D:A06 | IF97-26811-NormBPHProstate |
| 20 | 402904 | 2504.J02.GZ43_366007 | F | M00072968D:E05 | IF97-26811-NormBPHProstate |
| 21 | 845171 | 2504.J11.GZ43_366016 | F | M00072970C:B07 | IF97-26811-NormBPHProstate |
| 22 | 471272 | 2504.K01.GZ43_366030 | F | M00072971A:E04 | IF97-26811-NormBPHProstate |
| 23 | 660842 | 2504.K02.GZ43_366031 | F | M00072971A:F11 | IF97-26811-NormBPHProstate |
| 24 | 764473 | 2504.K07.GZ43_366036 | F | M00072971C:B07 | IF97-26811-NormBPHProstate |
| 25 | 406416 | 2504.K14.GZ43_366043 | F | M00072972A:C03 | IF97-26811-NormBPHProstate |
| 26 | 842403 | 2504.L16.GZ43_366069 | F | M00072974A:A11 | IF97-26811-NormBPHProstate |
| 27 | 401809 | 2504.M12.GZ43_366089 | F | M00072974D:B04 | IF97-26811-NormBPHProstate |
| 28 | 28050 | 2504.M18.GZ43_366095 | F | M00072975A:D11 | IF97-26811-NormBPHProstate |
| 29 | 37758 | 2504.M19.GZ43_366096 | F | M00072975A:E02 | IF97-26811-NormBPHProstate |
| 30 | 85792 | 2504.O09.GZ43_366134 | F | M00072977A:F06 | IF97-26811-NormBPHProstate |
| 31 | 400258 | 2504.O12.GZ43_366137 | F | M00072977B:C05 | IF97-26811-NormBPHProstate |
| 32 | 9934 | 2505.B02.GZ43_366199 | F | M00072980B:C05 | IF97-26811-NormBPHProstate |
| 33 | 448503 | 2505.B05.GZ43_366202 | F | M00072980B:G01 | IF97-26811-NormBPHProstate |
| 34 | 731371 | 2505.B17.GZ43_366214 | F | M00073001A:F07 | IF97-26811-NormBPHProstate |
| 35 | 171148 | 2505.B18.GZ43_366215 | F | M00073001B:E07 | IF97-26811-NormBPHProstate |
| 36 | 49090 | 2505.C06.GZ43_366227 | F | M00073002B:B12 | IF97-26811-NormBPHProstate |
| 37 | 57638 | 2505.C17.GZ43_366238 | F | M00073002D:B08 | IF97-26811-NormBPHProstate |
| 38 | 523261 | 2505.C21.GZ43_366242 | F | M00073003A:E06 | IF97-26811-NormBPHProstate |
| 39 | 85192 | 2505.D01.GZ43_366246 | F | M00073003B:E10 | IF97-26811-NormBPHProstate |
| 40 | 696086 | 2505.D03.GZ43_366248 | F | M00073003B:H01 | IF97-26811-NormBPHProstate |
| 41 | 41455 | 2505.D04.GZ43_366249 | F | M00073003C:C05 | IF97-26811-NormBPHProstate |
| 42 | 336576 | 2505.E09.GZ43_366278 | F | M00073006A:H08 | IF97-26811-NormBPHProstate |
| 43 | 36407 | 2505.E15.GZ43_366284 | F | M00073006C:D07 | IF97-26811-NormBPHProstate |
| 44 | 397652 | 2505.F09.GZ43_366302 | F | M00073007D:E05 | IF97-26811-NormBPHProstate |
| 45 | 85792 | 2505.G06.GZ43_366323 | F | M00073009B:C08 | IF97-26811-NormBPHProstate |
| 46 | 376516 | 2505.G16.GZ43_366333 | F | M00073009D:A02 | IF97-26811-NormBPHProstate |
| 47 | 588996 | 2505.H14.GZ43_366355 | F | M00073012A:C11 | IF97-26811-NormBPHProstate |
| 48 | 8401 | 2505.I04.GZ43_366369 | F | M00073013A:D10 | IF97-26811-NormBPHProstate |
| 49 | 11561 | 2505.I06.GZ43_366371 | F | M00073013A:F10 | IF97-26811-NormBPHProstate |
| 50 | 726937 | 2505.I14.GZ43_366379 | F | M00073013C:B10 | IF97-26811-NormBPHProstate |
| 51 | 672233 | 2505.I16.GZ43_366381 | F | M00073013C:G05 | IF97-26811-NormBPHProstate |
| 52 | 31453 | 2505.J15.GZ43_366404 | F | M00073014D:F01 | IF97-26811-NormBPHProstate |
| 53 | 40330 | 2505.J20.GZ43_366409 | F | M00073015A:E12 | IF97-26811-NormBPHProstate |
| 54 | 38454 | 2505.J22.GZ43_366411 | F | M00073015A:H06 | IF97-26811-NormBPHProstate |
| 55 | 666927 | 2505.J23.GZ43_366412 | F | M00073015B:A05 | IF97-26811-NormBPHProstate |
| 56 | 163500 | 2505.K09.GZ43_366422 | F | M00073015C:E10 | IF97-26811-NormBPHProstate |
| 57 | 42034 | 2505.L07.GZ43_366444 | F | M00073017A:D06 | IF97-26811-NormBPHProstate |
| 58 | 455662 | 2505.L09.GZ43_366446 | F | M00073017A:F03 | IF97-26811-NormBPHProstate |
| 59 | 985835 | 2505.M09.GZ43_366470 | F | M00073019A:H12 | IF97-26811-NormBPHProstate |
| 60 | 502358 | 2505.M10.GZ43_366471 | F | M00073019B:B12 | IF97-26811-NormBPHProstate |
| 61 | 189993 | 2505.N19.GZ43_366504 | F | M00073020C:F07 | IF97-26811-NormBPHProstate |
| 62 | 605923 | 2505.N21.GZ43_366506 | F | M00073020D:C06 | IF97-26811-NormBPHProstate |
| 63 | 935908 | 2505.O09.GZ43_366518 | F | M00073021C:E04 | IF97-26811-NormBPHProstate |
| 64 | 568204 | 2505.O12.GZ43_366521 | F | M00073021D:C03 | IF97-26811-NormBPHProstate |
| 65 | 640970 | 2505.O19.GZ43_366528 | F | M00073023A:D10 | IF97-26811-NormBPHProstate |
| 66 | 558581 | 2505.P09.GZ43_366542 | F | M00073025A:E11 | IF97-26811-NormBPHProstate |
| 67 | 823 | 2505.P23.GZ43_366556 | F | M00073026B:F01 | IF97-26811-NormBPHProstate |
| 68 | 195498 | 2510.A11.GZ43_369036 | F | M00073026D:G04 | IF97-26811-NormBPHProstate |
| 69 | 7885 | 2510.A19.GZ43_369044 | F | M00073027B:H12 | IF97-26811-NormBPHProstate |
| 70 | 63363 | 2510.C06.GZ43_369079 | F | M00073030A:G05 | IF97-26811-NormBPHProstate |
| 71 | 558602 | 2510.C07.GZ43_369080 | F | M00073030B:C02 | IF97-26811-NormBPHProstate |
| 72 | 38454 | 2510.C10.GZ43_369083 | F | M00073030C:A02 | IF97-26811-NormBPHProstate |
| 73 | 21546 | 2510.E13.GZ43_369134 | F | M00073036C:H10 | IF97-26811-NormBPHProstate |
| 74 | 846506 | 2510.E16.GZ43_369137 | F | M00073037A:C06 | IF97-26811-NormBPHProstate |
| 75 | 62816 | 2510.F11.GZ43_369156 | F | M00073037D:H02 | IF97-26811-NormBPHProstate |
| 76 | 134226 | 2510.F23.GZ43_369168 | F | M00073038C:C07 | IF97-26811-NormBPHProstate |
| 77 | 63363 | 2510.G05.GZ43_369174 | F | M00073038D:D12 | IF97-26811-NormBPHProstate |
| 78 | 85192 | 2510.G06.GZ43_369175 | F | M00073038D:F10 | IF97-26811-NormBPHProstate |
| 79 | 9048 | 2510.G09.GZ43_369178 | F | M00073039A:D09 | IF97-26811-NormBPHProstate |
| 80 | 480019 | 2510.G14.GZ43_369183 | F | M00073039C:B10 | IF97-26811-NormBPHProstate |
| 81 | 58429 | 2510.G21.GZ43_369190 | F | M00073040A:B02 | IF97-26811-NormBPHProstate |
| 82 | 115787 | 2510.H03.GZ43_369196 | F | M00073040D:F05 | IF97-26811-NormBPHProstate |
| 83 | 42891 | 2510.I08.GZ43_369225 | F | M00073043B:C10 | IF97-26811-NormBPHProstate |
| 84 | 469837 | 2510.I10.GZ43_369227 | F | M00073043B:E08 | IF97-26811-NormBPHProstate |
| 85 | 54634 | 2510.I16.GZ43_369233 | F | M00073043C:F04 | IF97-26811-NormBPHProstate |
| 86 | 648899 | 2510.I23.GZ43_369240 | F | M00073043D:H09 | IF97-26811-NormBPHProstate |
| 87 | 778001 | 2510.J06.GZ43_369247 | F | M00073044B:F08 | IF97-26811-NormBPHProstate |
| 88 | 452714 | 2510.J10.GZ43_369251 | F | M00073044C:C12 | IF97-26811-NormBPHProstate |
| 89 | 142502 | 2510.J11.GZ43_369252 | F | M00073044C:D08 | IF97-26811-NormBPHProstate |
| 90 | 668962 | 2510.J12.GZ43_369253 | F | M00073044C:G12 | IF97-26811-NormBPHProstate |
| 91 | 210229 | 2510.J14.GZ43_369255 | F | M00073044D:F08 | IF97-26811-NormBPHProstate |
| 92 | 483211 | 2510.J18.GZ43_369259 | F | M00073045B:A03 | IF97-26811-NormBPHProstate |
| 93 | 7307 | 2510.J22.GZ43_369263 | F | M00073045B:D06 | IF97-26811-NormBPHProstate |
| 94 | 99399 | 2510.K05.GZ43_369270 | F | M00073045C:E06 | IF97-26811-NormBPHProstate |
| 95 | 421869 | 2510.K06.GZ43_369271 | F | M00073045C:E07 | IF97-26811-NormBPHProstate |
| 96 | 21827 | 2510.K11.GZ43_369276 | F | M00073045D:B04 | IF97-26811-NormBPHProstate |
| 97 | 88462 | 2510.K15.GZ43_369280 | F | M00073046A:A05 | IF97-26811-NormBPHProstate |
| 98 | 16176 | 2510.K16.GZ43_369281 | F | M00073046A:A06 | IF97-26811-NormBPHProstate |
| 99 | 138646 | 2510.K21.GZ43_369286 | F | M00073046B:A12 | IF97-26811-NormBPHProstate |
| 100 | 513744 | 2510.L10.GZ43_369299 | F | M00073046D:F04 | IF97-26811-NormBPHProstate |
| 101 | 15951 | 2510.L17.GZ43_369306 | F | M00073047B:E10 | IF97-26811-NormBPHProstate |
| 102 | 40270 | 2510.L21.GZ43_369310 | F | M00073047C:G01 | IF97-26811-NormBPHProstate |
| 103 | 73796 | 2510.M14.GZ43_369327 | F | M00073048A:H05 | IF97-26811-NormBPHProstate |
| 104 | 18508 | 2510.M20.GZ43_369333 | F | M00073048C:A11 | IF97-26811-NormBPHProstate |
| 105 | 18629 | 2510.M21.GZ43_369334 | F | M00073048C:B01 | IF97-26811-NormBPHProstate |
| 106 | 405925 | 2510.N01.GZ43_369338 | F | M00073048C:E11 | IF97-26811-NormBPHProstate |
| 107 | 455862 | 2510.N12.GZ43_369349 | F | M00073049A:H04 | IF97-26811-NormBPHProstate |
| 108 | 582134 | 2510.N13.GZ43_369350 | F | M00073049B:B03 | IF97-26811-NormBPHProstate |
| 109 | 727966 | 2510.N14.GZ43_369351 | F | M00073049B:B06 | IF97-26811-NormBPHProstate |
| 110 | 644299 | 2510.N24.GZ43_369361 | F | M00073049C:C09 | IF97-26811-NormBPHProstate |
| 111 | 208449 | 2510.O07.GZ43_369368 | F | M00073049C:H07 | IF97-26811-NormBPHProstate |
| 112 | 44480 | 2510.O14.GZ43_369375 | F | M00073050A:D09 | IF97-26811-NormBPHProstate |
| 113 | 148227 | 2510.O21.GZ43_369382 | F | M00073051A:D07 | IF97-26811-NormBPHProstate |
| 114 | 197343 | 2510.O22.GZ43_369383 | F | M00073051A:F12 | IF97-26811-NormBPHProstate |
| 115 | 20571 | 2510.O23.GZ43_369384 | F | M00073051A:F07 | IF97-26811-NormBPHProstate |
| 116 | 724818 | 2510.P08.GZ43_369393 | F | M00073052B:H12 | IF97-26811-NormBPHProstate |
| 117 | 9051 | 2365.A13.GZ43_345239 | F | M00073054A:A06 | IF97-26811-NormBPHProstate |
| 118 | 77849 | 2365.A14.GZ43_345240 | F | M00073054A:C10 | IF97-26811-NormBPHProstate |
| 119 | 5823 | 2365.A23.GZ43_345249 | F | M00073054B:E07 | IF97-26811-NormBPHProstate |
| 120 | 41430 | 2365.B02.GZ43_345252 | F | M00073054C:E02 | IF97-26811-NormBPHProstate |
| 121 | 24115 | 2365.B20.GZ43_345270 | F | M00073055D:E11 | IF97-26811-NormBPHProstate |
| 122 | 573764 | 2365.C10.GZ43_345284 | F | M00073056C:A09 | IF97-26811-NormBPHProstate |
| 123 | 44480 | 2365.C13.GZ43_345287 | F | M00073056C:C12 | IF97-26811-NormBPHProstate |
| 124 | 15604 | 2365.C20.GZ43_345294 | F | M00073057A:F09 | IF97-26811-NormBPHProstate |
| 125 | 54203 | 2365.D03.GZ43_345301 | F | M00073057D:A12 | IF97-26811-NormBPHProstate |
| 126 | 756337 | 2365.D10.GZ43_345308 | F | M00073060B:C06 | IF97-26811-NormBPHProstate |
| 127 | 16852 | 2365.E03.GZ43_345325 | F | M00073061B:F10 | IF97-26811-NormBPHProstate |
| 128 | 59018 | 2365.E08.GZ43_345330 | F | M00073061C:G08 | IF97-26811-NormBPHProstate |
| 129 | 61166 | 2365.E11.GZ43_345333 | F | M00073062B:D09 | IF97-26811-NormBPHProstate |
| 130 | 119614 | 2365.E12.GZ43_345334 | F | M00073062C:D09 | IF97-26811-NormBPHProstate |
| 131 | 806992 | 2365.F07.GZ43_345353 | F | M00073064C:A11 | IF97-26811-NormBPHProstate |
| 132 | 659483 | 2365.F12.GZ43_345358 | F | M00073064C:H09 | IF97-26811-NormBPHProstate |
| 133 | 34077 | 2365.F13.GZ43_345359 | F | M00073064D:B11 | IF97-26811-NormBPHProstate |
| 134 | 404081 | 2365.F24.GZ43_345370 | F | M00073065D:D11 | IF97-26811-NormBPHProstate |
| 135 | 752623 | 2365.G09.GZ43_345379 | F | M00073066B:G03 | IF97-26811-NormBPHProstate |
| 136 | 531505 | 2365.G11.GZ43_345381 | F | M00073066C:D02 | IF97-26811-NormBPHProstate |
| 137 | 588059 | 2365.G17.GZ43_345387 | F | M00073067A:E09 | IF97-26811-NormBPHProstate |
| 138 | 271456 | 2365.G19.GZ43_345389 | F | M00073067B:D04 | IF97-26811-NormBPHProstate |
| 139 | 5791 | 2365.G22.GZ43_345392 | F | M00073067D:B02 | IF97-26811-NormBPHProstate |
| 140 | 725987 | 2365.I04.GZ43_345422 | F | M00073069D:G03 | IF97-26811-NormBPHProstate |
| 141 | 58218 | 2365.I06.GZ43_345424 | F | M00073070A:B12 | IF97-26811-NormBPHProstate |
| 142 | 453526 | 2365.I11.GZ43_345429 | F | M00073070B:B06 | IF97-26811-NormBPHProstate |
| 143 | 141010 | 2365.J14.GZ43_345456 | F | M00073071D:D02 | IF97-26811-NormBPHProstate |
| 144 | 558342 | 2365.J19.GZ43_345461 | F | M00073072A:A10 | IF97-26811-NormBPHProstate |
| 145 | 682065 | 2365.L07.GZ43_345497 | F | M00073074B:G04 | IF97-26811-NormBPHProstate |
| 146 | 466312 | 2365.L08.GZ43_345498 | F | M00073074D:A04 | IF97-26811-NormBPHProstate |
| 147 | 204211 | 2365.L23.GZ43_345513 | F | M00073078B:F08 | IF97-26811-NormBPHProstate |
| 148 | 158853 | 2365.M03.GZ43_345517 | F | M00073080B:A07 | IF97-26811-NormBPHProstate |
| 149 | 633646 | 2365.M09.GZ43_345523 | F | M00073081A:F08 | IF97-26811-NormBPHProstate |
| 150 | 375488 | 2365.M13.GZ43_345527 | F | M00073081D:C07 | IF97-26811-NormBPHProstate |
| 151 | 228149 | 2365.M20.GZ43_345534 | F | M00073084C:E02 | IF97-26811-NormBPHProstate |
| 152 | 599028 | 2365.N12.GZ43_345550 | F | M00073085D:B01 | IF97-26811-NormBPHProstate |
| 153 | 691653 | 2365.N23.GZ43_345561 | F | M00073086D:B05 | IF97-26811-NormBPHProstate |
| 154 | 8231 | 2365.O07.GZ43_345569 | F | M00073088C:B04 | IF97-26811-NormBPHProstate |
| 155 | 397652 | 2365.O13.GZ43_345575 | F | M00073088D:F07 | IF97-26811-NormBPHProstate |
| 156 | 20863 | 2365.O20.GZ43_345582 | F | M00073091B:C04 | IF97-26811-NormBPHProstate |
| 157 | 11121 | 2365.O24.GZ43_345586 | F | M00073091D:B06 | IF97-26811-NormBPHProstate |
| 158 | 33725 | 2365.P04.GZ43_345590 | F | M00073092A:D03 | IF97-26811-NormBPHProstate |
| 159 | 37420 | 2365.P10.GZ43_345596 | F | M00073092D:B03 | IF97-26811-NormBPHProstate |
| 160 | 236390 | 2366.A01.GZ43_345611 | F | M00073094B:A01 | IF97-26811-NormBPHProstate |
| 161 | 831518 | 2366.F02.GZ43_345632 | F | M00073412A:C03 | IF97-26811-NormBPHProstate |
| 162 | 89912 | 2366.E03.GZ43_345647 | F | M00073408C:F06 | IF97-26811-NormBPHProstate |
| 163 | 853371 | 2366.J03.GZ43_345652 | F | M00073424D:C03 | IF97-26811-NormBPHProstate |
| 164 | 401741 | 2366.C04.GZ43_345661 | F | M00073403B:F06 | IF97-26811-NormBPHProstate |
| 165 | 50062 | 2366.D04.GZ43_345662 | F | M00073407A:E12 | IF97-26811-NormBPHProstate |
| 166 | 377367 | 2366.F04.GZ43_345664 | F | M00073412A:H09 | IF97-26811-NormBPHProstate |
| 167 | 9741 | 2366.I04.GZ43_345667 | F | M00073421C:B07 | IF97-26811-NormBPHProstate |
| 168 | 13951 | 2366.H05.GZ43_345682 | F | M00073416B:F01 | IF97-26811-NormBPHProstate |
| 169 | 497520 | 2366.J05.GZ43_345684 | F | M00073425A:G10 | IF97-26811-NormBPHProstate |
| 170 | 136530 | 2366.J06.GZ43_345700 | F | M00073425A:H12 | IF97-26811-NormBPHProstate |
| 171 | 403134 | 2366.C07.GZ43_345709 | F | M00073403C:C10 | IF97-26811-NormBPHProstate |
| 172 | 379939 | 2366.L07.GZ43_345718 | F | M00073428D:H03 | IF97-26811-NormBPHProstate |
| 173 | 128835 | 2366.C08.GZ43_345725 | F | M00073403C:E11 | IF97-26811-NormBPHProstate |
| 174 | 34475 | 2366.P08.GZ43_345738 | F | M00073435B:E11 | IF97-26811-NormBPHProstate |
| 175 | 427808 | 2366.M09.GZ43_345751 | F | M00073431A:G02 | IF97-26811-NormBPHProstate |
| 176 | 450472 | 2366.F10.GZ43_345760 | F | M00073412C:E07 | IF97-26811-NormBPHProstate |
| 177 | 31060 | 2366.P11.GZ43_345786 | F | M00073435C:E06 | IF97-26811-NormBPHProstate |
| 178 | 734776 | 2366.F12.GZ43_345792 | F | M00073412D:B07 | IF97-26811-NormBPHProstate |
| 179 | 47789 | 2366.L12.GZ43_345798 | F | M00073429B:H10 | IF97-26811-NormBPHProstate |
| 180 | 559440 | 2366.C13.GZ43_345805 | F | M00073403C:H09 | IF97-26811-NormBPHProstate |
| 181 | 169728 | 2366.F13.GZ43_345808 | F | M00073412D:E02 | IF97-26811-NormBPHProstate |
| 182 | 137023 | 2366.K13.GZ43_345813 | F | M00073427B:C08 | IF97-26811-NormBPHProstate |
| 183 | 732434 | 2366.I14.GZ43_345827 | F | M00073423C:E01 | IF97-26811-NormBPHProstate |
| 184 | 529 | 2366.K14.GZ43_345829 | F | M00073427B:E04 | IF97-26811-NormBPHProstate |
| 185 | 32624 | 2366.J15.GZ43_345844 | F | M00073425D:F08 | IF97-26811-NormBPHProstate |
| 186 | 378965 | 2366.A17.GZ43_345867 | F | M00073096B:A12 | IF97-26811-NormBPHProstate |
| 187 | 16009 | 2366.L19.GZ43_345910 | F | M00073430C:A01 | IF97-26811-NormBPHProstate |
| 188 | 134637 | 2366.H20.GZ43_345922 | F | M00073418B:B09 | IF97-26811-NormBPHProstate |
| 189 | 1959 | 2366.L21.GZ43_345942 | F | M00073430C:B02 | IF97-26811-NormBPHProstate |
| 190 | 805118 | 2366.A22.GZ43_345947 | F | M00073097C:A03 | IF97-26811-NormBPHProstate |
| 191 | 411952 | 2366.H22.GZ43_345954 | F | M00073418B:H09 | IF97-26811-NormBPHProstate |
| 192 | 887 | 2366.D23.GZ43_345966 | F | M00073408A:D06 | IF97-26811-NormBPHProstate |
| 193 | 172916 | 2367.A21.GZ43_346015 | F | M00073438A:A08 | IF97-26811-NormBPHProstate |
| 194 | 929222 | 2367.A22.GZ43_346016 | F | M00073438A:B02 | IF97-26811-NormBPHProstate |
| 195 | 968417 | 2367.B10.GZ43_346028 | F | M00073438D:G05 | IF97-26811-NormBPHProstate |
| 196 | 588996 | 2367.C06.GZ43_346048 | F | M00073442A:F07 | IF97-26811-NormBPHProstate |
| 197 | 560612 | 2367.C08.GZ43_346050 | F | M00073442B:D12 | IF97-26811-NormBPHProstate |
| 198 | 15307 | 2367.C12.GZ43_346054 | F | M00073442D:E11 | IF97-26811-NormBPHProstate |
| 199 | 88462 | 2367.D11.GZ43_346077 | F | M00073446C:A03 | IF97-26811-NormBPHProstate |
| 200 | 923732 | 2367.D18.GZ43_346084 | F | M00073447B:A03 | IF97-26811-NormBPHProstate |
| 201 | 423085 | 2367.D21.GZ43_346087 | F | M00073447D:F01 | IF97-26811-NormBPHProstate |
| 202 | 483211 | 2367.E03.GZ43_346093 | F | M00073448B:F11 | IF97-26811-NormBPHProstate |
| 203 | 465814 | 2367.E04.GZ43_346094 | F | M00073448B:F07 | IF97-26811-NormBPHProstate |
| 204 | 244504 | 2367.E23.GZ43_346113 | F | M00073453C:C09 | IF97-26811-NormBPHProstate |
| 205 | 395761 | 2367.F06.GZ43_346120 | F | M00073455C:G09 | IF97-26811-NormBPHProstate |
| 206 | 514044 | 2367.F13.GZ43_346127 | F | M00073457A:G09 | IF97-26811-NormBPHProstate |
| 207 | 227227 | 2367.G11.GZ43_346149 | F | M00073462C:H12 | IF97-26811-NormBPHProstate |
| 208 | 691653 | 2367.G13.GZ43_346151 | F | M00073462D:D12 | IF97-26811-NormBPHProstate |
| 209 | 416124 | 2367.G17.GZ43_346155 | F | M00073464B:E01 | IF97-26811-NormBPHProstate |
| 210 | 452486 | 2367.G20.GZ43_346158 | F | M00073464D:G12 | IF97-26811-NormBPHProstate |
| 211 | 486366 | 2367.G22.GZ43_346160 | F | M00073465A:H08 | IF97-26811-NormBPHProstate |
| 212 | 417672 | 2367.I09.GZ43_346195 | F | M00073469B:A09 | IF97-26811-NormBPHProstate |
| 213 | 4481 | 2367.I15.GZ43_346201 | F | M00073469D:A06 | IF97-26811-NormBPHProstate |
| 214 | 11528 | 2367.I22.GZ43_346208 | F | M00073470D:A01 | IF97-26811-NormBPHProstate |
| 215 | 552537 | 2367.K06.GZ43_346240 | F | M00073474A:G11 | IF97-26811-NormBPHProstate |
| 216 | 1049007 | 2367.K13.GZ43_346247 | F | M00073474C:F08 | IF97-26811-NormBPHProstate |
| 217 | 14533 | 2367.K24.GZ43_346258 | F | M00073475D:E05 | IF97-26811-NormBPHProstate |
| 218 | 192060 | 2367.L11.GZ43_346269 | F | M00073478C:A07 | IF97-26811-NormBPHProstate |
| 219 | 571816 | 2367.M06.GZ43_346288 | F | M00073483B:C07 | IF97-26811-NormBPHProstate |
| 220 | 660248 | 2367.M14.GZ43_346296 | F | M00073484B:A05 | IF97-26811-NormBPHProstate |
| 221 | 192060 | 2367.M16.GZ43_346298 | F | M00073484C:B04 | IF97-26811-NormBPHProstate |
| 222 | 606908 | 2367.M19.GZ43_346301 | F | M00073486A:A12 | IF97-26811-NormBPHProstate |
| 223 | 466749 | 2367.N05.GZ43_346311 | F | M00073487A:C07 | IF97-26811-NormBPHProstate |
| 224 | 396325 | 2367.N16.GZ43_346322 | F | M00073489B:A07 | IF97-26811-NormBPHProstate |
| 225 | 400167 | 2367.O08.GZ43_346338 | F | M00073493A:E12 | IF97-26811-NormBPHProstate |
| 226 | 446968 | 2367.O16.GZ43_346346 | F | M00073493D:F05 | IF97-26811-NormBPHProstate |
| 227 | 160534 | 2367.O21.GZ43_346351 | F | M00073495B:G11 | IF97-26811-NormBPHProstate |
| 228 | 621397 | 2367.P12.GZ43_346366 | F | M00073497C:D03 | IF97-26811-NormBPHProstate |
| 229 | 391679 | 2368.A13.GZ43_346391 | F | M00073504D:F03 | IF97-26811-NormBPHProstate |
| 230 | 605923 | 2368.A23.GZ43_346401 | F | M00073505D:F01 | IF97-26811-NormBPHProstate |
| 231 | 416124 | 2368.B18.GZ43_346420 | F | M00073509B:B11 | IF97-26811-NormBPHProstate |
| 232 | 464200 | 2368.B20.GZ43_346422 | F | M00073509B:E03 | IF97-26811-NormBPHProstate |
| 233 | 640970 | 2368.C15.GZ43_346441 | F | M00073513A:G07 | IF97-26811-NormBPHProstate |
| 234 | 858675 | 2368.C19.GZ43_346445 | F | M00073513D:A11 | IF97-26811-NormBPHProstate |
| 235 | 467877 | 2368.D08.GZ43_346458 | F | M00073515A:F09 | IF97-26811-NormBPHProstate |
| 236 | 752831 | 2368.D20.GZ43_346470 | F | M00073517A:A06 | IF97-26811-NormBPHProstate |
| 237 | 423085 | 2368.E06.GZ43_346480 | F | M00073517D:F11 | IF97-26811-NormBPHProstate |
| 238 | 474125 | 2368.F12.GZ43_346510 | F | M00073520D:A04 | IF97-26811-NormBPHProstate |
| 239 | 70469 | 2368.F22.GZ43_346520 | F | M00073524A:A03 | IF97-26811-NormBPHProstate |
| 240 | 39999 | 2368.G01.GZ43_346523 | F | M00073524A:G05 | IF97-26811-NormBPHProstate |
| 241 | 847088 | 2368.H07.GZ43_346553 | F | M00073529A:F03 | IF97-26811-NormBPHProstate |
| 242 | 510539 | 2368.H12.GZ43_346558 | F | M00073530B:A02 | IF97-26811-NormBPHProstate |
| 243 | 402167 | 2368.H15.GZ43_346561 | F | M00073531B:H02 | IF97-26811-NormBPHProstate |
| 244 | 389538 | 2368.H17.GZ43_346563 | F | M00073531C:F12 | IF97-26811-NormBPHProstate |
| 245 | 858540 | 2368.I04.GZ43_346574 | F | M00073537B:A12 | IF97-26811-NormBPHProstate |
| 246 | 113786 | 2368.I23.GZ43_346593 | F | M00073539C:H05 | IF97-26811-NormBPHProstate |
| 247 | 468400 | 2368.J18.GZ43_346612 | F | M00073541B:C10 | IF97-26811-NormBPHProstate |
| 248 | 605923 | 2368.K19.GZ43_346637 | F | M00073547B:F04 | IF97-26811-NormBPHProstate |
| 249 | 1796 | 2368.K21.GZ43_346639 | F | M00073547C:D02 | IF97-26811-NormBPHProstate |
| 250 | 15951 | 2368.L06.GZ43_346648 | F | M00073549B:B03 | IF97-26811-NormBPHProstate |
| 251 | 43907 | 2368.L24.GZ43_346666 | F | M00073551B:E10 | IF97-26811-NormBPHProstate |
| 252 | 48738 | 2368.M19.GZ43_346685 | F | M00073552A:F06 | IF97-26811-NormBPHProstate |
| 253 | 597681 | 2368.N03.GZ43_346693 | F | M00073554A:C01 | IF97-26811-NormBPHProstate |
| 254 | 821039 | 2368.N05.GZ43_346695 | F | M00073554A:G04 | IF97-26811-NormBPHProstate |
| 255 | 954391 | 2368.N06.GZ43_346696 | F | M00073554B:A08 | IF97-26811-NormBPHProstate |
| 256 | 404368 | 2368.N08.GZ43_346698 | F | M00073554B:D11 | IF97-26811-NormBPHProstate |
| 257 | 460493 | 2368.N15.GZ43_346705 | F | M00073555A:B09 | IF97-26811-NormBPHProstate |
| 258 | 778001 | 2368.N23.GZ43_346713 | F | M00073555D:B04 | IF97-26811-NormBPHProstate |
| 259 | 404081 | 2368.O03.GZ43_346717 | F | M00073557A:A05 | IF97-26811-NormBPHProstate |
| 260 | 368947 | 2368.O11.GZ43_346725 | F | M00073558A:A02 | IF97-26811-NormBPHProstate |
| 261 | 421869 | 2368.P13.GZ43_346751 | F | M00073561C:A04 | IF97-26811-NormBPHProstate |
| 262 | 621573 | 2535.A08.GZ43_370095 | F | M00073565D:E05 | IF97-26811-NormBPHProstate |
| 263 | 640911 | 2535.A10.GZ43_370097 | F | M00073566A:G01 | IF97-26811-NormBPHProstate |
| 264 | 450754 | 2535.B09.GZ43_370120 | F | M00073568A:G06 | IF97-26811-NormBPHProstate |
| 265 | 455862 | 2535.B12.GZ43_370123 | F | M00073568C:G07 | IF97-26811-NormBPHProstate |
| 266 | 22339 | 2535.B20.GZ43_370131 | F | M00073569A:H02 | IF97-26811-NormBPHProstate |
| 267 | 372750 | 2535.C23.GZ43_370158 | F | M00073571A:F12 | IF97-26811-NormBPHProstate |
| 268 | 677530 | 2535.E22.GZ43_370205 | F | M00073575B:H12 | IF97-26811-NormBPHProstate |
| 269 | 605923 | 2535.F05.GZ43_370212 | F | M00073576B:E03 | IF97-26811-NormBPHProstate |
| 270 | 35578 | 2535.F07.GZ43_370214 | F | M00073576C:C11 | IF97-26811-NormBPHProstate |
| 271 | 568661 | 2535.F11.GZ43_370218 | F | M00073577B:D12 | IF97-26811-NormBPHProstate |
| 272 | 64401 | 2535.G02.GZ43_370233 | F | M00073579B:A04 | IF97-26811-NormBPHProstate |
| 273 | 76555 | 2535.G13.GZ43_370244 | F | M00073580A:D08 | IF97-26811-NormBPHProstate |
| 274 | 36568 | 2535.J20.GZ43_370323 | F | M00073587D:E12 | IF97-26811-NormBPHProstate |
| 275 | 533888 | 2535.K01.GZ43_370328 | F | M00073588B:H07 | IF97-26811-NormBPHProstate |
| 276 | 13301 | 2535.L03.GZ43_370354 | F | M00073590C:F07 | IF97-26811-NormBPHProstate |
| 277 | 52735 | 2535.L18.GZ43_370369 | F | M00073592B:D09 | IF97-26811-NormBPHProstate |
| 278 | 33508 | 2535.M11.GZ43_370386 | F | M00073594B:B11 | IF97-26811-NormBPHProstate |
| 279 | 436659 | 2535.N06.GZ43_370405 | F | M00073595D:A11 | IF97-26811-NormBPHProstate |
| 280 | 451707 | 2535.O07.GZ43_370430 | F | M00073598D:E11 | IF97-26811-NormBPHProstate |
| 281 | 481445 | 2535.O13.GZ43_370436 | F | M00073599C:E08 | IF97-26811-NormBPHProstate |
| 282 | 135469 | 2535.P02.GZ43_370449 | F | M00073601A:B06 | IF97-26811-NormBPHProstate |
| 283 | 36102 | 2535.P06.GZ43_370453 | F | M00073601A:F07 | IF97-26811-NormBPHProstate |
| 284 | 6712 | 2535.P14.GZ43_370461 | F | M00073601D:D08 | IF97-26811-NormBPHProstate |
| 285 | 87043 | 2536.A06.GZ43_370477 | F | M00073603A:F04 | IF97-26811-NormBPHProstate |
| 286 | 375483 | 2536.A07.GZ43_370478 | F | M00073603B:C03 | IF97-26811-NormBPHProstate |
| 287 | 415500 | 2536.A08.GZ43_370479 | F | M00073603C:A11 | IF97-26811-NormBPHProstate |
| 288 | 7368 | 2536.A09.GZ43_370480 | F | M00073603C:C02 | IF97-26811-NormBPHProstate |
| 289 | 553460 | 2536.A14.GZ43_370485 | F | M00073603D:E07 | IF97-26811-NormBPHProstate |
| 290 | 210361 | 2536.A19.GZ43_370490 | F | M00073604B:B07 | IF97-26811-NormBPHProstate |
| 291 | 260521 | 2536.A20.GZ43_370491 | F | M00073604B:H06 | IF97-26811-NormBPHProstate |
| 292 | 70406 | 2536.A22.GZ43_370493 | F | M00073604C:H09 | IF97-26811-NormBPHProstate |
| 293 | 21817 | 2536.B06.GZ43_370501 | F | M00073605B:F10 | IF97-26811-NormBPHProstate |
| 294 | 62816 | 2536.B07.GZ43_370502 | F | M00073605B:F11 | IF97-26811-NormBPHProstate |
| 295 | 10376 | 2536.B15.GZ43_370510 | F | M00073606D:F12 | IF97-26811-NormBPHProstate |
| 296 | 35707 | 2536.C12.GZ43_370531 | F | M00073610A:F06 | IF97-26811-NormBPHProstate |
| 297 | 738158 | 2536.D17.GZ43_370560 | F | M00073614B:A12 | IF97-26811-NormBPHProstate |
| 298 | 974091 | 2536.D20.GZ43_370563 | F | M00073614B:G09 | IF97-26811-NormBPHProstate |
| 299 | 374280 | 2536.D22.GZ43_370565 | F | M00073614C:F06 | IF97-26811-NormBPHProstate |
| 300 | 375209 | 2536.E08.GZ43_370575 | F | M00073615D:E03 | IF97-26811-NormBPHProstate |
| 301 | 176266 | 2536.E11.GZ43_370578 | F | M00073616A:F06 | IF97-26811-NormBPHProstate |
| 302 | 31475 | 2536.E21.GZ43_370588 | F | M00073617A:H04 | IF97-26811-NormBPHProstate |
| 303 | 235423 | 2536.G05.GZ43_370620 | F | M00073620A:G05 | IF97-26811-NormBPHProstate |
| 304 | 88462 | 2536.G20.GZ43_370635 | F | M00073621D:A04 | IF97-26811-NormBPHProstate |
| 305 | 186007 | 2536.G21.GZ43_370636 | F | M00073621D:D02 | IF97-26811-NormBPHProstate |
| 306 | 12346 | 2536.G22.GZ43_370637 | F | M00073621D:H05 | IF97-26811-NormBPHProstate |
| 307 | 98685 | 2536.H08.GZ43_370647 | F | M00073623D:H10 | IF97-26811-NormBPHProstate |
| 308 | 861172 | 2536.H20.GZ43_370659 | F | M00073625C:D09 | IF97-26811-NormBPHProstate |
| 309 | 164426 | 2536.I05.GZ43_370668 | F | M00073626D:A01 | IF97-26811-NormBPHProstate |
| 310 | 428727 | 2536.I15.GZ43_370678 | F | M00073628A:E03 | IF97-26811-NormBPHProstate |
| 311 | 573 | 2536.J05.GZ43_370692 | F | M00073630A:C03 | IF97-26811-NormBPHProstate |
| 312 | 883034 | 2536.J09.GZ43_370696 | F | M00073630B:E09 | IF97-26811-NormBPHProstate |
| 313 | 856743 | 2536.J11.GZ43_370698 | F | M00073630C:D02 | IF97-26811-NormBPHProstate |
| 314 | 60888 | 2536.K12.GZ43_370723 | F | M00073632A:B12 | IF97-26811-NormBPHProstate |
| 315 | 207397 | 2536.K21.GZ43_370732 | F | M00073632C:A03 | IF97-26811-NormBPHProstate |
| 316 | 177456 | 2536.L18.GZ43_370753 | F | M00073633D:A04 | IF97-26811-NormBPHProstate |
| 317 | 47454 | 2536.L22.GZ43_370757 | F | M00073633D:G04 | IF97-26811-NormBPHProstate |
| 318 | 33967 | 2536.M10.GZ43_370769 | F | M00073634C:H08 | IF97-26811-NormBPHProstate |
| 319 | 402043 | 2536.N05.GZ43_370788 | F | M00073635D:C10 | IF97-26811-NormBPHProstate |
| 320 | 831101 | 2536.N20.GZ43_370803 | F | M00073636C:F03 | IF97-26811-NormBPHProstate |
| 321 | 736938 | 2536.O12.GZ43_370819 | F | M00073637C:B01 | IF97-26811-NormBPHProstate |
| 322 | 40144 | 2536.O14.GZ43_370821 | F | M00073637C:E04 | IF97-26811-NormBPHProstate |
| 323 | 13473 | 2536.O22.GZ43_370829 | F | M00073638A:A12 | IF97-26811-NormBPHProstate |
| 324 | 23951 | 2536.P14.GZ43_370845 | F | M00073638D:D10 | IF97-26811-NormBPHProstate |
| 325 | 72334 | 2536.P17.GZ43_370848 | F | M00073639A:G08 | IF97-26811-NormBPHProstate |
| 326 | 140322 | 2536.P22.GZ43_370853 | F | M00073639B:F02 | IF97-26811-NormBPHProstate |
| 327 | 42714 | 2536.M04.GZ43_370763 | F | M00073634B:C12 | IF97-26811-NormBPHProstate |
| 328 | 25714 | 2537.A21.GZ43_370876 | F | M00073640B:G08 | IF97-26811-NormBPHProstate |
| 329 | 177456 | 2537.A23.GZ43_370878 | F | M00073640C:A03 | IF97-26811-NormBPHProstate |
| 330 | 7546 | 2537.B07.GZ43_370886 | F | M00073640D:A11 | IF97-26811-NormBPHProstate |
| 331 | 21102 | 2537.B14.GZ43_370893 | F | M00073640D:G07 | IF97-26811-NormBPHProstate |
| 332 | 375856 | 2537.C10.GZ43_370913 | F | M00073641B:G07 | IF97-26811-NormBPHProstate |
| 333 | 15080 | 2537.C18.GZ43_370921 | F | M00073641C:E04 | IF97-26811-NormBPHProstate |
| 334 | 44198 | 2537.D11.GZ43_370938 | F | M00073643B:E11 | IF97-26811-NormBPHProstate |
| 335 | 598913 | 2537.D20.GZ43_370947 | F | M00073644A:G12 | IF97-26811-NormBPHProstate |
| 336 | 374952 | 2537.F01.GZ43_370976 | F | M00073646A:C01 | IF97-26811-NormBPHProstate |
| 337 | 374839 | 2537.F18.GZ43_370993 | F | M00073647B:H07 | IF97-26811-NormBPHProstate |
| 338 | 21817 | 2537.G05.GZ43_371004 | F | M00073649A:A03 | IF97-26811-NormBPHProstate |
| 339 | 3211 | 2537.G09.GZ43_371008 | F | M00073649A:G08 | IF97-26811-NormBPHProstate |
| 340 | 397144 | 2537.H24.GZ43_371047 | F | M00073651C:F06 | IF97-26811-NormBPHProstate |
| 341 | 379025 | 2537.I03.GZ43_371050 | F | M00073651C:H07 | IF97-26811-NormBPHProstate |
| 342 | 7368 | 2537.I08.GZ43_371055 | F | M00073652D:B11 | IF97-26811-NormBPHProstate |
| 343 | 350 | 2537.J07.GZ43_371078 | F | M00073655B:A04 | IF97-26811-NormBPHProstate |
| 344 | 55140 | 2537.J23.GZ43_371094 | F | M00073657B:D05 | IF97-26811-NormBPHProstate |
| 345 | 4031 | 2537.K17.GZ43_371112 | F | M00073659C:D03 | IF97-26811-NormBPHProstate |
| 346 | 48711 | 2537.L23.GZ43_371142 | F | M00073663A:E02 | IF97-26811-NormBpHProstate |
| 347 | 744278 | 2537.M11.GZ43_371154 | F | M00073663D:G06 | IF97-26811-NormBPHProstate |
| 348 | 436755 | 2537.M14.GZ43_371157 | F | M00073664A:E03 | IF97-26811-NormBPHProstate |
| 349 | 148227 | 2537.N12.GZ43_371179 | F | M00073666B:B01 | IF97-26811-NormBPHProstate |
| 350 | 402325 | 2537.N23.GZ43_371190 | F | M00073668A:H03 | IF97-26811-NormBPHProstate |
| 351 | 14002 | 2537.N24.GZ43_371191 | F | M00073668B:A08 | IF97-26811-NormBPHProstate |
| 352 | 714906 | 2537.O05.GZ43_371196 | F | M00073668D:D10 | IF97-26811-NormBPHProstate |
| 353 | 557739 | 2537.O10.GZ43_371201 | F | M00073669A:F04 | IF97-26811-NormBPHProstate |
| 354 | 296 | 2537.O13.GZ43_371204 | F | M00073669B:E12 | IF97-26811-NormBPHProstate |
| 355 | 373515 | 2537.O21.GZ43_371212 | F | M00073669D:G10 | IF97-26811-NormBPHProstate |
| 356 | 455443 | 2537.P14.GZ43_371229 | F | M00073671B:D09 | IF97-26811-NormBPHProstate |
| 357 | 12272 | 2538.F24.GZ43_371383 | F | M00073687A:D11 | IF97-26811-NormBPHProstate |
| 358 | 380624 | 2538.M23.GZ43_371550 | F | M00073699C:E02 | IF97-26811-NormBPHProstate |
| 359 | 4442 | 2538.N23.GZ43_371574 | F | M00073701D:G10 | IF97-26811-NormBPHProstate |
| 360 | 556517 | 2538.A08.GZ43_371247 | F | M00073672D:B07 | IF97-26811-NormBPHProstate |
| 361 | 530582 | 2538.A10.GZ43_371249 | F | M00073672D:E09 | IF97-26811-NormBPHProstate |
| 362 | 8126 | 2538.A12.GZ43_371251 | F | M00073673A:D11 | IF97-26811-NormBPHProstate |
| 363 | 733673 | 2538.B03.GZ43_371266 | F | M00073673D:H03 | IF97-26811-NormBPHProstate |
| 364 | 446 | 2538.B15.GZ43_371278 | F | M00073674D:F10 | IF97-26811-NormBPHProstate |
| 365 | 449576 | 2538.B20.GZ43_371283 | F | M00073676A:G08 | IF97-26811-NormBPHProstate |
| 366 | 555630 | 2538.C07.GZ43_371294 | F | M00073676D:H04 | IF97-26811-NormBPHProstate |
| 367 | 19627 | 2538.C14.GZ43_371301 | F | M00073677B:F01 | IF97-26811-NormBPHProstate |
| 368 | 401402 | 2538.D03.GZ43_371314 | F | M00073678B:E08 | IF97-26811-NormBPHProstate |
| 369 | 296 | 2538.D04.GZ43_371315 | F | M00073678B:H02 | IF97-26811-NormBPHProstate |
| 370 | 3843 | 2538.D11.GZ43_371322 | F | M00073679A:D06 | IF97-26811-NormBPHProstate |
| 371 | 1239 | 2538.E01.GZ43_371336 | F | M00073680D:F11 | IF97-26811-NormBPHProstate |
| 372 | 676448 | 2538.E05.GZ43_371340 | F | M00073681A:F12 | IF97-26811-NormBPHProstate |
| 373 | 423064 | 2538.E22.GZ43_371357 | F | M00073684B:F10 | IF97-26811-NormBPHProstate |
| 374 | 449749 | 2538.F03.GZ43_371362 | F | M00073685A:F07 | IF97-26811-NormBPHProstate |
| 375 | 72417 | 2538.H02.GZ43_371409 | F | M00073688C:A12 | IF97-26811-NormBPHProstate |
| 376 | 4650 | 2538.H08.GZ43_371415 | F | M00073688D:C11 | IF97-26811-NormBPHProstate |
| 377 | 673484 | 2538.H19.GZ43_371426 | F | M00073689C:C09 | IF97-26811-NormBPHProstate |
| 378 | 134226 | 2538.I06.GZ43_371437 | F | M00073690B:G04 | IF97-26811-NormBPHProstate |
| 379 | 9516 | 2538.I17.GZ43_371448 | F | M00073691A:G02 | IF97-26811-NormBPHProstate |
| 380 | 400463 | 2538.J10.GZ43_371465 | F | M00073692D:H02 | IF97-26811-NormBPHProstate |
| 381 | 48289 | 2538.K17.GZ43_371496 | F | M00073695C:D11 | IF97-26811-NormBPHProstate |
| 382 | 35380 | 2538.L09.GZ43_371512 | F | M00073696C:D11 | IF97-26811-NormBPHProstate |
| 383 | 375810 | 2538.L11.GZ43_371514 | F | M00073696D:A08 | IF97-26811-NormBPHProstate |
| 384 | 640911 | 2538.L20.GZ43_371523 | F | M00073697C:F11 | IF97-26811-NormBPHProstate |
| 385 | 374382 | 2538.M16.GZ43_371543 | F | M00073699B:D02 | IF97-26811-NormBPHProstate |
| 386 | 448604 | 2538.M17.GZ43_371544 | F | M00073699B:D09 | IF97-26811-NormBPHProstate |
| 387 | 447798 | 2538.N06.GZ43_371557 | F | M00073700A:C09 | IF97-26811-NormBPHProstate |
| 388 | 452289 | 2538.N11.GZ43_371562 | F | M00073700B:D12 | IF97-26811-NormBPHProstate |
| 389 | 518084 | 2538.P16.GZ43_371615 | F | M00073707B:G08 | IF97-26811-NormBPHProstate |
| 390 | 706359 | 2554.A04.GZ43_375851 | F | M00073708D:E10 | IF97-26811-NormBPHProstate |
| 391 | 901160 | 2554.A06.GZ43_375853 | F | M00073708D:F03 | IF97-26811-NormBPHProstate |
| 392 | 510479 | 2554.A12.GZ43_375859 | F | M00073709B:F01 | IF97-26811-NormBPHProstate |
| 393 | 149529 | 2554.A15.GZ43_375862 | F | M00073709C:A01 | IF97-26811-NormBPHProstate |
| 394 | 727966 | 2554.A16.GZ43_375863 | F | M00073709C:A02 | IF97-26811-NormBPHProstate |
| 395 | 398682 | 2554.A23.GZ43_375870 | F | M00073710B:A09 | IF97-26811-NormBPHProstate |
| 396 | 57638 | 2554.B12.GZ43_375883 | F | M00073710D:G06 | IF97-26811-NormBPHProstate |
| 397 | 8956 | 2554.B17.GZ43_375888 | F | M00073711C:E12 | IF97-26811-NormBPHProstate |
| 398 | 599028 | 2554.D02.GZ43_375921 | F | M00073713D:E07 | IF97-26811-NormBPHProstate |
| 399 | 497138 | 2554.D09.GZ43_375928 | F | M00073715A:F05 | IF97-26811-NormBPHProstate |
| 400 | 735042 | 2554.D12.GZ43_375931 | F | M00073715B:B06 | IF97-26811-NormBPHProstate |
| 401 | 42867 | 2554.E10.GZ43_375953 | F | M00073717C:A12 | IF97-26811-NormBPHProstate |
| 402 | 29906 | 2554.E17.GZ43_375960 | F | M00073718A:F11 | IF97-26811-NormBPHProstate |
| 403 | 560612 | 2554.F20.GZ43_375987 | F | M00073720D:H11 | IF97-26811-NormBPHProstate |
| 404 | 980 | 2554.G22.GZ43_376013 | F | M00073724D:F04 | IF97-26811-NormBPHProstate |
| 405 | 642041 | 2554.I10.GZ43_376049 | F | M00073732C:B09 | IF97-26811-NormBPHProstate |
| 406 | 163500 | 2554.I15.GZ43_376054 | F | M00073733A:A05 | IF97-26811-NormBPHProstate |
| 407 | 1522 | 2554.I18.GZ43_376057 | F | M00073733A:E03 | IF97-26811-NormBPHProstate |
| 408 | 573764 | 2554.J15.GZ43_376078 | F | M00073735C:E04 | IF97-26811-NormBPHProstate |
| 409 | 40330 | 2554.K08.GZ43_376095 | F | M00073737A:C12 | IF97-26811-NormBPHProstate |
| 410 | 525011 | 2554.L09.GZ43_376120 | F | M00073739D:B04 | IF97-26811-NormBPHProstate |
| 411 | 847088 | 2554.L18.GZ43_376129 | F | M00073740B:F08 | IF97-26811-NormBPHProstate |
| 412 | 36174 | 2554.M14.GZ43_376149 | F | M00073741C:D05 | IF97-26811-NormBPHProstate |
| 413 | 455254 | 2554.N09.GZ43_376168 | F | M00073743C:F03 | IF97-26811-NormBPHProstate |
| 414 | 89912 | 2554.O17.GZ43_376200 | F | M00073746A:H03 | IF97-26811-NormBPHProstate |
| 415 | 451707 | 2554.P16.GZ43_376223 | F | M00073748A:F09 | IF97-26811-NormBPHProstate |
| 416 | 43900 | 2554.P17.GZ43_376224 | F | M00073748B:A12 | IF97-26811-NormBPHProstate |
| 417 | 752831 | 2554.P23.GZ43_376230 | F | M00073748B:F07 | IF97-26811-NormBPHProstate |
| 418 | 558581 | 2565.B13.GZ43_398139 | F | M00073750A:E08 | IF97-26811-NormBPHProstate |
| 419 | 7307 | 2565.B15.GZ43_398171 | F | M00073750A:H08 | IF97-26811-NormBPHProstate |
| 420 | 403109 | 2565.B18.GZ43_398219 | F | M00073750B:D05 | IF97-26811-NormBPHProstate |
| 421 | 60809 | 2565.C02.GZ43_397964 | F | M00073750C:G06 | IF97-26811-NormBPHProstate |
| 422 | 375711 | 2565.C17.GZ43_398204 | F | M00073751D:A06 | IF97-26811-NormBPHProstate |
| 423 | 1371 | 2565.D06.GZ43_398029 | F | M00073753B:B05 | IF97-26811-NormBPHProstate |
| 424 | 402399 | 2565.D22.GZ43_398285 | F | M00073754B:D05 | IF97-26811-NormBPHProstate |
| 425 | 18508 | 2565.E03.GZ43_397982 | F | M00073754B:H02 | IF97-26811-NormBPHProstate |
| 426 | 617 | 2565.E05.GZ43_398014 | F | M00073754C:C01 | IF97-26811-NormBPHProstate |
| 427 | 147634 | 2565.F18.GZ43_398223 | F | M00073758C:G03 | IF97-26811-NormBPHProstate |
| 428 | 10334 | 2565.G20.GZ43_398256 | F | M00073760B:B11 | IF97-26811-NormBPHProstate |
| 429 | 1530 | 2565.H01.GZ43_397953 | F | M00073760D:F04 | IF97-26811-NormBPHProstate |
| 430 | 373261 | 2565.H12.GZ43_398129 | F | M00073762A:B09 | IF97-26811-NormBPHProstate |
| 431 | 18746 | 2565.H21.GZ43_398273 | F | M00073762D:C02 | IF97-26811-NormBPHProstate |
| 432 | 524083 | 2565.H24.GZ43_398321 | F | M00073763A:D06 | IF97-26811-NormBPHProstate |
| 433 | 724819 | 2565.I22.GZ43_398290 | F | M00073764B:B09 | IF97-26811-NormBPHProstate |
| 434 | 401809 | 2565.J08.GZ43_398067 | F | M00073764D:A07 | IF97-26811-NormBPHProstate |
| 435 | 424776 | 2565.J09.GZ43_398083 | F | M00073764D:B12 | IF97-26811-NormBPHProstate |
| 436 | 648899 | 2565.J13.GZ43_398147 | F | M00073765A:E02 | IF97-26811-NormBPHProstate |
| 437 | 752623 | 2565.J19.GZ43_398243 | F | M00073765C:B01 | IF97-26811-NormBPHProstate |
| 438 | 193333 | 2565.K04.GZ43_398004 | F | M00073766A:B07 | IF97-26811-NormBPHProstate |
| 439 | 493811 | 2565.K07.GZ43_398052 | F | M00073766B:B07 | IF97-26811-NormBPHProstate |
| 440 | 46581 | 2565.K09.GZ43_398084 | F | M00073766B:C04 | IF97-26811-NormBPHProstate |
| 441 | 19736 | 2565.L21.GZ43_398277 | F | M00073769D:G10 | IF97-26811-NormBPHProstate |
| 442 | 449073 | 2565.M14.GZ43_398166 | F | M00073772B:E07 | IF97-26811-NormBPHProstate |
| 443 | 42891 | 2565.M24.GZ43_398326 | F | M00073773A:F05 | IF97-26811-NormBPHProstate |
| 444 | 456043 | 2565.N02.GZ43_397975 | F | M00073773A:G04 | IF97-26811-NormBPHProstate |
| 445 | 70411 | 2565.N03.GZ43_397991 | F | M00073773B:A09 | IF97-26811-NormBPHProstate |
| 446 | 174228 | 2565.N20.GZ43_398263 | F | M00073774C:G12 | IF97-26811-NormBPHProstate |
| 447 | 448795 | 2565.O07.GZ43_398056 | F | M00073776C:F11 | IF97-26811-NormBPHProstate |
| 448 | 452714 | 2565.O12.GZ43_398136 | F | M00073777A:A01 | IF97-26811-NormBPHProstate |
| 449 | 70908 | 2565.O16.GZ43_398200 | F | M00073777A:H03 | IF97-26811-NormBPHProstate |
| 450 | 562386 | 2565.P08.GZ43_398073 | F | M00073779B:B11 | IF97-26811-NormBPHProstate |
| 451 | 21817 | 2565.P24.GZ43_398329 | F | M00073784A:A12 | IF97-26811-NormBPHProstate |
| 452 | 696086 | 2540.A24.GZ43_372031 | F | M00073785C:A05 | IF97-26811-NormBPHProstate |
| 453 | 36174 | 2540.B02.GZ43_372033 | F | M00073785D:D01 | IF97-26811-NormBPHProstate |
| 454 | 481445 | 2540.C04.GZ43_372059 | F | M00073787D:H12 | IF97-26811-NormBPHProstate |
| 455 | 552537 | 2540.C10.GZ43_372065 | F | M00073788C:A10 | IF97-26811-NormBPHProstate |
| 456 | 507628 | 2540.D02.GZ43_372081 | F | M00073790C:E07 | IF97-26811-NormBPHProstate |
| 457 | 113786 | 2540.E09.GZ43_372112 | F | M00073793C:E09 | IF97-26811-NormBPHProstate |
| 458 | 454796 | 2540.F03.GZ43_372130 | F | M00073795A:F03 | IF97-26811-NormBPHProstate |
| 459 | 134637 | 2540.F05.GZ43_372132 | F | M00073795B:B05 | IF97-26811-NormBPHProstate |
| 460 | 450227 | 2540.F06.GZ43_372133 | F | M00073795B:B09 | IF97-26811-NormBPHProstate |
| 461 | 23300 | 2540.F13.GZ43_372140 | F | M00073796A:C03 | IF97-26811-NormBPHProstate |
| 462 | 57350 | 2540.G11.GZ43_372162 | F | M00073798A:H03 | IF97-26811-NormBPHProstate |
| 463 | 633752 | 2540.H07.GZ43_372182 | F | M00073800D:F08 | IF97-26811-NormBPHProstate |
| 464 | 516985 | 2540.H13.GZ43_372188 | F | M00073801B:A10 | IF97-26811-NormBPHProstate |
| 465 | 376272 | 2540.I10.GZ43_372209 | F | M00073802D:B11 | IF97-26811-NormBPHProstate |
| 466 | 39862 | 2540.K12.GZ43_372259 | F | M00073806D:C09 | IF97-26811-NormBPHProstate |
| 467 | 525801 | 2540.M05.GZ43_372300 | F | M00073809C:E09 | IF97-26811-NormBPHProstate |
| 468 | 830453 | 2540.M22.GZ43_372317 | F | M00073810C:F05 | IF97-26811-NormBPHProstate |
| 469 | 454796 | 2540.P02.GZ43_372369 | F | M00073813D:B06 | IF97-26811-NormBPHProstate |
| 470 | 572170 | 2540.P13.GZ43_372380 | F | M00073814C:B04 | IF97-26811-NormBPHProstate |
| 471 | 44044 | 2540.B15.GZ43_372046 | F | M00073786D:B03 | IF97-26811-NormBPHProstate |
| 472 | 553297 | 2540.C19.GZ43_372074 | F | M00073789C:B06 | IF97-26811-NormBPHProstate |
| 473 | 402167 | 2540.C21.GZ43_372076 | F | M00073790A:A12 | IF97-26811-NormBPHprostate |
| 474 | 38334 | 2540.D19.GZ43_372098 | F | M00073792B:A03 | IF97-26811-NormBPHProstate |
| 475 | 477271 | 2540.E17.GZ43_372120 | F | M00073794B:G09 | IF97-26811-NormBPHProstate |
| 476 | 519354 | 2540.F01.GZ43_372128 | F | M00073794D:G07 | IF97-26811-NormBPHProstate |
| 477 | 528957 | 2540.F15.GZ43_372142 | F | M00073796A:D08 | IF97-26811-NormBPHProstate |
| 478 | 89912 | 2540.F17.GZ43_372144 | F | M00073796B:A03 | IF97-26811-NormBPHProstate |
| 479 | 495563 | 2540.G16.GZ43_372167 | F | M00073799A:A09 | IF97-26811-NormBPHProstate |
| 480 | 626993 | 2540.G19.GZ43_372170 | F | M00073799A:G02 | IF97-26811-NormBPHProstate |
| 481 | 429609 | 2540.H01.GZ43_372176 | F | M00073799D:G04 | IF97-26811-NormBPHProstate |
| 482 | 932437 | 2540.I17.GZ43_372216 | F | M00073803B:B03 | IF97-26811-NormBPHProstate |
| 483 | 427559 | 2540.I20.GZ43_372219 | F | M00073803B:C06 | IF97-26811-NormBPHProstate |
| 484 | 14214 | 2540.M15.GZ43_372310 | F | M00073810B:G10 | IF97-26811-NormBPHProstate |
| 485 | 379689 | 2540.M18.GZ43_372313 | F | M00073810C:A06 | IF97-26811-NormBPHProstate |
| 486 | 552374 | 2540.O16.GZ43_372359 | F | M00073813A:E06 | IF97-26811-NormBPHProstate |
| 487 | 743053 | 2540.O19.GZ43_372362 | F | M00073813B:A01 | IF97-26811-NormBPHProstate |
| 488 | 474125 | 2541.A06.GZ43_372397 | F | M00073815D:E02 | IF97-26811-NormBPHProstate |
| 489 | 498886 | 2541.B15.GZ43_372430 | F | M00073818A:A06 | IF97-26811-NormBPHProstate |
| 490 | 993554 | 2541.D03.GZ43_372466 | F | M00073819D:C11 | IF97-26811-NormBPHProstate |
| 491 | 7170 | 2541.D14.GZ43_372477 | F | M00073821A:B10 | IF97-26811-NormBPHProstate |
| 492 | 36866 | 2541.D21.GZ43_372484 | F | M00073821B:H03 | IF97-26811-NormBPHProstate |
| 493 | 451707 | 2541.E16.GZ43_372503 | F | M00073822C:E02 | IF97-26811-NormBPHProstate |
| 494 | 948383 | 2541.F05.GZ43_372516 | F | M00073824A:C04 | IF97-26811-NormBPHProstate |
| 495 | 454796 | 2541.F18.GZ43_372529 | F | M00073826B:C01 | IF97-26811-NormBPHProstate |
| 496 | 821039 | 2541.I08.GZ43_372591 | F | M00073831B:H09 | IF97-26811-NormBPHProstate |
| 497 | 568204 | 2541.I17.GZ43_372600 | F | M00073832A:A06 | IF97-26811-NormBPHProstate |
| 498 | 652099 | 2541.I23.GZ43_372606 | F | M00073832A:G01 | IF97-26811-NormBPHProstate |
| 499 | 723822 | 2541.I24.GZ43_372607 | F | M00073832B:B05 | IF97-26811-NormBPHProstate |
| 500 | 207018 | 2541.J17.GZ43_372624 | F | M00073834A:H10 | IF97-26811-NormBPHProstate |
| 501 | 2745 | 2541.J23.GZ43_372630 | F | M00073834D:E07 | IF97-26811-NormBPHProstate |
| 502 | 1049007 | 2541.K02.GZ43_372633 | F | M00073834D:H06 | IF97-26811-NormBPHProstate |
| 503 | 558463 | 2541.K15.GZ43_372646 | F | M00073836D:E05 | IF97-26811-NormBPHProstate |
| 504 | 20052 | 2541.K18.GZ43_372649 | F | M00073837B:D12 | IF97-26811-NormBPHProstate |
| 505 | 208449 | 2541.L02.GZ43_372657 | F | M00073838A:H07 | IF97-26811-NormBPHProstate |
| 506 | 853371 | 2541.L06.GZ43_372661 | F | M00073838B:F09 | IF97-26811-NormBPHProstate |
| 507 | 398682 | 2541.L08.GZ43_372663 | F | M00073838B:H06 | IF97-26811-NormBPHProstate |
| 508 | 40241 | 2541.L12.GZ43_372667 | F | M00073838D:E01 | IF97-26811-NormBPHProstate |
| 509 | 423085 | 2541.L21.GZ43_372676 | F | M00073839A:D05 | IF97-26811-NormBPHProstate |
| 510 | 640911 | 2541.M24.GZ43_372703 | F | M00073840D:C08 | IF97-26811-NormBPHProstate |
| 511 | 520370 | 2541.N01.GZ43_372704 | F | M00073841A:A03 | IF97-26811-NormBPHProstate |
| 512 | 643828 | 2541.P14.GZ43_372765 | F | M00073845D:F05 | IF97-26811-NormBPHProstate |
| 513 | 384776 | 2506.C08.GZ43_366613 | F | M00073850A:H09 | IF97-26811-NormBPHProstate |
| 514 | 765 | 2506.C15.GZ43_366620 | F | M00073850D:G04 | IF97-26811-NormBPHProstate |
| 515 | 3188 | 2506.C18.GZ43_366623 | F | M00073851A:C05 | IF97-26811-NormBPHProstate |
| 516 | 20818 | 2506.C20.GZ43_366625 | F | M00073851A:E04 | IF97-26811-NormBPHProstate |
| 517 | 401067 | 2506.E01.GZ43_366654 | F | M00073853C:A01 | IF97-26811-NormBPHProstate |
| 518 | 382 | 2506.E12.GZ43_366665 | F | M00073854B:B04 | IF97-26811-NormBPHProstate |
| 519 | 237334 | 2506.E18.GZ43_366671 | F | M00073854C:F08 | IF97-26811-NormBPHProstate |
| 520 | 379913 | 2506.G01.GZ43_366702 | F | M00073857A:B12 | IF97-26811-NormBPHProstate |
| 521 | 663109 | 2506.G24.GZ43_366725 | F | M00073859A:C09 | IF97-26811-NormBPHProstate |
| 522 | 702885 | 2506.H20.GZ43_366745 | F | M00073860B:F12 | IF97-26811-NormBPHProstate |
| 523 | 374164 | 2506.I12.GZ43_366761 | F | M00073861D:A09 | IF97-26811-NormBPHProstate |
| 524 | 402325 | 2506.I14.GZ43_366763 | F | M00073861D:D08 | IF97-26811-NormBPHProstate |
| 525 | 2660 | 2506.I24.GZ43_366773 | F | M00073862B:D11 | IF97-26811-NormBPHProstate |
| 526 | 373578 | 2506.J12.GZ43_366785 | F | M00073862D:F06 | IF97-26811-NormBPHProstate |
| 527 | 403773 | 2506.J20.GZ43_366793 | F | M00073863B:G09 | IF97-26811-NormBPHProstate |
| 528 | 4290 | 2506.J22.GZ43_366795 | F | M00073863C:D04 | IF97-26811-NormBPHProstate |
| 529 | 117060 | 2506.K20.GZ43_366817 | F | M00073865B:G04 | IF97-26811-NormBPHProstate |
| 530 | 42794 | 2506.L08.GZ43_366829 | F | M00073866A:G07 | IF97-26811-NormBPHProstate |
| 531 | 40541 | 2506.M05.GZ43_366850 | F | M00073867B:E01 | IF97-26811-NormBPHProstate |
| 532 | 401013 | 2506.M13.GZ43_366858 | F | M00073867D:F10 | IF97-26811-NormBPHProstate |
| 533 | 374406 | 2506.O11.GZ43_366904 | F | M00073871B:C12 | IF97-26811-NormBPHProstate |
| 534 | 40094 | 2506.P07.GZ43_366924 | F | M00073872C:B09 | IF97-26811-NormBPHProstate |
| 535 | 374280 | 2506.P11.GZ43_366928 | F | M00073872D:B01 | IF97-26811-NormBPHProstate |
| 536 | 376054 | 2506.P13.GZ43_366930 | F | M00073872D:E10 | IF97-26811-NormBPHProstate |
| 537 | 172474 | 2506.P19.GZ43_366936 | F | M00073873C:A06 | IF97-26811-NormBPHProstate |
| 538 | 8159 | 2542.A15.GZ43_372790 | F | M00073875A:B03 | IF97-26811-NormBPHProstate |
| 539 | 51272 | 2542.B01.GZ43_372800 | F | M00073875C:G02 | IF97-26811-NormBPHProstate |
| 540 | 709796 | 2542.C20.GZ43_372843 | F | M00073878C:A03 | IF97-26811-NormBPHProstate |
| 541 | 380482 | 2542.D09.GZ43_372856 | F | M00073879D:B08 | IF97-26811-NormBPHProstate |
| 542 | 573764 | 2542.D18.GZ43_372865 | F | M00073880B:B02 | IF97-26811-NormBPHProstate |
| 543 | 5105 | 2542.D19.GZ43_372866 | F | M00073880B:B09 | IF97-26811-NormBPHProstate |
| 544 | 551379 | 2542.F05.GZ43_372900 | F | M00073883B:D03 | IF97-26811-NormBPHProstate |
| 545 | 615999 | 2542.F08.GZ43_372903 | F | M00073883B:H03 | IF97-26811-NormBPHProstate |
| 546 | 464200 | 2542.H02.GZ43_372945 | F | M00073886C:C12 | IF97-26811-NormBPHProstate |
| 547 | 743053 | 2542.I14.GZ43_372981 | F | M00073889B:G08 | IF97-26811-NormBPHProstate |
| 548 | 483211 | 2542.J12.GZ43_373003 | F | M00073891A:A06 | IF97-26811-NormBPHProstate |
| 549 | 519354 | 2542.K05.GZ43_373020 | F | M00073892A:E02 | IF97-26811-NormBPHProstate |
| 550 | 595883 | 2542.K08.GZ43_373023 | F | M00073892B:F12 | IF97-26811-NormBPHProstate |
| 551 | 374817 | 2542.L03.GZ43_373042 | F | M00073893D:A04 | IF97-26811-NormBPHProstate |
| 552 | 604822 | 2542.M05.GZ43_373068 | F | M00073895C:F02 | IF97-26811-NormBPHProstate |
| 553 | 454509 | 2542.M09.GZ43_373072 | F | M00073896A:F07 | IF97-26811-NormBPHProstate |
| 554 | 184489 | 2542.O05.GZ43_373116 | F | M00073899C:E12 | IF97-26811-NormBPHProstate |
| 555 | 565709 | 2542.P02.GZ43_373137 | F | M00073905B:A03 | IF97-26811-NormBPHProstate |
| 556 | 13301 | 2542.P08.GZ43_373143 | F | M00073905D:C11 | IF97-26811-NormBPHProstate |
| 557 | 723485 | 2542.P19.GZ43_373154 | F | M00073907B:B06 | IF97-26811-NormBPHProstate |
| 558 | 418723 | 2542.F24.GZ43_372919 | F | M00073884D:B06 | IF97-26811-NormBPHProstate |
| 559 | 847088 | 2542.H23.GZ43_372966 | F | M00073888C:C10 | IF97-26811-NormBPHProstate |
| 560 | 534076 | 2542.J21.GZ43_373012 | F | M00073891C:A12 | IF97-26811-NormBPHProstate |
| 561 | 240 | 2542.K21.GZ43_373036 | F | M00073893B:C08 | IF97-26811-NormBPHProstate |
| 562 | 58218 | 2542.M24.GZ43_373087 | F | M00073897B:B11 | IF97-26811-NormBPHProstate |
| 563 | 641662 | 2542.N21.GZ43_373108 | F | M00073899A:C02 | IF97-26811-NormBPHProstate |
| 564 | 398642 | 2542.N22.GZ43_373109 | F | M00073899A:D06 | IF97-26811-NormBPHProstate |
| 565 | 452289 | 2555.B08.GZ43_373191 | F | M00073911B:G10 | IF97-26811-NormBPHProstate |
| 566 | 621397 | 2555.B20.GZ43_373203 | F | M00073912B:C04 | IF97-26811-NormBPHProstate |
| 567 | 641662 | 2555.D22.GZ43_373253 | F | M00073916A:B07 | IF97-26811-NormBPHProstate |
| 568 | 13903 | 2555.E20.GZ43_373275 | F | M00073917B:B07 | IF97-26811-NormBPHProstate |
| 569 | 727966 | 2555.F16.GZ43_373295 | F | M00073918C:B03 | IF97-26811-NormBPHProstate |
| 570 | 702885 | 2555.H18.GZ43_373345 | F | M00073921B:H12 | IF97-26811-NormBPHProstate |
| 571 | 525801 | 2555.I05.GZ43_373356 | F | M00073922C:E02 | IF97-26811-NormBPHProstate |
| 572 | 11561 | 2555.I21.GZ43_373372 | F | M00073923C:A04 | IF97-26811-NormBPHProstate |
| 573 | 602052 | 2555.J07.GZ43_373382 | F | M00073924B:H03 | IF97-26811-NormBPHProstate |
| 574 | 453398 | 2555.K17.GZ43_373416 | F | M00073927D:E09 | IF97-26811-NormBPHProstate |
| 575 | 528957 | 2555.M18.GZ43_373465 | F | M00073931D:E02 | IF97-26811-NormBPHProstate |
| 576 | 652099 | 2555.N05.GZ43_373476 | F | M00073932D:G05 | IF97-26811-NormBPHProstate |
| 577 | 16641 | 2555.P05.GZ43_373524 | F | M00073936D:E05 | IF97-26811-NormBPHProstate |
| 578 | 517481 | 2555.P22.GZ43_373541 | F | M00073938B:D11 | IF97-26811-NormBPHProstate |
| 579 | 411128 | 2555.A11.GZ43_373170 | F | M00073908C:D09 | IF97-26811-NormBPHProstate |
| 580 | 558342 | 2555.E11.GZ43_373266 | F | M00073916C:H11 | IF97-26811-NormBPHProstate |
| 581 | 692282 | 2555.F09.GZ43_373288 | F | M00073918A:F07 | IF97-26811-NormBPHProstate |
| 582 | 520370 | 2555.F10.GZ43_373289 | F | M00073918A:G12 | IF97-26811-NormBPHProstate |
| 583 | 271 | 2555.G11.GZ43_373314 | F | M00073919C:B04 | IF97-26811-NormBPHProstate |
| 584 | 525801 | 2555.H12.GZ43_373339 | F | M00073920D:F08 | IF97-26811-NormBPHProstate |
| 585 | 467877 | 2555.I12.GZ43_373363 | F | M00073922D:G04 | IF97-26811-NormBPHProstate |
| 586 | 502358 | 2555.J10.GZ43_373385 | F | M00073924C:G05 | IF97-26811-NormBPHProstate |
| 587 | 15935 | 2555.K10.GZ43_373409 | F | M00073927C:B07 | IF97-26811-NormBPHProstate |
| 588 | 451821 | 2555.N09.GZ43_373480 | F | M00073933B:B12 | IF97-26811-NormBPHProstate |
| 589 | 604822 | 2556.A02.GZ43_373545 | F | M00073938B:F09 | IF97-26811-NormBPHProstate |
| 590 | 50391 | 2556.B22.GZ43_373589 | F | M00073941B:A06 | IF97-26811-NormBPHProstate |
| 591 | 139789 | 2556.C11.GZ43_373602 | F | M00073941D:H09 | IF97-26811-NormBPHProstate |
| 592 | 649670 | 2556.C19.GZ43_373610 | F | M00073942B:C01 | IF97-26811-NormBPHProstate |
| 593 | 20563 | 2556.D02.GZ43_373617 | F | M00073942C:E04 | IF97-26811-NormBPHProstate |
| 594 | 113786 | 2556.D06.GZ43_373621 | F | M00073942D:D09 | IF97-26811-NormBPHProstate |
| 595 | 420371 | 2556.D09.GZ43_373624 | F | M00073942D:G05 | IF97-26811-NormBPHProstate |
| 596 | 1607 | 2556.E07.GZ43_373646 | F | M00073944A:E10 | IF97-26811-NormBPHProstate |
| 597 | 60888 | 2556.E11.GZ43_373650 | F | M00073944A:H05 | IF97-26811-NormBPHProstate |
| 598 | 472262 | 2556.F11.GZ43_373674 | F | M00073944C:H07 | IF97-26811-NormBPHProstate |
| 599 | 171595 | 2556.F14.GZ43_373677 | F | M00073944D:A07 | IF97-26811-NormBPHProstate |
| 600 | 17855 | 2556.F15.GZ43_373678 | F | M00073944D:E12 | IF97-26811-NormBPHProstate |
| 601 | 842551 | 2556.G19.GZ43_373706 | F | M00073946D:F07 | IF97-26811-NormBPHProstate |
| 602 | 87051 | 2556.H15.GZ43_373726 | F | M00073947C:B01 | IF97-26811-NormBPHProstate |
| 603 | 297358 | 2556.H19.GZ43_373730 | F | M00073947C:E09 | IF97-26811-NormBPHProstate |
| 604 | 22884 | 2556.I05.GZ43_373740 | F | M00073948A:G05 | IF97-26811-NormBPHProstate |
| 605 | 48896 | 2556.J03.GZ43_373762 | F | M00073949A:C09 | IF97-26811-NormBPHProstate |
| 606 | 9047 | 2556.J15.GZ43_373774 | F | M00073949D:C11 | IF97-26811-NormBPHProstate |
| 607 | 1409 | 2556.J18.GZ43_373777 | F | M00073950C:A05 | IF97-26811-NormBPHProstate |
| 608 | 63551 | 2556.K03.GZ43_373786 | F | M00073950D:H12 | IF97-26811-NormBPHProstate |
| 609 | 13629 | 2556.K07.GZ43_373790 | F | M00073952A:G04 | IF97-26811-NormBPHProstate |
| 610 | 850377 | 2556.L21.GZ43_373828 | F | M00073956D:F02 | IF97-26811-NormBPHProstate |
| 611 | 448319 | 2556.M11.GZ43_373842 | F | M00073960A:B12 | IF97-26811-NormBPHProstate |
| 612 | 582134 | 2556.M16.GZ43_373847 | F | M00073960B:A09 | IF97-26811-NormBPHProstate |
| 613 | 946181 | 2556.N05.GZ43_373860 | F | M00073961B:G01 | IF97-26811-NormBPHProstate |
| 614 | 782981 | 2556.O05.GZ43_373884 | F | M00073962D:E04 | IF97-26811-NormBPHProstate |
| 615 | 43910 | 2556.O11.GZ43_373890 | F | M00073963A:G08 | IF97-26811-NormBPHProstate |
| 616 | 154120 | 2556.O16.GZ43_373895 | F | M00073963B:F04 | IF97-26811-NormBPHProstate |
| 617 | 550104 | 2556.P03.GZ43_373906 | F | M00073964B:H07 | IF97-26811-NormBPHProstate |
| 618 | 471364 | 2557.B09.GZ43_373960 | F | M00073967A:A10 | IF97-26811-NormBPHProstate |
| 619 | 398642 | 2557.B11.GZ43_373962 | F | M00073967C:A01 | IF97-26811-NormBPHProstate |
| 620 | 572170 | 2557.B22.GZ43_373973 | F | M00073968B:B06 | IF97-26811-NormBPHProstate |
| 621 | 780111 | 2557.C11.GZ43_373986 | F | M00073968D:F11 | IF97-26811-NormBPHProstate |
| 622 | 472262 | 2557.D14.GZ43_374013 | F | M00073970B:G01 | IF97-26811-NormBPHProstate |
| 623 | 40330 | 2557.G10.GZ43_374081 | F | M00073977D:B10 | IF97-26811-NormBPHProstate |
| 624 | 218375 | 2557.G20.GZ43_374091 | F | M00073978D:A02 | IF97-26811-NormBPHProstate |
| 625 | 520370 | 2557.H11.GZ43_374106 | F | M00073979C:G07 | IF97-26811-NormBPHProstate |
| 626 | 621573 | 2557.I17.GZ43_374136 | F | M00073981C:F08 | IF97-26811-NormBPHProstate |
| 627 | 551744 | 2557.J14.GZ43_374157 | F | M00073983B:D03 | IF97-26811-NormBPHProstate |
| 628 | 35049 | 2557.J16.GZ43_374159 | F | M00073983C:C07 | IF97-26811-NormBPHProstate |
| 629 | 8268 | 2557.J21.GZ43_374164 | F | M00073984B:D04 | IF97-26811-NormBPHProstate |
| 630 | 697955 | 2557.J22.GZ43_374165 | F | M00073984B:E01 | IF97-26811-NormBPHProstate |
| 631 | 727968 | 2557.K11.GZ43_374178 | F | M00073985C:A05 | IF97-26811-NormBPHProstate |
| 632 | 839437 | 2557.L12.GZ43_374203 | F | M00073987B:A09 | IF97-26811-NormBPHProstate |
| 633 | 533888 | 2557.L23.GZ43_374214 | F | M00073988B:C08 | IF97-26811-NormBPHProstate |
| 634 | 555867 | 2557.M10.GZ43_374225 | F | M00073988D:F09 | IF97-26811-NormBPHProstate |
| 635 | 709796 | 2557.N14.GZ43_374253 | F | M00073993A:A05 | IF97-26811-NormBPHProstate |
| 636 | 736938 | 2557.A03.GZ43_373930 | F | M00073965D:A12 | IF97-26811-NormBPHProstate |
| 637 | 867511 | 2557.B01.GZ43_373952 | F | M00073966C:F08 | IF97-26811-NormBPHProstate |
| 638 | 531505 | 2557.C04.GZ43_373979 | F | M00073968C:C09 | IF97-26811-NormBPHProstate |
| 639 | 401809 | 2557.C05.GZ43_373980 | F | M00073968C:F02 | IF97-26811-NormBPHProstate |
| 640 | 796532 | 2557.F03.GZ43_374050 | F | M00073975A:A12 | IF97-26811-NormBPHProstate |
| 641 | 572170 | 2557.H03.GZ43_374098 | F | M00073979B:B05 | IF97-26811-NormBPHProstate |
| 642 | 644299 | 2557.H05.GZ43_374100 | F | M00073979C:B01 | IF97-26811-NormBPHProstate |
| 643 | 633646 | 2557.J06.GZ43_374149 | F | M00073982B:H01 | IF97-26811-NormBPHProstate |
| 644 | 558581 | 2557.L01.GZ43_374192 | F | M00073986C:D07 | IF97-26811-NormBPHProstate |
| 645 | 558579 | 2557.M06.GZ43_374221 | F | M00073988C:G08 | IF97-26811-NormBPHProstate |
| 646 | 448604 | 2558.A07.GZ43_374318 | F | M00074000C:D06 | IF97-26811-NormBPHProstate |
| 647 | 404482 | 2558.B13.GZ43_374348 | F | M00074003C:H06 | IF97-26811-NormBPHProstate |
| 648 | 847088 | 2558.B24.GZ43_374359 | F | M00074004A:H01 | IF97-26811-NormBPHProstate |
| 649 | 451981 | 2558.C04.GZ43_374363 | F | M00074004C:F03 | IF97-26811-NormBPHProstate |
| 650 | 660842 | 2558.C18.GZ43_374377 | F | M00074006C:B12 | IF97-26811-NormBPHProstate |
| 651 | 558569 | 2558.D03.GZ43_374386 | F | M00074007B:A02 | IF97-26811-NormBPHProstate |
| 652 | 640319 | 2558.E21.GZ43_374428 | F | M00074010B:D07 | IF97-26811-NormBPHProstate |
| 653 | 556827 | 2558.E24.GZ43_374431 | F | M00074011A:F08 | IF97-26811-NormBPHProstate |
| 654 | 10354 | 2558.F06.GZ43_374437 | F | M00074011D:C05 | IF97-26811-NormBPHProstate |
| 655 | 993554 | 2558.F19.GZ43_374450 | F | M00074013B:F07 | IF97-26811-NormBPHProstate |
| 656 | 643828 | 2558.F21.GZ43_374452 | F | M00074013C:C09 | IF97-26811-NormBPHProstate |
| 657 | 48289 | 2558.G07.GZ43_374462 | F | M00074014A:G03 | IF97-26811-NormBPHProstate |
| 658 | 682 | 2558.G13.GZ43_374468 | F | M00074014D:F04 | IF97-26811-NormBPHProstate |
| 659 | 132559 | 2558.G17.GZ43_374472 | F | M00074015A:C03 | IF97-26811-NormBPHProstate |
| 660 | 23300 | 2558.H13.GZ43_374492 | F | M00074017B:G10 | IF97-26811-NormBPHProstate |
| 661 | 510539 | 2558.H17.GZ43_374496 | F | M00074017D:C01 | IF97-26811-NormBPHProstate |
| 662 | 388450 | 2558.J01.GZ43_374528 | F | M00074019D:H05 | IF97-26811-NormBPHProstate |
| 663 | 50661 | 2558.J03.GZ43_374530 | F | M00074020B:G11 | IF97-26811-NormBPHProstate |
| 664 | 715752 | 2558.J04.GZ43_374531 | F | M00074020C:A05 | IF97-26811-NormBPHProstate |
| 665 | 752831 | 2558.J09.GZ43_374536 | F | M00074020D:G10 | IF97-26811-NormBPHProstate |
| 666 | 505984 | 2558.K02.GZ43_374553 | F | M00074021C:H07 | IF97-26811-NormBPHProstate |
| 667 | 672233 | 2558.K08.GZ43_374559 | F | M00074022A:C06 | IF97-26811-NormBPHProstate |
| 668 | 733132 | 2558.L15.GZ43_374590 | F | M00074024B:G07 | IF97-26811-NormBPHProstate |
| 669 | 1037152 | 2558.L19.GZ43_374594 | F | M00074025A:F06 | IF97-26811-NormBPHProstate |
| 670 | 8268 | 2558.L21.GZ43_374596 | F | M00074025B:A12 | IF97-26811-NormBPHProstate |
| 671 | 918867 | 2558.M11.GZ43_374610 | F | M00074026C:H09 | IF97-26811-NormBPHProstate |
| 672 | 64589 | 2558.M18.GZ43_374617 | F | M00074027D:B03 | IF97-26811-NormBPHProstate |
| 673 | 217122 | 2558.N22.GZ43_374645 | F | M00074030D:A12 | IF97-26811-NormBPHProstate |
| 674 | 559336 | 2558.O09.GZ43_374656 | F | M00074032B:H08 | IF97-26811-NormBPHProstate |
| 675 | 535996 | 2558.O10.GZ43_374657 | F | M00074032C:E02 | IF97-26811-NormBPHProstate |
| 676 | 553342 | 2558.O11.GZ43_374658 | F | M00074032C:H07 | IF97-26811-NormBPHProstate |
| 677 | 404368 | 2558.P16.GZ43_374687 | F | M00074036B:C08 | IF97-26811-NormBPHProstate |
| 678 | 823296 | 2558.P20.GZ43_374691 | F | M00074036D:B05 | IF97-26811-NormBPHProstate |
| 679 | 48738 | 2559.A01.GZ43_374696 | F | M00074037A:B03 | IF97-26811-NormBPHProstate |
| 680 | 948383 | 2559.A09.GZ43_374704 | F | M00074038A:G08 | IF97-26811-NormBPHProstate |
| 681 | 738784 | 2559.A13.GZ43_374708 | F | M00074038C:B08 | IF97-26811-NormBPHProstate |
| 682 | 588996 | 2559.B05.GZ43_374724 | F | M00074040A:B06 | IF97-26811-NormBPHProstate |
| 683 | 5013 | 2559.D05.GZ43_374772 | F | M00074043C:A05 | IF97-26811-NormBPHProstate |
| 684 | 954558 | 2559.G18.GZ43_374857 | F | M00074050B:H07 | IF97-26811-NormBPHProstate |
| 685 | 424776 | 2559.H08.GZ43_374871 | F | M00074051C:F05 | IF97-26811-NormBPHProstate |
| 686 | 519176 | 2559.H20.GZ43_374883 | F | M00074052C:E03 | IF97-26811-NormBPHProstate |
| 687 | 448221 | 2559.I12.GZ43_374899 | F | M00074053C:E05 | IF97-26811-NormBPHProstate |
| 688 | 184489 | 2559.I13.GZ43_374900 | F | M00074053C:G11 | IF97-26811-NormBPHProstate |
| 689 | 404482 | 2559.I17.GZ43_374904 | F | M00074053D:D05 | IF97-26811-NormBPHProstate |
| 690 | 13903 | 2559.J02.GZ43_374913 | F | M00074054C:B04 | IF97-26811-NormBPHProstate |
| 691 | 204255 | 2559.J13.GZ43_374924 | F | M00074055A:G08 | IF97-26811-NormBPHProstate |
| 692 | 551744 | 2559.K12.GZ43_374947 | F | M00074057A:B12 | IF97-26811-NormBPHProstate |
| 693 | 395953 | 2559.L08.GZ43_374967 | F | M00074058A:H02 | IF97-26811-NormBPHProstate |
| 694 | 63891 | 2559.L09.GZ43_374968 | F | M00074058B:A10 | IF97-26811-NormBPHProstate |
| 695 | 406961 | 2559.M02.GZ43_374985 | F | M00074059B:G10 | IF97-26811-NormBPHProstate |
| 696 | 23951 | 2559.M21.GZ43_375004 | F | M00074060D:A10 | IF97-26811-NormBPHProstate |
| 697 | 34391 | 2559.N05.GZ43_375012 | F | M00074061B:E01 | IF97-26811-NormBPHProstate |
| 698 | 16978 | 2559.N13.GZ43_375020 | F | M00074063A:B03 | IF97-26811-NormBPHProstate |
| 699 | 13565 | 2559.N15.GZ43_375022 | F | M00074063A:D09 | IF97-26811-NormBPHProstate |
| 700 | 402267 | 2559.N18.GZ43_375025 | F | M00074063B:B12 | IF97-26811-NormBPHProstate |
| 701 | 35578 | 2559.P19.GZ43_375074 | F | M00074069D:C11 | IF97-26811-NormBPHProstate |
| 702 | 459865 | 2560.A08.GZ43_375087 | F | M00074070D:G05 | IF97-26811-NormBPHProstate |
| 703 | 37848 | 2560.B11.GZ43_375114 | F | M00074075B:A09 | IF97-26811-NormBPHProstate |
| 704 | 66923 | 2560.B15.GZ43_375118 | F | M00074075C:H04 | IF97-26811-NormBPHProstate |
| 705 | 400258 | 2560.B20.GZ43_375123 | F | M00074076B:F04 | IF97-26811-NormBPHProstate |
| 706 | 404368 | 2560.C15.GZ43_375142 | F | M00074079A:E07 | IF97-26811-NormBPHProstate |
| 707 | 333093 | 2560.E19.GZ43_375194 | F | M00074084C:E01 | IF97-26811-NormBPHProstate |
| 708 | 676448 | 2560.E22.GZ43_375197 | F | M00074084D:B04 | IF97-26811-NormBPHProstate |
| 709 | 554127 | 2560.F07.GZ43_375206 | F | M00074085A:H10 | IF97-26811-NormBPHProstate |
| 710 | 171148 | 2560.F10.GZ43_375209 | F | M00074085B:E06 | IF97-26811-NormBPHProstate |
| 711 | 946181 | 2560.F16.GZ43_375215 | F | M00074085D:E08 | IF97-26811-NormBPHProstate |
| 712 | 697955 | 2560.G13.GZ43_375236 | F | M00074087B:C09 | IF97-26811-NormBPHProstate |
| 713 | 453476 | 2560.G18.GZ43_375241 | F | M00074087C:G05 | IF97-26811-NormBPHProstate |
| 714 | 833580 | 2560.H01.GZ43_375248 | F | M00074088B:A03 | IF97-26811-NormBPHProstate |
| 715 | 531583 | 2560.H12.GZ43_375259 | F | M00074088C:E07 | IF97-26811-NormBPHProstate |
| 716 | 558342 | 2560.H21.GZ43_375268 | F | M00074089A:B09 | IF97-26811-NormBPHProstate |
| 717 | 455862 | 2560.I09.GZ43_375280 | F | M00074089D:E03 | IF97-26811-NormBPHProstate |
| 718 | 19627 | 2560.I16.GZ43_375287 | F | M00074090A:E09 | IF97-26811-NormBPHProstate |
| 719 | 9134 | 2560.K02.GZ43_375321 | F | M00074093A:A06 | IF97-26811-NormBPHProstate |
| 720 | 41346 | 2560.K08.GZ43_375327 | F | M00074093B:A03 | IF97-26811-NormBPHProstate |
| 721 | 756337 | 2560.K10.GZ43_375329 | F | M00074093B:C07 | IF97-26811-NormBPHProstate |
| 722 | 397115 | 2560.K18.GZ43_375337 | F | M00074094B:F10 | IF97-26811-NormBPHProstate |
| 723 | 805118 | 2560.L14.GZ43_375357 | F | M00074096D:G12 | IF97-26811-NormBPHProstate |
| 724 | 456113 | 2560.L15.GZ43_375358 | F | M00074097A:F10 | IF97-26811-NormBPHProstate |
| 725 | 677530 | 2560.L22.GZ43_375365 | F | M00074097C:B09 | IF97-26811-NormBPHProstate |
| 726 | 697955 | 2560.M11.GZ43_375378 | F | M00074098C:B09 | IF97-26811-NormBPHProstate |
| 727 | 493811 | 2560.M23.GZ43_375390 | F | M00074099C:B09 | IF97-26811-NormBPHProstate |
| 728 | 127471 | 2560.N09.GZ43_375400 | F | M00074100B:E01 | IF97-26811-NormBPHProstate |
| 729 | 559267 | 2560.O08.GZ43_375423 | F | M00074101D:D07 | IF97-26811-NormBPHProstate |
| 730 | 691653 | 2560.O12.GZ43_375427 | F | M00074102A:C04 | IF97-26811-NormBPHProstate |
| 731 | 966599 | 2560.P24.GZ43_375463 | F | M00074105A:D02 | IF97-26811-NormBPHProstate |
| 732 | 139979 | 2561.B03.GZ43_376258 | F | M00074106C:E03 | IF97-26811-NormBPHProstate |
| 733 | 668962 | 2561.B12.GZ43_376267 | F | M00074107C:C08 | IF97-26811-NormBPHProstate |
| 734 | 217122 | 2561.C13.GZ43_376292 | F | M00074111C:B02 | IF97-26811-NormBPHProstate |
| 735 | 70908 | 2561.C15.GZ43_376294 | F | M00074111C:G11 | IF97-26811-NormBPHProstate |
| 736 | 557771 | 2561.D14.GZ43_376317 | F | M00074116C:A03 | IF97-26811-NormBPHProstate |
| 737 | 629125 | 2561.E10.GZ43_376337 | F | M00074120A:A12 | IF97-26811-NormBPHProstate |
| 738 | 626993 | 2561.F09.GZ43_376360 | F | M00074123B:A03 | IF97-26811-NormBPHProstate |
| 739 | 69779 | 2561.F13.GZ43_376364 | F | M00074123B:G07 | IF97-26811-NormBPHProstate |
| 740 | 752623 | 2561.I07.GZ43_376430 | F | M00074130B:F06 | IF97-26811-NormBPHProstate |
| 741 | 692282 | 2561.I11.GZ43_376434 | F | M00074131A:H09 | IF97-26811-NormBPHProstate |
| 742 | 685244 | 2561.J01.GZ43_376448 | F | M00074132C:F10 | IF97-26811-NormBPHProstate |
| 743 | 597681 | 2561.K03.GZ43_376474 | F | M00074135A:G09 | IF97-26811-NormBPHProstate |
| 744 | 1037152 | 2561.K10.GZ43_376481 | F | M00074135C:E09 | IF97-26811-NormBPHProstate |
| 745 | 533888 | 2561.L02.GZ43_376497 | F | M00074137C:E05 | IF97-26811-NormBPHProstate |
| 746 | 378561 | 2561.L13.GZ43_376508 | F | M00074138D:A01 | IF97-26811-NormBPHProstate |
| 747 | 415520 | 2561.L14.GZ43_376509 | F | M00074138D:A08 | IF97-26811-NormBPHProstate |
| 748 | 415520 | 2561.L15.GZ43_376510 | F | M00074138D:B07 | IF97-26811-NormBPHProstate |
| 749 | 455254 | 2561.M03.GZ43_376522 | F | M00074142B:C11 | IF97-26811-NormBPHProstate |
| 750 | 315533 | 2561.M09.GZ43_376528 | F | M00074142D:A10 | IF97-26811-NormBPHProstate |
| 751 | 10585 | 2561.O10.GZ43_376577 | F | M00074148B:D09 | IF97-26811-NormBPHProstate |
| 752 | 20052 | 2561.B18.GZ43_376273 | F | M00074108B:C04 | IF97-26811-NormBPHProstate |
| 753 | 558602 | 2561.E22.GZ43_376349 | F | M00074122A:B02 | IF97-26811-NormBPHProstate |
| 754 | 559336 | 2561.G20.GZ43_376395 | F | M00074126B:E12 | IF97-26811-NormBPHProstate |
| 755 | 163602 | 2561.H17.GZ43_376416 | F | M00074128D:C09 | IF97-26811-NormBPHProstate |
| 756 | 756337 | 2561.I19.GZ43_376442 | F | M00074132A:E11 | IF97-26811-NormBPHProstate |
| 757 | 452194 | 2561.I24.GZ43_376447 | F | M00074132B:B07 | IF97-26811-NormBPHProstate |
| 758 | 31453 | 2561.J18.GZ43_376465 | F | M00074134A:G11 | IF97-26811-NormBPHProstate |
| 759 | 220845 | 2561.O17.GZ43_376584 | F | M00074149A:B10 | IF97-26811-NormBPHProstate |
| 760 | 1022935 | 2561.O19.GZ43_376586 | F | M00074149A:F12 | IF97-26811-NormBPHProstate |
| 761 | 396325 | 2561.P16.GZ43_376607 | F | M00074153A:E07 | IF97-26811-NormBPHProstate |
| 762 | 835488 | 2561.P19.GZ43_376610 | F | M00074153D:A05 | IF97-26811-NormBPHProstate |
| 763 | 119614 | 2561.P23.GZ43_376614 | F | M00074154A:D03 | IF97-26811-NormBPHProstate |
| 764 | 400258 | 2456.A08.GZ43_355836 | F | M00074155B:G09 | IF97-26811-NormBPHProstate |
| 765 | 165378 | 2456.B09.GZ43_355861 | F | M00074157C:G08 | IF97-26811-NormBPHProstate |
| 766 | 641662 | 2456.B12.GZ43_355864 | F | M00074157D:G05 | IF97-26811-NormBPHProstate |
| 767 | 648899 | 2456.B17.GZ43_355869 | F | M00074158C:F12 | IF97-26811-NormBPHProstate |
| 768 | 128596 | 2456.B18.GZ43_355870 | F | M00074158C:H10 | IF97-26811-NormBPHProstate |
| 769 | 452194 | 2456.C01.GZ43_355877 | F | M00074159C:A05 | IF97-26811-NormBPHProstate |
| 770 | 534076 | 2456.C05.GZ43_355881 | F | M00074160A:D12 | IF97-26811-NormBPHProstate |
| 771 | 372750 | 2456.D04.GZ43_355904 | F | M00074161C:F04 | IF97-26811-NormBPHProstate |
| 772 | 391508 | 2456.D05.GZ43_355905 | F | M00074162A:B03 | IF97-26811-NormBPHProstate |
| 773 | 7105 | 2456.E17.GZ43_355941 | F | M00074165D:A11 | IF97-26811-NormBPHProstate |
| 774 | 177808 | 2456.F16.GZ43_355964 | F | M00074170A:D09 | IF97-26811-NormBPHProstate |
| 775 | 516526 | 2456.F23.GZ43_355971 | F | M00074170D:F05 | IF97-26811-NormBPHProstate |
| 776 | 372710 | 2456.G10.GZ43_355982 | F | M00074172B:D12 | IF97-26811-NormBPHProstate |
| 777 | 540142 | 2456.H02.GZ43_355998 | F | M00074174A:C02 | IF97-26811-NormBPHProstate |
| 778 | 1041923 | 2456.H07.GZ43_356003 | F | M00074174C:C03 | IF97-26811-NormBPHProstate |
| 779 | 136276 | 2456.I05.GZ43_356025 | F | M00074175D:E04 | IF97-26811-NormBPHProstate |
| 780 | 568661 | 2456.I09.GZ43_356029 | F | M00074176A:A06 | IF97-26811-NormBPHProstate |
| 781 | 403242 | 2456.110.GZ43_356030 | F | M00074176A:B10 | IF97-26811-NormBPHProstate |
| 782 | 41455 | 2456.J06.GZ43_356050 | F | M00074177B:H08 | IF97-26811-NormBPHProstate |
| 783 | 853431 | 2456.J18.GZ43_356062 | F | M00074178B:G07 | IF97-26811-NormBPHProstate |
| 784 | 423303 | 2456.J24.GZ43_356068 | F | M00074179A:A01 | IF97-26811-NormBPHProstate |
| 785 | 41455 | 2456.K07.GZ43_356075 | F | M00074179C:B01 | IF97-26811-NormBPHProstate |
| 786 | 568204 | 2456.M05.GZ43_356121 | F | M00074184D:A04 | IF97-26811-NormBPHProstate |
| 787 | 642041 | 2456.M06.GZ43_356122 | F | M00074184D:B01 | IF97-26811-NormBPHProstate |
| 788 | 427449 | 2456.N23.GZ43_356163 | F | M00074190B:F09 | IF97-26811-NormBPHProstate |
| 789 | 565709 | 2456.O10.GZ43_356174 | F | M00074191C:D08 | IF97-26811-NormBPHProstate |
| 790 | 676448 | 2456.O18.GZ43_356182 | F | M00074192C:C10 | IF97-26811-NormBPHProstate |
| 791 | 99399 | 2456.P23.GZ43_356211 | F | M00074195D:B09 | IF97-26811-NormBPHProstate |
| 792 | 222887 | 2457.A21.GZ43_356233 | F | M00074197C:A12 | IF97-26811-NormBPHProstate |
| 793 | 778001 | 2457.B07.GZ43_356243 | F | M00074198C:A12 | IF97-26811-NormBPHProstate |
| 794 | 806992 | 2457.B10.GZ43_356246 | F | M00074198D:D10 | IF97-26811-NormBPHProstate |
| 795 | 217122 | 2457.B13.GZ43_356249 | F | M00074199A:C10 | IF97-26811-NormBPHProstate |
| 796 | 733673 | 2457.C19.GZ43_356279 | F | M00074201A:F03 | IF97-26811-NormBPHProstate |
| 797 | 37375 | 2457.C23.GZ43_356283 | F | M00074201C:E12 | IF97-26811-NormBPHProstate |
| 798 | 41702 | 2457.D05.GZ43_356289 | F | M00074202A:A05 | IF97-26811-NormBPHProstate |
| 799 | 13903 | 2457.D12.GZ43_356296 | F | M00074202B:D03 | IF97-26811-NormBPHProstate |
| 800 | 626993 | 2457.E05.GZ43_356313 | F | M00074203D:F01 | IF97-26811-NormBPHProstate |
| 801 | 474125 | 2457.E23.GZ43_356331 | F | M00074206A:G02 | IF97-26811-NormBPHProstate |
| 802 | 552374 | 2457.E24.GZ43_356332 | F | M00074206A:H12 | IF97-26811-NormBPHProstate |
| 803 | 220576 | 2457.F02.GZ43_356334 | F | M00074206B:F04 | IF97-26811-NormBPHProstate |
| 804 | 450754 | 2457.F17.GZ43_356349 | F | M00074207D:E07 | IF97-26811-NormBPHProstate |
| 805 | 732950 | 2457.F20.GZ43_356352 | F | M00074208B:B05 | IF97-26811-NormBPHProstate |
| 806 | 948383 | 2457.F23.GZ43_356355 | F | M00074208B:F09 | IF97-26811-NormBPHProstate |
| 807 | 218833 | 2457.G03.GZ43_356359 | F | M00074208D:E08 | IF97-26811-NormBPHProstate |
| 808 | 192830 | 2457.G13.GZ43_356369 | F | M00074209D:H11 | IF97-26811-NormBPHProstate |
| 809 | 1017557 | 2457.G17.GZ43_356373 | F | M00074210B:G12 | IF97-26811-NormBPHProstate |
| 810 | 557507 | 2457.H17.GZ43_356397 | F | M00074213A:C06 | IF97-26811-NormBPHProstate |
| 811 | 551338 | 2457.I12.GZ43_356416 | F | M00074215A:F09 | IF97-26811-NormBPHProstate |
| 812 | 839437 | 2457.J13.GZ43_356441 | F | M00074216C:C11 | IF97-26811-NormBPHProstate |
| 813 | 376516 | 2457.J23.GZ43_356451 | F | M00074216D:H03 | IF97-26811-NormBPHProstate |
| 814 | 397140 | 2457.K03.GZ43_356455 | F | M00074217A:H01 | IF97-26811-NormBPHProstate |
| 815 | 28050 | 2457.K07.GZ43_356459 | F | M00074217C:B04 | IF97-26811-NormBPHProstate |
| 816 | 640582 | 2457.K08.GZ43_356460 | F | M00074217C:C09 | IF97-26811-NormBPHProstate |
| 817 | 993554 | 2457.L04.GZ43_356480 | F | M00074219D:F03 | IF97-26811-NormBPHProstate |
| 818 | 465446 | 2457.L21.GZ43_356497 | F | M00074221B:F12 | IF97-26811-NormBPHProstate |
| 819 | 429609 | 2457.M11.GZ43_356511 | F | M00074223B:D12 | IF97-26811-NormBPHProstate |
| 820 | 449482 | 2457.M20.GZ43_356520 | F | M00074224A:G06 | IF97-26811-NormBPHProstate |
| 821 | 31453 | 2457.N07.GZ43_356531 | F | M00074225A:H12 | IF97-26811-NormBPHProstate |
| 822 | 16641 | 2457.O02.GZ43_356550 | F | M00074226C:E06 | IF97-26811-NormBPHProstate |
| 823 | 130924 | 2458.A10.GZ43_356618 | F | M00074230D:B05 | IF97-26811-NormBPHProstate |
| 824 | 184653 | 2458.A13.GZ43_356621 | F | M00074231A:D10 | IF97-26811-NormBPHProstate |
| 825 | 20858 | 2458.A24.GZ43_356632 | F | M00074231D:G11 | IF97-26811-NormBPHProstate |
| 826 | 140585 | 2458.B08.GZ43_356640 | F | M00074232B:G06 | IF97-26811-NormBPHProstate |
| 827 | 547023 | 2458.B23.GZ43_356655 | F | M00074234A:C05 | IF97-26811-NormBPHProstate |
| 828 | 53675 | 2458.B24.GZ43_356656 | F | M00074234A:E07 | IF97-26811-NormBPHProstate |
| 829 | 498886 | 2458.C06.GZ43_356662 | F | M00074234B:F07 | IF97-26811-NormBPHProstate |
| 830 | 10354 | 2458.C12.GZ43_356668 | F | M00074234D:F12 | IF97-26811-NormBPHProstate |
| 831 | 12906 | 2458.C23.GZ43_356679 | F | M00074235C:D06 | IF97-26811-NormBPHProstate |
| 832 | 184489 | 2458.D06.GZ43_356686 | F | M00074236B:E06 | IF97-26811-NormBPHProstate |
| 833 | 37634 | 2458.D07.GZ43_356687 | F | M00074236C:E11 | IF97-26811-NormBPHProstate |
| 834 | 72628 | 2458.F01.GZ43_356729 | F | M00074242D:F09 | IF97-26811-NormBPHProstate |
| 835 | 23957 | 2458.F06.GZ43_356734 | F | M00074243A:H08 | IF97-26811-NormBPHProstate |
| 836 | 29906 | 2458.G01.GZ43_356753 | F | M00074244C:B11 | IF97-26811-NormBPHProstate |
| 837 | 453526 | 2458.G20.GZ43_356772 | F | M00074247B:G11 | IF97-26811-NormBPHProstate |
| 838 | 18644 | 2458.G21.GZ43_356773 | F | M00074247C:E02 | IF97-26811-NormBPHProstate |
| 839 | 8956 | 2458.H07.GZ43_356783 | F | M00074248C:E12 | IF97-26811-NormBPHProstate |
| 840 | 9710 | 2458.H16.GZ43_356792 | F | M00074249C:B11 | IF97-26811-NormBPHProstate |
| 841 | 390274 | 2458.H20.GZ43_356796 | F | M00074249C:H08 | IF97-26811-NormBPHProstate |
| 842 | 112224 | 2458.I09.GZ43_356809 | F | M00074250D:EO6 | IF97-26811-NormBPHProstate |
| 843 | 20915 | 2458.I10.GZ43_356810 | F | M00074250D:F06 | IF97-26811-NormBPHProstate |
| 844 | 77670 | 2458.I15.GZ43_356815 | F | M00074251B:F08 | IF97-26811-NormBPHProstate |
| 845 | 32366 | 2458.I17.GZ43_356817 | F | M00074251C:B06 | IF97-26811-NormBPHProstate |
| 846 | 11031 | 2458.I20.GZ43_356820 | F | M00074251C:E03 | IF97-26811-NormBPHProstate |
| 847 | 112224 | 2458.I21.GZ43_356821 | F | M00074251D:E03 | IF97-26811-NormBPHProstate |
| 848 | 40164 | 2458.J03.GZ43_356827 | F | M00074252C:E02 | IF97-26811-NormBPHProstate |
| 849 | 72825 | 2458.J21.GZ43_356845 | F | M00074253C:F03 | IF97-26811-NormBPHProstate |
| 850 | 36407 | 2458.K07.GZ43_356855 | F | M00074255B:A01 | IF97-26811-NormBPHProstate |
| 851 | 63902 | 2458.L06.GZ43_356878 | F | M00074258A:H12 | IF97-26811-NormBPHProstate |
| 852 | 954558 | 2458.L07.GZ43_356879 | F | M00074258A:H09 | IF97-26811-NormBPHProstate |
| 853 | 447270 | 2458.L23.GZ43_356895 | F | M00074259C:G08 | IF97-26811-NormBPHProstate |
| 854 | 16174 | 2458.M06.GZ43_356901 | F | M00074260B:A11 | IF97-26811-NormBPHProstate |
| 855 | 139173 | 2458.N06.GZ43_356926 | F | M00074265B:C07 | IF97-26811-NormBPHProstate |
| 856 | 217122 | 2458.N10.GZ43_356930 | F | M00074266A:D01 | IF97-26811-NormBPHProstate |
| 857 | 497138 | 2458.N19.GZ43_356939 | F | M00074267A:B04 | IF97-26811-NormBPHProstate |
| 858 | 559336 | 2458.O09.GZ43_356953 | F | M00074268A:D08 | IF97-26811-NormBPHProstate |
| 859 | 507628 | 2458.O17.GZ43_356961 | F | M00074268C:G03 | IF97-26811-NormBPHProstate |
| 860 | 14453 | 2458.P06.GZ43_356974 | F | M00074270B:A01 | IF97-26811-NormBPHProstate |
| 861 | 858675 | 2458.P18.GZ43_356986 | F | M00074271B:E11 | IF97-26811-NormBPHProstate |
| 862 | 597681 | 2459.A04.GZ43_356996 | F | M00074273B:B03 | IF97-26811-NormBPHProstate |
| 863 | 715752 | 2459.A24.GZ43_357016 | F | M00074275A:B04 | IF97-26811-NormBPHProstate |
| 864 | 14049 | 2459.B10.GZ43_357026 | F | M00074276A:A12 | IF97-26811-NormBPHprostate |
| 865 | 830453 | 2459.B11.GZ43_357027 | F | M00074276A:E02 | IF97-26811-NormBPHProstate |
| 866 | 63551 | 2459.C05.GZ43_357045 | F | M00074278B:D07 | IF97-26811-NormBPHProstate |
| 867 | 456211 | 2459.C09.GZ43_357049 | F | M00074278D:E07 | IF97-26811-NormBPHProstate |
| 868 | 682065 | 2459.C16.GZ43_357056 | F | M00074279C:C11 | IF97-26811-NormBPHProstate |
| 869 | 1049007 | 2459.D07.GZ43_357071 | F | M00074280D:H03 | IF97-26811-NormBPHProstate |
| 870 | 415520 | 2459.E11.GZ43_357099 | F | M00074284B:B03 | IF97-26811-NormBPHProstate |
| 871 | 136276 | 2459.E16.GZ43_357104 | F | M00074284C:B06 | IF97-26811-NormBPHProstate |
| 872 | 532090 | 2459.E19.GZ43_357107 | F | M00074284C:E12 | IF97-26811-NormBPHProstate |
| 873 | 165378 | 2459.F20.GZ43_357132 | F | M00074288A:F11 | IF97-26811-NormBPHProstate |
| 874 | 523261 | 2459.G01.GZ43_357137 | F | M00074290A:G10 | IF97-26811-NormBPHProstate |
| 875 | 22351 | 2459.G07.GZ43_357143 | F | M00074290C:B05 | IF97-26811-NormBPHProstate |
| 876 | 573764 | 2459.G23.GZ43_357159 | F | M00074292D:B04 | IF97-26811-NormBPHProstate |
| 877 | 552996 | 2459.H09.GZ43_357169 | F | M00074293D:B05 | IF97-26811-NormBPHProstate |
| 878 | 923732 | 2459.H10.GZ43_357170 | F | M00074293D:H07 | IF97-26811-NormBPHProstate |
| 879 | 375712 | 2459.I10.GZ43_357194 | F | M00074296C:G09 | IF97-26811-NormBPHProstate |
| 880 | 8342 | 2459.J12.GZ43_357220 | F | M00074299B:F01 | IF97-26811-NormBPHProstate |
| 881 | 446975 | 2459.K15.GZ43_357247 | F | M00074302D:G10 | IF97-26811-NormBPHProstate |
| 882 | 747429 | 2459.L07.GZ43_357263 | F | M00074304B:C09 | IF97-26811-NormBPHProstate |
| 883 | 697955 | 2459.L13.GZ43_357269 | F | M00074304D:D07 | IF97-26811-NormBPHProstate |
| 884 | 2594 | 2459.L18.GZ43_357274 | F | M00074306A:B09 | IF97-26811-NormBPHProstate |
| 885 | 19812 | 2459.L23.GZ43_357279 | F | M00074306B:H01 | IF97-26811-NormBPHProstate |
| 886 | 38435 | 2459.N09.GZ43_357313 | F | M00074310D:D02 | IF97-26811-NormBPHProstate |
| 887 | 4526 | 2459.O12.GZ43_357340 | F | M00074314A:C06 | IF97-26811-NormBPHProstate |
| 888 | 61211 | 2459.O23.GZ43_357351 | F | M00074315B:A03 | IF97-26811-NormBPHProstate |
| 889 | 558789 | 2459.P24.GZ43_357376 | F | M00074317C:C01 | IF97-26811-NormBPHProstate |
| 890 | 676448 | 2464.B01.GZ43_357705 | F | M00074319C:H03 | IF97-26811-NormBPHProstate |
| 891 | 18780 | 2464.C08.GZ43_357736 | F | M00074832B:E05 | IF97-26811-NormBPHProstate |
| 892 | 35553 | 2464.D18.GZ43_357770 | F | M00074835A:H10 | IF97-26811-NormBPHProstate |
| 893 | 797055 | 2464.D23.GZ43_357775 | F | M00074835B:F12 | IF97-26811-NormBPHProstate |
| 894 | 595523 | 2464.E21.GZ43_357797 | F | M00074837A:B06 | IF97-26811-NormBPHProstate |
| 895 | 97523 | 2464.E23.GZ43_357799 | F | M00074837A:E01 | IF97-26811-NormBPHProstate |
| 896 | 22970 | 2464.F12.GZ43_357812 | F | M00074838B:E11 | IF97-26811-NormBPHProstate |
| 897 | 743862 | 2464.F19.GZ43_357819 | F | M00074838D:B06 | IF97-26811-NormBPHProstate |
| 898 | 551338 | 2464.G18.GZ43_357842 | F | M00074843A:C06 | IF97-26811-NormBPHProstate |
| 899 | 524917 | 2464.H05.GZ43_357853 | F | M00074843D:D02 | IF97-26811-NormBPHProstate |
| 900 | 10663 | 2464.H07.GZ43_357855 | F | M00074844B:B02 | IF97-26811-NormBPHProstate |
| 901 | 453526 | 2464.H14.GZ43_357862 | F | M00074844D:F09 | IF97-26811-NormBPHProstate |
| 902 | 459310 | 2464.H17.GZ43_357865 | F | M00074845A:D12 | IF97-26811-NormBPHProstate |
| 903 | 215935 | 2464.H22.GZ43_357870 | F | M00074845B:F07 | IF97-26811-NormBPHProstate |
| 904 | 158853 | 2464.I04.GZ43_357876 | F | M00074845D:D07 | IF97-26811-NormBPHProstate |
| 905 | 465814 | 2464.I20.GZ43_357892 | F | M00074847B:G03 | IF97-26811-NormBPHProstate |
| 906 | 558463 | 2464.I23.GZ43_357895 | F | M00074847D:E07 | IF97-26811-NormBPHProstate |
| 907 | 323112 | 2464.J17.GZ43_357913 | F | M00074849C:A04 | IF97-26811-NormBPHProstate |
| 908 | 813848 | 2464.K14.GZ43_357934 | F | M00074852A:B01 | IF97-26811-NormBPHProstate |
| 909 | 517954 | 2464.K18.GZ43_357938 | F | M00074852B:A02 | IF97-26811-NormBPHProstate |
| 910 | 532090 | 2464.L02.GZ43_357946 | F | M00074852D:D08 | IF97-26811-NormBPHProstate |
| 911 | 365634 | 2464.L06.GZ43_357950 | F | M00074853A:D05 | IF97-26811-NormBPHProstate |
| 912 | 560612 | 2464.L15.GZ43_357959 | F | M00074854A:C11 | IF97-26811-NormBPHProstate |
| 913 | 419172 | 2464.M02.GZ43_357970 | F | M00074855B:A05 | IF97-26811-NormBPHProstate |
| 914 | 932437 | 2464.N05.GZ43_357997 | F | M00074857D:B02 | IF97-26811-NormBPHProstate |
| 915 | 411524 | 2464.N06.GZ43_357998 | F | M00074858B:E05 | IF97-26811-NormBPHProstate |
| 916 | 558959 | 2464.O15.GZ43_358031 | F | M00074861D:D01 | IF97-26811-NormBPHProstate |
| 917 | 528957 | 2464.P10.GZ43_358050 | F | M00074863D:F07 | IF97-26811-NormBPHProstate |
| 918 | 85702 | 2464.P17.GZ43_358057 | F | M00074864C:B09 | IF97-26811-NormBPHProstate |
| 919 | 88413 | 2464.A05.GZ43_357685 | F | M00074317D:B08 | IF97-26811-NormBPHProstate |
| 920 | 549017 | 2464.B11.GZ43_357715 | F | M00074320C:A06 | IF97-26811-NormBPHProstate |
| 921 | 582134 | 2465.A03.GZ43_358067 | F | M00074865A:F05 | IF97-26811-NormBPHProstate |
| 922 | 482747 | 2465.B11.GZ43_358099 | F | M00074869C:D04 | IF97-26811-NormBPHProstate |
| 923 | 545694 | 2465.C01.GZ43_358113 | F | M00074871C:G05 | IF97-26811-NormBPHProstate |
| 924 | 853085 | 2465.C24.GZ43_358136 | F | M00074874A:G07 | IF97-26811-NormBPHProstate |
| 925 | 146695 | 2465.D10.GZ43_358146 | F | M00074875B:E08 | IF97-26811-NormBPHProstate |
| 926 | 935908 | 2465.E03.GZ43_358163 | F | M00074879A:A02 | IF97-26811-NormBPHProstate |
| 927 | 726585 | 2465.E08.GZ43_358168 | F | M00074879C:D02 | IF97-26811-NormBPHProstate |
| 928 | 647607 | 2465.F11.GZ43_358195 | F | M00074884C:F10 | IF97-26811-NormBPHProstate |
| 929 | 464200 | 2465.G06.GZ43_358214 | F | M00074887A:F03 | IF97-26811-NormBPHProstate |
| 930 | 672079 | 2465.H11.GZ43_358243 | F | M00074890A:E03 | IF97-26811-NormBPHProstate |
| 931 | 498886 | 2465.I12.GZ43_358268 | F | M00074895D:H12 | IF97-26811-NormBPHProstate |
| 932 | 542693 | 2465.I17.GZ43_358273 | F | M00074898B:B01 | IF97-26811-NormBPHProstate |
| 933 | 47795 | 2465.J11.GZ43_358291 | F | M00074900C:E10 | IF97-26811-NormBPHProstate |
| 934 | 725257 | 2465.J19.GZ43_358299 | F | M00074901C:E05 | IF97-26811-NormBPHProstate |
| 935 | 376516 | 2465.K20.GZ43_358324 | F | M00074903D:C04 | IF97-26811-NormBPHProstate |
| 936 | 659483 | 2465.L02.GZ43_358330 | F | M00074904A:E11 | IF97-26811-NormBPHProstate |
| 937 | 41346 | 2465.L06.GZ43_358334 | F | M00074904B:B07 | IF97-26811-NormBPHProstate |
| 938 | 498886 | 2465.L22.GZ43_358350 | F | M00074905D:A01 | IF97-26811-NormBPHProstate |
| 939 | 447525 | 2465.M11.GZ43_358363 | F | M00074906B:H12 | IF97-26811-NormBPHProstate |
| 940 | 672079 | 2465.M18.GZ43_358370 | F | M00074906D:G02 | IF97-26811-NormBPHProstate |
| 941 | 738784 | 2465.P14.GZ43_358438 | F | M00074912B:A10 | IF97-26811-NormBPHProstate |
| 942 | 402167 | 2466.A02.GZ43_360083 | F | M00074912D:H08 | IF97-26811-NormBPHProstate |
| 943 | 11686 | 2466.B02.GZ43_360107 | F | M00074916A:H03 | IF97-26811-NormBPHProstate |
| 944 | 709796 | 2466.C15.GZ43_360144 | F | M00074919C:A08 | IF97-26811-NormBPHProstate |
| 945 | 553629 | 2466.D19.GZ43_360172 | F | M00074921C:E05 | IF97-26811-NormBPHProstate |
| 946 | 627263 | 2466.D20.GZ43_360173 | F | M00074922A:D06 | IF97-26811-NormBPHProstate |
| 947 | 20975 | 2466.F16.GZ43_360217 | F | M00074927A:D02 | IF97-26811-NormBPHProstate |
| 948 | 861172 | 2466.F19.GZ43_360220 | F | M00074927B:G08 | IF97-26811-NormBPHProstate |
| 949 | 588996 | 2466.G06.GZ43_360231 | F | M00074927D:G09 | IF97-26811-NormBPHProstate |
| 950 | 993554 | 2466.H07.GZ43_360256 | F | M00074929D:D04 | IF97-26811-NormBPHProstate |
| 951 | 652099 | 2466.H19.GZ43_360268 | F | M00074930C:D11 | IF97-26811-NormBPHProstate |
| 952 | 281 | 2466.I08.GZ43_360281 | F | M00074933A:D04 | IF97-26811-NormBPHProstate |
| 953 | 407944 | 2466.J01.GZ43_360298 | F | M00074935A:C01 | IF97-26811-NormBPHProstate |
| 954 | 644299 | 2466.J24.GZ43_360321 | F | M00074936B:E10 | IF97-26811-NormBPHProstate |
| 955 | 374829 | 2466.L07.GZ43_360352 | F | M00074939B:A06 | IF97-26811-NormBPHProstate |
| 956 | 12885 | 2466.M02.GZ43_360371 | F | M00074940C-H08 | IF97-26811-NormBPHProstate |
| 957 | 123563 | 2466.P11.GZ43_360452 | F | M00074950A:D01 | IF97-26811-NormBPHProstate |
| 958 | 540142 | 2467.B24.GZ43_360513 | F | M00074958D:H10 | IF97-26811-NormBPHProstate |
| 959 | 806992 | 2467.D20.GZ43_360557 | F | M00074966D:E08 | IF97-26811-NormBPHProstate |
| 960 | 61211 | 2467.D23.GZ43_360560 | F | M00074967B:A11 | IF97-26811-NormBPHProstate |
| 961 | 682065 | 2467.E19.GZ43_360580 | F | M00074968D:A02 | IF97-26811-NormBPHProstate |
| 962 | 449521 | 2467.G19.GZ43_360628 | F | M00074974C:E11 | IF97-26811-NormBPHProstate |
| 963 | 19342 | 2467.H18.GZ43_360651 | F | M00074980D:E07 | IF97-26811-NormBPHProstate |
| 964 | 373888 | 2467.A03.GZ43_360468 | F | M00074954A:H06 | IF97-26811-NormBPHProstate |
| 965 | 417672 | 2467.A05.GZ43_360470 | F | M00074954B:E03 | IF97-26811-NormBPHProstate |
| 966 | 376630 | 2467.B11.GZ43_360500 | F | M00074957D:F11 | IF97-26811-NormBPHProstate |
| 967 | 733132 | 2467.D10.GZ43_360547 | F | M00074962B:F08 | IF97-26811-NormBPHProstate |
| 968 | 189951 | 2467.E12.GZ43_360573 | F | M00074968A:D09 | IF97-26811-NormBPHProstate |
| 969 | 59884 | 2467.G01.GZ43_360610 | F | M00074973A:H03 | IF97-26811-NormBPHProstate |
| 970 | 16011 | 2467.K17.GZ43_360722 | F | M00072987B:A03 | IF97-26811-ProstateCancer3 + 3 |
| 971 | 2081 | 2467.N22.GZ43_360799 | F | M00072997B:H03 | IF97-26811-ProstateCancer3 + 3 |
| 972 | 377134 | 2467.I02.GZ43_360659 | F | M00072951C:C11 | IF97-26811-ProstateCancer3 + 3 |
| 973 | 3581 | 2467.I12.GZ43_360669 | F | M00072953B:G03 | IF97-26811-ProstateCancer3 + 3 |
| 974 | 21702 | 2467.J09.GZ43_360690 | F | M00072982D:B03 | IF97-26811-ProstateCancer3 + 3 |
| 975 | 1409 | 2467.K03.GZ43_360708 | F | M00072985A:C12 | IF97-26811-ProstateCancer3 + 3 |
| 976 | 36814 | 2467.K08.GZ43_360713 | F | M00072985B:D03 | IF97-26811-ProstateCancer3 + 3 |
| 977 | 448841 | 2467.K14.GZ43_360719 | F | M00072986A:C03 | IF97-26811-ProstateCancer3 + 3 |
| 978 | 568661 | 2467.M07.GZ43_360760 | F | M00072993B:D06 | IF97-26811-ProstateCancer3 + 3 |
| 979 | 388450 | 2467.N03.GZ43_360780 | F | M00072995C:D07 | IF97-26811-ProstateCancer3 + 3 |
| 980 | 129409 | 2467.N07.GZ43_360784 | F | M00072995D:C09 | IF97-26811-ProstateCancer3 + 3 |
| 981 | 14464 | 2467.N09.GZ43_360786 | F | M00072996B:A10 | IF97-26811-ProstateCancer3 + 3 |
| 982 | 1005804 | 2467.N12.GZ43_360789 | F | M00072996C:C04 | IF97-26811-ProstateCancer3 + 3 |
| 983 | 470032 | 2467.O04.GZ43_360805 | F | M00072997D:F08 | IF97-26811-ProstateCancer3 + 3 |
| 984 | 10354 | 2467.O05.GZ43_360806 | F | M00072997D:H06 | IF97-26811-ProstateCancer3 + 3 |
| 985 | 376972 | 2472.A03.GZ43_360852 | F | M00074323D:F09 | IF97-26811-ProstateCancer3 + 3 |
| 986 | 18338 | 2472.C18.GZ43_360915 | F | M00074333D:A11 | IF97-26811-ProstateCancer3 + 3 |
| 987 | 378269 | 2472.D06.GZ43_360927 | F | M00074335A:H08 | IF97-26811-ProstateCancer3 + 3 |
| 988 | 385300 | 2472.D16.GZ43_360937 | F | M00074337A:G08 | IF97-26811-ProstateCancer3 + 3 |
| 989 | 571 | 2472.E02.GZ43_360947 | F | M00074340B:D06 | IF97-26811-ProstateCancer3 + 3 |
| 990 | 377667 | 2472.E22.GZ43_360967 | F | M00074343C:A03 | IF97-26811-ProstateCancer3 + 3 |
| 991 | 450657 | 2472.F22.GZ43_360991 | F | M00074346A:H09 | IF97-26811-ProstateCancer3 + 3 |
| 992 | 15619 | 2472.G03.GZ43_360996 | F | M00074347B:F11 | IF97-26811-ProstateCancer3 + 3 |
| 993 | 185791 | 2472.G13.GZ43_361006 | F | M00074349A:E08 | IF97-26811-ProstateCancer3 + 3 |
| 994 | 193306 | 2472.I14.GZ43_361055 | F | M00074355D:H06 | IF97-26811-ProstateCancer3 + 3 |
| 995 | 377967 | 2472.K13.GZ43_361102 | F | M00074361C:B01 | IF97-26811-ProstateCancer3 + 3 |
| 996 | 373149 | 2472.L11.GZ43_361124 | F | M00074365A:E09 | IF97-26811-ProstateCancer3 + 3 |
| 997 | 612171 | 2472.L15.GZ43_361128 | F | M00074366A:D07 | IF97-26811-ProstateCancer3 + 3 |
| 998 | 560365 | 2472.L16.GZ43_361129 | F | M00074366A:H07 | IF97-26811-ProstateCancer3 + 3 |
| 999 | 217476 | 2472.M22.GZ43_361159 | F | M00074370D:G09 | IF97-26811-ProstateCancer3 + 3 |
| 1000 | 40043 | 2472.O04.GZ43_361189 | F | M00074375D:E05 | IF97-26811-ProstateCancer3 + 3 |
| 1001 | 374588 | 2472.P14.GZ43_361223 | F | M00074382D:F04 | IF97-26811-ProstateCancer3 + 3 |
| 1002 | 15692 | 2472.P22.GZ43_361231 | F | M00074384D:G07 | IF97-26811-ProstateCancer3 + 3 |
| 1003 | 378507 | 2473.A01.GZ43_361234 | F | M00074388B:E07 | IF97-26811-ProstateCancer3 + 3 |
| 1004 | 374382 | 2473.C08.GZ43_361284 | F | M00074392C:D02 | IF97-26811-ProstateCancer3 + 3 |
| 1005 | 372993 | 2473.F08.GZ43_361361 | F | M00074405B:A04 | IF97-26811-ProstateCancer3 + 3 |
| 1006 | 235268 | 2473.F14.GZ43_361367 | F | M00074417D:F07 | IF97-26811-ProstateCancer3 + 3 |
| 1007 | 387530 | 2473.G03.GZ43_361380 | F | M00074392D:D01 | IF97-26811-ProstateCancer3 + 3 |
| 1008 | 375786 | 2473.G09.GZ43_361386 | F | M00074406B:F10 | IF97-26811-ProstateCancer3 + 3 |
| 1009 | 401120 | 2473.H18.GZ43_361419 | F | M00074430D:G09 | IF97-26811-ProstateCancer3 + 3 |
| 1010 | 4885 | 2473.I04.GZ43_361429 | F | M00074395A:B11 | IF97-26811-ProstateCancer3 + 3 |
| 1011 | 5810 | 2473.I08.GZ43_361433 | F | M00074404B:H01 | IF97-26811-ProstateCancer3 + 3 |
| 1012 | 556192 | 2473.K02.GZ43_361475 | F | M00074391B:D02 | IF97-26811-ProstateCancer3 + 3 |
| 1013 | 392161 | 2473.L01.GZ43_361498 | F | M00074390C:E04 | IF97-26811-ProstateCancer3 + 3 |
| 1014 | 971463 | 2473.L11.GZ43_361508 | F | M00074411B:G07 | IF97-26811-ProstateCancer3 + 3 |
| 1015 | 1338 | 2473.O13.GZ43_361582 | F | M00074415B:A01 | IF97-26811-ProstateCancer3 + 3 |
| 1016 | 470032 | 2474.C01.GZ43_361666 | F | M00074453B:H03 | IF97-26811-ProstateCancer3 + 3 |
| 1017 | 565709 | 2474.C04.GZ43_361669 | F | M00074453C:E09 | IF97-26811-ProstateCancer3 + 3 |
| 1018 | 966482 | 2474.C08.GZ43_361673 | F | M00074454A:D08 | IF97-26811-ProstateCancer3 + 3 |
| 1019 | 549017 | 2474.E09.GZ43_361722 | F | M00074461D:E04 | IF97-26811-ProstateCancer3 + 3 |
| 1020 | 32016 | 2474.E18.GZ43_361731 | F | M00074463B:C03 | IF97-26811-ProstateCancer3 + 3 |
| 1021 | 477010 | 2474.G17.GZ43_361778 | F | M00074468B:C03 | IF97-26811-ProstateCancer3 + 3 |
| 1022 | 837214 | 2474.I02.GZ43_361811 | F | M00074473D:H09 | IF97-26811-ProstateCancer3 + 3 |
| 1023 | 861902 | 2474.I06.GZ43_361815 | F | M00074474B:F02 | IF97-26811-ProstateCancer3 + 3 |
| 1024 | 1084307 | 2474.J18.GZ43_361851 | F | M00074488C:C10 | IF97-26811-ProstateCancer3 + 3 |
| 1025 | 715573 | 2474.J19.GZ43_361852 | F | M00074488C:C08 | IF97-26811-ProstateCancer3 + 3 |
| 1026 | 402167 | 2474.K20.GZ43_361877 | F | M00074492A:F11 | IF97-26811-ProstateCancer3 + 3 |
| 1027 | 287803 | 2474.M19.GZ43_361924 | F | M00074501A:G07 | IF97-26811-ProstateCancer3 + 3 |
| 1028 | 421298 | 2474.N01.GZ43_361930 | F | M00074502C:B08 | IF97-26811-ProstateCancer3 + 3 |
| 1029 | 558463 | 2474.P19.GZ43_361996 | F | M00074515A:E02 | IF97-26811-ProstateCancer3 + 3 |
| 1030 | 187860 | 2474.P22.GZ43_361999 | F | M00074515C:A11 | IF97-26811-ProstateCancer3 + 3 |
| 1031 | 474947 | 2475.A05.GZ43_362006 | F | M00074516B:H03 | IF97-26811-ProstateCancer3 + 3 |
| 1032 | 161012 | 2475.C18.GZ43_362067 | F | M00074525A:B05 | IF97-26811-ProstateCancer3 + 3 |
| 1033 | 823296 | 2475.E18.GZ43_362115 | F | M00074533A:D07 | IF97-26811-ProstateCancer3 + 3 |
| 1034 | 176266 | 2475.G16.GZ43_362161 | F | M00074539D:A10 | IF97-26811-ProstateCancer3 + 3 |
| 1035 | 385843 | 2475.H06.GZ43_362175 | F | M00074540B:H07 | IF97-26811-ProstateCancer3 + 3 |
| 1036 | 1009284 | 2475.H13.GZ43_362182 | F | M00074541D:E07 | IF97-26811-ProstateCancer3 + 3 |
| 1037 | 428883 | 2475.J15.GZ43_362232 | F | M00074549B:A06 | IF97-26811-ProstateCancer3 + 3 |
| 1038 | 732950 | 2475.L17.GZ43_362282 | F | M00074557A:G08 | IF97-26811-ProstateCancer3 + 3 |
| 1039 | 387530 | 2475.N08.GZ43_362321 | F | M00074561D:D12 | IF97-26811-ProstateCancer3 + 3 |
| 1040 | 27991 | 2475.O11.GZ43_362348 | F | M00074566B:A04 | IF97-26811-ProstateCancer3 + 3 |
| 1041 | 485653 | 2475.P12.GZ43_362373 | F | M00074569D:D04 | IF97-26811-ProstateCancer3 + 3 |
| 1042 | 540379 | 2475.B20.GZ43_362045 | F | M00074521D:F01 | IF97-26811-ProstateCancer3 + 3 |
| 1043 | 732950 | 2475.J19.GZ43_362236 | F | M00074549C:H08 | IF97-26811-ProstateCancer3 + 3 |
| 1044 | 187860 | 2475.K24.GZ43_362265 | F | M00074555A:E10 | IF97-26811-ProstateCancer3 + 3 |
| 1045 | 570804 | 2475.M20.GZ43_362309 | F | M00074561A:B09 | IF97-26811-ProstateCancer3 + 3 |
| 1046 | 449889 | 2475.N21.GZ43_362334 | F | M00074565A:D08 | IF97-26811-ProstateCancer3 + 3 |
| 1047 | 724905 | 2480.A13.GZ43_358516 | F | M00074571D:F02 | IF97-26811-ProstateCancer3 + 3 |
| 1048 | 21702 | 2480.A20.GZ43_358523 | F | M00074573A:H02 | IF97-26811-ProstateCancer3 + 3 |
| 1049 | 83576 | 2480.B22.GZ43_358549 | F | M00074577B:B12 | IF97-26811-ProstateCancer3 + 3 |
| 1050 | 649404 | 2480.C01.GZ43_358552 | F | M00074577C:A05 | IF97-26811-ProstateCancer3 + 3 |
| 1051 | 635332 | 2480.D13.GZ43_358588 | F | M00074582C:C02 | IF97-26811-ProstateCancer3 + 3 |
| 1052 | 805118 | 2480.D16.GZ43_358591 | F | M00074582D:B09 | IF97-26811-ProstateCancer3 + 3 |
| 1053 | 549507 | 2480.E19.GZ43_358618 | F | M00074584D:C01 | IF97-26811-ProstateCancer3 + 3 |
| 1054 | 838155 | 2480.G04.GZ43_358651 | F | M00074588C:H06 | IF97-26811-ProstateCancer3 + 3 |
| 1055 | 529381 | 2480.G11.GZ43_358658 | F | M00074589A:E10 | IF97-26811-ProstateCancer3 + 3 |
| 1056 | 29273 | 2480.H06.GZ43_358677 | F | M00074593A:F05 | IF97-26811-ProstateCancer3 + 3 |
| 1057 | 963580 | 2480.I08.GZ43_358703 | F | M00074596D:B12 | IF97-26811-ProstateCancer3 + 3 |
| 1058 | 104204 | 2480.K20.GZ43_358763 | F | M00074606C:G02 | IF97-26811-ProstateCancer3 + 3 |
| 1059 | 20580 | 2480.L02.GZ43_358769 | F | M00074607D:A12 | IF97-26811-ProstateCancer3 + 3 |
| 1060 | 899126 | 2480.M15.GZ43_358806 | F | M00074613D:F01 | IF97-26811-ProstateCancer3 + 3 |
| 1061 | 14214 | 2480.M20.GZ43_358811 | F | M00074614B:D10 | IF97-26811-ProstateCancer3 + 3 |
| 1062 | 47888 | 2480.P07.GZ43_358870 | F | M00074625A:C12 | IF97-26811-ProstateCancer3 + 3 |
| 1063 | 486512 | 2480.P22.GZ43_358885 | F | M00074628C:C11 | IF97-26811-ProstateCancer3 + 3 |
| 1064 | 597201 | 2480.P23.GZ43_358886 | F | M00074628C:D03 | IF97-26811-ProstateCancer3 + 3 |
| 1065 | 134597 | 2481.B06.GZ43_358917 | F | M00074633A:B09 | IF97-26811-ProstateCancer3 + 3 |
| 1066 | 933128 | 2481.C22.GZ43_358957 | F | M00074636D:C01 | IF97-26811-ProstateCancer3 + 3 |
| 1067 | 8997 | 2481.D04.GZ43_358963 | F | M00074637A:C02 | IF97-26811-ProstateCancer3 + 3 |
| 1068 | 20863 | 2481.D10.GZ43_358969 | F | M00074638D:C12 | IF97-26811-ProstateCancer3 + 3 |
| 1069 | 58496 | 2481.D13.GZ43_358972 | F | M00074639A:C08 | IF97-26811-ProstateCancer3 + 3 |
| 1070 | 372993 | 2481.E03.GZ43_358986 | F | M00074640D:F07 | IF97-26811-ProstateCancer3 + 3 |
| 1071 | 558581 | 2481.F24.GZ43_359031 | F | M00074645C:B07 | IF97-26811-ProstateCancer3 + 3 |
| 1072 | 471364 | 2481.I05.GZ43_359084 | F | M00074654D:B05 | IF97-26811-ProstateCancer3 + 3 |
| 1073 | 234423 | 2481.J23.GZ43_359126 | F | M00074662B:A05 | IF97-26811-ProstateCancer3 + 3 |
| 1074 | 469837 | 2481.J24.GZ43_359127 | F | M00074662D:D01 | IF97-26811-ProstateCancer3 + 3 |
| 1075 | 449749 | 2481.K12.GZ43_359139 | F | M00074664C:G09 | IF97-26811-ProstateCancer3 + 3 |
| 1076 | 35578 | 2481.L13.GZ43_359164 | F | M00074668D:D04 | IF97-26811-ProstateCancer3 + 3 |
| 1077 | 464200 | 2481.N10.GZ43_359209 | F | M00074674D:D02 | IF97-26811-ProstateCancer3 + 3 |
| 1078 | 555867 | 2481.O05.GZ43_359228 | F | M00074676D:H07 | IF97-26811-ProstateCancer3 + 3 |
| 1079 | 218833 | 2482.A05.GZ43_359276 | F | M00074681C:G11 | IF97-26811-ProstateCancer3 + 3 |
| 1080 | 782981 | 2482.A06.GZ43_359277 | F | M00074681D:A02 | IF97-26811-ProstateCancer3 + 3 |
| 1081 | 475054 | 2482.B22.GZ43_359317 | F | M00074687B:E01 | IF97-26811-ProstateCancer3 + 3 |
| 1082 | 468400 | 2482.E07.GZ43_359374 | F | M00074699B:C03 | IF97-26811-ProstateCancer3 + 3 |
| 1083 | 16641 | 2482.E17.GZ43_359384 | F | M00074701D:H09 | IF97-26811-ProstateCancer3 + 3 |
| 1084 | 460493 | 2482.E20.GZ43_359387 | F | M00074702B:F12 | IF97-26811-ProstateCancer3 + 3 |
| 1085 | 922 | 2482.F01.GZ43_359392 | F | M00074702D:H05 | IF97-26811-ProstateCancer3 + 3 |
| 1086 | 1037152 | 2482.I05.GZ43_359468 | F | M00074713B:F02 | IF97-26811-ProstateCancer3 + 3 |
| 1087 | 540379 | 2482.J06.GZ43_359493 | F | M00074716C:H07 | IF97-26811-ProstateCancer3 + 3 |
| 1088 | 475054 | 2482.L14.GZ43_359549 | F | M00074723D:C06 | IF97-26811-ProstateCancer3 + 3 |
| 1089 | 452194 | 2482.L15.GZ43_359550 | F | M00074723D:D05 | IF97-26811-ProstateCancer3 + 3 |
| 1090 | 7292 | 2482.N01.GZ43_359584 | F | M00074728C:B08 | IF97-26811-ProstateCancer3 + 3 |
| 1091 | 375712 | 2482.N09.GZ43_359592 | F | M00074730B:A04 | IF97-26811-ProstateCancer3 + 3 |
| 1092 | 450119 | 2483.A13.GZ43_359668 | F | M00074740B:F06 | IF97-26811-ProstateCancer3 + 3 |
| 1093 | 549507 | 2483.B23.GZ43_359702 | F | M00074744B:B12 | IF97-26811-ProstateCancer3 + 3 |
| 1094 | 448319 | 2483.D03.GZ43_359730 | F | M00074748C:G02 | IF97-26811-ProstateCancer3 + 3 |
| 1095 | 402591 | 2483.E11.GZ43_359762 | F | M00074752A:D08 | IF97-26811-ProstateCancer3 + 3 |
| 1096 | 654181 | 2483.F04.GZ43_359779 | F | M00074753C:E10 | IF97-26811-ProstateCancer3 + 3 |
| 1097 | 379774 | 2483.F14.GZ43_359789 | F | M00074755A:B10 | IF97-26811-ProstateCancer3 + 3 |
| 1098 | 587168 | 2483.F15.GZ43_359790 | F | M00074755A:E07 | IF97-26811-ProstateCancer3 + 3 |
| 1099 | 187860 | 2483.I21.GZ43_359868 | F | M00074765D:F06 | IF97-26811-ProstateCancer3 + 3 |
| 1100 | 437748 | 2483.J07.GZ43_359878 | F | M00074766C:F12 | IF97-26811-ProstateCancer3 + 3 |
| 1101 | 404081 | 2483.K02.GZ43_359897 | F | M00074768C:A05 | IF97-26811-ProstateCancer3 + 3 |
| 1102 | 545694 | 2483.L15.GZ43_359934 | F | M00074773C:G03 | IF97-26811-ProstateCancer3 + 3 |
| 1103 | 474947 | 2483.L22.GZ43_359941 | F | M00074774A:D03 | IF97-26811-ProstateCancer3 + 3 |
| 1104 | 528957 | 2483.M09.GZ43_359952 | F | M00074777A:E01 | IF97-26811-ProstateCancer3 + 3 |
| 1105 | 597201 | 2483.N15.GZ43_359982 | F | M00074780C:C02 | IF97-26811-ProstateCancer3 + 3 |
| 1106 | 460493 | 2483.O07.GZ43_359998 | F | M00074782A:E04 | IF97-26811-ProstateCancer3 + 3 |
| 1107 | 135899 | 2488.B07.GZ43_362475 | F | M00074808B:H02 | IF97-26811-ProstateCancer3 + 3 |
| 1108 | 839006 | 2488.C19.GZ43_362511 | F | M00074996C:D07 | IF97-26811-ProstateCancer3 + 3 |
| 1109 | 1022081 | 2488.D15.GZ43_362531 | F | M00074981C:C09 | IF97-26811-ProstateCancer3 + 3 |
| 1110 | 423303 | 2488.E20.GZ43_362560 | F | M00075000A:D06 | IF97-26811-ProstateCancer3 + 3 |
| 1111 | 387530 | 2488.F06.GZ43_362570 | F | M00074805A:C12 | IF97-26811-ProstateCancer3 + 3 |
| 1112 | 667872 | 2488.F15.GZ43_362579 | F | M00074981D:A03 | IF97-26811-ProstateCancer3 + 3 |
| 1113 | 22334 | 2488.G02.GZ43_362590 | F | M00074794C:H02 | IF97-26811-ProstateCancer3 + 3 |
| 1114 | 524917 | 2488.G05.GZ43_362593 | F | M00074801C:E06 | IF97-26811-ProstateCancer3 + 3 |
| 1115 | 453981 | 2488.G12.GZ43_362600 | F | M00074821B:B03 | IF97-26811-ProstateCancer3 + 3 |
| 1116 | 423664 | 2488.H12.GZ43_362624 | F | M00074823A:E03 | IF97-26811-ProstateCancer3 + 3 |
| 1117 | 1009284 | 2488.K04.GZ43_362688 | F | M00074800B:H01 | IF97-26811-ProstateCancer3 + 3 |
| 1118 | 1009284 | 2488.L04.GZ43_362712 | F | M00074800D:G09 | IF97-26811-ProstateCancer3 + 3 |
| 1119 | 597201 | 2488.N08.GZ43_362764 | F | M00074812A:F03 | IF97-26811-ProstateCancer3 + 3 |
| 1120 | 724818 | 2488.N13.GZ43_362769 | F | M00074825C:E06 | IF97-26811-ProstateCancer3 + 3 |
| 1121 | 534076 | 2488.P01.GZ43_362805 | F | M00074794A:G10 | IF97-26811-ProstateCancer3 + 3 |
| 1122 | 901160 | 2489.A03.GZ43_362831 | F | M00075018A:G04 | IF97-26811-ProstateCancer3 + 3 |
| 1123 | 448680 | 2489.A04.GZ43_362832 | F | M00075020D:B04 | IF97-26811-ProstateCancer3 + 3 |
| 1124 | 13903 | 2489.A13.GZ43_362841 | F | M00075049A:C09 | IF97-26811-ProstateCancer3 + 3 |
| 1125 | 214762 | 2489.B07.GZ43_362859 | F | M00075032A:F02 | IF97-26811-ProstateCancer3 + 3 |
| 1126 | 21662 | 2489.D06.GZ43_362906 | F | M00075029B:E03 | IF97-26811-ProstateCancer3 + 3 |
| 1127 | 379301 | 2489.D18.GZ43_362918 | F | M00075069C:C01 | IF97-26811-ProstateCancer3 + 3 |
| 1128 | 727966 | 2489.F09.GZ43_362957 | F | M00075039A:E01 | IF97-26811-ProstateCancer3 + 3 |
| 1129 | 13071 | 2489.G05.GZ43_362977 | F | M00075024C:G05 | IF97-26811-ProstateCancer3 + 3 |
| 1130 | 60089 | 2489.G20.GZ43_362992 | F | M00075074D:G11 | IF97-26811-ProstateCancer3 + 3 |
| 1131 | 13091 | 2489.G24.GZ43_362996 | F | M00075011A:C11 | IF97-26811-ProstateCancer3 + 3 |
| 1132 | 32367 | 2489.H15.GZ43_363011 | F | M00075061A:B03 | IF97-26811-ProstateCancer3 + 3 |
| 1133 | 1135 | 2489.I11.GZ43_363031 | F | M00075043B:H05 | IF97-26811-ProstateCancer3 + 3 |
| 1134 | 779428 | 2489.J08.GZ43_363052 | F | M00075035C:C09 | IF97-26811-ProstateCancer3 + 3 |
| 1135 | 560612 | 2489.J11.GZ43_363055 | F | M00075045D:H03 | IF97-26811-ProstateCancer3 + 3 |
| 1136 | 726937 | 2489.J21.GZ43_363065 | F | M00075078C:A07 | IF97-26811-ProstateCancer3 + 3 |
| 1137 | 13182 | 2489.K20.GZ43_363088 | F | M00075075A:D12 | IF97-26811-ProstateCancer3 + 3 |
| 1138 | 1037152 | 2489.K21.GZ43_363089 | F | M00075077C:F09 | IF97-26811-ProstateCancer3 + 3 |
| 1139 | 782981 | 2489.L05.GZ43_363097 | F | M00075026A:D11 | IF97-26811-ProstateCancer3 + 3 |
| 1140 | 20975 | 2489.M11.GZ43_363127 | F | M00075044A:C10 | IF97-26811-ProstateCancer3 + 3 |
| 1141 | 1097678 | 2489.M20.GZ43_363136 | F | M00075075A:E09 | IF97-26811-ProstateCancer3 + 3 |
| 1142 | 22208 | 2489.N03.GZ43_363143 | F | M00075020C:D12 | IF97-26811-ProstateCancer3 + 3 |
| 1143 | 625055 | 2490.A07.GZ43_363219 | F | M00075117B:B06 | IF97-26811-ProstateCancer3 + 3 |
| 1144 | 6544 | 2490.B06.GZ43_363242 | F | M00075114C:G11 | IF97-26811-ProstateCancer3 + 3 |
| 1145 | 19627 | 2490.B20.GZ43_363256 | F | M00075153C:C11 | IF97-26811-ProstateCancer3 + 3 |
| 1146 | 779428 | 2490.C23.GZ43_363283 | F | M00075161A:E05 | IF97-26811-ProstateCancer3 + 3 |
| 1147 | 395603 | 2490.D10.GZ43_363294 | F | M00075126B:A06 | IF97-26811-ProstateCancer3 + 3 |
| 1148 | 43907 | 2490.E11.GZ43_363319 | F | M00075126D:H07 | IF97-26811-ProstateCancer3 + 3 |
| 1149 | 782981 | 2490.F01.GZ43_363333 | F | M00075092C:F04 | IF97-26811-ProstateCancer3 + 3 |
| 1150 | 428699 | 2490.H05.GZ43_363385 | F | M00075110C:B03 | IF97-26811-ProstateCancer3 + 3 |
| 1151 | 1005804 | 2490.H12.GZ43_363392 | F | M00075132C:A03 | IF97-26811-ProstateCancer3 + 3 |
| 1152 | 72334 | 2490.I20.GZ43_363424 | F | M00075152D:C06 | IF97-26811-ProstateCancer3 + 3 |
| 1153 | 40517 | 2490.J09.GZ43_363437 | F | M00075125B:C07 | IF97-26811-ProstateCancer3 + 3 |
| 1154 | 13495 | 2490.J12.GZ43_363440 | F | M00075132C:E07 | IF97-26811-ProstateCancer3 + 3 |
| 1155 | 1009284 | 2490.J22.GZ43_363450 | F | M00075160A:E04 | IF97-26811-ProstateCancer3 + 3 |
| 1156 | 60866 | 2490.L17.GZ43_363493 | F | M00075149B:A01 | IF97-26811-ProstateCancer3 + 3 |
| 1157 | 14453 | 2490.M08.GZ43_363508 | F | M00075120C:H04 | IF97-26811-ProstateCancer3 + 3 |
| 1158 | 659483 | 2490.N01.GZ43_363525 | F | M00075093B:F10 | IF97-26811-ProstateCancer3 + 3 |
| 1159 | 792 | 2490.N03.GZ43_363527 | F | M00075102A:D02 | IF97-26811-ProstateCancer3 + 3 |
| 1160 | 380136 | 2490.N24.GZ43_363548 | F | M00075090D:B07 | IF97-26811-ProstateCancer3 + 3 |
| 1161 | 62319 | 2490.O23.GZ43_363571 | F | M00075161D:G06 | IF97-26811-ProstateCancer3 + 3 |
| 1162 | 842403 | 2491.A04.GZ43_363600 | F | M00075165B:D04 | IF97-26811-ProstateCancer3 + 3 |
| 1163 | 779428 | 2491.C13.GZ43_363657 | F | M00075174D:D06 | IF97-26811-ProstateCancer3 + 3 |
| 1164 | 697943 | 2491.D12.GZ43_363680 | F | M00075180D:F05 | IF97-26811-ProstateCancer3 + 3 |
| 1165 | 35486 | 2491.D19.GZ43_363687 | F | M00075181D:G10 | IF97-26811-ProstateCancer3 + 3 |
| 1166 | 311745 | 2491.F16.GZ43_363732 | F | M00075189C:G05 | IF97-26811-ProstateCancer3 + 3 |
| 1167 | 640911 | 2491.H09.GZ43_363773 | F | M00075199D:D11 | IF97-26811-ProstateCancer3 + 3 |
| 1168 | 470032 | 2491.H23.GZ43_363787 | F | M00075201D:A05 | IF97-26811-ProstateCancer3 + 3 |
| 1169 | 853371 | 2491.I06.GZ43_363794 | F | M00075203A:G06 | IF97-26811-ProstateCancer3 + 3 |
| 1170 | 56899 | 2491.J14.GZ43_363826 | F | M00075211D:F09 | IF97-26811-ProstateCancer3 + 3 |
| 1171 | 414887 | 2491.L20.GZ43_363880 | F | M00075221C:E02 | IF97-26811-ProstateCancer3 + 3 |
| 1172 | 540379 | 2491.O02.GZ43_363934 | F | M00075228D:G09 | IF97-26811-ProstateCancer3 + 3 |
| 1173 | 558579 | 2491.P07.GZ43_363963 | F | M00075232C:A06 | IF97-26811-ProstateCancer3 + 3 |
| 1174 | 467877 | 2491.P10.GZ43_363966 | F | M00075232D:C06 | IF97-26811-ProstateCancer3 + 3 |
| 1175 | 379077 | 2491.P20.GZ43_363976 | F | M00075234C:E06 | IF97-26811-ProstateCancer3 + 3 |
| 1176 | 209378 | 2496.B09.GZ43_364116 | F | M00075239C:D06 | IF97-26811-ProstateCancer3 + 3 |
| 1177 | 16204 | 2496.C08.GZ43_364139 | F | M00075242A:G04 | IF97-26811-ProstateCancer3 + 3 |
| 1178 | 137552 | 2496.C18.GZ43_364149 | F | M00075243D:F04 | IF97-26811-ProstateCancer3 + 3 |
| 1179 | 625055 | 2496.D03.GZ43_364158 | F | M00075245A:A06 | IF97-26811-ProstateCancer3 + 3 |
| 1180 | 29921 | 2496.E14.GZ43_364193 | F | M00075249A:B08 | IF97-26811-ProstateCancer3 + 3 |
| 1181 | 831469 | 2496.F14.GZ43_364217 | F | M00075252B:F10 | IF97-26811-ProstateCancer3 + 3 |
| 1182 | 649404 | 2496.G15.GZ43_364242 | F | M00075255A:G11 | IF97-26811-ProstateCancer3 + 3 |
| 1183 | 129139 | 2496.I06.GZ43_364281 | F | M00075259C:G02 | IF97-26811-ProstateCancer3 + 3 |
| 1184 | 72712 | 2496.K15.GZ43_364338 | F | M00075270D:A02 | IF97-26811-ProstateCancer3 + 3 |
| 1185 | 83576 | 2496.L09.GZ43_364356 | F | M00075273C:E01 | IF97-26811-ProstateCancer3 + 3 |
| 1186 | 452194 | 2496.L17.GZ43_364364 | F | M00075274B:F06 | IF97-26811-ProstateCancer3 + 3 |
| 1187 | 625055 | 2496.L22.GZ43_364369 | F | M00075275B:H07 | IF97-26811-ProstateCancer3 + 3 |
| 1188 | 400152 | 2496.M22.GZ43_364393 | F | M00075279C:E08 | IF97-26811-ProstateCancer3 + 3 |
| 1189 | 558463 | 2496.N15.GZ43_364410 | F | M00075283A:F04 | IF97-26811-ProstateCancer3 + 3 |
| 1190 | 411524 | 2497.C11.GZ43_364526 | F | M00075302B:C07 | IF97-26811-ProstateCancer3 + 3 |
| 1191 | 715573 | 2497.D11.GZ43_364550 | F | M00075305C:C07 | IF97-26811-ProstateCancer3 + 3 |
| 1192 | 23000 | 2497.E09.GZ43_364572 | F | M00075309C:A06 | IF97-26811-ProstateCancer3 + 3 |
| 1193 | 9386 | 2497.I15.GZ43_364674 | F | M00075323B:B12 | IF97-26811-ProstateCancer3 + 3 |
| 1194 | 61725 | 2497.I21.GZ43_364680 | F | M00075324B:C10 | IF97-26811-ProstateCancer3 + 3 |
| 1195 | 142924 | 2497.J05.GZ43_364688 | F | M00075324D:E02 | IF97-26811-ProstateCancer3 + 3 |
| 1196 | 160424 | 2497.J23.GZ43_364706 | F | M00075326C:B01 | IF97-26811-ProstateCancer3 + 3 |
| 1197 | 741521 | 2497.K02.GZ43_364709 | F | M00075326D:A09 | IF97-26811-ProstateCancer3 + 3 |
| 1198 | 175903 | 2497.K22.GZ43_364729 | F | M00075329B:E10 | IF97-26811-ProstateCancer3 + 3 |
| 1199 | 388450 | 2497.L05.GZ43_364736 | F | M00075330D:F11 | IF97-26811-ProstateCancer3 + 3 |
| 1200 | 31500 | 2497.L21.GZ43_364752 | F | M00075333D:B07 | IF97-26811-ProstateCancer3 + 3 |
| 1201 | 52245 | 2497.L22.GZ43_364753 | F | M00075333D:D10 | IF97-26811-ProstateCancer3 + 3 |
| 1202 | 18761 | 2497.M17.GZ43_364772 | F | M00075336B:B04 | IF97-26811-ProstateCancer3 + 3 |
| 1203 | 449839 | 2497.O09.GZ43_364812 | F | M00075344D:A08 | IF97-26811-ProstateCancer3 + 3 |
| 1204 | 715573 | 2497.P04.GZ43_364831 | F | M00075347D:D01 | IF97-26811-ProstateCancer3 + 3 |
| 1205 | 212364 | 2562.B05.GZ43_375492 | F | M00075354A:D11 | IF97-26811-ProstateCancer3 + 3 |
| 1206 | 1024470 | 2562.B06.GZ43_375493 | F | M00075354A:G12 | IF97-26811-ProstateCancer3 + 3 |
| 1207 | 40517 | 2562.B09.GZ43_375496 | F | M00075354C:B12 | IF97-26811-ProstateCancer3 + 3 |
| 1208 | 13585 | 2562.D02.GZ43_375537 | F | M00075360D:D04 | IF97-26811-ProstateCancer3 + 3 |
| 1209 | 598388 | 2562.E03.GZ43_375562 | F | M00075365B:B06 | IF97-26811-ProstateCancer3 + 3 |
| 1210 | 185903 | 2562.I01.GZ43_375656 | F | M00075384A:B03 | IF97-26811-ProstateCancer3 + 3 |
| 1211 | 475054 | 2562.J02.GZ43_375681 | F | M00075389B:C06 | IF97-26811-ProstateCancer3 + 3 |
| 1212 | 6136 | 2562.K03.GZ43_375706 | F | M00075391D:D07 | IF97-26811-ProstateCancer3 + 3 |
| 1213 | 60741 | 2562.N02.GZ43_375777 | F | M00075402A:F01 | IF97-26811-ProstateCancer3 + 3 |
| 1214 | 218833 | 2562.O01.GZ43_375800 | F | M00075405B:C07 | IF97-26811-ProstateCancer3 + 3 |
| 1215 | 372710 | 2562.O06.GZ43_375805 | F | M00075405D:A10 | IF97-26811-ProstateCancer3 + 3 |
| 1216 | 465446 | 2562.E14.GZ43_375573 | F | M00075365D:B08 | IF97-26811-ProstateCancer3 + 3 |
| 1217 | 130289 | 2562.H11.GZ43_375642 | F | M00075380D:F06 | IF97-26811-ProstateCancer3 + 3 |
| 1218 | 65337 | 2562.B24.GZ43_375511 | F | M00075356D:C03 | IF97-26811-ProstateCancer3 + 3 |
| 1219 | 743053 | 2562.A22.GZ43_375485 | F | M00075352D:F09 | IF97-26811-ProstateCancer3 + 3 |
| 1220 | 733229 | 2562.C18.GZ43_375529 | F | M00075359D:E09 | IF97-26811-ProstateCancer3 + 3 |
| 1221 | 185886 | 2562.E16.GZ43_375575 | F | M00075365D:H01 | IF97-26811-ProstateCancer3 + 3 |
| 1222 | 11035 | 2562.F17.GZ43_375600 | F | M00075373C:B09 | IF97-26811-ProstateCancer3 + 3 |
| 1223 | 135008 | 2562.G19.GZ43_375626 | F | M00075378B:C07 | IF97-26811-ProstateCancer3 + 3 |
| 1224 | 715573 | 2562.G21.GZ43_375628 | F | M00075379A:E07 | IF97-26811-ProstateCancer3 + 3 |
| 1225 | 376516 | 2562.H18.GZ43_375649 | F | M00075383A:B11 | IF97-26811-ProstateCancer3 + 3 |
| 1226 | 154672 | 2562.O20.GZ43_375819 | F | M00075407A:B05 | IF97-26811-ProstateCancer3 + 3 |
| 1227 | 550132 | 2562.P16.GZ43_375839 | F | M00075409A:E04 | IF97-26811-ProstateCancer3 + 3 |
| 1228 | 452806 | 2562.P18.GZ43_375841 | F | M00075409B:G12 | IF97-26811-ProstateCancer3 + 3 |
| 1229 | 34977 | 2498.A02.GZ43_364853 | F | M00075416C:B02 | IF97-26811-ProstateCancer3 + 3 |
| 1230 | 1759 | 2498.A19.GZ43_364870 | F | M00075458B:F09 | IF97-26811-ProstateCancer3 + 3 |
| 1231 | 743862 | 2498.B22.GZ43_364897 | F | M00075464C:A07 | IF97-26811-ProstateCancer3 + 3 |
| 1232 | 180990 | 2498.C19.GZ43_364918 | F | M00075458C:F01 | IF97-26811-ProstateCancer3 + 3 |
| 1233 | 137835 | 2498.C22.GZ43_364921 | F | M00075463C:E07 | IF97-26811-ProstateCancer3 + 3 |
| 1234 | 396148 | 2498.D22.GZ43_364945 | F | M00075464C:C04 | IF97-26811-ProstateCancer3 + 3 |
| 1235 | 442923 | 2498.G15.GZ43_365010 | F | M00075448B:G11 | IF97-26811-ProstateCancer3 + 3 |
| 1236 | 480410 | 2498.H08.GZ43_365027 | F | M00075434A:D06 | IF97-26811-ProstateCancer3 + 3 |
| 1237 | 395603 | 2498.H18.GZ43_365037 | F | M00075457C:A06 | IF97-26811-ProstateCancer3 + 3 |
| 1238 | 821859 | 2498.I17.GZ43_365060 | F | M00075454C:D06 | IF97-26811-ProstateCancer3 + 3 |
| 1239 | 1082121 | 2498.K20.GZ43_365111 | F | M00075460C:B06 | IF97-26811-ProstateCancer3 + 3 |
| 1240 | 96136 | 2498.M19.GZ43_365158 | F | M00075459A:C02 | IF97-26811-ProstateCancer3 + 3 |
| 1241 | 20460 | 2498.O01.GZ43_365188 | F | M00075414A:D10 | IF97-26811-ProstateCancer3 + 3 |
| 1242 | 6305 | 2498.P07.GZ43_365218 | F | M00075433A:C06 | IF97-26811-ProstateCancer3 + 3 |
| 1243 | 28050 | 2507.B18.GZ43_366983 | F | M00075505B:A04 | IF97-26811-ProstateCancer3 + 3 |
| 1244 | 436755 | 2507.C03.GZ43_366992 | F | M00075474D:B07 | IF97-26811-ProstateCancer3 + 3 |
| 1245 | 691653 | 2507.C18.GZ43_367007 | F | M00075504B:A10 | IF97-26811-ProstateCancer3 + 3 |
| 1246 | 839006 | 2507.H02.GZ43_367111 | F | M00075473C:E08 | IF97-26811-ProstateCancer3 + 3 |
| 1247 | 187223 | 2507.J14.GZ43_367171 | F | M00075499A:H02 | IF97-26811-ProstateCancer3 + 3 |
| 1248 | 966599 | 2507.L12.GZ43_367217 | F | M00075495D:D11 | IF97-26811-ProstateCancer3 + 3 |
| 1249 | 961781 | 2507.M13.GZ43_367242 | F | M00075496D:G05 | IF97-26811-ProstateCancer3 + 3 |
| 1250 | 726937 | 2507.N22.GZ43_367275 | F | M00075514A:G12 | IF97-26811-ProstateCancer3 + 3 |
| 1251 | 379470 | 2507.O12.GZ43_367289 | F | M00075495B:C12 | IF97-26811-ProstateCancer3 + 3 |
| 1252 | 37881 | 2507.P13.GZ43_367314 | F | M00075497D:H03 | IF97-26811-ProstateCancer3 + 3 |
| 1253 | 855568 | 2511.A03.GZ43_369412 | F | M00075529A:A02 | IF97-26811-ProstateCancer3 + 3 |
| 1254 | 625055 | 2511.A07.GZ43_369416 | F | M00075538C:E03 | IF97-26811-ProstateCancer3 + 3 |
| 1255 | 720671 | 2511.H08.GZ43_369585 | F | M00075544A:C03 | IF97-26811-ProstateCancer3 + 3 |
| 1256 | 375488 | 2511.D23.GZ43_369504 | F | M00075598B:A09 | IF97-26811-ProstateCancer3 + 3 |
| 1257 | 958 | 2511.D24.GZ43_369505 | F | M00075521B:E11 | IF97-26811-ProstateCancer3 + 3 |
| 1258 | 20614 | 2511.I23.GZ43_369624 | F | M00075597C:G01 | IF97-26811-ProstateCancer3 + 3 |
| 1259 | 217230 | 2511.J18.GZ43_369643 | F | M00075584D:B05 | IF97-26811-ProstateCancer3 + 3 |
| 1260 | 51189 | 2511.N20.GZ43_369741 | F | M00075590B:G04 | IF97-26811-ProstateCancer3 + 3 |
| 1261 | 377044 | 2499.A22.GZ43_365257 | F | M00075603D:D09 | IF97-26811-ProstateCancer3 + 3 |
| 1262 | 4655 | 2499.B16.GZ43_365275 | F | M00075607B:D05 | IF97-26811-ProstateCancer3 + 3 |
| 1263 | 395761 | 2499.C09.GZ43_365292 | F | M00075609A:H06 | IF97-26811-ProstateCancer3 + 3 |
| 1264 | 135675 | 2499.D16.GZ43_365323 | F | M00075613D:F01 | IF97-26811-ProstateCancer3 + 3 |
| 1265 | 779428 | 2499.E18.GZ43_365349 | F | M00075619C:D08 | IF97-26811-ProstateCancer3 + 3 |
| 1266 | 224580 | 2499.F08.GZ43_365363 | F | M00075621A:F06 | IF97-26811-ProstateCancer3 + 3 |
| 1267 | 13182 | 2499.I09.GZ43_365436 | F | M00075639A:D12 | IF97-26811-ProstateCancer3 + 3 |

TABLE 3

| SEQ | | |
|---|---|---|
| ID | CONSENSUS SEQ NAME | POLYNTD SEQ NAME |
| 1268 | Clu1009284.1 | 2490.J22.GZ43_363450 |
| 1269 | Clu1022935.2 | 2561.O19.GZ43_376586 |
| 1270 | Clu1037152.1 | 2558.L19.GZ43_374594 |
| 1271 | Clu13903.1 | 2489.A13.GZ43_362841 |
| 1272 | Clu139979.2 | 2504.B21.GZ43_365834 |
| 1273 | Clu163602.2 | 2561.H17.GZ43_376416 |
| 1274 | Clu187860.2 | 2474.P22.GZ43_361999 |
| 1275 | Clu189993.1 | 2505.N19.GZ43_366504 |
| 1276 | Clu20975.1 | 2466.F16.GZ43_360217 |
| 1277 | Clu217122.1 | 2458.N10.GZ43_356930 |
| 1278 | Clu218833.1 | 2562.O01.GZ43_375800 |
| 1279 | Clu244504.2 | 2367.E23.GZ43_346113 |
| 1280 | Clu271456.1 | 2365.G19.GZ43_345389 |
| 1281 | Clu376516.1 | 2457.J23.GZ43_356451 |
| 1282 | Clu376630.1 | 2467.B11.GZ43_360500 |
| 1283 | Clu377044.2 | 2499.A22.GZ43_365257 |
| 1284 | Clu379689.1 | 2540.M18.GZ43_372313 |
| 1285 | Clu380482.2 | 2542.D09.GZ43_372856 |
| 1286 | Clu387530.4 | 2475.N08.GZ43_362321 |
| 1287 | Clu388450.2 | 2497.L05.GZ43_364736 |
| 1288 | Clu396325.1 | 2561.P16.GZ43_376607 |
| 1289 | Clu397115.3 | 2560.K18.GZ43_375337 |
| 1290 | Clu398642.2 | 2452.N22.GZ43_373109 |
| 1291 | Clu400258.1 | 2504.O12.GZ43_366137 |
| 1292 | Clu402167.1 | 2540.C21.GZ43_372076 |
| 1293 | Clu402591.3 | 2483.E11.GZ43_359762 |
| 1294 | Clu402904.1 | 2504.J02.GZ43_366007 |
| 1295 | Clu404081.2 | 2483.K02.GZ43_359897 |
| 1296 | Clu411524.1 | 2497.C11.GZ43_364526 |
| 1297 | Clu41346.1 | 2560.K08.GZ43_375327 |
| 1298 | Clu415520.1 | 2561.L14.GZ43_376509 |
| 1299 | Clu416124.1 | 2367.G17.GZ43_346155 |
| 1300 | Clu417672.1 | 2367.I09.GZ43_346195 |
| 1301 | Clu423664.1 | 2488.H12.GZ43_362624 |
| 1302 | Clu429609.1 | 2457.M11.GZ43_356511 |
| 1303 | Clu442923.3 | 2498.G15.GZ43_365010 |
| 1304 | Clu446975.1 | 2459.K15.GZ43_357247 |
| 1305 | Clu449839.2 | 2497.O09.GZ43_364812 |
| 1306 | Clu449889.1 | 2475.N21.GZ43_362334 |
| 1307 | Clu451707.2 | 2554.P16.GZ43_376223 |
| 1308 | Clu454509.3 | 2542.M09.GZ43_373072 |
| 1309 | Clu454796.1 | 2540.P02.GZ43_372369 |
| 1310 | Clu455862.1 | 2560.I09.GZ43_375280 |
| 1311 | Clu460493.1 | 2483.O07.GZ43_359998 |
| 1312 | Clu464200.1 | 2465.G06.GZ43_358214 |
| 1313 | Clu465446.2 | 2457.L21.GZ43_356497 |
| 1314 | Clu470032.1 | 2474.C01.GZ43_361666 |
| 1315 | Clu474125.1 | 2457.E23.GZ43_356331 |
| 1316 | Clu474125.2 | 2541.A06.GZ43_372397 |
| 1317 | Clu477271.1 | 2540.E17.GZ43_372120 |
| 1318 | Clu480410.1 | 2498.H08.GZ43_365027 |
| 1319 | Clu483211.2 | 2510.J18.GZ43_369259 |
| 1320 | Clu497138.1 | 2458.N19.GZ43_356939 |
| 1321 | Clu498886.1 | 2465.L22.GZ43_358350 |
| 1322 | Clu498886.2 | 2541.B15.GZ43_372430 |
| 1323 | Clu5013.2 | 2559.D05.GZ43_374772 |
| 1324 | Clu5105.2 | 2542.D19.GZ43_372866 |
| 1325 | Clu510539.2 | 2558.H17.GZ43_374496 |
| 1326 | Clu514044.1 | 2367.F13.GZ43_346127 |
| 1327 | Clu516526.1 | 2456.F23.GZ43_355971 |
| 1328 | Clu519176.2 | 2559.H20.GZ43_374883 |
| 1329 | Clu520370.1 | 2541.N01.GZ43_372704 |
| 1330 | Clu524917.1 | 2464.H05.GZ43_357853 |
| 1331 | Clu528957.1 | 2540.F15.GZ43_372142 |
| 1332 | Clu533888.1 | 2557.L23.GZ43_374214 |
| 1333 | Clu534076.1 | 2456.C05.GZ43_355881 |
| 1334 | Clu540142.2 | 2456.H02.GZ43_355998 |
| 1335 | Clu540379.2 | 2491.O02.GZ43_363934 |
| 1336 | Clu549507.1 | 2483.B23.GZ43_359702 |
| 1337 | Clu551338.3 | 2457.I12.GZ43_356416 |
| 1338 | Clu552537.2 | 2540.C10.GZ43_372065 |
| 1339 | Clu556827.3 | 2558.E24.GZ43_374431 |
| 1340 | Clu558569.2 | 2558.D03.GZ43_374386 |
| 1341 | Clu565709.1 | 2542.P02.GZ43_373137 |
| 1342 | Clu568204.1 | 2456.M05.GZ43_356121 |
| 1343 | Clu570804.1 | 2475.M20.GZ43_362309 |
| 1344 | Clu572170.2 | 2557.H03.GZ43_374098 |
| 1345 | Clu573764.1 | 2365.C10.GZ43_345284 |
| 1346 | Clu587168.1 | 2483.F15.GZ43_359790 |
| 1347 | Clu588996.1 | 2466.G06.GZ43_360231 |
| 1348 | Clu597681.1 | 2459.A04.GZ43_356996 |
| 1349 | Clu598388.1 | 2562.E03.GZ43_375562 |
| 1350 | Clu604822.2 | 2504.F20.GZ43_365929 |
| 1351 | Clu621573.1 | 2535.A08.GZ43_370095 |
| 1352 | Clu625055.1 | 2511.A07.GZ43_369416 |
| 1353 | Clu627263.1 | 2466.D20.GZ43_360173 |
| 1354 | Clu635332.1 | 2480.D13.GZ43_358588 |
| 1355 | Clu640911.2 | 2541.M24.GZ43_372703 |
| 1356 | Clu641662.2 | 2555.D22.GZ43_373253 |
| 1357 | Clu659483.1 | 2365.F12.GZ43_345358 |
| 1358 | Clu6712.1 | 2535.P14.GZ43_370461 |
| 1359 | Clu676448.3 | 2464.B01.GZ43_357705 |
| 1360 | Clu682065.2 | 2467.E19.GZ43_360580 |
| 1361 | Clu685244.2 | 2561.J01.GZ43_376448 |
| 1362 | Clu691653.1 | 2560.O12.GZ43_375427 |
| 1363 | Clu692282.1 | 2561.I11.GZ43_376434 |
| 1364 | Clu697955.1 | 2557.J22.GZ43_374165 |
| 1365 | Clu702885.3 | 2555.H18.GZ43_373345 |
| 1366 | Clu70908.1 | 2561.C15.GZ43_376294 |
| 1367 | Clu709796.2 | 2542.C20.GZ43_372843 |
| 1368 | Clu715752.1 | 2459.A24.GZ43_357016 |
| 1369 | Clu727966.1 | 2489.F09.GZ43_362957 |
| 1370 | Clu732950.2 | 2475.L17.GZ43_362282 |
| 1371 | Clu752623.2 | 2561.I07.GZ43_376430 |
| 1372 | Clu756337.1 | 2561.I19.GZ43_376442 |
| 1373 | Clu782981.1 | 2489.L05.GZ43_363097 |
| 1374 | Clu805118.3 | 2480.D16.GZ43_358591 |
| 1375 | Clu806992.2 | 2467.D20.GZ43_360557 |
| 1376 | Clu823296.3 | 2558.P20.GZ43_374691 |
| 1377 | Clu830453.2 | 2540.M22.GZ43_372317 |
| 1378 | Clu839006.1 | 2507.H02.GZ43_367111 |
| 1379 | Clu847088.1 | 2542.H23.GZ43_372966 |
| 1380 | Clu853371.2 | 2491.I06.GZ43_363794 |
| 1381 | Clu88462.1 | 2510.K15.GZ43_369280 |
| 1382 | Clu935908.2 | 2505.O09.GZ43_366518 |
| 1383 | Clu948383.1 | 2541.F05.GZ43_372516 |
| 1384 | Clu966599.3 | 2507.L12.GZ43_367217 |
| 1385 | Clu993554.1 | 2558.F19.GZ43_374450 |

TABLE 4

| SEQ ID | cDNA SEQ NAME | POLYNTD SEQ NAME | GENE | CHROM |
|---|---|---|---|---|
| 1386 | DTT00087024.1 | 2467.H18.GZ43_360651 | DTG00087008.1 | 1 |
| 1387 | DTT00089020.1 | 2367.I15.GZ43_346201 | DTG00089002.1 | 1 |
| 1388 | DTT00171014.1 | 2473.F14.GZ43_361367 | DTG00171001.1 | 1 |
| 1389 | DTT00514029.1 | 2488.G02.GZ43_362590 | DTG00514005.1 | 1 |
| 1390 | DTT00740010.1 | 2466.I08.GZ43_360281 | DTG00740003.1 | 1 |
| 1391 | DTT00945030.1 | 2466.D19.GZ43_360172 | DTG00945008.1 | 1 |
| 1392 | DTT01169022.1 | 2464.N05.GZ43_357997 | DTG01169003.1 | 2 |
| 1393 | DTT01178009.1 | 2510.O21.GZ43_369382 | DTG01178002.1 | 2 |
| 1394 | DTT01315010.1 | 2496.F14.GZ43_364217 | DTG01315001.1 | 2 |
| 1395 | DTT01503016.1 | 2538.M17.GZ43_371544 | DTG01503005.1 | 2 |
| 1396 | DTT01555018.1 | 2538.C07.GZ43_371294 | DTG01555002.1 | 2 |
| 1397 | DTT01685047.1 | 2496.C08.GZ43_364139 | DTG01685007.1 | 2 |
| 1398 | DTT01764019.1 | 2535.C23.GZ43_370158 | DTG01764003.1 | 2 |
| 1399 | DTT01890015.1 | 2482.J06.GZ43_359493 | DTG01890004.1 | 2 |
| 1400 | DTT02243008.1 | 2474.J19.GZ43_361852 | DTG02243002.1 | 3 |
| 1401 | DTT02367007.1 | 2366.P08.GZ43_345738 | DTG02367002.1 | 3 |
| 1402 | DTT02671007.1 | 2464.H22.GZ43_357870 | DTG02671002.1 | 3 |
| 1403 | DTT02737017.1 | 2538.M16.GZ43_371543 | DTG02737001.1 | 3 |
| 1404 | DTT02850005.1 | 2472.G03.GZ43_360996 | DTG02850001.1 | 3 |
| 1405 | DTT02966016.1 | 2510.M14.GZ43_369327 | DTG02966003.1 | 4 |
| 1406 | DTT03037029.1 | 2504.D16.GZ43_365877 | DTG03037005.1 | 4 |
| 1407 | DTT03150008.1 | 2491.P10.GZ43_363966 | DTG03150002.1 | 4 |
| 1408 | DTT03367008.1 | 2542.P19.GZ43_373154 | DTG03367003.1 | 4 |
| 1409 | DTT03630013.1 | 2510.O22.GZ43_369383 | DTG03630002.1 | 4 |
| 1410 | DTT03881017.1 | 2507.O12.GZ43_367289 | DTG03881007.1 | 5 |
| 1411 | DTT03913023.1 | 2459.P24.GZ43_357376 | DTG03913005.1 | 5 |
| 1412 | DTT03978010.1 | 2367.G22.GZ43_346160 | DTG03978001.1 | 5 |
| 1413 | DTT04070014.1 | 2540.H07.GZ43_372182 | DTG04070007.1 | 5 |
| 1414 | DTT04084010.1 | 2542.D19.GZ43_372866 | DTG04084001.1 | 5 |
| 1415 | DTT04160007.1 | 2472.M22.GZ43_361159 | DTG04160003.1 | 5 |
| 1416 | DTT04302021.1 | 2483.O07.GZ43_359998 | DTG04302002.1 | 5 |
| 1417 | DTT04378009.1 | 2368.O11.GZ43_346725 | DTG04378001.1 | 5 |
| 1418 | DTT04403013.1 | 2506.M05.GZ43_366850 | DTG04403003.1 | 5 |
| 1419 | DTT04414015.1 | 2368.D20.GZ43_346470 | DTG04414005.1 | 5 |
| 1420 | DTT04660017.1 | 2507.C03.GZ43_366992 | DTG04660003.1 | 6 |
| 1421 | DTT04956054.1 | 2538.I17.GZ43_371448 | DTG04956020.1 | 6 |
| 1422 | DTT04970018.1 | 2365.F24.GZ43_345370 | DTG04970007.1 | 6 |
| 1423 | DTT05205007.1 | 2459.J12.GZ43_357220 | DTG05205001.1 | 6 |
| 1424 | DTT05571010.1 | 2555.J10.GZ43_373385 | DTG05571004.1 | 7 |
| 1425 | DTT05650008.1 | 2557.L01.GZ43_374192 | DTG05650003.1 | 7 |
| 1426 | DTT05742029.1 | 2560.K10.GZ43_375329 | DTG05742002.1 | 7 |
| 1427 | DTT06137030.1 | 2565.B15.GZ43_398171 | DTG06137001.1 | 8 |
| 1428 | DTT06161014.1 | 2367.F06.GZ43_346120 | DTG06161007.1 | 8 |
| 1429 | DTT06706019.1 | 2467.D10.GZ43_360547 | DTG06706003.1 | 9 |
| 1430 | DTT06837021.1 | 2540.I10.GZ43_372209 | DTG06837002.1 | 9 |
| 1431 | DTT07040015.1 | 2504.E23.GZ43_365908 | DTG07040006.1 | 9 |
| 1432 | DTT07088009.1 | 2565.H01.GZ43_397953 | DTG07088001.1 | 9 |
| 1433 | DTT07182014.1 | 2536.G22.GZ43_370637 | DTG07182006.1 | 10 |
| 1434 | DTT07405044.1 | 2560.B11.GZ43_375114 | DTG07405010.1 | 10 |
| 1435 | DTT07408020.1 | 2466.M02.GZ43_360371 | DTG07408005.1 | 10 |
| 1436 | DTT07498014.1 | 2506.K20.GZ43_366817 | DTG07498002.1 | 10 |
| 1437 | DTT07600010.1 | 2464.H17.GZ43_357865 | DTG07600001.1 | 10 |
| 1438 | DTT08005024.1 | 2475.N21.GZ43_362334 | DTG08005009.1 | 11 |
| 1439 | DTT08098020.1 | 2540.M18.GZ43_372313 | DTG08098001.1 | 11 |
| 1440 | DTT08167018.1 | 2542.F05.GZ43_372900 | DTG08167002.1 | 11 |
| 1441 | DTT08249022.1 | 2498.G15.GZ43_365010 | DTG08249008.1 | 11 |
| 1442 | DTT08499022.1 | 2540.A24.GZ43_372031 | DTG08499009.1 | 12 |
| 1443 | DTT08514022.1 | 2541.L12.GZ43_372667 | DTG08514006.1 | 12 |
| 1444 | DTT08527013.1 | 2489.F09.GZ43_362957 | DTG08527005.1 | 12 |
| 1445 | DTT08595020.1 | 2554.N09.GZ43_376168 | DTG08595003.1 | 12 |
| 1446 | DTT08711019.1 | 2540.C19.GZ43_372074 | DTG08711001.1 | 12 |
| 1447 | DTT08773020.1 | 2559.I12.GZ43_374899 | DTG08773008.1 | 12 |
| 1448 | DTT08874012.1 | 2537.P14.GZ43_371229 | DTG08874001.1 | 12 |
| 1449 | DTT09387018.1 | 2561.P19.GZ43_376610 | DTG09387001.1 | 14 |
| 1450 | DTT09396022.1 | 2489.M11.GZ43_363127 | DTG09396001.1 | 14 |
| 1451 | DTT09553027.1 | 2505.J22.GZ43_366411 | DTG09553007.1 | 14 |
| 1452 | DTT09604016.1 | 2483.J07.GZ43_359878 | DTG09604006.1 | 14 |
| 1453 | DTT09705033.1 | 2536.O22.GZ43_370829 | DTG09705006.1 | 14 |
| 1454 | DTT09742009.1 | 2542.N21.GZ43_373108 | DTG09742002.1 | 15 |
| 1455 | DTT09753017.1 | 2464.L02.GZ43_357946 | DTG09753002.1 | 15 |
| 1456 | DTT09793019.1 | 2464.I04.GZ43_357876 | DTG09793004.1 | 15 |
| 1457 | DTT09796028.1 | 2366.L21.GZ43_345942 | DTG09796002.1 | 15 |
| 1458 | DTT10221016.1 | 2556.C19.GZ43_373610 | DTG10221004.1 | 16 |
| 1459 | DTT10360040.1 | 2475.M20.GZ43_362309 | DTG10360016.1 | 16 |
| 1460 | DTT10539016.1 | 2506.J20.GZ43_366793 | DTG10539005.1 | 17 |
| 1461 | DTT10564022.1 | 2475.H06.GZ43_362175 | DTG10564006.1 | 17 |
| 1462 | DTT10683041.1 | 2542.K21.GZ43_373036 | DTG10683007.1 | 17 |
| 1463 | DTT10819011.1 | 2474.I06.GZ43_361815 | DTG10819003.1 | 17 |
| 1464 | DTT11363027.1 | 2542.C20.GZ43_372843 | DTG11363008.1 | 19 |
| 1465 | DTT11479018.1 | 2506.G24.GZ43_366725 | DTG11479007.1 | 19 |
| 1466 | DTT11483012.1 | 2459.H09.GZ43_357169 | DTG11483001.1 | 19 |
| 1467 | DTT11548015.1 | 2565.C17.GZ43_398204 | DTG11548002.1 | 19 |
| 1468 | DTT11730017.1 | 2535.B09.GZ43_370120 | DTG11730004.1 | 20 |
| 1469 | DTT11791010.1 | 2506.E12.GZ43_366665 | DTG11791003.1 | 20 |
| 1470 | DTT11864036.1 | 2456.H07.GZ43_356003 | DTG11864011.1 | 21 |
| 1471 | DTT11902028.1 | 2490.B06.GZ43_363242 | DTG11902009.1 | 21 |
| 1472 | DTT11915017.1 | 2474.G17.GZ43_361778 | DTG11915002.1 | 21 |
| 1473 | DTT11966040.1 | 2457.L21.GZ43_356497 | DTG11966014.1 | 22 |
| 1474 | DTT12042027.1 | 2459.G01.GZ43_357137 | DTG12042005.1 | 22 |
| 1475 | DTT12201062.1 | 2562.B09.GZ43_375496 | DTG12201018.1 | X |
| 1476 | DTT12470020.1 | 2489.A13.GZ43_362841 | DTG12470004.1 | X |
| 1477 | DTT12550009.1 | 2504.G01.GZ43_365934 | DTG12550003.1 | X |

TABLE 5

| SEQ | PROTEIN SEQ | | | | DBL TWIST |
|---|---|---|---|---|---|
| ID | NAME | POLYNTD SEQ NAME | GENE | CHROM | LOCUS ID |
| 1478 | DTP00087033.1 | 2467.H18.GZ43_360651 | DTG00087008.1 | 1 | DTL00087012.1 |
| 1479 | DTP00089029.1 | 2367.I15.GZ43_346201 | DTG00089002.1 | 1 | DTL00089002.1 |
| 1480 | DTP00171023.1 | 2473.F14.GZ43_361367 | DTG00171001.1 | 1 | DTL00171013.1 |
| 1481 | DTP00514038.1 | 2488.G02.GZ43_362590 | DTG00514005.1 | 1 | DTL00514023.1 |
| 1482 | DTP00740019.1 | 2466.I08.GZ43_360281 | DTG00740003.1 | 1 | DTL00740006.1 |
| 1483 | DTP00945039.1 | 2466.D19.GZ43_360172 | DTG00945008.1 | 1 | |
| 1484 | DTP01169031.1 | 2464.N05.GZ43_357997 | DTG01169003.1 | 2 | DTL01169014.1 |
| 1485 | DTP01178018.1 | 2510.O21.GZ43_369382 | DTG01178002.1 | 2 | DTL01178007.1 |
| 1486 | DTP01315019.1 | 2496.F14.GZ43_364217 | DTG01315001.1 | 2 | DTL01315004.1 |
| 1487 | DTP01503025.1 | 2538.M17.GZ43_371544 | DTG01503005.1 | 2 | DTL01503007.1 |
| 1488 | DTP01555027.1 | 2538.C07.GZ43_371294 | DTG01555002.1 | 2 | DTL01555003.1 |
| 1489 | DTP01685056.1 | 2496.C08.GZ43_364139 | DTG01685007.1 | 2 | DTL01685004.1 |
| 1490 | DTP01764028.1 | 2535.C23.GZ43_370158 | DTG01764003.1 | 2 | DTL01764005.1 |
| 1491 | DTP01890024.1 | 2482.J06.GZ43_359493 | DTG01890004.1 | 2 | DTL01890001.1 |
| 1492 | DTP02243017.1 | 2474.J19.GZ43_361852 | DTG02243002.1 | 3 | DTL02243002.1 |
| 1493 | DTP02367016.1 | 2366.P08.GZ43_345738 | DTG02367002.1 | 3 | DTL02367004.1 |
| 1494 | DTP02671016.1 | 2464.H22.GZ43_357870 | DTG02671002.1 | 3 | DTL02671002.1 |
| 1495 | DTP02737026.1 | 2538.M16.GZ43_371543 | DTG02737001.1 | 3 | DTL02737012.1 |
| 1496 | DTP02850014.1 | 2472.G03.GZ43_360996 | DTG02850001.1 | 3 | DTL02850004.1 |
| 1497 | DTP02966025.1 | 2510.M14.GZ43_369327 | DTG02966003.1 | 4 | DTL02966001.1 |
| 1498 | DTP03037038.1 | 2504.D16.GZ43_365877 | DTG03037005.1 | 4 | DTL03037004.1 |
| 1499 | DTP03150017.1 | 2491.P10.GZ43_363966 | DTG03150002.1 | 4 | DTL03149001.1 |
| 1500 | DTP03367017.1 | 2542.P19.GZ43_373154 | DTG03367003.1 | 4 | DTL03367005.1 |
| 1501 | DTP03630022.1 | 2510.O22.GZ43_369383 | DTG03630002.1 | 4 | DTL03630006.1 |
| 1502 | DTP03881026.1 | 2507.O12.GZ43_367289 | DTG03881007.1 | 5 | DTL03881006.1 |
| 1503 | DTP03913032.1 | 2459.P24.GZ43_357376 | DTG03913005.1 | 5 | DTL03913012.1 |
| 1504 | DTP03978019.1 | 2367.G22.GZ43_346160 | DTG03978001.1 | 5 | DTL03978003.1 |
| 1505 | DTP04070023.1 | 2540.H07.GZ43_372182 | DTG04070007.1 | 5 | |
| 1506 | DTP04084019.1 | 2542.D19.GZ43_372866 | DTG04084001.1 | 5 | DTL04084001.1 |
| 1507 | DTP04160016.1 | 2472.M22.GZ43_361159 | DTG04160003.1 | 5 | DTL04160003.1 |
| 1508 | DTP04302030.1 | 2483.O07.GZ43_359998 | DTG04302002.1 | 5 | DTL04302006.1 |
| 1509 | DTP04378018.1 | 2368.O11.GZ43_346725 | DTG04378001.1 | 5 | |
| 1510 | DTP04403022.1 | 2506.M05.GZ43_366850 | DTG04403003.1 | 5 | DTL04403004.1 |
| 1511 | DTP04414024.1 | 2368.D20.GZ43_346470 | DTG04414005.1 | 5 | DTL04414004.1 |
| 1512 | DTP04660026.1 | 2507.C03.GZ43_366992 | DTG04660003.1 | 6 | DTL04660002.1 |
| 1513 | DTP04956063.1 | 2538.I17.GZ43_371448 | DTG04956020.1 | 6 | DTL04956028.1 |
| 1514 | DTP04970027.1 | 2365.F24.GZ43_345370 | DTG04970007.1 | 6 | DTL04970008.1 |
| 1515 | DTP05205016.1 | 2459.J12.GZ43_357220 | DTG05205001.1 | 6 | DTL05205002.1 |
| 1516 | DTP05571019.1 | 2555.J10.GZ43_373385 | DTG05571004.1 | 7 | DTL05571003.1 |
| 1517 | DTP05650017.1 | 2557.L01.GZ43_374192 | DTG05650003.1 | 7 | DTL05650004.1 |
| 1518 | DTP05742038.1 | 2560.K10.GZ43_375329 | DTG05742002.1 | 7 | DTL05742003.1 |
| 1519 | DTP06137039.1 | 2565.B15.GZ43_398171 | DTG06137001.1 | 8 | DTL06137003.1 |
| 1520 | DTP06161023.1 | 2367.F06.GZ43_346120 | DTG06161007.1 | 8 | DTL06161006.1 |
| 1521 | DTP06706028.1 | 2467.D10.GZ43_360547 | DTG06706003.1 | 9 | DTL06705001.1 |
| 1522 | DTP06837030.1 | 2540.I10.GZ43_372209 | DTG06837002.1 | 9 | DTL06837010.1 |
| 1523 | DTP07040024.1 | 2504.E23.GZ43_365908 | DTG07040006.1 | 9 | DTL07040004.1 |
| 1524 | DTP07088018.1 | 2565.H01.GZ43_397953 | DTG07088001.1 | 9 | DTL07088004.1 |
| 1525 | DTP07405053.1 | 2560.B11.GZ43_375114 | DTG07405010.1 | 10 | DTL07405034.1 |
| 1526 | DTP07408029.1 | 2466.M02.GZ43_360371 | DTG07408005.1 | 10 | DTL07408005.1 |
| 1527 | DTP07498023.1 | 2506.K20.GZ43_366817 | DTG07498002.1 | 10 | DTL07498007.1 |
| 1528 | DTP07600019.1 | 2464.H17.GZ43_357865 | DTG07600001.1 | 10 | DTL07600004.1 |
| 1529 | DTP08005033.1 | 2475.N21.GZ43_362334 | DTG08005009.1 | 11 | DTL08005010.1 |
| 1530 | DTP08098029.1 | 2540.M18.GZ43_372313 | DTG08098001.1 | 11 | DTL08098013.1 |
| 1531 | DTP08167027.1 | 2542.F05.GZ43_372900 | DTG08167002.1 | 11 | DTL08167003.1 |
| 1532 | DTP08249031.1 | 2498.G15.GZ43_365010 | DTG08249008.1 | 11 | DTL08249005.1 |
| 1533 | DTP08499031.1 | 2540.A24.GZ43_372031 | DTG08499009.1 | 12 | DTL08499012.1 |
| 1534 | DTP08514031.1 | 2541.L12.GZ43_372667 | DTG08514006.1 | 12 | DTL08514015.1 |
| 1535 | DTP08527022.1 | 2489.F09.GZ43_362957 | DTG08527005.1 | 12 | DTL08527008.1 |
| 1536 | DTP08595029.1 | 2554.N09.GZ43_376168 | DTG08595003.1 | 12 | DTL08595002.1 |
| 1537 | DTP08711028.1 | 2540.C19.GZ43_372074 | DTG08711001.1 | 12 | DTL08710003.1 |
| 1538 | DTP08773029.1 | 2559.I12.GZ43_374899 | DTG08773008.1 | 12 | DTL08773011.1 |
| 1539 | DTP08874021.1 | 2537.P14.GZ43_371229 | DTG08874001.1 | 12 | DTL08874009.1 |
| 1540 | DTP09387027.1 | 2561.P19.GZ43_376610 | DTG09387001.1 | 14 | DTL09387002.1 |
| 1541 | DTP09396031.1 | 2489.M11.GZ43_363127 | DTG09396001.1 | 14 | DTL09396016.1 |
| 1542 | DTP09553036.1 | 2505.J22.GZ43_366411 | DTG09553007.1 | 14 | DTL09553018.1 |
| 1543 | DTP09604025.1 | 2483.J07.GZ43_359878 | DTG09604006.1 | 14 | DTL09604010.1 |
| 1544 | DTP09705042.1 | 2536.O22.GZ43_370829 | DTG09705006.1 | 14 | DTL09705005.1 |
| 1545 | DTP09742018.1 | 2542.N21.GZ43_373108 | DTG09742002.1 | 15 | DTL09742007.1 |
| 1546 | DTP09753026.1 | 2464.L02.GZ43_357946 | DTG09753002.1 | 15 | DTL09753011.1 |
| 1547 | DTP09793028.1 | 2464.I04.GZ43_357876 | DTG09793004.1 | 15 | DTL09793004.1 |
| 1548 | DTP09796037.1 | 2366.L21.GZ43_345942 | DTG09796002.1 | 15 | DTL09796021.1 |
| 1549 | DTP10221025.1 | 2556.C19.GZ43_373610 | DTG10221004.1 | 16 | DTL10221002.1 |
| 1550 | DTP10360049.1 | 2475.M20.GZ43_362309 | DTG10360016.1 | 16 | DTL10360003.1 |
| 1551 | DTP10539025.1 | 2506.J20.GZ43_366793 | DTG10539005.1 | 17 | DTL10539004.1 |
| 1552 | DTP10564031.1 | 2475.H06.GZ43_362175 | DTG10564006.1 | 17 | DTL10564006.1 |
| 1553 | DTP10683050.1 | 2542.K21.GZ43_373036 | DTG10683007.1 | 17 | DTL10683002.1 |
| 1554 | DTP10819020.1 | 2474.I06.GZ43_361815 | DTG10819003.1 | 17 | DTL10819002.1 |
| 1555 | DTP11363036.1 | 2542.C20.GZ43_372843 | DTG11363008.1 | 19 | DTL11363017.1 |
| 1556 | DTP11479027.1 | 2506.G24.GZ43_366725 | DTG11479007.1 | 19 | DTL11479006.1 |
| 1557 | DTP11483021.1 | 2459.H09.GZ43_357169 | DTG11483001.1 | 19 | DTL11483006.1 |
| 1558 | DTP11548024.1 | 2565.C17.GZ43_398204 | DTG11548002.1 | 19 | DTL11548003.1 |
| 1559 | DTP11730026.1 | 2535.B09.GZ43_370120 | DTG11730004.1 | 20 | DTL11730009.1 |
| 1560 | DTP11791019.1 | 2506.E12.GZ43_366665 | DTG11791003.1 | 20 | DTL11791005.1 |
| 1561 | DTP11864045.1 | 2456.H07.GZ43_356003 | DTG11864011.1 | 21 | DTL11864023.1 |
| 1562 | DTP11902037.1 | 2490.B06.GZ43_363242 | DTG11902009.1 | 21 | DTL11902002.1 |
| 1563 | DTP11915026.1 | 2474.G17.GZ43_361778 | DTG11915002.1 | 21 | DTL11915001.1 |
| 1564 | DTP11966049.1 | 2457.L21.GZ43_356497 | DTG11966014.1 | 22 | DTL11966006.1 |
| 1565 | DTP12042036.1 | 2459.G01.GZ43_357137 | DTG12042005.1 | 22 | DTL12042001.1 |
| 1566 | DTP12201071.1 | 2562.B09.GZ43_375496 | DTG12201018.1 | X | DTL12201023.1 |
| 1567 | DTP12470029.1 | 2489.A13.GZ43_362841 | DTG12470004.1 | X | DTL12470016.1 |
| 1568 | DTP12550018.1 | 2504.G01.GZ43_365934 | DTG12550003.1 | X | DTL12550005.1 |

TABLE 6

| cDNA | cDNA SEQ | PROTEIN | PROTEIN SEQ | POLYNTD | |
|---|---|---|---|---|---|
| SEQ ID | NAME | SEQ ID | NAME | SEQ ID | POLYNTD SEQ NAME |
| 1386 | DTT00087024.1 | 1478 | DTP00087033.1 | 963 | 2467.H18.GZ43_360651 |
| 1386 | DTT00087024.1 | 1478 | DTP00087033.1 | 33 | 2505.B05.GZ43_366202 |
| 1387 | DTT00089020.1 | 1479 | DTP00089029.1 | 213 | 2367.I15.GZ43_346201 |
| 1388 | DTT00171014.1 | 1480 | DTP00171023.1 | 1006 | 2473.F14.GZ43_361367 |
| 1388 | DTT00171014.1 | 1480 | DTP00171023.1 | 1122 | 2489.A03.GZ43_362831 |
| 1389 | DTT00514029.1 | 1481 | DTP00514038.1 | 1113 | 2488.G02.GZ43_362590 |
| 1390 | DTT00740010.1 | 1482 | DTP00740019.1 | 952 | 2466.I08.GZ43_360281 |
| 1391 | DTT00945030.1 | 1483 | DTP00945039.1 | 945 | 2466.D19.GZ43_360172 |
| 1392 | DTT01169022.1 | 1484 | DTP01169031.1 | 482 | 2540.I17.GZ43_372216 |
| 1392 | DTT01169022.1 | 1484 | DTP01169031.1 | 914 | 2464.N05.GZ43_357997 |
| 1393 | DTT01178009.1 | 1485 | DTP01178018.1 | 113 | 2510.O21.GZ43_369382 |
| 1394 | DTT01315010.1 | 1486 | DTP01315019.1 | 1181 | 2496.F14.GZ43_364217 |
| 1395 | DTT01503016.1 | 1487 | DTP01503025.1 | 386 | 2538.M17.GZ43_371544 |
| 1396 | DTT01555018.1 | 1488 | DTP01555027.1 | 366 | 2538.C07.GZ43_371294 |
| 1396 | DTT01555018.1 | 1488 | DTP01555027.1 | 368 | 2538.D03.GZ43_371314 |
| 1396 | DTT01555018.1 | 1488 | DTP01555027.1 | 369 | 2538.D04.GZ43_371315 |
| 1397 | DTT01685047.1 | 1489 | DTP01685056.1 | 1177 | 2496.C08.GZ43_364139 |
| 1398 | DTT01764019.1 | 1490 | DTP01764028.1 | 267 | 2535.C23.GZ43_370158 |
| 1398 | DTT01764019.1 | 1490 | DTP01764028.1 | 771 | 2456.D04.GZ43_355904 |
| 1399 | DTT01890015.1 | 1491 | DTP01890024.1 | 1087 | 2482.J06.GZ43_359493 |
| 1399 | DTT01890015.1 | 1491 | DTP01890024.1 | 1042 | 2475.B20.GZ43_362045 |
| 1399 | DTT01890015.1 | 1491 | DTP01890024.1 | 1200 | 2497.L21.GZ43_364752 |
| 1400 | DTT02243008.1 | 1492 | DTP02243017.1 | 1224 | 2562.G21.GZ43_375628 |
| 1400 | DTT02243008.1 | 1492 | DTP02243017.1 | 1204 | 2497.P04.GZ43_364831 |
| 1400 | DTT02243008.1 | 1492 | DTP02243017.1 | 1025 | 2474.J19.GZ43_361852 |
| 1400 | DTT02243008.1 | 1492 | DTP02243017.1 | 1191 | 2497.D11.GZ43_364550 |
| 1401 | DTT02367007.1 | 1493 | DTP02367016.1 | 174 | 2366.P08.GZ43_345738 |
| 1402 | DTT02671007.1 | 1494 | DTP02671016.1 | 903 | 2464.H22.GZ43_357870 |
| 1402 | DTT02671007.1 | 1494 | DTP02671016.1 | 1055 | 2480.G11.GZ43_358658 |
| 1403 | DTT02737017.1 | 1495 | DTP02737026.1 | 385 | 2538.M16.GZ43_371543 |
| 1404 | DTT02850005.1 | 1496 | DTP02850014.1 | 992 | 2472.G03.GZ43_360996 |
| 1404 | DTT02850005.1 | 1496 | DTP02850014.1 | 1111 | 2488.F06.GZ43_362570 |
| 1404 | DTT02850005.1 | 1496 | DTP02850014.1 | 1039 | 2475.N08.GZ43_362321 |
| 1405 | DTT02966016.1 | 1497 | DTP02966025.1 | 103 | 2510.M14.GZ43_369327 |
| 1406 | DTT03037029.1 | 1498 | DTP03037038.1 | 9 | 2504.D16.GZ43_365877 |
| 1407 | DTT03150008.1 | 1499 | DTP03150017.1 | 428 | 2565.G20.GZ43_398256 |
| 1407 | DTT03150008.1 | 1499 | DTP03150017.1 | 585 | 2555.I12.GZ43_373363 |
| 1407 | DTT03150008.1 | 1499 | DTP03150017.1 | 235 | 2368.D08.GZ43_346458 |
| 1407 | DTT03150008.1 | 1499 | DTP03150017.1 | 1174 | 2491.P10.GZ43_363966 |
| 1408 | DTT03367008.1 | 1500 | DTP03367017.1 | 519 | 2506.E18.GZ43_366671 |
| 1408 | DTT03367008.1 | 1500 | DTP03367017.1 | 557 | 2542.P19.GZ43_373154 |
| 1409 | DTT03630013.1 | 1501 | DTP03630022.1 | 114 | 2510.O22.GZ43_369383 |
| 1410 | DTT03881017.1 | 1502 | DTP03881026.1 | 1251 | 2507.O12.GZ43_367289 |
| 1411 | DTT03913023.1 | 1503 | DTP03913032.1 | 889 | 2459.P24.GZ43_357376 |
| 1412 | DTT03978010.1 | 1504 | DTP03978019.1 | 211 | 2367.G22.GZ43_346160 |
| 1413 | DTT04070014.1 | 1505 | DTP04070023.1 | 423 | 2565.D06.GZ43_398029 |
| 1413 | DTT04070014.1 | 1505 | DTP04070023.1 | 374 | 2538.F03.GZ43_371362 |
| 1413 | DTT04070014.1 | 1505 | DTP04070023.1 | 17 | 2504.I13.GZ43_365994 |
| 1413 | DTT04070014.1 | 1505 | DTP04070023.1 | 692 | 2559.K12.GZ43_374947 |
| 1413 | DTT04070014.1 | 1505 | DTP04070023.1 | 43 | 2505.E15.GZ43_366284 |
| 1413 | DTT04070014.1 | 1505 | DTP04070023.1 | 750 | 2561.M09.GZ43_376528 |
| 1413 | DTT04070014.1 | 1505 | DTP04070023.1 | 463 | 2540.H07.GZ43_372182 |
| 1413 | DTT04070014.1 | 1505 | DTP04070023.1 | 1069 | 2481.D13.GZ43_358972 |
| 1414 | DTT04084010.1 | 1506 | DTP04084019.1 | 543 | 2542.D19.GZ43_372866 |
| 1415 | DTT04160007.1 | 1507 | DTP04160016.1 | 999 | 2472.M22.GZ43_361159 |
| 1416 | DTT04302021.1 | 1508 | DTP04302030.1 | 1106 | 2483.O07.GZ43_359998 |
| 1417 | DTT04378009.1 | 1509 | DTP04378018.1 | 260 | 2368.O11.GZ43_346725 |
| 1418 | DTT04403013.1 | 1510 | DTP04403022.1 | 531 | 2506.M05.GZ43_366850 |
| 1419 | DTT04414015.1 | 1511 | DTP04414024.1 | 236 | 2368.D20.GZ43_346470 |
| 1420 | DTT04660017.1 | 1512 | DTP04660026.1 | 334 | 2537.D11.GZ43_370938 |
| 1420 | DTT04660017.1 | 1512 | DTP04660026.1 | 1244 | 2507.C03.GZ43_366992 |
| 1421 | DTT04956054.1 | 1513 | DTP04956063.1 | 379 | 2538.I17.GZ43_371448 |
| 1422 | DTT04970018.1 | 1514 | DTP04970027.1 | 363 | 2538.B03.GZ43_371266 |
| 1422 | DTT04970018.1 | 1514 | DTP04970027.1 | 259 | 2368.O03.GZ43_346717 |
| 1422 | DTT04970018.1 | 1514 | DTP04970027.1 | 1101 | 2483.K02.GZ43_359897 |
| 1422 | DTT04970018.1 | 1514 | DTP04970027.1 | 134 | 2365.F24.GZ43_345370 |
| 1423 | DTT05205007.1 | 1515 | DTP05205016.1 | 880 | 2459.J12.GZ43_357220 |
| 1424 | DTT05571010.1 | 1516 | DTP05571019.1 | 586 | 2555.J10.GZ43_373385 |
| 1425 | DTT05650008.1 | 1517 | DTP05650017.1 | 644 | 2557.L01.GZ43_374192 |
| 1426 | DTT05742029.1 | 1518 | DTP05742038.1 | 721 | 2560.K10.GZ43_375329 |
| 1426 | DTT05742029.1 | 1518 | DTP05742038.1 | 126 | 2365.D10.GZ43_345308 |
| 1426 | DTT05742029.1 | 1518 | DTP05742038.1 | 756 | 2561.I19.GZ43_376442 |
| 1427 | DTT06137030.1 | 1519 | DTP06137039.1 | 419 | 2565.B15.GZ43_398171 |
| 1428 | DTT06161014.1 | 1520 | DTP06161023.1 | 205 | 2367.F06.GZ43_346120 |
| 1429 | DTT06706019.1 | 1521 | DTP06706028.1 | 967 | 2467.D10.GZ43_360547 |
| 1430 | DTT06837021.1 | 1522 | DTP06837030.1 | 465 | 2540.I10.GZ43_372209 |
| 1431 | DTT07040015.1 | 1523 | DTP07040024.1 | 10 | 2504.E23.GZ43_365908 |
| 1432 | DTT07088009.1 | 1524 | DTP07088018.1 | 170 | 2366.J06.GZ43_345700 |
| 1432 | DTT07088009.1 | 1524 | DTP07088018.1 | 429 | 2565.H01.GZ43_397953 |
| 1433 | DTT07182014.1 | | DTP07182023.1 | 306 | 2536.G22.GZ43_370637 |
| 1434 | DTT07405044.1 | 1525 | DTP07405053.1 | 703 | 2560.B11.GZ43_375114 |
| 1435 | DTT07408020.1 | 1526 | DTP07408029.1 | 956 | 2466.M02.GZ43_360371 |
| 1436 | DTT07498014.1 | 1527 | DTP07498023.1 | 529 | 2506.K20.GZ43_366817 |
| 1437 | DTT07600010.1 | 1528 | DTP07600019.1 | 902 | 2464.H17.GZ43_357865 |
| 1438 | DTT08005024.1 | 1529 | DTP08005033.1 | 1046 | 2475.N21.GZ43_362334 |
| 1439 | DTT08098020.1 | 1530 | DTP08098029.1 | 485 | 2540.M18.GZ43_372313 |
| 1440 | DTT08167018.1 | 1531 | DTP08167027.1 | 152 | 2365.N12.GZ43_345550 |
| 1440 | DTT08167018.1 | 1531 | DTP08167027.1 | 544 | 2542.F05.GZ43_372900 |
| 1441 | DTT08249022.1 | 1532 | DTP08249031.1 | 1235 | 2498.G15.GZ43_365010 |
| 1442 | DTT08499022.1 | 1533 | DTP08499031.1 | 452 | 2540.A24.GZ43_372031 |
| 1443 | DTT08514022.1 | 1534 | DTP08514031.1 | 508 | 2541.L12.GZ43_372667 |
| 1444 | DTT08527013.1 | 1535 | DTP08527022.1 | 109 | 2510.N14.GZ43_369351 |
| 1444 | DTT08527013.1 | 1535 | DTP08527022.1 | 394 | 2554.A16.GZ43_375863 |
| 1444 | DTT08527013.1 | 1535 | DTP08527022.1 | 1128 | 2489.F09.GZ43_362957 |
| 1444 | DTT08527013.1 | 1535 | DTP08527022.1 | 569 | 2555.F16.GZ43_373295 |
| 1445 | DTT08595020.1 | 1536 | DTP08595029.1 | 413 | 2554.N09.GZ43_376168 |
| 1446 | DTT08711019.1 | 1537 | DTP08711028.1 | 472 | 2540.C19.GZ43_372074 |
| 1447 | DTT08773020.1 | 1538 | DTP08773029.1 | 687 | 2559.I12.GZ43_374899 |
| 1448 | DTT08874012.1 | 1539 | DTP08874021.1 | 356 | 2537.P14.GZ43_371229 |
| 1449 | DTT09387018.1 | 1540 | DTP09387027.1 | 762 | 2561.P19.GZ43_376610 |
| 1450 | DTT09396022.1 | 1541 | DTP09396031.1 | 1140 | 2489.M11.GZ43_363127 |
| 1451 | DTT09553027.1 | 1542 | DTP09553036.1 | 54 | 2505.J22.GZ43_366411 |
| 1452 | DTT09604016.1 | 1543 | DTP09604025.1 | 1100 | 2483.J07.GZ43_359878 |
| 1453 | DTT09705033.1 | 1544 | DTP09705042.1 | 323 | 2536.O22.GZ43_370829 |
| 1454 | DTT09742009.1 | 1545 | DTP09742018.1 | 766 | 2456.B12.GZ43_355864 |
| 1454 | DTT09742009.1 | 1545 | DTP09742018.1 | 563 | 2542.N21.GZ43_373108 |
| 1455 | DTT09753017.1 | 1546 | DTP09753026.1 | 910 | 2464.L02.GZ43_357946 |
| 1456 | DTT09793019.1 | 1547 | DTP09793028.1 | 904 | 2464.I04.GZ43_357876 |
| 1457 | DTT09796028.1 | 1548 | DTP09796037.1 | 189 | 2366.L21.GZ43_345942 |
| 1458 | DTT10221016.1 | 1549 | DTP10221025.1 | 592 | 2556.C19.GZ43_373610 |
| 1459 | DTT10360040.1 | 1550 | DTP10360049.1 | 1045 | 2475.M20.GZ43_362309 |
| 1460 | DTT10539016.1 | 1551 | DTP10539025.1 | 527 | 2506.J20.GZ43_366793 |
| 1461 | DTT10564022.1 | 1552 | DTP10564031.1 | 1035 | 2475.H06.GZ43_362175 |
| 1462 | DTT10683041.1 | 1553 | DTP10683050.1 | 561 | 2542.K21.GZ43_373036 |
| 1463 | DTT10819011.1 | 1554 | DTP10819020.1 | 796 | 2457.C19.GZ43_356279 |
| 1463 | DTT10819011.1 | 1554 | DTP10819020.1 | 143 | 2365.J14.GZ43_345456 |
| 1463 | DTT10819011.1 | 1554 | DTP10819020.1 | 1023 | 2474.I06.GZ43_361815 |
| 1464 | DTT11363027.1 | 1555 | DTP11363036.1 | 540 | 2542.C20.GZ43_372843 |
| 1465 | DTT11479018.1 | 1556 | DTP11479027.1 | 521 | 2506.G24.GZ43_366725 |
| 1466 | DTT11483012.1 | 1557 | DTP11483021.1 | 877 | 2459.H09.GZ43_357169 |
| 1467 | DTT11548015.1 | 1558 | DTP11548024.1 | 422 | 2565.C17.GZ43_398204 |
| 1468 | DTT11730017.1 | 1559 | DTP11730026.1 | 264 | 2535.B09.GZ43_370120 |
| 1469 | DTT11791010.1 | 1560 | DTP11791019.1 | 518 | 2506.E12.GZ43_366665 |
| 1470 | DTT11864036.1 | 1561 | DTP11864045.1 | 778 | 2456.H07.GZ43_356003 |
| 1471 | DTT11902028.1 | 1562 | DTP11902037.1 | 1144 | 2490.B06.GZ43_363242 |
| 1472 | DTT11915017.1 | 1563 | DTP11915026.1 | 591 | 2556.C11.GZ43_373602 |
| 1472 | DTT11915017.1 | 1563 | DTP11915026.1 | 1021 | 2474.G17.GZ43_361778 |
| 1472 | DTT11915017.1 | 1563 | DTP11915026.1 | 1163 | 2491.C13.GZ43_363657 |
| 1473 | DTT11966040.1 | 1564 | DTP11966049.1 | 1216 | 2562.E14.GZ43_375573 |
| 1473 | DTT11966040.1 | 1564 | DTP11966049.1 | 818 | 2457.L21.GZ43_356497 |
| 1473 | DTT11966040.1 | 1564 | DTP11966049.1 | 532 | 2506.M13.GZ43_366858 |
| 1474 | DTT12042027.1 | 1565 | DTP12042036.1 | 874 | 2459.G01.GZ43_357137 |
| 1475 | DTT12201062.1 | 1566 | DTP12201071.1 | 759 | 2561.O17.GZ43_376584 |
| 1475 | DTT12201062.1 | 1566 | DTP12201071.1 | 1207 | 2562.B09.GZ43_375496 |
| 1476 | DTT12470020.1 | 1567 | DTP12470029.1 | 1124 | 2489.A13.GZ43_362841 |
| 1476 | DTT12470020.1 | 1567 | DTP12470029.1 | 799 | 2457.D12.GZ43_356296 |
| 1476 | DTT12470020.1 | 1567 | DTP12470029.1 | 690 | 2559.J02.GZ43_374913 |
| 1476 | DTT12470020.1 | 1567 | DTP12470029.1 | 568 | 2555.E20.GZ43_373275 |
| 1477 | DTT12550009.1 | 1568 | DTP12550018.1 | 12 | 2504.G01.GZ43_365934 |

TABLE 7

| | | | | GENBANK |
|---|---|---|---|---|
| SEQ ID | SEQ NAME | ACCESSION | GENBANK DESCRIPTION | SCORE |
| 6 | 2504.C08.GZ43_365845 | AP000321 | gi\|4835690\|dbj\|AP000321.1AP000321 | 1.6E−31 |
| | | | Homo sapiens genomic DNA, chromosome 21q22.1, | |
| | | | D21S226-AML region, | |
| | | | clone: Q82F5, complete sequence | |
| 7 | 2504.C11.GZ43_365848 | AP002938 | gi\|16267134\|dbj\|AP002938.1AP002938 | 4.8E−58 |
| | | | Hoplostethus japonicus mitochondrial DNA, | |
| | | | complete genome | |
| 9 | 2504.D16.GZ43_365877 | AK023496 | gi\|10435445\|dbj\|AK023496.1AK023496 | 0 |
| | | | Homo sapiens cDNA FLJ13434 fis, clone | |
| | | | PLACE1002578 | |
| 10 | 2504.E23.GZ43_365908 | M80340 | gi\|339767\|gb\|M80340.1HUMTNL12 Human | 6.1E−182 |
| | | | transposon L1.1 with a base deletion relative | |
| | | | to L1.2B resulting in a premature stop codon | |
| | | | in t | |
| 11 | 2504.F20.GZ43_365929 | AE007289 | gi\|14524175\|gb\|AE007289.1AE007289 | 2.1E−98 |
| | | | Sinorhizobium meliloti plasmid pSymA | |
| | | | section 95 of 121 of the complete plasmid | |
| | | | sequence | |
| 17 | 2504.I13.GZ43_365994 | AJ312523 | gi\|12830519\|emb\|AJ312523.1GGO312523 | 1.1E−44 |
| | | | Gorilla gorilla gorilla Xq13.3 chromosome | |
| | | | non-coding sequence, isolate G167W | |
| 31 | 2504.O12.GZ43_366137 | AF342020 | gi\|12961941\|gb\|AF342020.1AF342020 | 1.1E−90 |
| | | | Sclerotinia sclerotiorum strain LES-1 28S | |
| | | | ribosomal RNA gene, partial sequence; | |
| | | | intergenic spacer | |
| 33 | 2505.B05.GZ43_366202 | U93571 | gi\|2072968\|gb\|U93571.1HSU93571 Human | 1.1E−226 |
| | | | L1 element L1.24 p40 gene, complete cds | |
| 37 | 2505.C17.GZ43_366238 | AJ325713 | gi\|15870107\|emb\|AJ325713.1HSA325713 | 1.4E−21 |
| | | | Homo sapiens genomic sequence | |
| | | | surrounding NotI site, clone NB1-110S | |
| 40 | 2505.D03.GZ43_366248 | AJ224335 | gi\|3413799\|emb\|AJ224335.1HSAJ4335 | 5.2E−71 |
| | | | Homo sapien mRNA for putative secretory | |
| | | | protein, hBET3 | |
| 43 | 2505.E15.GZ43_366284 | AB030001 | gi\|7416074\|dbj\|AB030001.1AB030001 | 8.1E−55 |
| | | | Homo sapiens gene for SGRF, complete cds | |
| 46 | 2505.G16.GZ43_366333 | AE005683 | gi\|13421186\|gb\|AE005683.1AE005683 | 3.6E−63 |
| | | | Caulobacter crescentus section 9 of 359 of | |
| | | | the complete genome | |
| 48 | 2505.I04.GZ43_366369 | AF255613 | gi\|8925326\|gb\|AF255613.1AF255613 Homo | 7.9E−73 |
| | | | sapiens teratoma-associated tyrosine kinase | |
| | | | (TAPK) gene, exons 1 through 6 and partial | |
| | | | cds | |
| 63 | 2505.O09.GZ43_366518 | AF053644 | gi\|3598786\|gb\|AF053644.1HSCSE1G2 | 9.4E−45 |
| | | | Homo sapiens cellular apoptosis | |
| | | | susceptibility protein (CSE1) gene, exon 2 | |
| 72 | 2510.C10.GZ43_369083 | AB002353 | gi\|2224650\|dbj\|AB002353.1AB002353 | 1.4E−71 |
| | | | Human mRNA for KIAA0355 gene, | |
| | | | complete cds | |
| 78 | 2510.G06.GZ43_369175 | AF084935 | gi\|3603422\|gb\|AF084935.1AF084935 Homo | 8.9E−24 |
| | | | sapiens galactokinase (GALK1) gene, | |
| | | | partial cds | |
| 89 | 2510.J11.GZ43_369252 | AK024617 | gi\|10436933\|dbj\|AK024617.1AK024617 | 0 |
| | | | Homo sapiens cDNA: FLJ20964 fis, clone | |
| | | | ADSH00902 | |
| 102 | 2510.L21.GZ43_369310 | AK023677 | gi\|10435673\|dbj\|AK023677.1AK023677 | 1.2E−90 |
| | | | Homo sapiens cDNA FLJ13615 fis, clone | |
| | | | PLACE1010896, weakly similar to NUF1 | |
| | | | PROTEIN | |
| 109 | 2510.N14.GZ43_369351 | AF271388 | gi\|8515842\|gb\|AF271388.1AF271388 Homo | 0 |
| | | | sapiens CMP-N-acetylneuraminic acid | |
| | | | synthase mRNA, complete cds | |
| 115 | 2510.O23.GZ43_369384 | AF113169 | gi\|4164598\|gb\|AF113169.1AF113169 Homo | 2.2E−39 |
| | | | sapiens glandular kallikrein enhancer region, | |
| | | | complete sequence | |
| 124 | 2365.C20.GZ43_345294 | AF069489 | gi\|3560568\|gb\|AF069489.1HSPDE4A3 | 6.6E−24 |
| | | | Homo sapiens cAMP specific | |
| | | | phosphodiesterase 4A variant pde46 | |
| | | | (PDE4A) gene, exons 2 through 13 and | |
| 134 | 2365.F24.GZ43_345370 | AK012908 | gi\|12849956\|dbj\|AK012908.1AK012908 | 2.9E−224 |
| | | | Mus musculus 10, 11 days embryo cDNA, | |
| | | | RIKEN full-length enriched library, | |
| | | | clone: 2810046L04, full | |
| 143 | 2365.J14.GZ43_345456 | BC007999 | gi\|14124949\|gb\|BC007999.1BC007999 | 4.4E−56 |
| | | | Homo sapiens, hypothetical protein | |
| | | | FLJ10759, clone MGC: 15757 | |
| | | | IMAGE: 3357436, mRNA, complete cds | |
| 152 | 2365.N12.GZ43_345550 | U20391 | gi\|1483626\|gb\|U20391.1HSU20391 Human | 3.9E−41 |
| | | | folate receptor (FOLR1) gene, complete cds | |
| 162 | 2366.E03.GZ43_345647 | AB025285 | gi\|5917586\|dbj\|AB025285.1AB025285 | 4.3E−30 |
| | | | Homo sapiens c-ERBB-2 gene, exons 1′, 2′, | |
| | | | 3′, 4′ | |
| 163 | 2366.J03.GZ43_345652 | M15885 | gi\|338414\|gb\|M15885.1HUMSPP Human | 1.1E−68 |
| | | | prostate secreted seminal plasma protein | |
| | | | mRNA, complete cds | |
| 170 | 2366.J06.GZ43_345700 | AF326517 | gi\|15080738\|gb\|AF326517.1AF326517 | 0 |
| | | | Abies grandis pinene synthase gene, partial | |
| | | | cds | |
| 182 | 2366.K13.GZ43_345813 | U27333 | gi\|967202\|gb\|U27333.1HSU27333 Human | 2.5E−44 |
| | | | alpha (1,3) fucosyltransferase (FUT6) | |
| | | | mRNA, major transcript I, complete cds | |
| 189 | 2366.L21.GZ43_345942 | AF272390 | gi\|8705239\|gb\|AF272390.1AF272390 Homo | 1.4E−290 |
| | | | sapiens myosin 5c (MYO5C) mRNA, | |
| | | | complete cds | |
| 195 | 2367.B10.GZ43_346028 | AJ279823 | gi\|11932035\|emb\|AJ279823.1ASF279823 | 1.4E−231 |
| | | | Ascovirus SfAV1b partial pol gene for DNA | |
| | | | polymerase, Pol2-Pol3-Pol1 fragment | |
| 198 | 2367.C12.GZ43_346054 | BC014669 | gi\|15779227\|gb\|BC014669.1BC014669 | 2.9E−57 |
| | | | Homo sapiens, clone IMAGE: 4849317, | |
| | | | mRNA, partial cds | |
| 200 | 2367.D18.GZ43_346084 | AE008517 | gi\|15459138\|gb\|AE008517.1AE008517 | 1.4E−34 |
| | | | Streptococcus pneumoniae R6 section 133 | |
| | | | of 184 of the complete genome | |
| 205 | 2367.F06.GZ43_346120 | AJ330464 | gi\|15874882\|emb\|AJ330464.1HSA330464 | 3.1E−100 |
| | | | Homo sapiens genomic sequence | |
| | | | surrounding NotI site, clone NR1-IL7C | |
| 206 | 2367.F13.GZ43_346127 | AY035075 | gi\|14334803\|gb\|AY035075.1 Arabidopsis | 4.1E−229 |
| | | | thaliana putative H+-transporting ATPase | |
| | | | (AT4g30190) mRNA, complete cds | |
| 208 | 2367.G13.GZ43_346151 | AK025355 | gi\|10437854\|dbj\|AK025355.1AK025355 | 1.8E−58 |
| | | | Homo sapiens cDNA: FLJ21702 fis, clone | |
| | | | COL09874 | |
| 209 | 2367.G17.GZ43_346155 | AK000293 | gi\|7020278\|dbj\|AK000293.1AK000293 | 4.4E−34 |
| | | | Homo sapiens cDNA FLJ20286 fis, clone | |
| | | | HEP04358 | |
| 210 | 2367.G20.GZ43_346158 | AL137592 | gi\|6808332\|emb\|AL137592.1HSM802347 | 1.6E−60 |
| | | | Homo sapiens mRNA; cDNA | |
| | | | DKFZp434L0610 (from clone | |
| | | | DKFZp434L0610); partial cds | |
| 211 | 2367.G22.GZ43_346160 | BC015529 | gi\|15930193\|gb\|BC015529.1BC015529 | 9.7E−60 |
| | | | Homo sapiens, Similar to ribose 5-phosphate | |
| | | | isomerase A, clone MGC: 9441 | |
| | | | IMAGE: 3904718, mRNA, comp | |
| 213 | 2367.I15.GZ43_346201 | AF324172 | gi\|12958747\|gb\|AF324172.1AF324172 | 4.8E−65 |
| | | | Dictyophora indusiata strain ASI 32001 | |
| | | | internal transcribed spacer 1, partial | |
| | | | sequence; 5.8S ribo | |
| 217 | 2367.K24.GZ43_346258 | AF009251 | gi\|2352833\|gb\|AF009251.1CLCN6HUM05 | 3.8E−62 |
| | | | Homo sapiens putative chloride channel | |
| | | | gene (CLCN6), exon 6 | |
| 219 | 2367.M06.GZ43_346288 | AF178322 | gi\|13344845\|gb\|AF178322.1AF178322 | 1.5E−43 |
| | | | Schmidtea mediterranea cytochrome oxidase | |
| | | | C subunit I (COI) gene, partial cds; | |
| | | | mitochondrial gene | |
| 220 | 2367.M14.GZ43_346296 | AK026286 | gi\|10439097\|dbj\|AK026286.1AK026286 | 1E−300 |
| | | | Homo sapiens cDNA: FLJ22633 fis, clone | |
| | | | HSI06502 | |
| 221 | 2367.M16.GZ43_346298 | AF368920 | gi\|14039926\|gb\|AF368920.1AF368920 | 1.6E−83 |
| | | | Caenorhabditis elegans voltage-dependent | |
| | | | calcium channel alpha 13 subunit (cca-1) | |
| | | | mRNA, complete c | |
| 224 | 2367.N16.GZ43_346322 | Z78727 | gi\|1508005\|emb\|Z78727.1HSPA15B9 | 1.3E−37 |
| | | | H. sapiens flow-sorted chromosome 6 | |
| | | | HindIII fragment, SC6pA15B9 | |
| 231 | 2368.B18.GZ43_346420 | AK000293 | gi\|7020278\|dbj\|AK000293.1AK000293 | 5E−34 |
| | | | Homo sapiens cDNA FLJ20286 fis, clone | |
| | | | HEP04358 | |
| 235 | 2368.D08.GZ43_346458 | AJ276936 | gi\|12214232\|emb\|AJ276936.1NME276936 | 0 |
| | | | Neisseria meningitidis partial tbpB gene for | |
| | | | transferrin binding protein B subunit, allele | |
| | | | 66, | |
| 245 | 2368.I04.GZ43_346574 | AY042191 | gi\|15546022\|gb\|AY042191.1 Mus musculus | 3.1E−26 |
| | | | RF-amide G protein-coupled receptor | |
| | | | (MrgA1) mRNA, complete cds | |
| 249 | 2368.K21.GZ43_346639 | AJ310931 | gi\|15718363\|emb\|AJ310931.1HSA310931 | 7E−55 |
| | | | Homo sapiens mRNA for myosin heavy | |
| | | | chain | |
| 252 | 2368.M19.GZ43_346685 | AK025595 | gi\|10438161\|dbj\|AK025595.1AK025595 | 4.7E−21 |
| | | | Homo sapiens cDNA: FLJ21942 fis, clone | |
| | | | HEP04527 | |
| 257 | 2368.N15.GZ43_346705 | AK014328 | gi\|12852104\|dbj\|AK014328.1AK014328 | 3.1E−103 |
| | | | Mus musculus 14, 17 days embryo head | |
| | | | cDNA, RIKEN full-length enriched library, | |
| | | | clone: 3230401M21, | |
| 258 | 2368.N23.GZ43_346713 | AL391428 | gi\|9864373\|emb\|AL391428.1AL391428 | 4.8E−28 |
| | | | Human DNA sequence from clone RP11- | |
| | | | 60P19 on chromosome 1, complete | |
| | | | sequence [Homo sapiens] | |
| 259 | 2368.O03.GZ43_346717 | AK012908 | gi\|12849956\|dbj\|AK012908.1AK012908 | 2.1E−227 |
| | | | Mus musculus 10, 11 days embryo cDNA, | |
| | | | RIKEN full-length enriched library, | |
| | | | clone: 2810046L04, full | |
| 260 | 2368.O11: GZ43_346725 | AF102129 | gi\|5922722\|gb\|AF102129.1AF102129 Rattus | 2.5E−103 |
| | | | norvegicus KPL2 (Kpl2) mRNA, complete | |
| | | | cds | |
| 264 | 2535.B09.GZ43_370120 | AF292648 | gi\|12656358\|gb\|AF292648.1AF292648 Mus | 2E−39 |
| | | | musculus zinc finger 202 m1 (Znf202) | |
| | | | mRNA, complete cds | |
| 267 | 2535.C23.GZ43_370158 | AF307053 | gi\|12018057\|gb\|AF307053.1AF307053 | 0 |
| | | | Thermococcus litoralis sugar kinase, | |
| | | | trehalose/maltose binding protein (malE), | |
| | | | trehalose/maltose | |
| 269 | 2535.F05.GZ43_370212 | AF367433 | gi\|14486704\|gb\|AF367433.1AF367433 | 3.8E-38 |
| | | | Lotus japonicus phosphatidylinositol | |
| | | | transfer-like protein III (LjPLP-III) mRNA, | |
| | | | complete cds | |
| 276 | 2535.L03.GZ43_370354 | AK000099 | gi\|7019966\|dbj\|AK000099.1AK000099 | 7.1E−52 |
| | | | Homo sapiens cDNA FLJ20092 fis, clone | |
| | | | COL04215 | |
| 280 | 2535.O07.GZ43_370430 | BC008425 | gi\|14250051\|gb\|BC008425.1BC008425 | 3.8E−34 |
| | | | Homo sapiens, clone MGC: 14582 | |
| | | | IMAGE: 4246114, mRNA, complete cds | |
| 282 | 2535.P02.GZ43_370449 | NM_024074 | gi\|13129059\|ref\|NM_024074.1 Homo | 2.4E−23 |
| | | | sapiens hypothetical protein MGC3169 | |
| | | | (MGC3169), mRNA | |
| 292 | 2536.A22.GZ43_370493 | AF310311 | gi\|13517433\|gb\|AF310311.1AF310311 | 0 |
| | | | Homo sapiens isolate Nigeria 9 membrane | |
| | | | protein CH1 gene, partial cds | |
| 297 | 2536.D17.GZ43_370560 | AF015148 | gi\|2353128\|gb\|AF015148.1AF015148 Homo | 1.6E−46 |
| | | | sapiens clone HS19.2 Alu-Ya5 sequence | |
| 303 | 2536.G05.GZ43_370620 | AF045605 | gi\|3228525\|gb\|AF045605.1AF045605 Homo | 6.2E−77 |
| | | | sapiens germline chromosome 11, 11q13 | |
| | | | region | |
| 305 | 2536.G21.GZ43_370636 | AK026490 | gi\|10439363\|dbj\|AK026490.1AK026490 | 3.5E−143 |
| | | | Homo sapiens cDNA: FLJ22837 fis, clone | |
| | | | KAIA4417 | |
| 306 | 2536.G22.GZ43_370637 | NC_002707 | gi\|13540758\|ref\|NC_002707.1 Anguilla | 2.3E−39 |
| | | | japonica mitochondrion, complete genome | |
| 309 | 2536.I05.GZ43_370668 | AK000099 | gi\|7019966\|dbj\|AK000099.1AK000099 | 3.4E−63 |
| | | | Homo sapiens cDNA FLJ20092 fis, clone | |
| | | | COL04215 | |
| 310 | 2536.I15.GZ43_370678 | AB013897 | gi\|6177784\|dbj\|AB013897.1AB013897 | 5.1E−53 |
| | | | Homo sapiens mRNA for HKR1, partial cds | |
| 313 | 2536.J11.GZ43_370698 | AK023448 | gi\|10435386\|dbj\|AK023448.1AK023448 | 0 |
| | | | Homo sapiens cDNA FLJ13386 fis, clone | |
| | | | PLACE1001104, weakly similar to | |
| | | | MYOSIN HEAVY CHAIN, NON-MU | |
| 314 | 2536.K12.GZ43_370723 | U14573 | gi\|551542\|gb\|U14573.1HSU14573 ***ALU | 1E−96 |
| | | | WARNING: Human Alu-Sq subfamily | |
| | | | consensus sequence | |
| 319 | 2536.N05.GZ43_370788 | AK001347 | gi\|7022548\|dbj\|AK001347.1AK001347 | 6.7E−43 |
| | | | Homo sapiens cDNA FLJ10485 fis, clone | |
| | | | NT2RP2000195 | |
| 320 | 2536.N20.GZ43_370803 | Y15724 | gi\|3021395\|emb\|Y15724.1HSSERCA1 | 1.9E−27 |
| | | | Homo sapiens SERCA3 gene, exons 1-7 | |
| | | | (and joined CDS) | |
| 330 | 2537.B07.GZ43_370886 | X69516 | gi\|288876\|emb\|X69516.1HSFOLA | 2.8E−60 |
| | | | H. sapiens gene for folate receptor | |
| 334 | 2537.D11.GZ43_370938 | NM_025080 | gi\|13376633\|ref\|NM_025080.1 Homo | 8.7E−289 |
| | | | sapiens hypothetical protein FLJ22316 | |
| | | | (FLJ22316), mRNA | |
| 338 | 2537.G05.GZ43_371004 | L04193 | gi\|187144\|gb\|L04193.1HUMLIMGP Human | 7.4E−52 |
| | | | lens membrane protein (mp19) gene, exon | |
| | | | 11 | |
| 341 | 2537.I03.GZ43_371050 | Z78727 | gi\|1508005\|emb\|Z78727.1HSPA15B9 | 1.7E−37 |
| | | | H. sapiens flow-sorted chromosome 6 | |
| | | | HindIII fragment, SC6pA15B9 | |
| 345 | 2537.K17.GZ43_371112 | AL603947 | gi\|15384818\|emb\|AL603947.1UMA0006 | 9.3E−76 |
| | | | Ustilago maydis gene for predicted | |
| | | | plasmamembrane-ATPase | |
| 350 | 2537.N23.GZ43_371190 | AF242865 | gi\|9858570\|gb\|AF242865.1AF242862S4 | 2.4E−30 |
| | | | Homo sapiens coxsackie virus and | |
| | | | adenovirus receptor (CXADR) gene, exon 7 | |
| | | | and complete cds | |
| 352 | 2537.O05.GZ43_371196 | AB060827 | gi\|13874462\|dbj\|AB060827.1AB060827 | 2.2E−24 |
| | | | Macaca fascicularis brain cDNA clone: QtrA- | |
| | | | 10256, full insert sequence | |
| 356 | 2537.P14.GZ43_371229 | AK026442 | gi\|10439307\|dbj\|AK026442.1AK026442 | 6.3E−256 |
| | | | Homo sapiens cDNA: FLJ22789 fis, clone | |
| | | | KAIA2171 | |
| 361 | 2538.A10.GZ43_371249 | AK001432 | gi\|7022685\|dbj\|AK001432.1AK001432 | 1.9E−52 |
| | | | Homo sapiens cDNA FLJ10570 fis, clone | |
| | | | NT2RP2003117 | |
| 363 | 2538.B03.GZ43_371266 | AK013900 | gi\|12851449\|dbj\|AK013900.1AK013900 | 1.2E−201 |
| | | | Mus musculus 12 days embryo head cDNA, | |
| | | | RIKEN full-length enriched library, | |
| | | | clone: 3010026L22, ful | |
| 366 | 2538.C07.GZ43_371294 | AK022973 | gi\|10434673\|dbj\|AK022973.1AK022973 | 0 |
| | | | Homo sapiens cDNA FLJ12911 fis, clone | |
| | | | NT2RP2004425, highly similar to Mus | |
| | | | musculus axotrophin mR | |
| 367 | 2538.C14.GZ43_371301 | M87914 | gi\|174891\|gb\|M87914.1HUMALNE461 | 2E−89 |
| | | | Human carcinoma cell-derived Alu RNA | |
| | | | transcript, clone NE461 | |
| 368 | 2538.D03.GZ43_371314 | AK022973 | gi\|10434673\|dbj\|AK022973.1AK022973 | 4.3E−275 |
| | | | Homo sapiens cDNA FLJ12911 fis, clone | |
| | | | NT2RP2004425, highly similar to Mus | |
| | | | musculus axotrophin mR | |
| 369 | 2538.D04.GZ43_371315 | AK022973 | gi\|10434673\|dbj\|AK022973.1AK022973 | 1.3E−287 |
| | | | Homo sapiens cDNA FLJ12911 fis, clone | |
| | | | NT2RP2004425, highly similar to Mus | |
| | | | musculus axotrophin mR | |
| 371 | 2538.E01.GZ43_371336 | AF074397 | gi\|3916231\|gb\|AF074397.1AF074397 Homo | 4E−40 |
| | | | sapiens anti-mullerian hormone type II | |
| | | | receptor (AMHR2) gene, promoter region | |
| | | | and partial cds | |
| 374 | 2538.F03.GZ43_371362 | L34639 | gi\|598203\|gb\|L34639.1HUMPECAM09 | 1.5E−43 |
| | | | Homo sapiens platelet/endothelial cell | |
| | | | adhesion molecule-1 (PECAM-1) gene, | |
| | | | exon 6 | |
| 375 | 2538.H02.GZ43_371409 | AF220173 | gi\|9651700\|gb\|AF220173.1AF220172S2 | 2.5E−39 |
| | | | Homo sapiens acid ceramidase (ASAH) | |
| | | | gene, exons 2 through 4 | |
| 379 | 2538.I17.GZ43_371448 | AF050179 | gi\|3319283\|gb\|AF050179.1AF050179 Homo | 4.9E−41 |
| | | | sapiens CENP-C binding protein (DAXX) | |
| | | | mRNA, complete cds | |
| 380 | 2538.J10.GZ43_371465 | AY035075 | gi\|14334803\|gb\|AY035075.1 Arabidopsis | 3.5E−245 |
| | | | thaliana putative H+-transporting ATPase | |
| | | | (AT4g30190) mRNA, complete cds | |
| 381 | 2538.K17.GZ43_371496 | AK022749 | gi\|10434332\|dbj\|AK022749.1AK022749 | 1.5E−31 |
| | | | Homo sapiens cDNA FLJ12687 fis, clone | |
| | | | NT2RM4002532, weakly similar to | |
| | | | PROTEIN HOM1 | |
| 385 | 2538.M16.GZ43_371543 | AF375410 | gi\|14030638\|gb\|AF375410.1AF375410 | 1.9E−53 |
| | | | Arabidopsis thaliana At2g43970/F6E13.10 | |
| | | | gene, complete cds | |
| 386 | 2538.M17.GZ43_371544 | AK025473 | gi\|10437996\|dbj\|AK025473.1AK025473 | 3.2E−282 |
| | | | Homo sapiens cDNA: FLJ21820 fis, clone | |
| | | | HEP01232 | |
| 389 | 2538.P16.GZ43_371615 | AK026286 | gi\|10439097\|dbj\|AK026286.1AK026286 | 0 |
| | | | Homo sapiens cDNA: FLJ22633 fis, clone | |
| | | | HSI06502 | |
| 391 | 2554.A06.GZ43_375853 | AK001324 | gi\|7022509\|dbj\|AK001324.1AK001324 | 4E−44 |
| | | | Homo sapiens cDNA FLJ10462 fis, clone | |
| | | | NT2RP1001494, weakly similar to MALE | |
| | | | STERILITY PROTEIN 2 | |
| 394 | 2554.A16.GZ43_375863 | AF271388 | gi\|8515842\|gb\|AF271388.1AF271388 Homo | 0 |
| | | | sapiens CMP-N-acetylneuraminic acid | |
| | | | synthase mRNA, complete cds | |
| 406 | 2554.I15.GZ43_376054 | AY050376 | gi\|15215695\|gb\|AY050376.1 Arabidopsis | 8.8E−27 |
| | | | thaliana AT3g16950/K14A17_7 mRNA, | |
| | | | complete cds | |
| 415 | 2554.P16.GZ43_376223 | AK022368 | gi\|10433751\|dbj\|AK022368.1AK022368 | 6.7E−46 |
| | | | Homo sapiens cDNA FLJ12306 fis, clone | |
| | | | MAMMA1001907 | |
| 418 | 2565.B13.GZ43_398139 | AL050012 | gi\|4884261\|emb\|AL050012.1HSM800354 | 1E−44 |
| | | | Homo sapiens mRNA; cDNA | |
| | | | DKFZp564K133 (from clone | |
| | | | DKFZp564K133) | |
| 419 | 2565.B15.GZ43_398171 | AY049285 | gi\|15146287\|gb\|AY049285.1 Arabidopsis | 2.1E−62 |
| | | | thaliana AT3g58570/F14P22_160 mRNA, | |
| | | | complete cds | |
| 422 | 2565.C17.GZ43_398204 | M24543 | gi\|341200\|gb\|M24543.1HUMPSANTIG | 2.5E−49 |
| | | | Human prostate-specific antigen (PA) gene, | |
| | | | complete cds | |
| 423 | 2565.D06.GZ43_398029 | AF331321 | gi\|13095271\|gb\|AF331321.1AF331321 | 4.7E−30 |
| | | | HIV1 isolate T7C44 from the Netherlands | |
| | | | nonfunctional pol polyprotein gene, partial | |
| | | | sequence | |
| 428 | 2565.G20.GZ43_398256 | AJ276936 | gi\|12214232\|emb\|AJ276936.1NME276936 | 0 |
| | | | Neisseria meningitidis partial tbpB gene for | |
| | | | transferrin binding protein B subunit, allele | |
| | | | 66, | |
| 429 | 2565.H01.GZ43_397953 | AF326517 | gi\|15080738\|gb\|AF326517.1AF326517 | 1E−300 |
| | | | Abies grandis pinene synthase gene, partial | |
| | | | cds | |
| 433 | 2565.I22.GZ43_398290 | AK001926 | gi\|7023492\|dbj\|AK001926.1AK001926 | 8.9E−295 |
| | | | Homo sapiens cDNA FLJ11064 fis, clone | |
| | | | PLACE1004824 | |
| 442 | 2565.M14.GZ43_398166 | AF275699 | gi\|12275949\|gb\|AF275699.1AF275699 | 1.4E−21 |
| | | | Unidentified Hailaer soda lake bacterium | |
| | | | F16 16S ribosomal RNA gene, partial | |
| | | | sequence | |
| 447 | 2565.O07.GZ43_398056 | AK024752 | gi\|10437118\|dbj\|AK024752.1AK024752 | 4.3E−51 |
| | | | Homo sapiens cDNA: FLJ21099 fis, clone | |
| | | | CAS04610 | |
| 452 | 2540.A24.G743_372031 | Z69920 | gi\|1217632\|emb\|Z69920.1HS91K3D Human | 1.1E−41 |
| | | | DNA sequence from cosmid 91K3, | |
| | | | Huntington's Disease Region, chromosome | |
| | | | 4p16.3 | |
| 463 | 2540.H07.GZ43_372182 | AE008025 | gi\|15155943\|gb\|AE008025.1AE008025 | 1.7E−40 |
| | | | Agrobacterium tumefaciens strain C58 | |
| | | | circular chromosome, section 83 of 254 of | |
| | | | the complete seque | |
| 465 | 2540.I10.GZ43_372209 | AK000658 | gi\|7020892\|dbj\|AK000658.1AK000658 | 1.3E−53 |
| | | | Homo sapiens cDNA FLJ20651 fis, clone | |
| | | | KAT01814 | |
| 468 | 2540.M22.GZ43_372317 | AF375597 | gi\|14150816\|gb\|AF375597.1AF375596S2 | 0 |
| | | | Mus musculus medium and short chain L-3- | |
| | | | hydroxyacyl-Coenzyme A dehydrogenase | |
| | | | (Mschad) gene, exo | |
| 472 | 2540.C19.GZ43_372074 | AB019559 | gi\|4579750\|dbj\|AB019559.1AB019559 Sus | 3.1E−24 |
| | | | scrofa mRNA for 130 kDa regulatory | |
| | | | subunit of myosin phosphatase, partial cds | |
| 477 | 2540.F15.GZ43_372142 | AY016428 | gi\|13891961\|gb\|AY016428.1 Plasmodium | 2.2E−33 |
| | | | falciparum isolate Fas 30-6-7 apical | |
| | | | membrane antigen-1 (AMA-1) gene, partial | |
| | | | cds | |
| 485 | 2540.M18.GZ43_372313 | AJ331177 | gi\|15875595\|emb\|AJ331177.1HSA331177 | 7.7E−237 |
| | | | Homo sapiens genomic sequence | |
| | | | surrounding NotI site, clone NL1-ZF18RS | |
| 507 | 2541.L08.GZ43_372663 | BC003673 | gi\|13277537\|gb\|BC003673.1BC003673 | 2.6E−53 |
| | | | Homo sapiens, protamine 1, clone | |
| | | | MGC: 12307 IMAGE: 3935638, mRNA, | |
| | | | complete cds | |
| 508 | 2541.L12.GZ43_372667 | AJ297708 | gi\|12055486\|emb\|AJ297708.1RNO297708 | 9.4E−45 |
| | | | Rattus norvegicus RT6 gene for T cell | |
| | | | differentiation marker RT6.2, exons 1-8 | |
| 514 | 2506.C15.GZ43_366620 | AE007488 | gi\|14973493\|gb\|AE007488.1AE007488 | 1.4E−287 |
| | | | Streptococcus pneumoniae TIGR4 section | |
| | | | 171 of 194 of the complete genome | |
| 519 | 2506.E18.GZ43_366671 | AK025164 | gi\|10437625\|dbj\|AK025164.1AK025164 | 0 |
| | | | Homo sapiens cDNA: FLJ21511 fis, clone | |
| | | | COL05748 | |
| 521 | 2506.G24.GZ43_366725 | AY030962 | gi\|13736961\|gb\|AY030962.1 HIV-1 isolate | 9.1E−233 |
| | | | NC3964-1999 from USA pol polyprotein | |
| | | | (pol) gene, partial cds | |
| 527 | 2506.J20.GZ43_366793 | AF152924 | gi\|5453323\|gb\|AF152924.1AF152924 Mus | 2.3E−79 |
| | | | musculus syntaxin4-interacting protein synip | |
| | | | mRNA, complete cds | |
| 528 | 2506.J22.GZ43_366795 | AK000169 | gi\|7020080\|dbj\|AK000169.1AK000169 | 1.8E−99 |
| | | | Homo sapiens cDNA FLJ20162 fis, clone | |
| | | | COL09280 | |
| 531 | 2506.M05.GZ43_366850 | AE007580 | gi\|15023517\|gb\|AE007580.1AE007580 | 2.1E−217 |
| | | | Clostridium acetobutylicum ATCC824 | |
| | | | section 68 of 356 of the complete genome | |
| 534 | 2506.P07.GZ43_366924 | AF035442 | gi\|3142369\|gb\|AF035442.1AF035442 Homo | 1E−44 |
| | | | sapiens VAV-like protein mRNA, partial | |
| | | | cds | |
| 540 | 2542.C20.GZ43_372843 | AE007424 | gi\|14972724\|gb\|AE007424.1AE007424 | 2.3E−42 |
| | | | Streptococcus pneumoniae TIGR4 section | |
| | | | 107 of 194 of the complete genome | |
| 543 | 2542.D19.GZ43_372866 | BC008333 | gi\|14249906\|gb\|BC008333.1BC008333 | 5.3E−284 |
| | | | Homo sapiens, clone IMAGE: 3506145, | |
| | | | mRNA, partial cds | |
| 544 | 2542.F05.GZ43_372900 | AK024179 | gi\|10436495\|dbj\|AK024179.1AK024179 | 2.4E−41 |
| | | | Homo sapiens cDNA FLJ14117 fis, clone | |
| | | | MAMMA1001785 | |
| 553 | 2542.M09.GZ43_373072 | AK022973 | gi\|10434673\|dbj\|AK022973.1AK022973 | 5.8E−243 |
| | | | Homo sapiens cDNA FLJ12911 fis, clone | |
| | | | NT2RP2004425, highly similar to Mus | |
| | | | musculus axotrophin mR | |
| 557 | 2542.P19.GZ43_373154 | AK025164 | gi\|10437625\|dbj\|AK025164.1AK025164 | 0 |
| | | | Homo sapiens cDNA: FLJ21511 fis, clone | |
| | | | COL05748 | |
| 562 | 2542.M24.GZ43_373087 | AK022173 | gi\|10433509\|dbj\|AK022173.1AK022173 | 1.2E−284 |
| | | | Homo sapiens cDNA FLJ12111 fis, clone | |
| | | | MAMMA1000025 | |
| 563 | 2542.N21.GZ43_373108 | AF025409 | gi\|2582414\|gb\|AF025409.1AF025409 Homo | 2E−70 |
| | | | sapiens zinc transporter 4 (ZNT4) mRNA, | |
| | | | complete cds | |
| 567 | 2555.D22.GZ43_373253 | AL1576971 | gi\|11121002\|emb\|AL157697.11AL157697 | 1.1E−87 |
| | | | Human DNA sequence from clone RP5- | |
| | | | 1092C14 on chromosome 6, complete | |
| | | | sequence [Homo sapiens] | |
| 568 | 2555.E20.GZ43_373275 | AK026618 | gi\|10439509\|dbj\|AK026618.1AK026618 | 0 |
| | | | Homo sapiens cDNA: FLJ22965 fis, clone | |
| | | | KAT10418 | |
| 569 | 2555.F16.GZ43_373295 | AF271388 | gi\|8515842\|gb\|AF271388.1AF271388 Homo | 0 |
| | | | sapiens CMP-N-acetylneuraminic acid | |
| | | | synthase mRNA, complete cds | |
| 574 | 2555.K17.GZ43_373416 | AK026686 | gi\|10439593\|dbj\|AK026686.1AK026686 | 1.8E−23 |
| | | | Homo sapiens cDNA: FLJ23033 fis, clone | |
| | | | LNG02005 | |
| 578 | 2555.P22.GZ43_373541 | AF087913 | gi\|5081331\|gb\|AF087913.1AF087913 | 5.8E−74 |
| | | | Human endogenous retrovirus HERV-P- | |
| | | | T47D | |
| 579 | 2555.A11.GZ43_373170 | NC_000957 | gi\|11497445\|ref\|NC_000957.1 Borrelia | 1.3E−57 |
| | | | burgdorferi plasmid 1p5, complete sequence | |
| 585 | 2555.I12.GZ43_373363 | AJ276936 | gi\|12214232\|emb\|AJ276936.1NME276936 | 1.6E−237 |
| | | | Neisseria meningitidis partial tbpB gene for | |
| | | | transferrin binding protein B subunit, allele | |
| | | | 66, | |
| 589 | 2556.A02.GZ43_373545 | AE007289 | gi\|14524175\|gb\|AE007289.1AE007289 | 2E−55 |
| | | | Sinorhizobium meliloti plasmid pSymA | |
| | | | section 95 of 121 of the complete plasmid | |
| | | | sequence | |
| 591 | 2556.C11.GZ43_373602 | AY039252 | gi\|15418981\|gb\|AY039252.1 Macaca | 3.1E−29 |
| | | | mulatta immunoglobulin alpha heavy chain | |
| | | | constant region (IgA) gene, IgA-C.II allele, | |
| | | | partial cds | |
| 602 | 2556.H15.GZ43_373726 | AK021966 | gi\|10433275\|dbj\|AK0219666.1AK021966 | 1.6E−70 |
| | | | Homo sapiens cDNA FLJ11904 fis, clone | |
| | | | HEMBB1000048 | |
| 620 | 2557.B22.GZ43_373973 | AB071392 | gi\|15721873\|dbj\|AB071392.1AB071392 | 1.2E−25 |
| | | | Expression vector pAQ-EX1 DNA, | |
| | | | complete sequence | |
| 627 | 2557.J14.GZ43_374157 | AK023721 | gi\|10435737\|dbj\|AK023721.1AK023721 | 1.6E−209 |
| | | | Homo sapiens cDNA FLJ13659 fis, clone | |
| | | | PLACE1011576, moderately similar to | |
| | | | Human Kruppel related | |
| 635 | 2557.N14.GZ43_374253 | AB013897 | gi\|6177784\|dbj\|AB013897.1AB013897 | 1E−44 |
| | | | Homo sapiens mRNA for HKR1, partial cds | |
| 648 | 2558.B24.GZ43_374359 | AB064318 | gi\|14595115\|dbj\|AB064318.1AB064318 | 4.6E−28 |
| | | | Comamonas testosteroni gene for 16S | |
| | | | rRNA, partial sequence | |
| 657 | 2558.G07.GZ43_374462 | M92069 | gi\|337698\|gb\|M92069.1HUMRTVLC | 6.7E−46 |
| | | | Human retrovirus-like sequence-isoleucine c | |
| | | | (RTVL-Ic) gene, Alu repeats | |
| 661 | 2558.H17.GZ43_374496 | AK023812 | gi\|10435860\|dbj\|AK023812.1AK023812 | 5.2E−31 |
| | | | Homo sapiens cDNA FLJ13750 fis, clone | |
| | | | PLACE3000331 | |
| 662 | 2558.J01.GZ43_374528 | AK023448 | gi\|10435386\|dbj\|AK023448.1AK023448 | 4.8E−278 |
| | | | Homo sapiens cDNA FLJ13386 fis, clone | |
| | | | PLACE1001104, weakly similar to | |
| | | | MYOSIN HEAVY CHAIN, NON-MU | |
| 666 | 2558.K02.GZ43_374553 | U14573 | gi\|551542\|gb\|U14573.1HSU14573 ***ALU | 1.3E−62 |
| | | | WARNING: Human Alu-Sq subfamily | |
| | | | consensus sequence | |
| 683 | 2559.D05.GZ43_374772 | AF338713 | gi\|14039582\|gb\|AF338713.1AF338713 | 4E−297 |
| | | | Casuarius casuarius mitochondrion, partial | |
| | | | genome | |
| 687 | 2559.I12.GZ43_374899 | AY036096 | gi\|14486435\|gb\|AY036096.1 HIV-1 isolate | 1.4E−41 |
| | | | L2Q2P from Belgium reverse transcriptase | |
| | | | (pol) gene, partial cds | |
| 690 | 2559.J02.GZ43_374913 | AK026618 | gi\|10439509\|dbj\|AK026618.1AK026618 | 0 |
| | | | Homo sapiens cDNA: FLJ22965 fis, clone | |
| | | | KAT10418 | |
| 692 | 2559.K12.GZ43_374947 | Z96776 | gi\|2181853\|emb\|Z96776.1HS9QT023 | 5.1E−52 |
| | | | H. sapiens telomeric DNA sequence, clone | |
| | | | 9QTEL023, read 9QTELOO023.seq | |
| 694 | 2559.L09.GZ43_374968 | AE007426 | gi\|14972746\|gb\|AE007426.1AE007426 | 8.1E−21 |
| | | | Streptococcus pneumoniae TIGR4 section | |
| | | | 109 of 194 of the complete genome | |
| 696 | 2559.M21.GZ43_375004 | AJ414564 | gi\|15990852\|emb\|AJ414564.1HSA414564 | 9.2E−30 |
| | | | Homo sapiens mRNA for connexin40.1 | |
| | | | (CX40.1 gene) | |
| 698 | 2559.N13.GZ43_375020 | AL137330 | gi\|6807822\|emb\|AL137330.1HSM802010 | 4.1E−47 |
| | | | Homo sapiens mRNA; cDNA | |
| | | | DKFZp434F0272 (from clone | |
| | | | DKFZp434F0272) | |
| 714 | 2560.H01.GZ43_375248 | U14567 | gi\|551536\|gb\|U14567.1HSU14567 ***ALU | 2.7E−42 |
| | | | WARNING: Human Alu-J subfamily | |
| | | | consensus sequence | |
| 719 | 2560.K02.GZ43_375321 | AF178754.3 | gi\|7770069\|gb\|AF178754.3AF178754 Homo | 3.1E−51 |
| | | | sapiens lithium-sensitive myo-inositol | |
| | | | monophosphatase A1 (IMPA1) gene, | |
| | | | promoter region and p | |
| 720 | 2560.K08.GZ43_375327 | AK009327 | gi\|12844057\|dbj\|AK009327.1AK009327 | 6.3E−80 |
| | | | Mus musculus adult male tongue cDNA, | |
| | | | RIKEN full-length enriched library, | |
| | | | clone: 2310012P17, full | |
| 721 | 2560.K10.GZ43_375329 | AF344987 | gi\|13448249\|gb\|AF344987.1AF344987 | 1E−300 |
| | | | Hepatitis C virus isolate RDpostSC1c2 | |
| | | | polyprotein gene, partial cds | |
| 729 | 2560.O08.GZ43_375423 | AY037285 | gi\|15982643\|gb\|AY037285.1AY037284S2 | 5.2E−54 |
| | | | HIV-1 from Cameroon vpu protein (vpu) | |
| | | | and envelope glycoprotein (env) genes, | |
| | | | complete cds; and | |
| 732 | 2561.B03.GZ43_376258 | AF035968.2 | gi\|8714504\|gb\|AF035968.2AF035968 Homo | 3.9E−32 |
| | | | sapiens integrin alpha 2 (ITGA2) gene, | |
| | | | ITGA2-1 allele, exons 6-9, and partial cds | |
| 733 | 2561.B12.GZ43_376267 | AP000276 | gi\|4835645\|dbj\|AP000276.1AP000276 | 1.9E−27 |
| | | | Homo sapiens genomic DNA, chromosome | |
| | | | 21q22.1, D21S226-AML region, | |
| | | | clone: 55A9, complete sequence | |
| 750 | 2561.M09.GZ43_376528 | AF052684 | gi\|2995716\|gb\|AF052684.1HSPRCAD2 | 4.1E−41 |
| | | | Homo sapiens protocadherin 43 gene, exon 2 | |
| 753 | 2561.E22.GZ43_376349 | AF132952 | gi\|4680674\|gb\|AF132952.1AF132952 Homo | 3E−41 |
| | | | sapiens CGI-18 protein mRNA, complete | |
| | | | cds | |
| 754 | 2561.G20.GZ43_376395 | U14573 | gi\|551542\|gb\|U14573.1HSU14573 ***ALU | 1.5E−71 |
| | | | WARNING: Human Alu-Sq subfamily | |
| | | | consensus sequence | |
| 755 | 2561.H17.GZ43_376416 | AF052685 | gi\|2995717\|gb\|AF052685.1HSPRCAD3 | 2.1E−24 |
| | | | Homo sapiens protocadherin 43 gene, exon | |
| | | | 3, exon 4, and complete cds | |
| 756 | 2561.I19.GZ43_376442 | AF344987 | gi\|13448249\|gb\|AF344987.1AF344987 | 3.2E−201 |
| | | | Hepatitis C virus isolate RDpostSC1c2 | |
| | | | polyprotein gene, partial cds | |
| 761 | 2561.P16.GZ43_376607 | Z78727 | gi\|1508005\|emb\|Z78727.1HSPA15B9 | 1.6E−37 |
| | | | H. sapiens flow-sorted chromosome 6 | |
| | | | HindIII fragment, SC6pA15B9 | |
| 762 | 2561.P19.GZ43_376610 | U66535 | gi\|2270915\|gb\|U66535.1HSITGBF07 | 8.6E−41 |
| | | | Human beta4-integrin (ITGB4) gene, exons | |
| | | | 19, 20, 21, 22, 23, 24 and 25 | |
| 763 | 2561.P23.GZ43_376614 | AF167458 | gi\|6467463\|gb\|AF167458.1HSDSRPKR04 | 1E−22 |
| | | | Homo sapiens double stranded RNA | |
| | | | activated protein kinase (PKR) gene, intron 1 | |
| 771 | 2456.D04.GZ43_355904 | AF307053 | gi\|12018057\|gb\|AF307053.1AF307053 | 0 |
| | | | Thermococcus litoralis sugar kinase, | |
| | | | trehalose/maltose binding protein (malE), | |
| | | | trehalose/maltose | |
| 777 | 2456.H02.GZ43_355998 | AJ005821 | gi\|3123571\|emb\|AJ005821.1HSA5821 | 5.8E−37 |
| | | | Homo sapiens mRNA for X-like 1 protein | |
| 788 | 2456.N23.GZ43_356163 | AF188746 | gi\|6425045\|gb\|AF188746.1AF188746 Homo | 9.6E−63 |
| | | | sapiens prostrate kallikrein 2 (KLK2) | |
| | | | mRNA, complete cds | |
| 796 | 2457.C19.GZ43_356279 | AF368920 | gi\|14039926\|gb\|AF368920.1AF368920 | 1E−47 |
| | | | Caenorhabditis elegans voltage-dependent | |
| | | | calcium channel alpha13 subunit (cca-1) | |
| | | | mRNA, complete c | |
| 799 | 2457.D12.GZ43_356296 | AK026618 | gi\|10439509\|dbj\|AK026618.1AK026618 | 0 |
| | | | Homo sapiens cDNA: FLJ22965 fis, clone | |
| | | | KAT10418 | |
| 810 | 2457.H17.GZ43_356397 | AE007614 | gi\|15023883\|gb\|AE007614.1AE007614 | 9E−63 |
| | | | Clostridium acetobutylicum ATCC824 | |
| | | | section 102 of 356 of the complete genome | |
| 823 | 2458.A10.GZ43_356618 | AK026920 | gi\|10439892\|dbj\|AK026920.1AK026920 | 6.2E−84 |
| | | | Homo sapiens cDNA: FLJ23267 fis, clone | |
| | | | COL07266 | |
| 827 | 2458.B23.GZ43_356655 | AB050432 | gi\|10998295\|dbj\|AB050432.1AB050432 | 4.3E−129 |
| | | | Macaca fascicularis brain cDNA, | |
| | | | clone: QnpA-21861 | |
| 829 | 2458.C06.GZ43_356662 | U49973 | gi\|2226003\|gb\|U49973.1HSU49973 Human | 2E−24 |
| | | | Tigger1 transposable element, complete | |
| | | | consensus sequence | |
| 842 | 2458.I09.GZ43_356809 | AK023496 | gi\|10435445\|dbj\|AK023496.1AK023496 | 2.4E−39 |
| | | | Homo sapiens cDNA FLJ13434 fis, clone | |
| | | | PLACE1002578 | |
| 843 | 2458.I10.GZ43_356810 | AF031077 | gi\|6649934\|gb\|AF031077.1AF031077 Homo | 1.3E−52 |
| | | | sapiens chromosome X, cosmid | |
| | | | LLNLc110C1837, complete sequence | |
| 845 | 2458.I17.GZ43_356817 | AK026569 | gi\|10439451\|dbj\|AK026569.1AK026569 | 1.8E−38 |
| | | | Homo sapiens cDNA: FLJ22916 fis, clone | |
| | | | KAT06406, highly similar to HSCYCR | |
| | | | Human mRNA for T-cell | |
| 846 | 2458.I20.GZ43_356820 | AF184614 | gi\|6983939\|gb\|AF184614.1AF184614 Homo | 4.2E−33 |
| | | | sapiens p47-phox (NCF1) gene, complete | |
| | | | cds | |
| 855 | 2458.N06.GZ43_356926 | AF367251 | gi\|14161363\|gb\|AF367251.1AF367251 | 2.2E−70 |
| | | | Helicobacter pylori strain CAPM N93 | |
| | | | cytotoxin associated protein A (cagA) gene, | |
| | | | complete cds | |
| 865 | 2459.B11.GZ43_357027 | AF375597 | gi\|14150816\|gb\|AF375597.1AF375596S2 | 0 |
| | | | Mus musculus medium and short chain L-3- | |
| | | | hydroxyacyl-Coenzyme A dehydrogenase | |
| | | | (Mschad) gene, exo | |
| 866 | 2459.C05.GZ43_357045 | X04803.2 | gi\|6647297\|emb\|X04803.2HSYUBG1 Homo | 6.4E−52 |
| | | | sapiens ubiquitin gene | |
| 873 | 2459.F20.GZ43_357132 | AK025207 | gi\|10437672\|dbj\|AK025207.1AK025207 | 0 |
| | | | Homo sapiens cDNA: FLJ21554 fis, clone | |
| | | | COL06330 | |
| 877 | 2459.H09.GZ43_357169 | AB046623 | gi\|9651056\|dbj\|AB046623.1AB046623 | 1.7E−35 |
| | | | Macaca fascicularis brain cDNA, clone | |
| | | | QccE-10576 | |
| 888 | 2459.O23.GZ43_357351 | AL049301 | gi\|4500067\|emb\|AL049301.1HSM800086 | 1.3E−31 |
| | | | Homo sapiens mRNA; cDNA | |
| | | | DKFZp564P073 (from clone | |
| | | | DKFZp564P073) | |
| 889 | 2459.P24.GZ43_357376 | AK018110 | gi\|12857675\|dbj\|AK018110.1AK018110 | 1.5E−33 |
| | | | Mus musculus adult male medulla oblongata | |
| | | | cDNA, RIKEN full-length enriched library, | |
| | | | clone: 633040 | |
| 903 | 2464.H22.GZ43_357870 | AB035344 | gi\|8176599\|dbj\|AB035344.1AB035344S1 | 1.1E−127 |
| | | | Homo sapiens TCL6 gene, exon 1-10b | |
| 904 | 2464.I04.GZ43_357876 | AK025125 | gi\|10437578\|dbj\|AK025125.1AK025125 | 0 |
| | | | Homo sapiens cDNA: FLJ21472 fis, clone | |
| | | | COL04936 | |
| 905 | 2464.I20.GZ43_357892 | AK025966 | gi\|10438647\|dbj\|AK025966.1AK025966 | 2.8E−61 |
| | | | Homo sapiens cDNA: FLJ22313 fis, clone | |
| | | | HRC05216 | |
| 909 | 2464.K18.GZ43_357938 | AF287938 | gi\|12656333\|gb\|AF287938.1AF287938 | 8.3E−44 |
| | | | Guichenotia ledifolia NADH dehydrogenase | |
| | | | subunit F (ndhF) gene, partial cds; | |
| | | | chloroplast gene for | |
| 912 | 2464.L15.GZ43_357959 | AF141308 | gi\|5737754\|gb\|AF141308.1HSPMFG1 | 9.9E−76 |
| | | | Homo sapiens polyamine modulated factor-1 | |
| | | | (PMF1) gene, exon 1 | |
| 918 | 2464.P17.GZ43_358057 | AF052684 | gi\|2995716\|gb\|AF052684.1HSPRCAD2 | 3E−29 |
| | | | Homo sapiens protocadherin 43 gene, exon 2 | |
| 934 | 2465.J19.GZ43_358299 | X02571 | gi\|31870\|emb\|X02571.1HSGP5MOS Human | 2.7E−48 |
| | | | gene fragment related to oncogene c-mos | |
| | | | with Alu repeats (locus gp5, region NV-1) | |
| 935 | 2465.K20.GZ43_358324 | AK019509 | gi\|12859761\|dbj\|AK019509.1AK019509 | 2.5E−63 |
| | | | Mus musculus 0 day neonate skin cDNA, | |
| | | | RIKEN full-length enriched library, | |
| | | | clone: 4632435C11, full | |
| 937 | 2465.L06.GZ43_358334 | AK009327 | gi\|12844057\|dbj\|AK009327.1AK009327 | 7.9E−73 |
| | | | Mus musculus adult male tongue cDNA, | |
| | | | RIKEN full-length enriched library, | |
| | | | clone: 2310012P17, full | |
| 939 | 2465.M11.GZ43_358363 | AK022253 | gi\|10433611\|dbj\|AK022253.1AK022253 | 1.4E−112 |
| | | | Homo sapiens cDNA FLJ12191 fis, clone | |
| | | | MAMMA1000843 | |
| 943 | 2466.B02.GZ43_360107 | AK023055 | gi\|10434796\|dbj\|AK023055.1AK023055 | 7.5E−39 |
| | | | Homo sapiens cDNA FLJ12993 fis, clone | |
| | | | NT2RP3000197 | |
| 944 | 2466.C15.GZ43_360144 | AB013897 | gi\|6177784\|dbj\|AB013897.1AB013897 | 4.3E−53 |
| | | | Homo sapiens mRNA for HKR1, partial cds | |
| 945 | 2466.D19.GZ43_360172 | AL050141 | gi\|4884352\|emb\|AL050141.1HSM800441 | 3.4E−110 |
| | | | Homo sapiens mRNA; cDNA | |
| | | | DKFZp586O031 (from clone | |
| | | | DKFZp586O031) | |
| 952 | 2466.I08.GZ43_360281 | AJ271729 | gi\|6900103\|emb\|AJ271729.1HSA271729 | 6.2E−72 |
| | | | Homo sapiens mRNA for glucose-regulated | |
| | | | protein (HSPA5 gene) | |
| 953 | 2466.J01.GZ43_360298 | AY058527 | gi\|16197970\|gb\|AY058527.1 Drosophila | 9.4E−40 |
| | | | melanogaster LD23445 full length cDNA | |
| 954 | 2466.J24.GZ43_360321 | AF331425 | gi\|13375486\|gb\|AF331425.1AF331425 HIV- | 1.6E−77 |
| | | | 1 D311 from Australia envelope protein | |
| | | | (env) gene, partial cds | |
| 958 | 2467.B24.GZ43_360513 | AJ005821 | gi\|3123571\|emb\|AJ005821.1HSA5821 | 1.4E−34 |
| | | | Homo sapiens mRNA for X-like 1 protein | |
| 963 | 2467.H18.GZ43_360651 | AF036235 | gi\|2695679\|gb\|AF036235.1AF036235 | 2E−169 |
| | | | Gorilla gorilla L1 retrotransposon L1Gg-1A, | |
| | | | complete sequence | |
| 964 | 2467.A03.GZ43_360468 | BC012960 | gi\|15277963\|gb\|BC012960.1BC012960 Mus | 8.7E−36 |
| | | | musculus , ring finger protein 12, clone | |
| | | | MGC: 13712 IMAGE: 4193003, mRNA, | |
| | | | complte cds | |
| 965 | 2467.A05.GZ43_360470 | BC009113 | gi\|14318629\|gb\|BC009113.1BC009113 | 4.1E−167 |
| | | | Homo sapiens, clone MGC: 18122 | |
| | | | IMAGE: 4153377, mRNA, complete cds | |
| 969 | 2467.G01.GZ43_360610 | U14573 | gi\|551542\|gb\|U14573.1HSU14573 ***ALU | 2E−61 |
| | | | WARNING: Human Alu-Sq subfamily | |
| | | | consensus sequence | |
| 971 | 2467.N22.GZ43_360799 | AF117756 | gi\|4530440\|gb\|AF117756.1AF117756 Homo | 6.8E−77 |
| | | | sapiens thyroid hormone receptor-associated | |
| | | | protein complex component TRAP150 | |
| | | | mRNA, complete | |
| 973 | 2467.I12.GZ43_360669 | AK024049 | gi\|10436318\|dbj\|AK024049.1AK024049 | 2.1E−47 |
| | | | Homo sapiens cDNA FLJ13987 fis, clone | |
| | | | Y79AA1001963, weakly similar to | |
| | | | PUTATIVE PRE-MRNA SPLICING | |
| 977 | 2467.K14.GZ43_360719 | AB030001 | gi\|7416074\|dbj\|AB030001.1AB030001 | 7.2E−22 |
| | | | Homo sapiens gene for SGRF, complete cds | |
| 979 | 2467.N03.GZ43_360780 | AK023448 | gi\|10435386\|dbj\|AK023448.1AK023448 | 0 |
| | | | Homo sapiens cDNA FLJ13386 fis, clone | |
| | | | PLACE1001104, weakly similar to | |
| | | | MYOSIN HEAVY CHAIN, NON-MU | |
| 980 | 2467.N07.GZ43_360784 | AK001931 | gi\|7023502\|dbj\|AK001931.1AK001931 | 2.3E−54 |
| | | | Homo sapiens cDNA FLJ11069 fis, clone | |
| | | | PLACE1004930, highly similar to Homo | |
| | | | sapiens MDC-3.13 isofo | |
| 981 | 2467.N09.GZ43_360786 | AE008338 | gi\|15159908\|gb\|AE008338.1AE008338 | 3.7E−50 |
| | | | Agrobacterium tumefaciens strain C58 linear | |
| | | | chromosome, section 142 of 187 of the | |
| | | | complete sequen | |
| 986 | 2472.C18.GZ43_360915 | K01921 | gi\|339606\|gb\|K01921.1HUMTGNB Human | 3E−29 |
| | | | Asn-tRNA gene, clone pHt6-2, complete | |
| | | | sequence and flanks | |
| 992 | 2472.G03.GZ43_360996 | AF321082 | gi\|12958576\|gb\|AF321082.1AF321082 HIV- | 5.1E−28 |
| | | | 1 isolate DGOB from France envelope | |
| | | | glycoprotein (env) gene, complete cds | |
| 999 | 2472.M22.GZ43_361159 | AF338299 | gi\|12958808\|gb\|AF338299.1AF338299 | 1.4E−145 |
| | | | Amazona ochrocephala auropalliata | |
| | | | mitochondrial control region 1, partial | |
| | | | sequence | |
| 1002 | 2472.P22.GZ43_361231 | AJ330257 | gi\|15874675\|emb\|AJ330257.1HSA330257 | 1.1E−63 |
| | | | Homo sapiens genomic sequence | |
| | | | surrounding NotI site, clone NL1-FA14R | |
| 1005 | 2473.F08.GZ43_361361 | AF306355 | gi\|14573206\|gb\|AF306355.1AF306355 | 3.2E−29 |
| | | | Homo sapiens clone TF3.19 | |
| | | | immunoglobulin heavy chain variable region | |
| | | | mRNA, partial cds | |
| 1006 | 2473.F14.GZ43_361367 | AB050477 | gi\|11034759\|dbj\|AB050477.1AB050477 | 0 |
| | | | Homo sapiens NIBAN mRNA, complete cds | |
| 1011 | 2473.I08.GZ43_361433 | AF224341 | gi\|15982934\|gb\|AF224341.1AF224341 Mus | 8.7E−67 |
| | | | musculus thiamine transporter 1 (Slc19a2) | |
| | | | gene, exons 1 through 6 and complete cds | |
| 1015 | 2473.O13.GZ43_361582 | AF203815 | gi\|6979641\|gb\|AF203815.1AF203815 Homo | 5.4E−44 |
| | | | sapiens alpha gene sequence | |
| 1018 | 2474.C08.GZ43_361673 | AK000373 | gi\|7020417\|dbj\|AK000373.1AK000373 | 5.6E−47 |
| | | | Homo sapiens cDNA FLJ20366 fis, clone | |
| | | | HEP 18008 | |
| 1021 | 2474.G17.GZ43_361778 | U75285 | gi\|2315862\|gb\|U75285.1HSU75285 Homo | 1.1E−87 |
| | | | sapiens apoptosis inhibitor survivin gene, | |
| | | | complete cds | |
| 1023 | 2474.I06.GZ43_361815 | Z81315 | gi\|1644298\|emb\|Z81315.1HSF62D4 Human | 2.1E−67 |
| | | | DNA sequence from fosmid F62D4 on | |
| | | | chromosome 22q12-qter | |
| 1024 | 2474.J18.GZ43_361851 | AF029062 | gi\|3712662\|gb\|AF029062.1AF029062 Homo | 1.2E−28 |
| | | | sapiens DEAD-box protein (BAT1) gene, | |
| | | | partial cds | |
| 1030 | 2474.P22.GZ43_361999 | AL050204 | gi\|4884443\|emb\|AL050204.1HSM800501 | 8.9E−33 |
| | | | Homo sapiens mRNA; cDNA | |
| | | | DKFZp586F1223 (from clone | |
| | | | DKFZp586F1223) | |
| 1031 | 2475.A05.GZ43_362006 | AL109666 | gi\|5689800\|emb\|AL109666.1IRO35907 | 6.3E−43 |
| | | | Homo sapiens mRNA full length insert | |
| | | | cDNA clone EUROIMAGE 35907 | |
| 1032 | 2475.C18.GZ43_362067 | AK023739 | gi\|10435762\|dbj\|AK023739.1AK023739 | 2.8E−180 |
| | | | Homo sapiens cDNA FLJ13677 fis, clone | |
| | | | PLACE1011982 | |
| 1033 | 2475.E18.GZ43_362115 | AK024206 | gi\|10436527\|dbj\|AK024206.1AK024206 | 1.9E−21 |
| | | | Homo sapiens cDNA FLJ14144 fis, clone | |
| | | | MAMMA1002909 | |
| 1035 | 2475.H06.GZ43_362175 | AF322634 | gi\|12657820\|gb\|AF322634.1AF322634S1 | 1.2E−173 |
| | | | Human herpesvirus 3 strain VZV-Iceland | |
| | | | glycoprotein B gene, complete cds | |
| 1036 | 2475.H13.GZ43_362182 | AF026853 | gi\|3882436\|gb\|AF026853.1HSHADHSC 1 | 2.1E−30 |
| | | | Homo sapiens mitochondrial short-chain L-3 | |
| | | | hydroxyacyl-CoA dehydrogenase | |
| | | | (HADHSC) gene, nuclear | |
| 1039 | 2475.N08.GZ43_362321 | AK011295 | gi\|12847322\|dbj\|AK011295.1AK011295 | 1.1E−84 |
| | | | Mus musculus 10 days embryo cDNA, | |
| | | | RIKEN full-length enriched library, | |
| | | | clone: 2610002L04, full ins | |
| 1045 | 2475.M20.GZ43_362309 | AK023843 | gi\|10435902\|dbj\|AK023843.1AK023843 | 8.8E−42 |
| | | | Homo sapiens cDNA FLJ13781 fis, clone | |
| | | | PLACE4000465 | |
| 1046 | 2475.N21.GZ43_362334 | S45332 | gi\|255496\|gb\|S45332.1S45332 | 1.4E−101 |
| | | | erythropoietin receptor [human, placental, | |
| | | | Genomic, 8647 nt] | |
| 1055 | 2480.G11.GZ43_358658 | X83497 | gi\|603558\|emb\|X83497.1HSLTRERV9 | 6.1E−40 |
| | | | H. sapiens DNA for ZNF80-linked ERV9 | |
| | | | long terminal repeat | |
| 1056 | 2480.H06.GZ43_358677 | AB002070 | gi\|12862447\|dbj\|AB002070.1AB002070 | 5.5E−28 |
| | | | Aspergillus clavatus gene for 18S rRNA, | |
| | | | partial sequence, strain: NRRL 1 | |
| 1061 | 2480.M20.GZ43_358811 | AL1576971 | gi\|11121002\|emb\|AL157697.11AL157697 | 9.3E−36 |
| | | | Human DNA sequence from clone RP5- | |
| | | | 1092C14 on chromosome 6, complete | |
| | | | sequence [Homo sapiens] | |
| 1064 | 2480.P23.GZ43_358886 | AB037719 | gi\|7242950\|dbj\|AB037719.1AB037719 | 3.6E−35 |
| | | | Homo sapiens mRNA for KIAA1298 | |
| | | | protein, partial cds | |
| 1065 | 2481.B06.GZ43_358917 | AK023471 | gi\|10435415\|dbj\|AK023471.AK023471 | 0 |
| | | | Homo sapiens cDNA FLJ13409 fis, clone | |
| | | | PLACE1001716 | |
| 1068 | 2481.D10.GZ43_358969 | AL021306 | gi\|2808416\|emb\|AL021306.1HS1109B5 | 7E−52 |
| | | | Human DNA sequence from clone CTB- | |
| | | | 1109B5 on chromosome 22 Contains a GSS, | |
| | | | complete sequence [Homo | |
| 1069 | 2481.D13.GZ43_358972 | X64467 | gi\|28579\|emb\|X64467.1HSALADG | 4.2E−53 |
| | | | H. sapiens ALAD gene for porphobilinogen | |
| | | | synthase | |
| 1075 | 2481.K12.GZ43_359139 | AK026901 | gi\|10439868\|dbj\|AK026901.1AK026901 | 5.9E−52 |
| | | | Homo sapiens cDNA: FLJ23248 fis, clone | |
| | | | COL03555 | |
| 1083 | 2482.E17.GZ43_359384 | AK022821 | gi\|10434440\|dbj\|AK022821.1AK022821 | 9.4E−35 |
| | | | Homo sapiens cDNA FLJ12759 fis, clone | |
| | | | NT2RP2001347 | |
| 1084 | 2482.E20.GZ43_359387 | AK014328 | gi\|12852104\|dbj\|AK014328.1AK014328 | 5.2E−99 |
| | | | Mus musculus 14, 17 days embryo head | |
| | | | cDNA, RIKEN full-length enriched library, | |
| | | | clone: 3230401M21, | |
| 1091 | 2482.N09.GZ43_359592 | AE008514 | gi\|15459095\|gb\|AE008514.1AE008514 | 6.9E−107 |
| | | | Streptococcus pneumoniae R6 section 130 | |
| | | | of 184 of the complete genome | |
| 1100 | 2483.J07.GZ43_359878 | AK022722 | gi\|10434285\|dbj\|AK022722.1AK022722 | 1E−300 |
| | | | Homo sapiens cDNA FLJ12660 fis, clone | |
| | | | NT2RM4002174, moderately similar to | |
| | | | MRP PROTEIN | |
| 1101 | 2483.K02.GZ43_359897 | AK012908 | gi\|12849956\|dbj\|AK012908.1AK012908 | 3.7E−189 |
| | | | Mus musculus 10, 11 days embryo cDNA, | |
| | | | RIKEN full-length enriched library, | |
| | | | clone: 2810046L04, full | |
| 1106 | 2483.O07.GZ43_359998 | AK014328 | gi\|12852104\|dbj\|AK0143228.1AK0143218 | 3.2E−103 |
| | | | Mus musculus 14, 17 days embryo head | |
| | | | cDNA, RIKEN full-length enriched library, | |
| | | | clone: 3230401M21, | |
| 1108 | 2488.C19.GZ43_362511 | AB023199 | gi\|4589607\|dbj\|AB023199.1AB023199 | 1.1E−50 |
| | | | Homo sapiens mRNA for KIAA0982 | |
| | | | protein, complete cds | |
| 1110 | 2488.E20.GZ43_362560 | AK001136 | gi\|7022203\|dbj\|AK001136.1AK001136 | 1E−35 |
| | | | Homo sapiens cDNA FLJ10274 fis, clone | |
| | | | HEMBB1001169 | |
| 1111 | 2488.F06.GZ43_362570 | AK011295 | gi\|12847322\|dbj\|AK011295.1AK011295 | 8.1E−55 |
| | | | Mus musculus 10 days embryo cDNA, | |
| | | | RIKEN full-length enriched library, | |
| | | | clone: 2610002L04, full ins | |
| 1113 | 2488.G02.GZ43_362590 | X15723 | gi\|31481\|emb\|X15723.1HSFURIN Human | 1.8E−85 |
| | | | fur gene, exons 1 through 8 | |
| 1117 | 2488.K04.GZ43_362688 | AF026853 | gi\|3882436\|gb\|AF026853.1HSHADHSC 1 | 2.1E−30 |
| | | | Homo sapiens mitochondrial short-chain L-3 | |
| | | | hydroxyacyl-CoA dehydrogenase | |
| | | | (HADHSC) gene, nuclear | |
| 1122 | 2489.A03.GZ43_362831 | AB050477 | gi\|11034759\|dbj\|AB050477. 1AB050477 | 6.7E−46 |
| | | | Homo sapiens NIBAN mRNA, complete cds | |
| 1124 | 2489.A13.GZ43_362841 | AK026618 | gi\|10439509\|dbj\|AK026618.1AK026618 | 1.8E−178 |
| | | | Homo sapiens cDNA: FLJ22965 fis, clone | |
| | | | KAT10418 | |
| 1127 | 2489.D18.GZ43_362918 | AF086310 | gi\|3483655\|gb\|AF086310.1HUMZD51F08 | 2.5E−79 |
| | | | Homo sapiens full length insert cDNA clone | |
| | | | ZD51F08 | |
| 1128 | 2489.F09.GZ43_362957 | AF271388 | gi\|8515842\|gb\|AF271388.1AF271388 Homo | 0 |
| | | | sapiens CMP-N-acetylneuraminic acid | |
| | | | synthase mRNA, complete cds | |
| 1129 | 2489.G05.GZ43_362977 | AK023739 | gi\|10435762\|dbj\|AK023739.1AK023739 | 6.8E−209 |
| | | | Homo sapiens cDNA FLJ13677 fis, clone | |
| | | | PLACE1011982 | |
| 1140 | 2489.M11.GZ43_363127 | AE008029 | gi\|15155994\|gb\|AE008029.1AE008029 | 4.2E−44 |
| | | | Agrobacterium tumefaciens strain C58 | |
| | | | circular chromosome, section 87 of 254 of | |
| | | | the complete seque | |
| 1144 | 2490.B06.GZ43_363242 | AK001915 | gi\|7023475\|dbj\|AK001915.1AK001915 | 1.7E−43 |
| | | | Homo sapiens cDNA FLJ11053 fis, clone | |
| | | | PLACE1004664 | |
| 1155 | 2490.J22.GZ43_363450 | AF026853 | gi\|3882436\|gb\|AF026853.1HSHADHSC 1 | 2E−30 |
| | | | Homo sapiens mitochondrial short-chain L-3 | |
| | | | hydroxyacyl-CoA dehydrogenase | |
| | | | (HADHSC) gene, nuclear | |
| 1160 | 2490.N24.GZ43_363548 | AF167438 | gi\|9622123\|gb\|AF167438.1AF167438 Homo | 8.8E−74 |
| | | | sapiens androgen-regulated short-chain | |
| | | | dehydrogenase/reductase 1 (ARSDR1) | |
| | | | mRNA, complete cds | |
| 1163 | 2491.C13.GZ43_363657 | AK022338 | gi\|10433714\|dbj\|AK022338.1AK022338 | 6.2E−30 |
| | | | Homo sapiens cDNA FLJ12276 fis, clone | |
| | | | MAMMA1001692 | |
| 1174 | 2491.P10.GZ43_363966 | AJ276936 | gi\|12214232\|emb\|AJ276936.1NME276936 | 0 |
| | | | Neisseria meningitidis partial tbpB gene for | |
| | | | transferrin binding protein B subunit, allele | |
| | | | 66, | |
| 1175 | 2491.P20.GZ43_363976 | AY027632 | gi\|15418751\|gb\|AY027632.1 Measles virus | 7.8E−283 |
| | | | strain MVs/Masan.KOR/49.00/2 | |
| | | | hemagglutinin (H) mRNA, complete cds | |
| 1177 | 2496.C08.GZ43_364139 | U67829 | gi\|2289943\|gb\|U67829.1HSU67829 Human | 3.6E−90 |
| | | | primary Alu transcript | |
| 1181 | 2496.F14.GZ43_364217 | X16983 | gi\|33945\|emb\|X16983.1HSINTAL4 Human | 4.7E−53 |
| | | | mRNA for integrin alpha-4 subunit | |
| 1183 | 2496.I06.GZ43_364281 | BC004138 | gi\|13278716\|gb\|BC004138.1BC004138 | 8.3E−53 |
| | | | Homo sapiens, ribosomal protein L6, clone | |
| | | | MGC: 1635 IMAGE: 2823733, mRNA, | |
| | | | complete cds | |
| 1184 | 2496.K15.GZ43_364338 | NM_024711 | gi\|13376008\|ref\|NM_024711.1 Homo | 1.1E−28 |
| | | | sapiens hypothetical protein FLJ22690 | |
| | | | (FLJ22690), mRNA | |
| 1192 | 2497.E09.GZ43_364572 | AF284421 | gi\|15088516\|gb\|AF284421.1AF284421 | 4.1E−158 |
| | | | Homo sapiens complement factor MASP-3 | |
| | | | mRNA, complete cds | |
| 1195 | 2497.J05.GZ43_364688 | Z56298 | gi\|1027529\|emb\|Z56298.1HS10C4R | 2.5E−42 |
| | | | H. sapiens CpG island DNA genomic Mse1 | |
| | | | fragment, clone 10c4, reverse read | |
| | | | cpg10c4.rt1a | |
| 1199 | 2497.L05.GZ43_364736 | AK023448 | gi\|10435386\|dbj\|AK023448.1AK023448 | 0 |
| | | | Homo sapiens cDNA FLJ13386 fis, clone | |
| | | | PLACE1001104, weakly similar to | |
| | | | MYOSIN HEAVY CHAIN, NON-MU | |
| 1207 | 2562.B09.GZ43_375496 | M64241 | gi\|190813\|gb\|M64241.1HUMQM Human | 3.2E−52 |
| | | | Wilm's tumor-related protein (QM) mRNA, | |
| | | | complete cds | |
| 1210 | 2562.I01.GZ43_375656 | AF083247 | gi\|5106788\|gb\|AF083247.1AF083247 Homo | 2.4E−48 |
| | | | sapiens MDG1 mRNA, complete cds | |
| 1214 | 2562.O01.GZ43_375800 | AF223389 | gi\|11066459\|gb\|AF223389.1AF223389 | 8.7E−57 |
| | | | Homo sapiens PCGEM1 gene, non-coding | |
| | | | mRNA | |
| 1217 | 2562.H11.GZ43_375642 | AK023442 | gi\|10435378\|dbj\|AK023442.1AK023442 | 1.7E−64 |
| | | | Homo sapiens cDNA FLJ13380 fis, clone | |
| | | | PLACE1001007 | |
| 1218 | 2562.B24.GZ43_375511 | AF287932 | gi\|12656321\|gb\|AF287932.1AF287932 | 1.8E−31 |
| | | | Rayleya bahiensis NADH dehydrogenase | |
| | | | subunit F (ndhF) gene, partial cds; | |
| | | | chloroplast gene for ch1 | |
| 1229 | 2498.A02.GZ43_364853 | AY031766 | gi\|13738569\|gb\|AY031766.1 HIV-1 isolate | 1.3E−29 |
| | | | NC5203-1999 from USA pol polyprotein | |
| | | | (pol) gene, partial cds | |
| 1230 | 2498.A19.GZ43_364870 | AL122114 | gi\|6102936\|emb\|AL122114.1HSM801274 | 1E−59 |
| | | | Homo sapiens mRNA; cDNA | |
| | | | DKFZp434K0221 (from clone | |
| | | | DKFZp434K0221); partial cds | |
| 1235 | 2498.G15.GZ43_365010 | M86752 | gi\|184564\|gb\|M86752.1HUMIEF Human | 3.4E−54 |
| | | | transformation-sensitive protein (IEF SSP | |
| | | | 3521) mRNA, complete cds | |
| 1238 | 2498.I17.GZ43_365060 | AJ335654 | gi\|15880072\|emb\|AJ335654.1HSA335654 | 4.3E−41 |
| | | | Homo sapiens genomic sequence | |
| | | | surrounding NotI site, clone NR5-IJ21R | |
| 1239 | 2498.K20.GZ43_365111 | X15940 | gi\|36129\|emb\|X15940.1HSRPL31 Human | 1.7E−25 |
| | | | mRNA for ribosomal protein L31 | |
| 1240 | 2498.M19.GZ43_365158 | AF203815 | gi\|6979641\|gb\|AF203815.1AF203815 Homo | 4E−47 |
| | | | sapiens alpha gene sequence | |
| 1242 | 2498.P07.GZ43_365218 | AF410975 | gi\|15553753\|gb\|AF410975.1AF410975 | 3.5E−29 |
| | | | Measles virus genotype D4 strain | |
| | | | MVi/Montreal.CAN/12.89 hemagglutinin | |
| | | | gene, complete cds | |
| 1244 | 2507.C03.GZ43_366992 | NM_025080 | gi\|13376633\|ref\|NM _025080.1 Homo | 1E−232 |
| | | | sapiens hypothetical protein FLJ22316 | |
| | | | (FLJ22316), mRNA | |
| 1259 | 2511.J18.GZ43_369643 | M81806 | gi\|184406\|gb\|M81806.1HUMHSKPQZ7 | 4.7E−34 |
| | | | Human housekeeping (Q1Z 7F5) gene, | |
| | | | exons 2 through 7, complete cds | |
| 1261 | 2499.A22.GZ43_365257 | AK024860 | gi\|10437268\|dbj\|AK024860.1AK024860 | 6.4E−49 |
| | | | Homo sapiens cDNA: FLJ21207 fis, clone | |
| | | | COL00362 | |
| 1263 | 2499.C09.GZ43_365292 | AJ330464 | gi\|15874882\|emb\|AJ330464.1HSA330464 | 3.3E−100 |
| | | | Homo sapiens genomic sequence | |
| | | | surrounding NotI site, clone NR1-IL7C | |
| 1268 | Clu1009284.1 | AF026853 | gi\|3882436\|gb\|AF026853.1HSHADHSC 1 | 1.3E−30 |
| | | | Homo sapiens mitochondrial short-chain L-3 | |
| | | | hydroxyacyl-CoA dehydrogenase | |
| | | | (HADHSC) gene, nuclear | |
| 1269 | Clu1022935.2 | AL590711.7 | gi\|16304966\|emb\|AL590711.7AL590711 | 3.9E−118 |
| | | | Human DNA sequence from clone RP11- | |
| | | | 284O18 on chromosome 9, complete | |
| | | | sequence [Homo sapiens] | |
| 1270 | Clu1037152.1 | M87652 | gi\|182743\|gb\|M87652.1HUMFPRPR | 1.1E−21 |
| | | | Human formylpeptide receptor gene, | |
| | | | promoter region | |
| 1271 | Clu13903.1 | AK026618 | gi\|10439509\|dbj\|AK026618.1AK026618 | 1.5E−293 |
| | | | Homo sapiens cDNA: FLJ22965 fis, clone | |
| | | | KAT10418 | |
| 1272 | Clu139979.2 | AB056828 | gi\|13365953\|dbj\|AB056828.1AB056828 | 1.4E−33 |
| | | | Macaca fascicularis brain cDNA clone: QfLA- | |
| | | | 13447, full insert sequence | |
| 1274 | Clu187860.2 | AL050204 | gi\|4884443\|emb\|AL050204.1HSM800501 | 4.7E−33 |
| | | | Homo sapiens mRNA; cDNA | |
| | | | DKFZp586F1223 (from clone | |
| | | | DKFZp586F1223) | |
| 1275 | Clu189993.1 | AB030001 | gi\|7416074\|dbj\|AB030001.1AB030001 | 9.6E−87 |
| | | | Homo sapiens gene for SGRF, complete cds | |
| 1276 | Clu20975.1 | AF039687 | gi\|3170173\|gb\|AF039687.1AF039687 Homo | 2.7E−190 |
| | | | sapiens antigen NY-CO-1 (NY-CO-1) | |
| | | | mRNA, complete cds | |
| 1278 | Clu218833.1 | AF223389 | gi\|11066459\|gb\|AF223389.1AF223389 | 1E−139 |
| | | | Homo sapiens PCGEM1 gene, non-coding | |
| | | | mRNA | |
| 1279 | Clu244504.2 | Z59663 | gi\|1031576\|emb\|Z59663.1HS168F9F | 7.5E−22 |
| | | | H. sapiens CpG island DNA genomic Mse1 | |
| | | | fragment, clone 168f9, forward read | |
| | | | cpg168f9.ft1a | |
| 1281 | Clu376516.1 | AK018003 | gi\|12857525\|dbj\|AK018003.1AK018003 | 1.7E−63 |
| | | | Mus musculus adult male thymus cDNA, | |
| | | | RIKEN full-length enriched library, | |
| | | | clone: 5830450H20, full | |
| 1282 | Clu376630.1 | U93571 | gi\|2072968\|gb\|U93571.1HSU93571 Human | 8.7E−291 |
| | | | L1 element L1.24 p40 gene, complete cds | |
| 1283 | Clu377044.2 | AK024860 | gi\|10437268\|dbj\|AK024860.1AK024860 | 1.6E−49 |
| | | | Homo sapiens cDNA: FLJ21207 fis, clone | |
| | | | COL00362 | |
| 1284 | Clu379689.1 | BC007110 | gi\|13937991\|gb\|BC007110.1BC007110 | 0 |
| | | | Homo sapiens, clone MGC: 14768 | |
| | | | IMAGE: 4291902, mRNA, complete cds | |
| 1286 | Clu387530.4 | AK009770 | gi\|12844769\|dbj\|AK009770.1AK009770 | 1.5E−80 |
| | | | Mus musculus adult male tongue cDNA, | |
| | | | RIKEN full-length enriched library, | |
| | | | clone: 2310043C14, full | |
| 1287 | Clu388450.2 | AK023448 | gi\|10435386\|dbj\|AK023448.1AK023448 | 0 |
| | | | Homo sapiens cDNA FLJ13386 fis, clone | |
| | | | PLACE1001104, weakly similar to | |
| | | | MYOSIN HEAVY CHAIN, NON-MU | |
| 1288 | Clu396325.1 | Z78727 | gi\|1508005\|emb\|Z78727.1HSPA15B9 | 1.2E−38 |
| | | | H. sapiens flow-sorted chromosome 6 | |
| | | | HindIII fragment, SC6pA15B9 | |
| 1291 | Clu400258.1 | AB038971 | gi\|12862672\|dbj\|AB038971.1AB038965S7 | 4E−74 |
| | | | Homo sapiens CFLAR gene, exon 10, exon | |
| | | | 11 | |
| 1293 | Clu402591.3 | AF170811 | gi\|6715105\|gb\|AF170811.1AF170811 Homo | 7E−26 |
| | | | sapiens CaBP2 (CABP2) gene, complete cds | |
| 1295 | Clu404081.2 | AK011443 | gi\|12847570\|dbj\|AK011443.1AK011443 | 5E−153 |
| | | | Mus musculus 10 days embryo cDNA, | |
| | | | RIKEN full-length enriched library, | |
| | | | clone: 2610018B07, full ins | |
| 1297 | Clu41346.1 | AB042029 | gi\|16326128\|dbj\|AB042029.1AB042029 | 0 |
| | | | Homo sapiens DEPC-1 mRNA for prostate | |
| | | | cancer antigen-1, complete cds | |
| 1299 | Clu416124.1 | AK000293 | gi\|7020278\|dbj\|AK000293.1AK000293 | 3.3E−34 |
| | | | Homo sapiens cDNA FLJ20286 fis, clone | |
| | | | HEP04358 | |
| 1300 | Clu417672.1 | AK027667 | gi\|14042514\|dbj\|AK027667.1AK027667 | 1.6E−183 |
| | | | Homo sapiens cDNA FLJ14761 fis, clone | |
| | | | NT2RP3003302 | |
| 1301 | Clu423664.1 | AF287270 | gi\|9844925\|gb\|AF287270.1AF287270 Homo | 6.3E−34 |
| | | | sapiens mucolipin (MCOLN1) gene, | |
| | | | complete cds | |
| 1303 | Clu442923.3 | BC014256 | gi\|15559816\|gb\|BC014256.1BC014256 | 1.5E−236 |
| | | | Homo sapiens, Similar to guanine nucleotide | |
| | | | binding protein (G protein), beta | |
| | | | polypeptide 2-like | |
| 1304 | Clu446975.1 | AL022342.6 | gi\|7159715\|emb\|AL022342.6HS29M10 | 1.8E−74 |
| | | | Human DNA sequence from clone RP1- | |
| | | | 29M10 on chromosome 20, complete | |
| | | | sequence [Homo sapiens] | |
| 1305 | Clu449839.2 | BC001607 | gi\|12804410\|gb\|BC001607.1BC001607 | 1.9E−27 |
| | | | Homo sapiens, clone IMAGE: 3543874, | |
| | | | mRNA, partial cds | |
| 1306 | Clu449889.1 | S45332 | gi\|255496\|gb\|S45332.1S45332 | 8E−101 |
| | | | erythropoietin receptor [human, placental, | |
| | | | Genomic, 8647 nt] | |
| 1307 | Clu451707.2 | AJ004862 | gi\|4038586\|emb\|AJ004862.1HSAJ4862 | 4.7E−49 |
| | | | Homo sapiens partial MUC5B gene, exon 1-29 | |
| 1308 | Clu454509.3 | AK022973 | gi\|10434673\|dbj\|AK022973.1AK022973 | 1.7E−285 |
| | | | Homo sapiens cDNA FLJ12911 fis, clone | |
| | | | NT2RP2004425, highly similar to Mus | |
| | | | musculus axotrophin mR | |
| 1310 | Clu455862.1 | AK023951 | gi\|10436049\|dbj\|AK023951.1AK023951 | 3.3E−27 |
| | | | Homo sapiens cDNA FLJ13889 fis, clone | |
| | | | THYRO1001595 | |
| 1311 | Clu460493.1 | AK012865 | gi\|12849888\|dbj\|AK012865.1AK012865 | 1.7E−57 |
| | | | Mus musculus 10, 11 days embryo cDNA, | |
| | | | RIKEN full-length enriched library, | |
| | | | clone: 2810036K01, full | |
| 1314 | Clu470032.1 | AF223389 | gi\|11066459\|gb\|AF223389.1AF223389 | 1.2E−116 |
| | | | Homo sapiens PCGEM1 gene, non-coding | |
| | | | mRNA | |
| 1317 | Clu477271.1 | BC007307 | gi\|13938350\|gb\|BC007307.1BC007307 | 4.6E−56 |
| | | | Homo sapiens, Similar to zinc finger protein | |
| | | | 268, clone IMAGE: 3352268, mRNA, partial | |
| | | | cds | |
| 1318 | Clu480410.1 | AK000713 | gi\|7020973\|dbj\|AK000713.1AK000713 | 0 |
| | | | Homo sapiens cDNA FLJ20706 fis, clone | |
| | | | KAIA1273 | |
| 1320 | Clu497138.1 | AF270579 | gi\|9755121\|gb\|AF270579.1AF270579 Homo | 3.8E−29 |
| | | | sapiens clone 18ptel 481c6 sequence | |
| 1321 | Clu498886.1 | U49973 | gi\|2226003\|gb\|U49973.1HSU49973 Human | 1.4E−24 |
| | | | Tigger1 transposable element, complete | |
| | | | consensus sequence | |
| 1323 | Clu5013.2 | BC007458 | gi\|13938610\|gb\|BC007458.1BC007458 | 0 |
| | | | Homo sapiens, clone MGC: 12217 | |
| | | | IMAGE: 3828631, mRNA, complete cds | |
| 1324 | Clu5105.2 | AL512712 | gi\|12224956\|emb\|AL512712.1HSM802915 | 0 |
| | | | Homo sapiens mRNA; cDNA | |
| | | | DKFZp761J139 (from clone | |
| | | | DKFZp761J139) | |
| 1325 | Clu510539.2 | AK023812 | gi\|10435860\|dbj\|AK023812.1AK023812 | 1.4E−32 |
| | | | Homo sapiens cDNA FLJ13750 fis, clone | |
| | | | PLACE3000331 | |
| 1326 | Clu514044.1 | AJ403947 | gi\|14270388\|emb\|AJ403947.1HSA403947 | 4.4E−295 |
| | | | Homo sapiens partial SLC22A3 gene for | |
| | | | organic cation transporter 3, exon 2 | |
| 1329 | Clu520370.1 | AF093016 | gi\|5579305\|gb\|AF093016.1AF093016 Homo | 7.3E−67 |
| | | | sapiens 22k48 gene, 5′UTR | |
| 1330 | Clu524917.1 | AL1573620 | gi\|15028613\|emb\|AL157362.10AL157362 | 4.9E−23 |
| | | | Human DNA sequence from clone RP11- | |
| | | | 142D16 on chromosome 13q14.3-21.31, | |
| | | | complete sequence [Homo | |
| 1331 | Clu528957.1 | AB060919 | gi\|13874604\|dbj\|AB060919.1AB060919 | 1.5E−31 |
| | | | Macaca fascicularis brain cDNA clone: QtrA- | |
| | | | 14728, full insert sequence | |
| 1334 | Clu540142.2 | AJ005821 | gi\|3123571\|emb\|AJ005821.1HSA5821 | 3.5E−36 |
| | | | Homo sapiens mRNA for X-like 1 protein | |
| 1335 | Clu540379.2 | AF088011 | gi\|3523217\|gb\|AF088011.1HUMYY75G10 | 2.4E−49 |
| | | | Homo sapiens full length insert cDNA clone | |
| | | | YY75G10 | |
| 1336 | Clu549507.1 | U14571 | gi\|551540\|gb\|U14571.1HSU14571***ALU | 1.6E−48 |
| | | | WARNING: Human Alu-Sc subfamily | |
| | | | consensus sequence | |
| 1339 | Clu556827.3 | AB038163 | gi\|10280537\|dbj\|AB038163.1AB038163 | 9.7E−22 |
| | | | Homo sapiens NDUFV3 gene for | |
| | | | mitochondrial NADH-Ubiquinone | |
| | | | oxidoreductase, complete cds | |
| 1340 | Clu558569.2 | AF061258 | gi\|3108092\|gb\|AF061258.1AF061258 Homo | 1E−300 |
| | | | sapiens LIM protein mRNA, complete cds | |
| 1343 | Clu570804.1 | AK023843 | gi\|10435902\|dbj\|AK023843.1AK023843 | 4.4E−42 |
| | | | Homo sapiens cDNA FLJ13781 fis, clone | |
| | | | PLACE4000465 | |
| 1344 | Clu572170.2 | U18271 | gi\|885681\|gb\|U18271.1HSTMPO6 Human | 4.9E−57 |
| | | | thymopoietin (TMPO) gene, partial exon 6, | |
| | | | complete exon 7, partial exon 8, and partial | |
| | | | cds for t | |
| 1346 | Clu587168.1 | AJ276804 | gi\|10803412\|emb\|AJ276804.1HSA276804 | 5.8E−69 |
| | | | Homo sapiens mRNA for protocadherin | |
| | | | (PCDHX gene) | |
| 1347 | Clu588996.1 | U73166 | gi\|1613889\|gb\|U73166.1U73166 Homo | 9.3E−22 |
| | | | sapiens cosmid clone LUCA15 from 3p21.3, | |
| | | | complete sequence | |
| 1349 | Clu598388.1 | AF327178 | gi\|11878341\|gb\|AF327178.1AF327178 | 1.1E−26 |
| | | | Homo sapiens clone 20ptel_cA35_21t7 | |
| | | | sequence | |
| 1350 | Clu604822.2 | AB063021 | gi\|14388457\|dbj\|AB063021.1AB063021 | 2.6E−65 |
| | | | Macaca fascicularis brain cDNA | |
| | | | clone: QmoA-11389, full insert sequence | |
| 1353 | Clu627263.1 | AK021759 | gi\|10433005\|dbj\|AK021759.1AK021759 | 5.7E−30 |
| | | | Homo sapiens cDNA FLJ11697 fis, clone | |
| | | | HEMBA1005035 | |
| 1356 | Clu641662.2 | AL1576971 | gi\|11121002\|emb\|AL157697.11AL157697 | 7E−84 |
| | | | Human DNA sequence from clone RP5- | |
| | | | 1092C14 on chromosome 6, complete | |
| | | | sequence [Homo sapiens] | |
| 1358 | Clu6712.1 | AK024029 | gi\|10436287\|dbj\|AK024029.1AK024029 | 0 |
| | | | Homo sapiens cDNA FLJ13967 fis, clone | |
| | | | Y79AA1001402, weakly similar to Homo | |
| | | | sapiens paraneoplasti | |
| 1361 | Clu685244.2 | S56773 | gi\|298606\|gb\|S56773.1S56773 putative | 1.1E−35 |
| | | | serine-threonine protenine kinase {3′ UTR, | |
| | | | Alu repeats} [human, Genomic 1470 nt] | |
| 1362 | Clu691653.1 | D28126 | gi\|559316\|dbj\|D28126.1HUMATPSAS | 6.3E−37 |
| | | | Human gene for ATP synthase alpha | |
| | | | subunit, complete cds (exon 1 to 12) | |
| 1367 | Clu709796.2 | AB070013 | gi\|15207866\|dbj\|AB070013.1AB070013 | 8.4E−118 |
| | | | Macaca fascicularis testis cDNA clone: QtsA- | |
| | | | 11243, full insert sequence | |
| 1369 | Clu727966.1 | AF271388 | gi\|8515842\|gb\|AF271388.1AF271388 Homo | 0 |
| | | | sapiens CMP-N-acetylneuraminic acid | |
| | | | synthase mRNA, complete cds | |
| 1372 | Clu756337.1 | BC004923 | gi\|13436241\|gb\|BC004923.1BC004923 | 4.1E−250 |
| | | | Homo sapiens, clone IMAGE: 3605104, | |
| | | | mRNA, partial cds | |
| 1376 | Clu823296.3 | AK023179 | gi\|10434987\|dbj\|AK023179.1AK023179 | 6.4E−33 |
| | | | Homo sapiens cDNA FLJ13117 fis, clone | |
| | | | NT2RP3002660 | |
| 1377 | Clu830453.2 | AK027301 | gi\|14041890\|dbj\|AK027301.1AK027301 | 0 |
| | | | Homo sapiens cDNA FLJ14395 fis, clone | |
| | | | HEMBA1003250, weakly similar to | |
| | | | PROTEIN KINASE APK1A (EC 2 | |
| 1378 | Clu839006.1 | AB023199 | gi\|4589607\|dbj\|AB023199.1AB023199 | 3.3E−51 |
| | | | Homo sapiens mRNA for KIAA0982 | |
| | | | protein, complete cds | |
| 1379 | Clu847088.1 | AL078632.6 | gi\|6002309\|emb\|AL078632.6HSA255N20 | 4.2E−40 |
| | | | Human DNA sequence from clone 255N20 | |
| | | | on chromosome 22, complete sequence | |
| | | | [Homo sapiens] | |
| 1380 | Clu853371.2 | S79349 | gi\|1110571\|gb\|S79349.1S79349 Homo | 1.6E−48 |
| | | | sapiens type 1 iodothyronine deiodinase | |
| | | | (hdiol) gene, partial cds | |
| 1381 | Clu88462.1 | AF026855 | gi\|3882438\|gb\|AF026855.1HSHADHSC 3 | 1.1E−65 |
| | | | Homo sapiens mitochondrial short-chain L-3 | |
| | | | hydroxyacyl-CoA dehydrogenase | |
| | | | (HADHSC) gene, nuclear | |
| 1382 | Clu935908.2 | AK025271 | gi\|10437753\|dbj\|AK025271.1AK025271 | 8.2E−54 |
| | | | Homo sapiens cDNA: FLJ21618 fis, clone | |
| | | | COL07487 | |
| 1386 | DTT00087024.1 | AF036235 | gi\|2695679\|gb\|AF036235.1AF036235 | 0 |
| | | | Gorilla gorilla L1 retrotransposon L1Gg-1A, | |
| | | | complete sequence | |
| 1387 | DTT00089020.1 | AF324172 | gi\|12958747\|gb\|AF324172.1AF324172 | 1.1E−142 |
| | | | Dictyophora indusiata strain ASI 32001 | |
| | | | internal transcribed spacer 1, partial | |
| | | | sequence; 5.8S ribo | |
| 1388 | DTT00171014.1 | AB050477 | gi\|11034759\|dbj\|AB050477.1AB050477 | 0 |
| | | | Homo sapiens NIBAN mRNA, complete cds | |
| 1389 | DTT00514029.1 | BC001978 | gi\|12805042\|gb\|BC001978.1BC001978 | 6E−284 |
| | | | Homo sapiens, clone IMAGE: 3461487, | |
| | | | mRNA, partial cds | |
| 1390 | DTT00740010.1 | AF216292 | gi\|7229461\|gb\|AF216292.1AF216292 Homo | 9.5E−229 |
| | | | sapiens endoplasmic reticulum lumenal | |
| | | | Ca2+ binding protein grp78 mRNA, | |
| | | | complete cds | |
| 1391 | DTT00945030.1 | AL117237 | gi\|5834563\|emb\|AL117237.1HS328E191 | 0 |
| | | | Novel human gene mapping to chomosome 1 | |
| 1394 | DTT01315010.1 | X16983 | gi\|33945\|emb\|X16983.1HSINTAL4 Human | 0 |
| | | | mRNA for integrin alpha-4 subunit | |
| 1395 | DTT01503016.1 | AK025473 | gi\|10437996\|dbj\|AK025473.1AK025473 | 0 |
| | | | Homo sapiens cDNA: FLJ21820 fis, clone | |
| | | | HEP01232 | |
| 1396 | DTT01555018.1 | AE007613 | gi\|15023874\|gb\|AE007613.1AE007613 | 0 |
| | | | Clostridium acetobutylicum ATCC824 | |
| | | | section 101 of 356 of the complete genome | |
| 1397 | DTT01685047.1 | M54985 | gi\|177005\|gb\|M54985.1GIBBGLOETA | 6.8E−107 |
| | | | H.lar psi-eta beta-like globin pseudogene, | |
| | | | exon 1,2,3 | |
| 1398 | DTT01764019.1 | AF307053 | gi\|12018057\|gb\|AF307053.1AF307053 | 0 |
| | | | Thermococcus litoralis sugar kinase, | |
| | | | trehalose/maltose binding protein (malE), | |
| | | | trehalose/maltose | |
| 1401 | DTT02367007.1 | AK001580 | gi\|7022920\|dbj\|AK001580.1AK001580 | 0 |
| | | | Homo sapiens cDNA FLJ10718 fis, clone | |
| | | | NT2RP3001096, weakly similar to Rattus | |
| | | | norvegicus leprecan | |
| 1402 | DTT02671007.1 | AF384048 | gi\|14488027\|gb\|AF384048.1AF384048 | 1.8E−170 |
| | | | Homo sapiens interferon kappa precursor | |
| | | | gene, complete cds | |
| 1403 | DTT02737017.1 | AF182418 | gi\|10197635\|gb\|AF182418.1AF182418 | 9E−207 |
| | | | Homo sapiens MDS017 (MDS017) mRNA, | |
| | | | complete cds | |
| 1404 | DTT02850005.1 | AK011295 | gi\|12847322\|dbj\|AK011295.1AK011295 | 2.5E−141 |
| | | | Mus musculus 10 days embryo cDNA, | |
| | | | RIKEN full-length enriched library, | |
| | | | clone: 2610002L04, full ins | |
| 1406 | DTT03037029.1 | AE006916 | gi\|13879055\|gb\|AE006916.1AE006916 | 2.1E−129 |
| | | | Mycobacterium tuberculosis CDC1551, | |
| | | | section 2 of 280 of the complete genome | |
| 1407 | DTT03150008.1 | M83822 | gi\|1580780\|gb\|M83822.1HUMCDC4REL | 0 |
| | | | Human beige-like protein (BGL) mRNA, | |
| | | | partial cds | |
| 1408 | DTT03367008.1 | NM_012090.2 | gi\|15011903\|ref\|NM_012090.2 Homo | 0 |
| | | | sapiens actin cross-linking factor (ACF7), | |
| | | | transcript variant 1, mRNA | |
| 1411 | DTT03913023.1 | AK018110 | gi\|12857675\|dbj\|AK018110.1AK018110 | 2E−214 |
| | | | Mus musculus adult male medulla oblongata | |
| | | | cDNA, RIKEN full-length enriched library, | |
| | | | clone: 633040 | |
| 1412 | DTT03978010.1 | BC015529 | gi\|15930193\|gb\|BC015529.1BC015529 | 0 |
| | | | Homo sapiens, Similar to ribose 5-phosphate | |
| | | | isomerase A, clone MGC: 9441 | |
| | | | IMAGE: 3904718, mRNA, comp | |
| 1413 | DTT04070014.1 | L43411 | gi\|893273\|gb\|L43411.1HUM25DC1Z Homo | 4E−102 |
| | | | sapiens (subclone 5_g5 from P1 H25) DNA | |
| | | | sequence | |
| 1414 | DTT04084010.1 | AF259790 | gi\|12240019\|gb\|AF259790.1AF259790 | 2.2E−288 |
| | | | Desulfitobacterium sp. PCE-1 o- | |
| | | | chlorophenol reductive dehalogenase (cprA) | |
| | | | gene, complete cds | |
| 1415 | DTT04160007.1 | AF338299 | gi\|12958808\|gb\|AF338299.1AF338299 | 1.4E−181 |
| | | | Amazona ochrocephala auropalliata | |
| | | | mitochondrial control region 1, partial | |
| | | | sequence | |
| 1417 | DTT04378009.1 | AF102129 | gi\|5922722\|gb\|AF102129.1AF102129 Rattus | 4.7E−146 |
| | | | norvegicus KPL2 (Kpl2) mRNA, complete | |
| | | | cds | |
| 1418 | DTT04403013.1 | AE007580 | gi\|15023517\|gb\|AE007580.1AE007580 | 1.5E−199 |
| | | | Clostridium acetobutylicum ATCC824 | |
| | | | section 68 of 356 of the complete genome | |
| 1420 | DTT04660017.1 | NM_025079 | gi\|13376631\|ref\|NM_025079.1 Homo | 0 |
| | | | sapiens hypothetical protein FLJ23231 | |
| | | | (FLJ23231), mRNA | |
| 1421 | DTT04956054.1 | AF050179 | gi\|3319283\|gb\|AF050179.1AF050179 Homo | 0 |
| | | | sapiens CENP-C binding protein (DAXX) | |
| | | | mRNA, complete cds | |
| 1422 | DTT04970018.1 | AK015635 | gi\|12854041\|dbj\|AK015635.1AK015635 | 1.4E−84 |
| | | | Mus musculus adult male testis cDNA, | |
| | | | RIKEN full-length enriched library, | |
| | | | clone: 4930486L24, full | |
| 1424 | DTT05571010.1 | AB014533 | gi\|3327079\|dbj\|AB014533.1AB014533 | 1.8E−53 |
| | | | Homo sapiens mRNA for KIAA0633 | |
| | | | protein, partial cds | |
| 1426 | DTT05742029.1 | AF344987 | gi\|13448249\|gb\|AF344987.1AF344987 | 0 |
| | | | Hepatitis C virus isolate RDpostSC1c2 | |
| | | | polyprotein gene, partial cds | |
| 1427 | DTT06137030.1 | AY049285 | gi\|15146287\|gb\|AY049285.1 Arabidopsis | 2.2E−143 |
| | | | thaliana AT3g58570/F14P22_160 mRNA, | |
| | | | complete cds | |
| 1428 | DTT06161014.1 | AJ330465 | gi\|15874883\|emb\|AJ330465.1HSA330465 | 2.5E−28 |
| | | | Homo sapiens genomic sequence | |
| | | | surrounding NotI site, clone NR1-IM15C | |
| 1429 | DTT06706019.1 | AF226787 | gi\|12407487\|gb\|AF226787.1AF226787 | 0 |
| | | | Syrrhopodon confertus ribulose-1,5- | |
| | | | bisphosphate carboxylase large subunit | |
| | | | (rbcL) gene, partial cd | |
| 1430 | DTT06837021.1 | AK000658 | gi\|7020892\|dbj\|AK000658.1AK000658 | 0 |
| | | | Homo sapiens cDNA FLJ20651 fis, clone | |
| | | | KAT01814 | |
| 1431 | DTT07040015.1 | AF047347 | gi\|3005557\|gb\|AF047347.1AF047347 Homo | 0 |
| | | | sapiens adaptor protein X11 alpha mRNA, | |
| | | | complete cds | |
| 1432 | DTT07088009.1 | AF326517 | gi\|15080738\|gb\|AF326517.1AF326517 | 0 |
| | | | Abies grandis pinene synthase gene, partial | |
| | | | cds | |
| 1433 | DTT07182014.1 | AB035187 | gi\|9955412\|dbj\|AB035187.1AB035187 | 3.1E−84 |
| | | | Homo sapiens RHD gene, intron 1, complete | |
| | | | sequence | |
| 1434 | DTT07405044.1 | AP002946 | gi\|16267254\|dbj\|AP002946.1AP002946 | 0 |
| | | | Mastacembelus favus mitochondrial DNA, | |
| | | | complete genome | |
| 1435 | DTT07408020.1 | AE008061 | gi\|15156405\|gb\|AE008061.1AE008061 | 6.9E−245 |
| | | | Agrobacterium tumefaciens strain C58 | |
| | | | circular chromosome, section 119 of 254 of | |
| | | | the complete sequ | |
| 1438 | DTT08005024.1 | U18270 | gi\|885679\|gb\|U18270.1HSTMPO4 Human | 5.1E−108 |
| | | | thymopoietin (TMPO) gene, exons 4 and 5, | |
| | | | and complete cds for thymopoietin alpha | |
| 1439 | DTT08098020.1 | AF387946 | gi\|15021617\|gb\|AF387946.1AF387946 | 0 |
| | | | Homo sapiens clone J102 melanocortin 1 | |
| | | | receptor gene, promoter region | |
| 1440 | DTT08167018.1 | NM_020642 | gi\|11034852\|ref\|NM_020642.1 Homo | 1E−183 |
| | | | sapiens chromosome 11 open reading frame | |
| | | | 17 (C11orf17), mRNA | |
| 1441 | DTT08249022.1 | M86752 | gi\|184564\|gb\|M86752.1HUMIEF Human | 0 |
| | | | transformation-sensitive protein (IEF SSP | |
| | | | 3521) mRNA, complete cds | |
| 1443 | DTT08514022.1 | AK001927 | gi\|7023494\|dbj\|AK001927.1AK001927 | 0 |
| | | | Homo sapiens cDNA FLJ11065 fis, clone | |
| | | | PLACE1004868, weakly similar to MALE | |
| | | | STERILITY PROTEIN 2 | |
| 1444 | DTT08527013.1 | AF271388 | gi\|8515842\|gb\|AF271388.1AF271388 Homo | 0 |
| | | | sapiens CMP-N-acetylneuraminic acid | |
| | | | synthase mRNA, complete cds | |
| 1445 | DTT08595020.1 | L07758 | gi\|177764\|gb\|L07758.1HUM56KDAPR | 0 |
| | | | Human IEF SSP 9502 mRNA, complete cds | |
| 1446 | DTT08711019.1 | D87930 | gi\|2443337\|dbj\|D87930.1D87930 Homo | 0 |
| | | | sapiens mRNA for myosin phosphatase | |
| | | | target subunit 1 (MYPT1) | |
| 1447 | DTT08773020.1 | X15187 | gi\|37260\|emb\|X15187.1HSTRA1 Human | 6.8E−298 |
| | | | tra1 mRNA for human homologue of murine | |
| | | | tumor rejection antigen gp96 | |
| 1448 | DTT08874012.1 | AK026442 | gi\|10439307\|dbj\|AK026442.1AK026442 | 0 |
| | | | Homo sapiens cDNA: FLJ22789 fis, clone | |
| | | | KAIA2171 | |
| 1449 | DTT09387018.1 | AF273672 | gi\|15186755\|gb\|AF273672.1AF273672 Mus | 0 |
| | | | musculus RANBP9 isoform 1 (Ranbp9) | |
| | | | mRNA, complete cds | |
| 1450 | DTT09396022.1 | AK000913 | gi\|7021874\|dbj\|AK000913.1AK000913 | 0 |
| | | | Homo sapiens cDNA FLJ10051 fis, clone | |
| | | | HEMBA1001281 | |
| 1452 | DTT09604016.1 | AK022722 | gi\|10434285\|dbj\|AK022722.1AK022722 | 2.2E−198 |
| | | | Homo sapiens cDNA FLJ12660 fis, clone | |
| | | | NT2RM4002174, moderately similar to | |
| | | | MRP PROTEIN | |
| 1454 | DTT09742009.1 | AF025409 | gi\|2582414\|gb\|AF025409.1AF025409 Homo | 0 |
| | | | sapiens zinc transporter 4 (ZNT4) mRNA, | |
| | | | complete cds | |
| 1455 | DTT09753017.1 | L03532 | gi\|187280\|gb\|L03532.1HUMM4PRO | 5.7E−58 |
| | | | Human M4 protein mRNA, complete cds | |
| 1456 | DTT09793019.1 | AK025125 | gi\|10437578\|dbj\|AK025125.1AK025125 | 0 |
| | | | Homo sapiens cDNA: FLJ21472 fis, clone | |
| | | | COL04936 | |
| 1457 | DTT09796028.1 | AF272390 | gi\|8705239\|gb\|AF272390.1AF272390 Homo | 0 |
| | | | sapiens myosin 5c (MYO5C) mRNA, | |
| | | | complete cds | |
| 1459 | DTT10360040.1 | AJ133798 | gi\|6453351\|emb\|AJ133798.1HSA133798 | 0 |
| | | | Homo sapiens mRNA for copine VI protein | |
| 1460 | DTT10539016.1 | AF152924 | gi\|5453323\|gb\|AF152924.1AF152924 Mus | 2.6E−70 |
| | | | musculus syntaxin4-interacting protein synip | |
| | | | mRNA, complete cds | |
| 1461 | DTT10564022.1 | AF322634 | gi\|12657820\|gb\|AF322634.1AF322634S1 | 0 |
| | | | Human herpesvirus 3 strain VZV-Iceland | |
| | | | glycoprotein B gene, complete cds | |
| 1462 | DTT10683041.1 | X69392 | gi\|36114\|emb\|X69392. 1HSRP26AA | 3E−250 |
| | | | H. sapiens mRNA for ribosomal protein L26 | |
| 1463 | DTT10819011.1 | U14568 | gi\|551537\|gb\|U14568.1HSU14568 ***ALU | 2.6E−93 |
| | | | WARNING: Human Alu-Sb subfamily | |
| | | | consensus sequence | |
| 1465 | DTT11479018.1 | AF309561 | gi\|10954043\|gb\|AF309561.1AF309561 | 0 |
| | | | Homo sapiens KRAB zinc finger protein | |
| | | | ZFQR mRNA, complete cds | |
| 1466 | DTT11483012.1 | U57053 | gi\|1616674\|gb\|U57053.1HSU57053 Human | 3.1E−245 |
| | | | unconventional myosin-ID (MYO1F) gene, | |
| | | | partial cds | |
| 1467 | DTT11548015.1 | X05332 | gi\|35740\|emb\|X05332.1HSPSAR Human | 0 |
| | | | mRNA for prostate specific antigen | |
| 1468 | DTT11730017.1 | U14572 | gi\|551541\|gb\|U14572.1HSU14572 ***ALU | 4.7E−90 |
| | | | WARNING: Human Alu-Sp subfamily | |
| | | | consensus sequence | |
| 1471 | DTT11902028.1 | AK001915 | gi\|7023475\|dbj\|AK001915.1AK001915 | 0 |
| | | | Homo sapiens cDNA FLJ11053 fis, clone | |
| | | | PLACE1004664 | |
| 1472 | DTT11915017.1 | U66062 | gi\|1724068\|gb\|U66062.1HSU66062 Human | 5.9E−111 |
| | | | glp-1 receptor gene, promoter region and | |
| | | | partial cds | |
| 1475 | DTT12201062.1 | M73791 | gi\|189265\|gb\|M73791.1HUMNOVGENE | 0 |
| | | | Human novel gene mRNA, complete cds | |
| 1476 | DTT12470020.1 | AK026618 | gi\|10439509\|dbj\|AK026618.1AK026618 | 0 |
| | | | Homo sapiens cDNA: FLJ22965 fis, clone | |
| | | | KAT10418 | |

TABLE 8

| SEQ ID | SEQ NAME | PFAM ID | PFAM DESCRIPTION | SCORE | START | END |
|---|---|---|---|---|---|---|
| 7 | 2504.C11.GZ43_365848 | PF00179 | Ubiquitin-conjugating | 92.64 | 4 | 159 |
| | | | enzyme | | | |
| 10 | 2504.E23.GZ43_365908 | PF01260 | AP endonuclease family 1 | 88.28 | 222 | 481 |
| 46 | 2505.G16.GZ43_366333 | PF02594 | Uncharacterized ACR, YggU | 77.64 | 263 | 495 |
| | | | family COG1872 | | | |
| 109 | 2510.N14.GZ43_369351 | PF02348 | Cytidylyltransferase | 187.84 | 357 | 675 |
| 126 | 2365.D10.GZ43_345308 | PF01018 | GTP1/OBG family | 96.12 | 50 | 507 |
| 134 | 2365.F24.GZ43_345370 | PF00160 | Cyclophilin type peptidyl- | 120.2 | 251 | 522 |
| | | | prolyl cis-trans isomerase | | | |
| 189 | 2366.L21.GZ43_345942 | PF00612 | IQ calmodulin-binding motif | 33.96 | 415 | 477 |
| | 2366.L21.GZ43_345942 | PF00063 | Myosin head (motor domain) | 207.12 | 8 | 369 |
| 259 | 2368.O03.GZ43_346717 | PF00160 | Cyclophilin type peptidyl- | 120.2 | 242 | 513 |
| | | | prolyl cis-trans isomerase | | | |
| 267 | 2535.C23.GZ43_370158 | PF02114 | Phosducin | 32 | 152 | 589 |
| 334 | 2537.D11.GZ43_370938 | PF00083 | Sugar (and other) transporter | 122.88 | 4 | 288 |
| 335 | 2537.D20.GZ43_370947 | PF00131 | Metallothionein | 48.56 | 563 | 665 |
| 349 | 2537.N12.GZ43_371179 | PF01352 | KRAB box | 123.24 | 313 | 498 |
| 363 | 2538.B03.GZ43_371266 | PF00160 | Cyclophilin type peptidyl- | 117.68 | 320 | 591 |
| | | | prolyl cis-trans isomerase | | | |
| 391 | 2554.A06.GZ43_375853 | PF03015 | Male sterility protein | 44.96 | 605 | 749 |
| 394 | 2554.A16.GZ43_375863 | PF02348 | Cytidylyltransferase | 195.48 | 397 | 650 |
| 405 | 2554.I10.GZ43_376049 | PF03041 | lef-2 | 31.88 | 479 | 536 |
| 419 | 2565.B15.GZ43_398171 | PF02271 | Ubiquinol-cytochrome C | 70.76 | 29 | 188 |
| | | | reductase complex 14 kD | | | |
| | | | subunit | | | |
| 422 | 2565.C17.GZ43_398204 | PF00089 | Trypsin | 45.28 | 5 | 110 |
| 482 | 2540.I17.GZ43_372216 | PF00023 | Ank repeat | 75.44 | 444 | 542 |
| 507 | 2541.L08.GZ43_372663 | PF00499 | NADH- | 54.72 | 89 | 237 |
| | | | ubiquinone/plastoquinone | | | |
| | | | oxidoreductase chain 6 | | | |
| 514 | 2506.C15.GZ43_366620 | PF00076 | RNA recognition motif. | 44.44 | 70 | 276 |
| | | | (a.k.a. RRM, RBD, or RNP | | | |
| | | | domain) | | | |
| 521 | 2506.G24.GZ43_366725 | PF00096 | Zinc finger, C2H2 type | 46.68 | 156 | 224 |
| 527 | 2506.J20.GZ43_366793 | PF00595 | PDZ domain (Also known as | 34.16 | 290 | 502 |
| | | | DHR or GLGF). | | | |
| 543 | 2542.D19.GZ43_372866 | PF00098 | Zinc knuckle | 46.68 | 224 | 276 |
| 563 | 2542.N21.GZ43_373108 | PF01545 | Cation efflux family | 42.24 | 191 | 325 |
| 569 | 2555.F16.GZ43_373295 | PF02348 | Cytidylyltransferase | 215.04 | 357 | 713 |
| 716 | 2560.H21.GZ43_375268 | PF00510 | Cytochrome c oxidase | 37.28 | 224 | 436 |
| | | | subunit III | | | |
| 721 | 2560.K10.GZ43_375329 | PF01018 | GTP1/OBG family | 104.56 | 50 | 573 |
| 759 | 2561.O17.GZ43_376584 | PF00826 | Ribosomal L10 | 79.88 | 46 | 180 |
| 766 | 2456.B12.GZ43_355864 | PF01545 | Cation efflux family | 34.16 | 102 | 236 |
| 771 | 2456.D04.GZ43_355904 | PF02114 | Phosducin | 30.52 | 139 | 576 |
| 813 | 2457.J23.GZ43_356451 | PF02594 | Uncharacterized ACR, YggU | 77.64 | 189 | 421 |
| | | | family COG1872 | | | |
| 818 | 2457.L21.GZ43_356497 | PF00023 | Ank repeat | 38 | 208 | 306 |
| 910 | 2464.L02.GZ43_357946 | PF00076 | RNA recognition motif. | 34.84 | 244 | 350 |
| | | | (a.k.a. RRM, RBD, or RNP | | | |
| | | | domain) | | | |
| 914 | 2464.N05.GZ43_357997 | PF00023 | Ank repeat | 128.28 | 491 | 589 |
| 935 | 2465.K20.GZ43_358324 | PF02594 | Uncharacterized ACR, YggU | 77.64 | 210 | 442 |
| | | | family COG1872 | | | |
| 952 | 2466.I08.GZ43_360281 | PF00012 | Hsp70 protein | 120.92 | 16 | 208 |
| 967 | 2467.D10.GZ43_360547 | PF00008 | EGF-like domain | 31.04 | 63 | 113 |
| 1002 | 2472.P22.GZ43_361231 | PF00499 | NADH- | 64.72 | 81 | 209 |
| | | | ubiquinone/plastoquinone | | | |
| | | | oxidoreductase chain 6 | | | |
| 1011 | 2473.I08.GZ43_361433 | PF00895 | ATP synthase protein 8 | 66.88 | 5 | 148 |
| 1039 | 2475.N08.GZ43_362321 | PF00804 | Syntaxin | 53.08 | 226 | 601 |
| 1051 | 2480.D13.GZ43_358588 | PF03025 | Papillomavirus E5 | 33.56 | 583 | 749 |
| 1065 | 2481.B06.GZ43_358917 | PF00098 | Zinc knuckle | 35.88 | 79 | 133 |
| 1100 | 2483.J07.GZ43_359878 | PF00142 | 4Fe-4S iron sulfur cluster | 32.8 | 211 | 288 |
| | | | binding proteins, NifH/frxC | | | |
| | | | family | | | |
| 1101 | 2483.K02.GZ43_359897 | PF00160 | Cyclophilin type peptidyl- | 117.52 | 244 | 516 |
| | | | prolyl cis-trans isomerase | | | |
| 1107 | 2488.B07.GZ43_362475 | PF01260 | AP endonuclease family 1 | 79.88 | 251 | 614 |
| 1128 | 2489.F09.GZ43_362957 | PF02348 | Cytidylyltransferase | 174.36 | 347 | 591 |
| 1183 | 2496.I06.GZ43_364281 | PF02790 | Cytochrome C oxidase | 45.8 | 131 | 242 |
| | | | subunit II, transmembrane | | | |
| | | | domain | | | |
| 1207 | 2562.B09.GZ43_375496 | PF00826 | Ribosomal L10 | 106.28 | 49 | 341 |
| 1216 | 2562.E14.GZ43_375573 | PF00023 | Ank repeat | 87.04 | 230 | 328 |
| 1225 | 2562.H18.GZ43_375649 | PF02594 | Uncharacterized ACR, YggU | 65.44 | 206 | 437 |
| | | | family COG1872 | | | |
| 1244 | 2507.C03.GZ43_366992 | PF00083 | Sugar (and other) transporter | 95.52 | 107 | 355 |
| 1267 | 2499.I09.GZ43_365436 | PF00160 | Cyclophilin type peptidyl- | 43.24 | 139 | 238 |
| | | | prolyl cis-trans isomerase | | | |

TABLE 9

| SEQ | PROTEIN SEQ | | | | | |
|---|---|---|---|---|---|---|
| ID | NAME | PFAM ID | PFAM DESCRIPTION | SCORE | START | END |
| 1481 | DTP00514038.1 | PF00587 | tRNA synthetase class II core | 33.42 | 1 | 116 |
| | | | domain (G, H, P, S and T) | | | |
| 1482 | DTP00740019.1 | PF00012 | Hsp70 protein | 948.22 | 27 | 564 |
| 1484 | DTP01169031.1 | PF00023 | Ank repeat | 159.66 | 82 | 114 |
| 1484 | DTP01169031.1 | PF00023 | Ank repeat | 159.66 | 181 | 213 |
| 1484 | DTP01169031.1 | PF00023 | Ank repeat | 159.66 | 148 | 180 |
| 1484 | DTP01169031.1 | PF00023 | Ank repeat | 159.66 | 115 | 147 |
| 1484 | DTP01169031.1 | PF00023 | Ank repeat | 159.66 | 82 | 114 |
| 1484 | DTP01169031.1 | PF00023 | Ank repeat | 159.66 | 49 | 81 |
| 1484 | DTP01169031.1 | PF00023 | Ank repeat | 159.66 | 16 | 48 |
| 1484 | DTP01169031.1 | PF00023 | Ank repeat | 159.66 | 181 | 213 |
| 1484 | DTP01169031.1 | PF00023 | Ank repeat | 159.66 | 115 | 147 |
| 1484 | DTP01169031.1 | PF00023 | Ank repeat | 159.66 | 49 | 81 |
| 1484 | DTP01169031.1 | PF00023 | Ank repeat | 159.66 | 16 | 48 |
| 1484 | DTP01169031.1 | PF00023 | Ank repeat | 159.66 | 148 | 180 |
| 1486 | DTP01315019.1 | PF01839 | FG-GAP repeat | 255.09 | 427 | 479 |
| 1486 | DTP01315019.1 | PF01839 | FG-GAP repeat | 255.09 | 49 | 111 |
| 1486 | DTP01315019.1 | PF01839 | FG-GAP repeat | 255.09 | 248 | 300 |
| 1486 | DTP01315019.1 | PF01839 | FG-GAP repeat | 255.09 | 303 | 362 |
| 1486 | DTP01315019.1 | PF01839 | FG-GAP repeat | 255.09 | 365 | 424 |
| 1495 | DTP02737026.1 | PF01423 | Sm protein | 31.6 | 19 | 66 |
| 1496 | DTP02850014.1 | PF00804 | Syntaxin | 156.59 | 1 | 292 |
| 1496 | DTP02850014.1 | PF00804 | Syntaxin | 156.59 | 1 | 292 |
| 1496 | DTP02850014.1 | PF00804 | Syntaxin | 156.59 | 1 | 292 |
| 1510 | DTP04403022.1 | PF00400 | WD domain, G-beta repeat | 35.93 | 80 | 116 |
| 1510 | DTP04403022.1 | PF00400 | WD domain, G-beta repeat | 35.93 | 38 | 74 |
| 1510 | DTP04403022.1 | PF00400 | WD domain, G-beta repeat | 35.93 | 1 | 33 |
| 1512 | DTP04660026.1 | PF00083 | Sugar (and other) transporter | 234.43 | 1 | 484 |
| 1512 | DTP04660026.1 | PF00083 | Sugar (and other) transporter | 234.43 | 1 | 484 |
| 1518 | DTP05742038.1 | PF01018 | GTP1/OBG family | 133.76 | 105 | 208 |
| 1518 | DTP05742038.1 | PF01018 | GTP1/OBG family | 133.76 | 7 | 97 |
| 1518 | DTP05742038.1 | PF01018 | GTP1/OBG family | 133.76 | 105 | 208 |
| 1518 | DTP05742038.1 | PF01018 | GTP1/OBG family | 133.76 | 7 | 97 |
| 1518 | DTP05742038.1 | PF01018 | GTP1/OBG family | 133.76 | 105 | 208 |
| 1518 | DTP05742038.1 | PF01018 | GTP1/OBG family | 133.76 | 7 | 97 |
| 1519 | DTP06137039.1 | PF02271 | Ubiquinol-cytochrome C | 141.38 | 4 | 154 |
| | | | reductase complex 14 kD | | | |
| | | | subunit | | | |
| 1521 | DTP06706028.1 | PF00054 | Laminin G domain | 63.34 | 56 | 178 |
| 1521 | DTP06706028.1 | PF00054 | Laminin G domain | 63.34 | 281 | 292 |
| 1523 | DTP07040024.1 | PF00640 | Phosphotyrosine interaction | 233.89 | 461 | 618 |
| | | | domain (PTB/PID). | | | |
| 1523 | DTP07040024.1 | PF00595 | PDZ domain (Also known as | 85.47 | 656 | 742 |
| | | | DHR or GLGF). | | | |
| 1532 | DTP08249031.1 | PF00515 | TPR Domain | 115 | 4 | 37 |
| 1532 | DTP08249031.1 | PF00515 | TPR Domain | 115 | 72 | 105 |
| 1532 | DTP08249031.1 | PF00515 | TPR Domain | 115 | 38 | 71 |
| 1532 | DTP08249031.1 | PF00515 | TPR Domain | 115 | 259 | 292 |
| 1532 | DTP08249031.1 | PF00515 | TPR Domain | 115 | 300 | 333 |
| 1532 | DTP08249031.1 | PF00515 | TPR Domain | 115 | 225 | 258 |
| 1535 | DTP08527022.1 | PF02348 | Cytidylyltransferase | 48.59 | 1 | 166 |
| 1535 | DTP08527022.1 | PF02348 | Cytidylyltransferase | 48.59 | 1 | 166 |
| 1535 | DTP08527022.1 | PF02348 | Cytidylyltransferase | 48.59 | 1 | 166 |
| 1535 | DTP08527022.1 | PF02348 | Cytidylyltransferase | 48.59 | 1 | 166 |
| 1536 | DTP08595029.1 | PF00400 | WD domain, G-beta repeat | 80.04 | 183 | 221 |
| 1536 | DTP08595029.1 | PF00400 | WD domain, G-beta repeat | 80.04 | 236 | 273 |
| 1536 | DTP08595029.1 | PF00400 | WD domain, G-beta repeat | 80.04 | 365 | 402 |
| 1536 | DTP08595029.1 | PF00400 | WD domain, G-beta repeat | 80.04 | 279 | 316 |
| 1536 | DTP08595029.1 | PF00400 | WD domain, G-beta repeat | 80.04 | 325 | 357 |
| 1537 | DTP08711028.1 | PF00023 | Ank repeat | 81.96 | 22 | 54 |
| 1537 | DTP08711028.1 | PF00023 | Ank repeat | 81.96 | 55 | 87 |
| 1538 | DTP08773029.1 | PF00183 | Hsp90 protein | 100.71 | 104 | 173 |
| 1540 | DTP09387027.1 | PF00069 | Protein kinase domain | 224.56 | 76 | 342 |
| 1545 | DTP09742018.1 | PF01545 | Cation efflux family | 368.71 | 114 | 418 |
| 1545 | DTP09742018.1 | PF01545 | Cation efflux family | 368.71 | 114 | 418 |
| 1548 | DTP09796037.1 | PF00612 | IQ calmodulin-binding motif | 87.63 | 879 | 899 |
| 1548 | DTP09796037.1 | PF00612 | IQ calmodulin-binding motif | 87.63 | 856 | 876 |
| 1548 | DTP09796037.1 | PF00612 | IQ calmodulin-binding motif | 87.63 | 831 | 851 |
| 1548 | DTP09796037.1 | PF00612 | IQ calmodulin-binding motif | 87.63 | 808 | 828 |
| 1548 | DTP09796037.1 | PF00612 | IQ calmodulin-binding motif | 87.63 | 780 | 800 |
| 1548 | DTP09796037.1 | PF00612 | IQ calmodulin-binding motif | 87.63 | 757 | 777 |
| 1548 | DTP09796037.1 | PF01843 | DIL domain | 125.23 | 1574 | 1679 |
| 1548 | DTP09796037.1 | PF00063 | Myosin head (motor domain) | 1228.24 | 69 | 741 |
| 1550 | DTP10360049.1 | PF00168 | C2 domain | 50.07 | 26 | 114 |
| 1550 | DTP10360049.1 | PF00168 | C2 domain | 50.07 | 228 | 315 |
| 1551 | DTP10539025.1 | PF00595 | PDZ domain (Also known as | 32.34 | 5 | 84 |
| | | | DHR or GLGF). | | | |
| 1553 | DTP10683050.1 | PF00467 | KOW motif | 89.22 | 49 | 107 |
| 1556 | DTP11479027.1 | PF00096 | Zinc finger, C2H2 type | 209.31 | 402 | 424 |
| 1556 | DTP11479027.1 | PF01352 | KRAB box | 134.58 | 8 | 70 |
| 1556 | DTP11479027.1 | PF00096 | Zinc finger, C2H2 type | 209.31 | 374 | 396 |
| 1556 | DTP11479027.1 | PF00096 | Zinc finger, C2H2 type | 209.31 | 346 | 368 |
| 1556 | DTP11479027.1 | PF00096 | Zinc finger, C2H2 type | 209.31 | 318 | 340 |
| 1556 | DTP11479027.1 | PF00096 | Zinc finger, C2H2 type | 209.31 | 290 | 312 |
| 1556 | DTP11479027.1 | PF00096 | Zinc finger, C2H2 type | 209.31 | 262 | 284 |
| 1556 | DTP11479027.1 | PF00096 | Zinc finger, C2H2 type | 209.31 | 234 | 256 |
| 1556 | DTP11479027.1 | PF00096 | Zinc finger, C2H2 type | 209.31 | 206 | 228 |
| 1557 | DTP11483021.1 | PF00063 | Myosin head (motor domain) | 339.24 | 117 | 271 |
| 1557 | DTP11483021.1 | PF00063 | Myosin head (motor domain) | 339.24 | 34 | 115 |
| 1558 | DTP11548024.1 | PF00089 | Trypsin | 272.53 | 25 | 253 |
| 1564 | DTP11966049.1 | PF00023 | Ank repeat | 165.68 | 49 | 81 |
| 1564 | DTP11966049.1 | PF00023 | Ank repeat | 165.68 | 148 | 180 |
| 1564 | DTP11966049.1 | PF00023 | Ank repeat | 165.68 | 181 | 214 |
| 1564 | DTP11966049.1 | PF00023 | Ank repeat | 165.68 | 148 | 180 |
| 1564 | DTP11966049.1 | PF00023 | Ank repeat | 165.68 | 115 | 147 |
| 1564 | DTP11966049.1 | PF00023 | Ank repeat | 165.68 | 82 | 114 |
| 1564 | DTP11966049.1 | PF00023 | Ank repeat | 165.68 | 49 | 81 |
| 1564 | DTP11966049.1 | PF00023 | Ank repeat | 165.68 | 181 | 214 |
| 1564 | DTP11966049.1 | PF00023 | Ank repeat | 165.68 | 181 | 214 |
| 1564 | DTP11966049.1 | PF00023 | Ank repeat | 165.68 | 16 | 48 |
| 1564 | DTP11966049.1 | PF00023 | Ank repeat | 165.68 | 115 | 147 |
| 1564 | DTP11966049.1 | PF00023 | Ank repeat | 165.68 | 82 | 114 |
| 1564 | DTP11966049.1 | PF00023 | Ank repeat | 165.68 | 16 | 48 |
| 1564 | DTP11966049.1 | PF00023 | Ank repeat | 165.68 | 148 | 180 |
| 1564 | DTP11966049.1 | PF00023 | Ank repeat | 165.68 | 115 | 147 |
| 1564 | DTP11966049.1 | PF00023 | Ank repeat | 165.68 | 82 | 114 |
| 1564 | DTP11966049.1 | PF00023 | Ank repeat | 165.68 | 49 | 81 |
| 1564 | DTP11966049.1 | PF00023 | Ank repeat | 165.68 | 16 | 48 |
| 1566 | DTP12201071.1 | PF00826 | Ribosomal L10 | 467.36 | 1 | 176 |
| 1566 | DTP12201071.1 | PF00826 | Ribosomal L10 | 467.36 | 1 | 176 |

| | | | | | | | | | Lymph | Reg | Dist | Dist | |
|---|---|---|---|---|---|---|---|---|---|---|---|---|---|
| | Path | | Anatom | | | Histo | | Lymph | Met | Lymph | Met & | Met | |
| Pt ID | ID | Grp | Loc | Size | Grade | Grade | Local Invasion | Met | Incid | Grade | Loc | Grade | Comment |
| 15 | 21 | III | Ascending | 4.0 | T3 | G2 | Extending into | Pos | 3/8 | N1 | Neg | MX | invasive |
| | | | colon | | | | subserosal | | | | | | adeno- |
| | | | | | | | adipose | | | | | | carcinoma, |
| | | | | | | | tissue | | | | | | moderately |
| | | | | | | | | | | | | | differentiated; |
| | | | | | | | | | | | | | focal perineural |
| | | | | | | | | | | | | | invasion is seen |
| 52 | 71 | II | Cecum | 9.0 | T3 | G3 | Invasion | Neg | 0/12 | N0 | Neg | M0 | Hyperplastic |
| | | | | | | | through | | | | | | polyp in |
| | | | | | | | muscularis | | | | | | appendix. |
| | | | | | | | propria, | | | | | | |
| | | | | | | | subserosal | | | | | | |
| | | | | | | | involvement; | | | | | | |
| | | | | | | | ileocec. valve | | | | | | |
| | | | | | | | involvement | | | | | | |
| 121 | 140 | II | Sigmoid | 6 | T4 | G2 | Invasion of | Neg | 0/34 | N0 | Neg | M0 | Perineural |
| | | | | | | | muscularis | | | | | | invasion; donut |
| | | | | | | | propria | | | | | | anastomosis |
| | | | | | | | into serosa, | | | | | | Neg. One |
| | | | | | | | involving | | | | | | tubulovillous |
| | | | | | | | submucosa of | | | | | | and one tubular |
| | | | | | | | urinary bladder | | | | | | adenoma with |
| | | | | | | | | | | | | | no high grade |
| | | | | | | | | | | | | | dysplasia. |
| 125 | 144 | II | Cecum | 6 | T3 | G2 | Invasion | Neg | 0/19 | N0 | Neg | M0 | patient history |
| | | | | | | | through | | | | | | of metastatic |
| | | | | | | | the muscularis | | | | | | melanoma |
| | | | | | | | propria into | | | | | | |
| | | | | | | | suserosal | | | | | | |
| | | | | | | | adipose | | | | | | |
| | | | | | | | tissue. | | | | | | |
| | | | | | | | Ileocecal | | | | | | |
| | | | | | | | junction. | | | | | | |
| 128 | 147 | III | Transverse | 5.0 | T3 | G2 | Invasion of | Pos | 1/5 | N1 | Neg | M0 | |
| | | | colon | | | | muscularis | | | | | | |
| | | | | | | | propria into | | | | | | |
| | | | | | | | percolonic fat | | | | | | |
| 130 | 149 | | Splenic | 5.5 | T3 | | through wall | Pos | 10/24 | N2 | Neg | M1 | |
| | | | flexure | | | | and into | | | | | | |
| | | | | | | | surrounding | | | | | | |
| | | | | | | | adipose tissue | | | | | | |
| 133 | 152 | II | Rectum | 5.0 | T3 | G2 | Invasion | Neg | 0/9 | N0 | Neg | M0 | Small separate |
| | | | | | | | through | | | | | | tubular |
| | | | | | | | muscularis | | | | | | adenoma |
| | | | | | | | propria into | | | | | | |
| | | | | | | | non- | | | | | | |
| | | | | | | | peritonealized | | | | | | |
| | | | | | | | pericolic tissue; | | | | | | |
| | | | | | | | gross | | | | | | |
| | | | | | | | configuration is | | | | | | |
| | | | | | | | annular. | | | | | | |
| 141 | 160 | IV | Cecum | 5.5 | T3 | G2 | Invasion of | Pos | 7/21 | N2 | Pos - | M1 | Perineural |
| | | | | | | | muscularis | | | | Liver | | invasion |
| | | | | | | | propria into | | | | | | identified |
| | | | | | | | pericolonic | | | | | | adjacent to |
| | | | | | | | adipose tissue, | | | | | | metastatic |
| | | | | | | | but not | | | | | | adeno- |
| | | | | | | | through serosa. | | | | | | carcinoma |
| | | | | | | | Arising from | | | | | | |
| | | | | | | | tubular adenoma. | | | | | | |
| 156 | 175 | III | Hepatic | 3.8 | T3 | G2 | Invasion | Pos | 2/13 | N1 | Neg | M0 | Separate |
| | | | flexure | | | | through | | | | | | tubolovillous |
| | | | | | | | mucsularis | | | | | | and tubular |
| | | | | | | | propria into | | | | | | adenomas |
| | | | | | | | subserosa/ | | | | | | |
| | | | | | | | pericolicadipose, | | | | | | |
| | | | | | | | no serosal | | | | | | |
| | | | | | | | involvement. | | | | | | |
| | | | | | | | Gross | | | | | | |
| | | | | | | | configuration | | | | | | |
| | | | | | | | annular. | | | | | | |
| 228 | 247 | III | Rectum | 5.8 | T3 | G2 to | Invasion | Pos | 1/8 | N1 | Neg | MX | Hyperplastic |
| | | | | | | G3 | through | | | | | | polyps |
| | | | | | | | muscularis | | | | | | |
| | | | | | | | propria | | | | | | |
| | | | | | | | to involve | | | | | | |
| | | | | | | | subserosal, | | | | | | |
| | | | | | | | perirectoal | | | | | | |
| | | | | | | | adipose, and | | | | | | |
| | | | | | | | serosa | | | | | | |
| 264 | 283 | II | Ascending | 5.5 | T3 | G2 | Invasion | Neg | 0/10 | N0 | Neg | M0 | Tubulovillous |
| | | | colon | | | | through | | | | | | adenoma with |
| | | | | | | | muscularis | | | | | | high grade |
| | | | | | | | propria into | | | | | | dysplasia |
| | | | | | | | subserosal | | | | | | |
| | | | | | | | adipose tissue. | | | | | | |
| 266 | 285 | III | Transverse | 9 | T3 | G2 | Invades | Neg | 0/15 | N1 | Pos- | MX | |
| | | | colon | | | | through | | | | Mesen- | | |
| | | | | | | | muscularis | | | | teric | | |
| | | | | | | | propria to | | | | deposit | | |
| | | | | | | | involve | | | | | | |
| | | | | | | | pericolonic | | | | | | |
| | | | | | | | adipose, | | | | | | |
| | | | | | | | extends to | | | | | | |
| | | | | | | | serosa. | | | | | | |
| 268 | 287 | I | Cecum | 6.5 | T2 | G2 | Invades full | Neg | 0/12 | N0 | Neg | M0 | |
| | | | | | | | thickness of | | | | | | |
| | | | | | | | muscularis | | | | | | |
| | | | | | | | propria, but | | | | | | |
| | | | | | | | mesenteric | | | | | | |
| | | | | | | | adipose | | | | | | |
| | | | | | | | free of | | | | | | |
| | | | | | | | malignancy | | | | | | |
| 278 | 297 | III | Rectum | 4 | T3 | G2 | Invasion into | Pos | 7/10 | N2 | Neg | M0 | Descending |
| | | | | | | | perirectal | | | | | | colon polyps, |
| | | | | | | | adipose | | | | | | no HGD or |
| | | | | | | | tissue. | | | | | | carcinoma |
| | | | | | | | | | | | | | identified.. |
| 296 | 315 | III | Cecum | 5.5 | T3 | G2 | Invasion | Pos | 2/12 | N1 | Neg | M0 | Tubulovillous |
| | | | | | | | through | | | | | | adenoma |
| | | | | | | | muscularis | | | | | | (2.0 cm) |
| | | | | | | | propria and | | | | | | with no |
| | | | | | | | invades pericolic | | | | | | high grade |
| | | | | | | | adipose tissue. | | | | | | dysplasia. Neg. |
| | | | | | | | Ileocecal | | | | | | liver biopsy. |
| | | | | | | | junction. | | | | | | |
| 339 | 358 | II | Recto- | 6 | T3 | G2 | Extends into | Neg | 0/6 | N0 | Neg | M0 | 1 hyperplastic |
| | | | sigmoid | | | | perirectal fat | | | | | | polyp identified |
| | | | | | | | but does not | | | | | | |
| | | | | | | | reach serosa | | | | | | |
| 341 | 360 | II | Ascending | 2 cm | T3 | G2 | Invasion | Neg | 0/4 | N0 | Neg | MX | |
| | | | colon | in- | | | through | | | | | | |
| | | | | vasive | | | muscularis | | | | | | |
| | | | | | | | propria to | | | | | | |
| | | | | | | | involve | | | | | | |
| | | | | | | | pericolonic fat. | | | | | | |
| | | | | | | | Arising from | | | | | | |
| | | | | | | | villous | | | | | | |
| | | | | | | | adenoma. | | | | | | |
| 356 | 375 | II | Sigmoid | 6.5 | T3 | G2 | Through colon | Neg | 0/4 | N0 | Neg | M0 | |
| | | | | | | | wall into | | | | | | |
| | | | | | | | subserosal | | | | | | |
| | | | | | | | adipose tissue. | | | | | | |
| | | | | | | | No serosal | | | | | | |
| | | | | | | | spread seen. | | | | | | |
| 360 | 412 | III | Ascending | 4.3 | T3 | G2 | Invasion thru | Pos | 1/5 | N1 | Neg | M0 | Two mucosal |
| | | | colon | | | | muscularis | | | | | | polyps |
| | | | | | | | propria | | | | | | |
| | | | | | | | to pericolonic | | | | | | |
| | | | | | | | fat | | | | | | |
| 392 | 444 | IV | Ascending | 2 | T3 | G2 | Invasion | Pos | 1/6 | N1 | Pos - | M1 | Tumor arising |
| | | | colon | | | | through | | | | Liver | | at prior |
| | | | | | | | muscularis | | | | | | ileocolic |
| | | | | | | | propria into | | | | | | surgical |
| | | | | | | | subserosal | | | | | | anastomosis |
| | | | | | | | adipose | | | | | | |
| | | | | | | | not serosa. | | | | | | |
| 393 | 445 | II | Cecum | 6.0 | T3 | G2 | Cecum, | Neg | 0/21 | N0 | Neg | M0 | |
| | | | | | | | invades | | | | | | |
| | | | | | | | through | | | | | | |
| | | | | | | | muscularis | | | | | | |
| | | | | | | | propria | | | | | | |
| | | | | | | | to involve | | | | | | |
| | | | | | | | subserosal | | | | | | |
| | | | | | | | adipose | | | | | | |
| | | | | | | | tissue but not | | | | | | |
| | | | | | | | serosa. | | | | | | |
| 413 | 465 | IV | Cecum | 4.8 | T3 | G2 | Invasive | Neg | 0/7 | N0 | Pos - | M1 | rediagnosis of |
| | | | | | | | through | | | | Liver | | oophorectomy |
| | | | | | | | muscularis to | | | | | | path to |
| | | | | | | | involve | | | | | | metastatic |
| | | | | | | | periserosal | | | | | | colon cancer. |
| | | | | | | | fat; abutting | | | | | | |
| | | | | | | | ileocecal | | | | | | |
| | | | | | | | junction. | | | | | | |
| 505 | 383 | IV | | 7.5 | T3 | G2 | Invasion | Pos | 2/17 | N1 | Pos - | M1 | Anatomical |
| | | | | | | | through | | | | Liver | | location of |
| | | | | | | | muscularis | | | | | | primary not |
| | | | | | | | propria involving | | | | | | notated in |
| | | | | | | | pericolic adipose, | | | | | | report. |
| | | | | | | | serosal surface | | | | | | |
| | | | | | | | uninvolved | | | | | | |
| | | | | | | | | | | | | | Evidence of |
| | | | | | | | | | | | | | chronic colitis. |
| 517 | 395 | IV | Sigmoid | 3 | T3 | G2 | penetrates | Pos | 6/6 | N2 | Neg | M0 | No mention of |
| | | | | | | | muscularis | | | | | | distant met in |
| | | | | | | | propria, | | | | | | report |
| | | | | | | | involves | | | | | | |
| | | | | | | | pericolonic fat. | | | | | | |
| 534 | 553 | II | Ascending | 12 | T3 | G3 | Invasion | Neg | 0/8 | N0 | Neg | M0 | Omentum with |
| | | | colon | | | | through the | | | | | | fibrosis and fat |
| | | | | | | | muscularis | | | | | | necrosis. Small |
| | | | | | | | propria | | | | | | bowel with |
| | | | | | | | involving | | | | | | acute and |
| | | | | | | | pericolic fat. | | | | | | chronic |
| | | | | | | | Serosa free of | | | | | | serositis, focal |
| | | | | | | | tumor. | | | | | | abscess and |
| | | | | | | | | | | | | | adhesions. |
| 546 | 565 | IV | Ascending | 5.5 | T3 | G2 | Invasion | Pos | 6/12 | N2 | Pos - | M1 | |
| | | | colon | | | | through | | | | Liver | | |
| | | | | | | | muscularis | | | | | | |
| | | | | | | | propria | | | | | | |
| | | | | | | | extensively | | | | | | |
| | | | | | | | through | | | | | | |
| | | | | | | | submucosal and | | | | | | |
| | | | | | | | extending to | | | | | | |
| | | | | | | | serosa. | | | | | | |
| 577 | 596 | II | Cecum | 11.5 | T3 | G2 | Invasion | Neg | 0/58 | N0 | Neg | M0 | Appendix |
| | | | | | | | through the | | | | | | dilated and |
| | | | | | | | bowel wall, | | | | | | fibrotic, but not |
| | | | | | | | into suberosal | | | | | | involved by |
| | | | | | | | adipose. | | | | | | tumor |
| | | | | | | | Serosal | | | | | | |
| | | | | | | | surface free | | | | | | |
| | | | | | | | of tumor. | | | | | | |
| 695 | 714 | II | Cecum | 14.0 | T3 | G2 | extending | Neg | 0/22 | N0 | Neg | MX | moderately |
| | | | | | | | through | | | | | | differentiated |
| | | | | | | | bowel wall into | | | | | | adeno- |
| | | | | | | | serosal fat | | | | | | |
| | | | | | | | | | | | | | with |
| | | | | | | | | | | | | | mucinous |
| | | | | | | | | | | | | | diferentiation |
| | | | | | | | | | | | | | (% not stated), |
| | | | | | | | | | | | | | tubular |
| | | | | | | | | | | | | | adenoma and |
| | | | | | | | | | | | | | hyperplstic |
| | | | | | | | | | | | | | polyps present, |
| 784 | 803 | IV | Ascending | 3.5 | T3 | G3 | through | Pos | 5/17 | N2 | Pos - | M1 | invasive poorly |
| | | | colon | | | | muscularis | | | | Liver | | differentiated |
| | | | | | | | propria into | | | | | | adenosquamous |
| | | | | | | | pericolic soft | | | | | | carcinoma |
| | | | | | | | tissues | | | | | | |
| 786 | 805 | IV | Descending | 9.5 | T3 | G2 | through | Neg | 0/12 | N0 | Pos - | M1 | moderately |
| | | | colon | | | | muscularis | | | | Liver | | differentiated |
| | | | | | | | propria into | | | | | | invasive |
| | | | | | | | pericolic fat, | | | | | | adeno- |
| | | | | | | | but not at | | | | | | carcinoma |
| | | | | | | | serosal | | | | | | |
| | | | | | | | surface | | | | | | |
| 787 | 806 | II | Recto- | 2.5 | T3 | G2-G3 | Invasion of | Neg | | N0 | Neg | MX | Peritumoral |
| | | | sigmoid | | | | muscularis | | | | | | lymphocytic |
| | | | | | | | propria | | | | | | response; 5 LN |
| | | | | | | | into soft tissue | | | | | | examined in |
| | | | | | | | | | | | | | pericolic fat, no |
| | | | | | | | | | | | | | metastatases |
| | | | | | | | | | | | | | observed. |
| 789 | 808 | IV | Cecum | 5.0 | T3 | G2-G3 | Extending | Pos | 5/10 | N2 | Pos - | M1 | Three |
| | | | | | | | through | | | | Liver | | fungating |
| | | | | | | | muscularis | | | | | | lesions |
| | | | | | | | propria into | | | | | | examined |
| | | | | | | | pericolonic fat | | | | | | |
| 790 | 809 | IV | Rectum | 6.8 | T3 | G1-G2 | Invading | Pos | 3/13 | N1 | Pos - | M1 | |
| | | | | | | | through | | | | Liver | | |
| | | | | | | | muscularis | | | | | | |
| | | | | | | | propria into | | | | | | |
| | | | | | | | perirectal fat | | | | | | |
| 791 | 810 | IV | Ascending | 5.8 | T3 | G3 | Through the | Pos | 13/25 | N2 | Pos - | M1 | poorly |
| | | | colon | | | | muscularis | | | | Liver | | differentiated |
| | | | | | | | propria into | | | | | | invasive |
| | | | | | | | pericolic fat | | | | | | colonic |
| | | | | | | | | | | | | | adeno- |
| | | | | | | | | | | | | | carcinoma |
| 888 | 908 | IV | Ascending | 2.0 | T2 | G1 | Into muscularis | Pos | 3/21 | N0 | Pos - | M1 | well to |
| | | | colon | | | | propria | | | | Liver | | moderately |
| | | | | | | | | | | | | | differentiated |
| | | | | | | | | | | | | | adeno- |
| | | | | | | | | | | | | | carcinomas; |
| | | | | | | | | | | | | | this patient |
| | | | | | | | | | | | | | has tumors of |
| | | | | | | | | | | | | | the ascending |
| | | | | | | | | | | | | | colon and the |
| | | | | | | | | | | | | | sigmoid colon |
| 889 | 909 | IV | Cecum | 4.8 | T3 | G2 | Through | Pos | 1/4 | N1 | Pos - | M1 | moderately |
| | | | | | | | muscularis | | | | Liver | | differentiated |
| | | | | | | | propria int | | | | | | adeno- |
| | | | | | | | subserosal | | | | | | carcinoma |
| | | | | | | | tissue | | | | | | |
| 890 | 910 | IV | Ascending | | T3 | G2 | Through | Pos | 1 1/15 | N2 | Pos - | M1 | |
| | | | colon | | | | muscularis | | | | Liver | | |
| | | | | | | | propria | | | | | | |
| | | | | | | | into subserosa. | | | | | | |
| 891 | 911 | IV | Rectum | 5.2 | T3 | G2 | Invasion | Pos | 4/15 | N2 | Pos - | M1 | Perineural |
| | | | | | | | through | | | | Liver | | invasion |
| | | | | | | | muscularis | | | | | | present. |
| | | | | | | | propria into | | | | | | |
| | | | | | | | perirectal soft | | | | | | |
| | | | | | | | tissue | | | | | | |
| 892 | 912 | IV | Sigmoid | 5.0 | T3 | G2 | Invasion into | Pos | 1/28 | N1 | Pos - | M1 | Perineural |
| | | | | | | | pericolic sort | | | | Liver, | | invasion |
| | | | | | | | tissue. Tumor | | | | left and | | present, |
| | | | | | | | focally | | | | right | | extensive. |
| | | | | | | | invading | | | | lobe, | | Patient with a |
| | | | | | | | skeletal muscle | | | | omentum | | history of colon |
| | | | | | | | attached to | | | | | | cancer. |
| | | | | | | | colon. | | | | | | |
| 893 | 913 | IV | Transverse | 6.0 | T3 | G2-G3 | Through | Pos | 14/17 | N2 | Pos - | M1 | Perineural |
| | | | colon | | | | muscularis | | | | Liver | | invasion |
| | | | | | | | propria into | | | | | | focally |
| | | | | | | | pericolic fat | | | | | | present. |
| | | | | | | | | | | | | | Omentum |
| | | | | | | | | | | | | | mass, but |
| | | | | | | | | | | | | | resection with |
| | | | | | | | | | | | | | no tumor |
| | | | | | | | | | | | | | identified. |
| 989 | 1009 | IV | Sigmoid | 6.0 | T3 | G2 | Invasion | Pos | 1/7 | N1 | Pos - | M1 | Primary |
| | | | | | | | through | | | | Liver | | adeno- |
| | | | | | | | colon wall and | | | | | | carcinoma |
| | | | | | | | focally | | | | | | arising from |
| | | | | | | | involving | | | | | | tubulovillous |
| | | | | | | | subserosal | | | | | | adenoma. |
| | | | | | | | tissue. | | | | | | |

TABLE 13

| | | BREAST | BREAST | COLON | COLON | PROSTATE | PROSTATE |
|---|---|---|---|---|---|---|---|
| SEQ | | PATIENTS >= | PATIENTS | PATIENTS >= | PATIENTS | PATIENTS>= | PATIENTS |
| ID | CLONE ID | 2x | S <= halfx | 2x | <= halfx | 2x | <= halfx |
| 4 | M00072944A:C07 | | | | 35 | | |
| 8 | M00072947B:G04 | | | | 32.5 | | |
| 9 | M00072947D:G05 | | | | 27.5 | | |
| 15 | M00072963B:G11 | | | | 40 | | |
| 16 | M00072967A:G07 | | | | 25 | | |
| 18 | M00072968A:F08 | | | | 22.5 | | |
| 20 | M00072968D:E05 | | | | 32.5 | | |
| 21 | M00072970C:B07 | | | | 25 | | |
| 24 | M00072971C:B07 | | | | 22.5 | | |
| 28 | M00072975A:D11 | 23.5 | | | | | |
| 34 | M00073001A:F07 | | | | 27.5 | | |
| 38 | M00073003A:E06 | | | | 42.5 | | |
| 39 | M00073003B:E10 | | | | 27.5 | | |
| 42 | M00073006A:H08 | 23.5 | | | | | |
| 43 | M00073006C:D07 | | | | 27.5 | | |
| 45 | M00073009B:C08 | | | | 32.5 | | 52.4 |
| 48 | M00073013A:D10 | | | | 32.5 | | |
| 49 | M00073013A:F10 | | | | 20 | | |
| 50 | M00073013C:B10 | | | | 32.5 | | |
| 52 | M00073014D:F01 | | | | 40 | | |
| 54 | M00073015A:H06 | | | | 47.5 | | |
| 61 | M00073020C:F07 | | | | 32.5 | | |
| 62 | M00073020D:C06 | | | 37.5 | | | |
| 63 | M00073021C:E04 | | | | 30 | | |
| 71 | M00073030B:C02 | | | | 22.5 | | |
| 72 | M00073030C:A02 | | | | 20 | | |
| 73 | M00073036C:H10 | | | | 25 | | |
| 86 | M00073043D:H09 | | | | 32.5 | | |
| 90 | M00073044C:G12 | | | | 32.5 | | |
| 94 | M00073045C:E06 | | | | 22.5 | | |
| 96 | M00073045D:B04 | | | | 30 | | |
| 105 | M00073048C:B01 | | | | 20 | | |
| 107 | M00073049A:H04 | | | | 27.5 | | 49.2 |
| 108 | M00073049B:B03 | | 23.5 | | 40 | | 31.7 |
| 109 | M00073049B:B06 | | | | 20 | | |
| 110 | M00073049C:C09 | | | | 20 | | |
| 136 | M00073066C:D02 | | | | 27.5 | | |
| 142 | M00073070B:B06 | | | | 32.5 | | |
| 146 | M00073074D:A04 | | | | 20 | | |
| 153 | M00073086D:B05 | | | | 30 | | |
| 156 | M00073091B:C04 | | | | 20 | | |
| 163 | M00073424D:C03 | 52.9 | | | | | |
| 171 | M00073403C:C10 | | | | 30 | | |
| 173 | M00073403C:E11 | 29.4 | | | 52.5 | | |
| 176 | M00073412C:E07 | | | | 30 | | |
| 177 | M00073435C:E06 | | | | 27.5 | | |
| 178 | M00073412D:B07 | | 35.3 | 42.5 | | | |
| 189 | M00073430C:B02 | | | | 32.5 | | |
| 196 | M00073442A:F07 | | | | 25 | | |
| 197 | M00073442B:D12 | | | | 27.5 | | 20.6 |
| 199 | M00073446C:A03 | | | | 22.5 | | |
| 201 | M00073447D:F01 | | | | 45 | | 38.1 |
| 204 | M00073453C:C09 | 41.2 | | | | | |
| 212 | M00073469B:A09 | | | | 27.5 | | 36.5 |
| 216 | M00073474C:F08 | | | | 30 | | 22.2 |
| 220 | M00073484B:A05 | | 23.5 | | 30 | | 22.2 |
| 228 | M00073497C:D03 | | 29.4 | 30 | | | |
| 233 | M00073513A:G07 | 23.5 | | | | 25.4 | |
| 236 | M00073517A:A06 | | | | 32.5 | | |
| 241 | M00073529A:F03 | | | | 20 | | |
| 242 | M00073530B:A02 | | | | 20 | | 54.0 |
| 243 | M00073531B:H02 | | | | | | 50.8 |
| 246 | M00073539C:H05 | | | | 27.5 | | |
| 247 | M00073541B:C10 | | | | 30 | | |
| 248 | M00073547B:F04 | | | | 22.5 | | |
| 249 | M00073547C:D02 | | | | 35 | | |
| 256 | M00073554B:D11 | | | | 37.5 | | |
| 264 | M00073568A:G06 | | | | 32.5 | | |
| 265 | M00073568C:G07 | | | | 25 | | |
| 269 | M00073576B:E03 | | | | 22.5 | | |
| 270 | M00073576C:C11 | | | | 20 | | |
| 273 | M00073580A:D08 | | | | 32.5 | | |
| 280 | M00073598D:E11 | | | | 40 | | |
| 284 | M00073601D:D08 | | | | 32.5 | | |
| 286 | M00073603B:C03 | | | 30 | | | |
| 288 | M00073603C:C02 | | 76.5 | | 67.5 | | |
| 290 | M00073604B:B07 | | | | 30 | | |
| 294 | M00073605B:F11 | | 58.8 | | | | |
| 299 | M00073614C:F06 | | | 60 | | | |
| 300 | M00073615D:E03 | | | | 82.5 | | |
| 301 | M00073616A:F06 | | | | 32.5 | | 28.6 |
| 304 | M00073621D:A04 | | | | 27.5 | | |
| 316 | M00073633D:A04 | | 23.5 | 52.5 | | | |
| 318 | M00073634C:H08 | 23.5 | | | 85 | 39.7 | |
| 319 | M00073635D:C10 | | 35.3 | | | | |
| 323 | M00073638A:A12 | | | 47.5 | | | |
| 325 | M00073639A:G08 | | | | 27.5 | | |
| 340 | M00073651C:F06 | 29.4 | | | 27.5 | | 36.5 |
| 342 | M00073652D:B11 | | 64.7 | | 70 | | |
| 343 | M00073655B:A04 | | | 37.5 | | | |
| 353 | M00073669A:F04 | | | | 20 | | |
| 354 | M00073669B:E12 | 23.5 | | 27.5 | | | |
| 357 | M00073687A:D11 | | | 50 | | 22.2 | |
| 361 | M00073672D:E09 | | | | 35 | | 42.9 |
| 367 | M00073677B:F01 | | | | 32.5 | | |
| 369 | M00073678B:H02 | | | 35 | | | |
| 372 | M00073681A:F12 | | 29.4 | | | | 25.4 |
| 377 | M00073689C:C09 | | | | | | 41.3 |
| 382 | M00073696C:D11 | | 35.3 | | | | |
| 384 | M00073697C:F11 | | 29.4 | | | | 34.9 |
| 388 | M00073700B:D12 | | | | 30 | | |
| 390 | M00073708D:E10 | | | | | | 23.8 |
| 392 | M00073709B:F01 | | | | 25 | | |
| 394 | M00073709C:A02 | | | | 22.5 | | |
| 398 | M00073713D:E07 | | | | 27.5 | | |
| 399 | M00073715A:F05 | | | | 20 | | 31.7 |
| 400 | M00073715B:B06 | | | | 37.5 | | 27.0 |
| 401 | M00073717C:A12 | | | | 37.5 | | |
| 403 | M00073720D:H11 | | | | 27.5 | | 20.6 |
| 408 | M00073735C:E04 | | | | | | 23.8 |
| 413 | M00073743C:F03 | | | | 25 | | |
| 417 | M00073748B:F07 | | | | 35 | | |
| 424 | M00073754B:D05 | | | | 37.5 | | |
| 436 | M00073765A:E02 | | | | 32.5 | | |
| 439 | M00073766B:B07 | | | | 22.5 | | |
| 442 | M00073772B:E07 | | | | | | 22.2 |
| 450 | M00073779B:B11 | | | | 32.5 | | |
| 462 | M00073798A:H03 | | | | 35 | | |
| 464 | M00073801B:A10 | | | | 35 | | |
| 467 | M00073809C:E09 | | 23.5 | 45 | | 25.4 | |
| 469 | M00073813D:B06 | | | | | | 27.0 |
| 470 | M00073814C:B04 | | | | | | 71.4 |
| 473 | M00073790A:A12 | | | | | | 36.5 |
| 480 | M00073799A:G02 | | | | 37.5 | | |
| 481 | M00073799D:G04 | | | | 30 | | |
| 486 | M00073813A:E06 | | | | 32.5 | | |
| 487 | M00073813B:A01 | | | | 30 | | |
| 493 | M00073822C:E02 | | | | 35 | | |
| 494 | M00073824A:C04 | | | | | | 38.1 |
| 497 | M00073832A:A06 | | | | 20 | | 20.6 |
| 500 | M00073834A:H10 | | | | 35 | | |
| 502 | M00073834D:H06 | | | | 25 | | 31.7 |
| 503 | M00073836D:E05 | | | | | 23.8 | |
| 506 | M00073838B:F09 | | | | 25 | | |
| 509 | M00073839A:D05 | | 23.5 | | 47.5 | | 41.3 |
| 513 | M00073850A:H09 | | | | | | 54.0 |
| 532 | M00073867D:F10 | | | | | | 36.5 |
| 533 | M00073871B:C12 | | | | 32.5 | | |
| 534 | M00073872C:B09 | | | | 22.5 | | |
| 535 | M00073872D:B01 | | | | 32.5 | | |
| 536 | M00073872D:E10 | | | | 22.5 | | |
| 544 | M00073883B:D03 | | | | 22.5 | | |
| 550 | M00073892B:F12 | | | | 32.5 | | |
| 555 | M00073905B:A03 | | | | | | 55.6 |
| 562 | M00073897B:B11 | | | | 30 | | |
| 564 | M00073899A:D06 | | | | 32.5 | | |
| 565 | M00073911B:G10 | | | | | | 23.8 |
| 567 | M00073916A:B07 | | | | 42.5 | | 23.8 |
| 572 | M00073923C:A04 | 29.4 | | | 22.5 | | |
| 575 | M00073931D:E02 | | | | 27.5 | | |
| 577 | M00073936D:E05 | | | | 25 | | |
| 579 | M00073908C:D09 | | | | 40 | | 27.0 |
| 599 | M00073944D:A07 | | | | 27.5 | | |
| 620 | M00073968B:B06 | | | | 27.5 | | 57.1 |
| 625 | M00073979C:G07 | | | | 37.5 | | 44.4 |
| 634 | M00073988D:F09 | | | | | | 38.1 |
| 641 | M00073979B:B05 | | | | 27.5 | | 66.7 |
| 645 | M00073988C:G08 | | | | 40 | | |
| 654 | M00074011D:C05 | | | | 42.5 | | |
| 656 | M00074013C:C09 | | | | 20 | | |
| 659 | M00074015A:C03 | | | | 22.5 | | |
| 665 | M00074020D:G10 | | | | 40 | | |
| 669 | M00074025A:F06 | | | | 25 | | 36.5 |
| 670 | M00074025B:A12 | | | | | | 20.6 |
| 671 | M00074026C:H09 | | | | 32.5 | | |
| 687 | M00074053C:E05 | 25.0 | | 30 | | | |
| 695 | M00074059B:G10 | | | | 27.5 | | |
| 703 | M00074075B:A09 | | | 27.5 | | | |
| 706 | M00074079A:E07 | | | | 42.5 | | 31.7 |
| 708 | M00074084D:B04 | | | | | | 33.3 |
| 710 | M00074085B:E06 | | | | | | 23.8 |
| 712 | M00074087B:C09 | | | | | | 28.6 |
| 713 | M00074087C:G05 | | | | | | 23.8 |
| 717 | M00074089D:E03 | | | | 20 | | 54.0 |
| 720 | M00074093B:A03 | | 23.5 | 27.5 | | | |
| 722 | M00074094B:F10 | | | | | | 52.4 |
| 723 | M00074096D:G12 | | | | | | 25.4 |
| 726 | M00074098C:B09 | | | | | | 23.8 |
| 727 | M00074099C:B09 | | | | 20 | | |
| 729 | M00074101D:D07 | | | 35 | | | |
| 730 | M00074102A:C04 | | | | 37.5 | | |
| 733 | M00074107C:C08 | | | | 35 | | |
| 741 | M00074131A:H09 | | | | 37.5 | | 27.0 |
| 742 | M00074132C:F10 | | | | 32.5 | | 22.2 |
| 747 | M00074138D:A08 | | | | 45 | | 22.2 |
| 749 | M00074142B:C11 | | | | 32.5 | | |
| 750 | M00074142D:A10 | | | | 22.5 | | |
| 753 | M00074122A:B02 | | | | 37.5 | | |
| 756 | M00074132A:E11 | | | 22.5 | | | |
| 757 | M00074132B:B07 | | | | 35 | | 20.6 |
| 758 | M00074134A:G11 | | | | 27.5 | | |
| 759 | M00074149A:B10 | | 41.2 | 47.5 | | | |
| 762 | M00074153D:A05 | | | | 37.5 | | |
| 765 | M00074157C:G08 | | | | 25 | | |
| 767 | M00074158C:F12 | | | | 37.5 | | |
| 769 | M00074159C:A05 | | | | 25 | | |
| 777 | M00074174A:C02 | | | | 27.5 | | 27.0 |
| 782 | M00074177B:H08 | | | | 35 | | |
| 785 | M00074179C:B01 | | | | 27.5 | | 28.6 |
| 787 | M00074184D:B01 | | | | 37.5 | | 28.6 |
| 789 | M00074191C:D08 | | | | | | 57.1 |
| 790 | M00074192C:C10 | | | | | | 33.3 |
| 793 | M00074198C:A12 | 29.4 | | | 45 | | 31.7 |
| 794 | M00074198D:D10 | | | | | | 36.5 |
| 800 | M00074203D:F01 | | | | 40 | | |
| 802 | M00074206A:H12 | | | | 40 | | 22.2 |
| 806 | M00074208B:F09 | | | | 22.5 | | 41.3 |
| 811 | M00074215A:F09 | | | | 42.5 | | |
| 813 | M00074216D:H03 | | | | 35 | | |
| 819 | M00074223B:D12 | | | | 30 | | |
| 821 | M00074225A:H12 | | | | 25 | | |
| 827 | M00074234A:C05 | | | | 30 | | |
| 830 | M00074234D:F12 | | | | 37.5 | | |
| 834 | M00074242D:F09 | | | | 25 | | |
| 837 | M00074247B:G11 | | | | 27.5 | | |
| 839 | M00074248C:E12 | | | | | 25.4 | |
| 840 | M00074249C:B11 | | | | 27.5 | | |
| 846 | M00074251C:E03 | | | | 35 | | |
| 849 | M00074253C:F03 | | | | 32.5 | | |
| 850 | M00074255B:A01 | | | | 20 | | |
| 851 | M00074258A:H12 | | | | 32.5 | | |
| 861 | M00074271B:E11 | | | | 25 | | |
| 869 | M00074280D:H03 | | | | 20 | | 31.7 |
| 870 | M00074284B:B03 | | | | 27.5 | | 25.4 |
| 873 | M00074288A:F11 | | | | 45 | | 20.6 |
| 874 | M00074290A:G10 | | | | 37.5 | | |
| 875 | M00074290C:B05 | | | | | | 20.6 |
| 877 | M00074293D:B05 | | | | 20 | | |
| 878 | M00074293D:H07 | | | | 32.5 | | |
| 882 | M00074304B:C09 | | | | 22.5 | | 39.7 |
| 883 | M00074304D:D07 | | | | | | 36.5 |
| 884 | M00074306A:B09 | | | | 27.5 | | |
| 886 | M00074310D:D02 | | | | 35 | | 25.4 |
| 888 | M00074315B:A03 | | | | 22.5 | | |
| 892 | M00074835A:H10 | | | | 40 | | |
| 893 | M00074835B:F12 | | | | 22.5 | | |
| 895 | M00074837A:E01 | | | | 35 | | |
| 899 | M00074843D:D02 | | | | 25 | | 65.1 |
| 900 | M00074844B:B02 | | 58.8 | 20 | | | |
| 901 | M00074844D:F09 | | | | 30 | | 20.6 |
| 905 | M00074847B:G03 | | | | 30 | | |
| 909 | M00074852B:A02 | | | 37.5 | | | |
| 912 | M00074854A:C11 | | | | 40 | | |
| 913 | M00074855B:A05 | | | | 27.5 | | |
| 917 | M00074863D:F07 | | | | 27.5 | | |
| 919 | M00074317D:B08 | | | | | | 20.6 |
| 920 | M00074320C:A06 | | | | | | 54.0 |
| 921 | M00074865A:F05 | | | | 20 | | 50.8 |
| 923 | M00074871C:G05 | | | | 20 | | |
| 926 | M00074879A:A02 | | | | 35 | | 22.2 |
| 930 | M00074890A:E03 | | | | 20 | | 20.6 |
| 931 | M00074895D:H12 | | | | | | 20.6 |
| 934 | M00074901C:E05 | | | | 27.5 | | |
| 938 | M00074905D:A01 | | | | 35 | | 30.2 |
| 941 | M00074912B:A10 | | | | | | 65.1 |
| 943 | M00074916A:H03 | | | | 30 | | |
| 949 | M00074927D:G09 | | | | 22.5 | | |
| 954 | M00074936B:E10 | | | | 37.5 | | |
| 955 | M00074939B:A06 | | | | 32.5 | | |
| 959 | M00074966D:E08 | | | | | | 34.9 |
| 962 | M00074974C:E11 | | | | | | 22.2 |
| 964 | M00074954A:H06 | | | | 20 | | |
| 975 | M00072985A:C12 | | | | 20 | | |
| 981 | M00072996B:A10 | | | | 27.5 | | 20.6 |
| 984 | M00072997D:H06 | | | | 40 | | 20.6 |
| 986 | M00074333D:A11 | | 41.2 | 47.5 | | | |
| 990 | M00074343C:A03 | | | | 30 | | |
| 998 | M00074366A:H07 | | | | 27.5 | | 42.9 |
| 1004 | M00074392C:D02 | | | | 32.5 | | |
| 1006 | M00074417D:F07 | | 23.5 | 67.5 | | | |
| 1008 | M00074406B:F10 | | | | 27.5 | | |
| 1012 | M00074391B:D02 | | | 27.5 | | | |
| 1019 | M00074461D:E04 | | | | 47.5 | | 25.4 |
| 1025 | M00074488C:C08 | | | | 32.5 | | |
| 1027 | M00074501A:G07 | | | | | | 49.2 |
| 1029 | M00074515A:E02 | | | | | 25.4 | |
| 1030 | M00074515C:A11 | | | | 32.5 | | |
| 1031 | M00074516B:H03 | | | | | | 23.8 |
| 1032 | M00074525A:B05 | | | | | | 20.6 |
| 1039 | M00074561D:D12 | | | | 30 | 28.6 | |
| 1040 | M00074566B:A04 | | | | 35 | | |
| 1044 | M00074555A:E10 | | | | 27.5 | | |
| 1045 | M00074561A:B09 | | | | 40 | | |
| 1052 | M00074582D:B09 | | | | | | 25.4 |
| 1057 | M00074596D:B12 | | | | 20 | | 22.2 |
| 1058 | M00074606C:G02 | 29.4 | | | | | |
| 1064 | M00074628C:D03 | | | | 37.5 | | |
| 1067 | M00074637A:C02 | | | | 20 | | |
| 1068 | M00074638D:C12 | 29.4 | | | 35 | | |
| 1069 | M00074639A:C08 | | | | 30 | | |
| 1073 | M00074662B:A05 | | 35.3 | | | | |
| 1078 | M00074676D:H07 | | | | 22.5 | | |
| 1080 | M00074681D:A02 | | | | 32.5 | | |
| 1082 | M00074699B:C03 | | | | 32.5 | | |
| 1083 | M00074701D:H09 | | | | 25 | | |
| 1086 | M00074713B:F02 | | | | 20 | | 39.7 |
| 1089 | M00074723D:D05 | | | | 27.5 | | |
| 1092 | M00074740B:F06 | | | | 27.5 | | |
| 1095 | M00074752A:D08 | | | | 32.5 | | 20.6 |
| 1099 | M00074765D:F06 | | | | 40 | | |
| 1102 | M00074773C:G03 | | | | 20 | | |
| 1103 | M00074774A:D03 | | | | | | 31.7 |
| 1105 | M00074780C:C02 | | | | 20 | | |
| 1110 | M00075000A:D06 | | | | 32.5 | | |
| 1117 | M00074800B:H01 | | | | 35 | | |
| 1120 | M00074825C:E06 | | | | 30 | | |
| 1122 | M00075018A:G04 | | | | 30 | | |
| 1134 | M00075035C:C09 | | | | 32.5 | | |
| 1135 | M00075045D:H03 | | | | 25 | | |
| 1145 | M00075153C:C11 | | | | 22.5 | | |
| 1146 | M00075161A:E05 | | | | 30 | | |
| 1152 | M00075152D:C06 | | | | 30 | | |
| 1155 | M00075160A:E04 | | | | 42.5 | | |
| 1163 | M00075174D:D06 | | | | 27.5 | | |
| 1167 | M00075199D:D11 | | 29.4 | | | | 36.5 |
| 1168 | M00075201D:A05 | | | | 30 | | |
| 1169 | M00075203A:G06 | | | | 35 | | 20.6 |
| 1179 | M00075245A:A06 | | 41.2 | 37.5 | | 28.6 | |
| 1189 | M00075283A:F04 | | | | | 34.9 | — |
| 1198 | M00075329B:E10 | | 25.0 | 62.5 | | | |
| 1203 | M00075344D:A08 | | | | 22.5 | | |
| 1224 | M00075379A:E07 | | | | 27.5 | | |
| 1225 | M00075383A:B11 | | | | 25 | | |
| 1227 | M00075409A:E04 | | | | 25 | | |
| 1235 | M00075448B:G11 | | | | 35 | | 20.6 |
| 1239 | M00075460C:B06 | | 35.3 | 62.5 | | 20.6 | |
| 1245 | M00075504B:A10 | | | | 32.5 | | |
| 1250 | M00075514A:G12 | | | | 32.5 | | |
| 1266 | M00075621A:F06 | | | | 20 | | 20.6 |
| 1386 | | 23.5 | | | | | |
| 1387 | | | | 34.3 | | | |
| 1388 | | | 23.5 | 67.5 | | | |
| 1390 | | 35.3 | | 26.1 | | | |
| 1400 | | | | | 32.5 | | |
| 1402 | | | | | | | 41.3 |
| 1403 | | | | | | | |
| 1404 | | | | | 30.0 | 28.6 | |
| 1426 | | | | 36.6 | | | |
| 1427 | | | | | 42.9 | | 38.2 |
| 1429 | | | | | 31.6 | | |
| 1434 | | | | 55.0 | | | |
| 1438 | | | | | 21.3 | | 21.5 |
| 1439 | | | | | 30.0 | | |
| 1444 | | | | | | | |
| 1445 | | | | 27.5 | | | |
| 1447 | | 29.4 | | 32.6 | | | |
| 1449 | | 35.3 | | 60.9 | | | |
| 1461 | | | 29.4 | | | | |
| 1462 | | | 41.2 | 36.2 | | | |
| 1463 | | | | | 27.5 | | |
| 1472 | | | | | 23.4 | | |
| 1474 | | | | | 37.5 | | |
| 1475 | | | 35.3 | 54.3 | | | |

TABLE 15

| ES No. | CLONE ID | ATCC# |
|---|---|---|
| ES 210 | M00073054A:A06 | PTA-2376 |
| ES 210 | M00073054A:C10 | PTA-2376 |
| ES 210 | M00073054B:E07 | PTA-2376 |
| ES 210 | M00073054C:E02 | PTA-2376 |
| ES 210 | M00073055D:E11 | PTA-2376 |
| ES 210 | M00073056C:A09 | PTA-2376 |
| ES 210 | M00073056C:C12 | PTA-2376 |
| ES 210 | M00073057A:F09 | PTA-2376 |
| ES 210 | M00073057D:A12 | PTA-2376 |
| ES 210 | M00073060B:C06 | PTA-2376 |
| ES 210 | M00073061B:F10 | PTA-2376 |
| ES 210 | M00073061C:G08 | PTA-2376 |
| ES 210 | M00073062B:D09 | PTA-2376 |
| ES 210 | M00073062C:D09 | PTA-2376 |
| ES 210 | M00073064C:A11 | PTA-2376 |
| ES 210 | M00073064C:H09 | PTA-2376 |
| ES 210 | M00073064D:B11 | PTA-2376 |
| ES 210 | M00073065D:D11 | PTA-2376 |
| ES 210 | M00073066B:G03 | PTA-2376 |
| ES 210 | M00073066C:D02 | PTA-2376 |
| ES 210 | M00073067A:E09 | PTA-2376 |
| ES 210 | M00073067B:D04 | PTA-2376 |
| ES 210 | M00073067D:B02 | PTA-2376 |
| ES 210 | M00073069D:G03 | PTA-2376 |
| ES 210 | M00073070A:B12 | PTA-2376 |
| ES 210 | M00073070B:B06 | PTA-2376 |
| ES 210 | M00073071D:D02 | PTA-2376 |
| ES 210 | M00073072A:A10 | PTA-2376 |
| ES 210 | M00073074B:G04 | PTA-2376 |
| ES 210 | M00073074D:A04 | PTA-2376 |
| ES 210 | M00073078B:F08 | PTA-2376 |
| ES 210 | M00073080B:A07 | PTA-2376 |
| ES 210 | M00073081A:F08 | PTA-2376 |
| ES 210 | M00073081D:C07 | PTA-2376 |
| ES 210 | M00073084C:E02 | PTA-2376 |
| ES 210 | M00073085D:B01 | PTA-2376 |
| ES 210 | M00073086D:B05 | PTA-2376 |
| ES 210 | M00073088C:B04 | PTA-2376 |
| ES 210 | M00073088D:F07 | PTA-2376 |
| ES 210 | M00073091B:C04 | PTA-2376 |
| ES 210 | M00073091D:B06 | PTA-2376 |
| ES 210 | M00073092A:D03 | PTA-2376 |
| ES 210 | M00073092D:B03 | PTA-2376 |
| ES 210 | M00073094B:A01 | PTA-2376 |
| ES 210 | M00073412A:C03 | PTA-2376 |
| ES 210 | M00073408C:F06 | PTA-2376 |
| ES 210 | M00073424D:C03 | PTA-2376 |
| ES 210 | M00073403B:F06 | PTA-2376 |
| ES 210 | M00073407A:E12 | PTA-2376 |
| ES 210 | M00073412A:H09 | PTA-2376 |
| ES 210 | M00073421C:B07 | PTA-2376 |
| ES 210 | M00073416B:F01 | PTA-2376 |
| ES 210 | M00073425A:G10 | PTA-2376 |
| ES 210 | M00073425A:H12 | PTA-2376 |
| ES 210 | M00073403C:C10 | PTA-2376 |
| ES 210 | M00073428D:H03 | PTA-2376 |
| ES 210 | M00073403C:E11 | PTA-2376 |
| ES 210 | M00073435B:E11 | PTA-2376 |
| ES 210 | M00073431A:G02 | PTA-2376 |
| ES 210 | M00073412C:E07 | PTA-2376 |
| ES 210 | M00073435C:E06 | PTA-2376 |
| ES 210 | M00073412D:B07 | PTA-2376 |
| ES 210 | M00073429B:H10 | PTA-2376 |
| ES 210 | M00073403C:H09 | PTA-2376 |
| ES 210 | M00073412D:E02 | PTA-2376 |
| ES 210 | M00073427B:C08 | PTA-2376 |
| ES 210 | M00073423C:E01 | PTA-2376 |
| ES 210 | M00073427B:E04 | PTA-2376 |
| ES 210 | M00073425D:F08 | PTA-2376 |
| ES 210 | M00073096B:A12 | PTA-2376 |
| ES 210 | M00073430C:A01 | PTA-2376 |
| ES 210 | M00073418B:B09 | PTA-2376 |
| ES 210 | M00073430C:B02 | PTA-2376 |
| ES 210 | M00073097C:A03 | PTA-2376 |
| ES 210 | M00073418B:H09 | PTA-2376 |
| ES 210 | M00073408A:D06 | PTA-2376 |
| ES 210 | M00073438A:A08 | PTA-2376 |
| ES 210 | M00073438A:B02 | PTA-2376 |
| ES 210 | M00073438D:G05 | PTA-2376 |
| ES 210 | M00073442A:F07 | PTA-2376 |
| ES 210 | M00073442B:D12 | PTA-2376 |
| ES 210 | M00073442D:E11 | PTA-2376 |
| ES 210 | M00073446C:A03 | PTA-2376 |
| ES 210 | M00073447B:A03 | PTA-2376 |
| ES 210 | M00073447D:F01 | PTA-2376 |
| ES 210 | M00073448B:F11 | PTA-2376 |
| ES 210 | M00073448B:F07 | PTA-2376 |
| ES 210 | M00073453C:C09 | PTA-2376 |
| ES 210 | M00073455C:G09 | PTA-2376 |
| ES 210 | M00073457A:G09 | PTA-2376 |
| ES 210 | M00073462C:H12 | PTA-2376 |
| ES 210 | M00073462D:D12 | PTA-2376 |
| ES 210 | M00073464B:E01 | PTA-2376 |
| ES 210 | M00073464D:G12 | PTA-2376 |
| ES 210 | M00073465A:H08 | PTA-2376 |
| ES 210 | M00073469B:A09 | PTA-2376 |
| ES 210 | M00073469D:A06 | PTA-2376 |
| ES 210 | M00073470D:A01 | PTA-2376 |
| ES 210 | M00073474A:G11 | PTA-2376 |
| ES 210 | M00073474C:F08 | PTA-2376 |
| ES 210 | M00073475D:E05 | PTA-2376 |
| ES 210 | M00073478C:A07 | PTA-2376 |
| ES 210 | M00073483B:C07 | PTA-2376 |
| ES 210 | M00073484B:A05 | PTA-2376 |
| ES 210 | M00073484C:B04 | PTA-2376 |
| ES 210 | M00073486A:A12 | PTA-2376 |
| ES 210 | M00073487A:C07 | PTA-2376 |
| ES 210 | M00073489B:A07 | PTA-2376 |
| ES 210 | M00073493A:E12 | PTA-2376 |
| ES 210 | M00073493D:F05 | PTA-2376 |
| ES 210 | M00073495B:G11 | PTA-2376 |
| ES 210 | M00073497C:D03 | PTA-2376 |
| ES 210 | M00073504D:F03 | PTA-2376 |
| ES 210 | M00073505D:F01 | PTA-2376 |
| ES 210 | M00073509B:B11 | PTA-2376 |
| ES 210 | M00073509B:E03 | PTA-2376 |
| ES 210 | M00073513A:G07 | PTA-2376 |
| ES 210 | M00073513D:A11 | PTA-2376 |
| ES 210 | M00073515A:F09 | PTA-2376 |
| ES 210 | M00073517A:A06 | PTA-2376 |
| ES 210 | M00073517D:F11 | PTA-2376 |
| ES 210 | M00073520D:A04 | PTA-2376 |
| ES 210 | M00073524A:A03 | PTA-2376 |
| ES 210 | M00073524A:G05 | PTA-2376 |
| ES 210 | M00073529A:F03 | PTA-2376 |
| ES 210 | M00073530B:A02 | PTA-2376 |
| ES 210 | M00073531B:H02 | PTA-2376 |
| ES 210 | M00073531C:F12 | PTA-2376 |
| ES 210 | M00073537B:A12 | PTA-2376 |
| ES 210 | M00073539C:H05 | PTA-2376 |
| ES 210 | M00073541B:C10 | PTA-2376 |
| ES 210 | M00073547B:F04 | PTA-2376 |
| ES 210 | M00073547C:D02 | PTA-2376 |
| ES 210 | M00073549B:B03 | PTA-2376 |
| ES 210 | M00073551B:E10 | PTA-2376 |
| ES 210 | M00073552A:F06 | PTA-2376 |
| ES 210 | M00073554A:C01 | PTA-2376 |
| ES 210 | M00073554A:G04 | PTA-2376 |
| ES 210 | M00073554B:A08 | PTA-2376 |
| ES 210 | M00073554B:D11 | PTA-2376 |
| ES 210 | M00073555A:B09 | PTA-2376 |
| ES 210 | M00073555D:B04 | PTA-2376 |
| ES 210 | M00073557A:A05 | PTA-2376 |
| ES 210 | M00073558A:A02 | PTA-2376 |
| ES 210 | M00073561C:A04 | PTA-2376 |
| ES 210 | M00073565D:E05 | PTA-2376 |
| ES 210 | M00073566A:G01 | PTA-2376 |
| ES 210 | M00073568A:G06 | PTA-2376 |
| ES 210 | M00073568C:G07 | PTA-2376 |
| ES 210 | M00073569A:H02 | PTA-2376 |
| ES 210 | M00073571A:F12 | PTA-2376 |
| ES 210 | M00073575B:H12 | PTA-2376 |
| ES 210 | M00073576B:E03 | PTA-2376 |
| ES 210 | M00073576C:C11 | PTA-2376 |
| ES 210 | M00073577B:D12 | PTA-2376 |
| ES 210 | M00073579B:A04 | PTA-2376 |
| ES 210 | M00073580A:D08 | PTA-2376 |
| ES 210 | M00073587D:E12 | PTA-2376 |
| ES 210 | M00073588B:H07 | PTA-2376 |
| ES 210 | M00073590C:F07 | PTA-2376 |
| ES 210 | M00073592B:D09 | PTA-2376 |
| ES 210 | M00073594B:B11 | PTA-2376 |
| ES 210 | M00073595D:A11 | PTA-2376 |
| ES 210 | M00073598D:E11 | PTA-2376 |
| ES 210 | M00073599C:E08 | PTA-2376 |
| ES 210 | M00073601A:B06 | PTA-2376 |
| ES 210 | M00073601A:F07 | PTA-2376 |
| ES 210 | M00073601D:D08 | PTA-2376 |
| ES 210 | M00073603A:F04 | PTA-2376 |
| ES 210 | M00073603B:C03 | PTA-2376 |
| ES 210 | M00073603C:A11 | PTA-2376 |
| ES 210 | M00073603C:C02 | PTA-2376 |
| ES 210 | M00073603D:E07 | PTA-2376 |
| ES 210 | M00073604B:B07 | PTA-2376 |
| ES 210 | M00073604B:H06 | PTA-2376 |
| ES 210 | M00073604C:H09 | PTA-2376 |
| ES 210 | M00073605B:F10 | PTA-2376 |
| ES 210 | M00073605B:F11 | PTA-2376 |
| ES 210 | M00073606D:F12 | PTA-2376 |
| ES 210 | M00073610A:F06 | PTA-2376 |
| ES 210 | M00073614B:A12 | PTA-2376 |
| ES 210 | M00073614B:G09 | PTA-2376 |
| ES 210 | M00073614C:F06 | PTA-2376 |
| ES 210 | M00073615D:E03 | PTA-2376 |
| ES 210 | M00073616A:F06 | PTA-2376 |
| ES 210 | M00073617A:H04 | PTA-2376 |
| ES 210 | M00073620A:G05 | PTA-2376 |
| ES 210 | M00073621D:A04 | PTA-2376 |
| ES 210 | M00073621D:D02 | PTA-2376 |
| ES 210 | M00073621D:H05 | PTA-2376 |
| ES 210 | M00073623D:H10 | PTA-2376 |
| ES 210 | M00073625C:D09 | PTA-2376 |
| ES 211 | M00073626D:A01 | PTA-2377 |
| ES 211 | M00073628A:E03 | PTA-2377 |
| ES 211 | M00073630A:C03 | PTA-2377 |
| ES 211 | M00073630B:E09 | PTA-2377 |
| ES 211 | M00073630C:D02 | PTA-2377 |
| ES 211 | M00073632A:B12 | PTA-2377 |
| ES 211 | M00073632C:A03 | PTA-2377 |
| ES 211 | M00073633D:A04 | PTA-2377 |
| ES 211 | M00073633D:G04 | PTA-2377 |
| ES 211 | M00073634C:H08 | PTA-2377 |
| ES 211 | M00073635D:C10 | PTA-2377 |
| ES 211 | M00073636C:F03 | PTA-2377 |
| ES 211 | M00073637C:B01 | PTA-2377 |
| ES 211 | M00073637C:E04 | PTA-2377 |
| ES 211 | M00073638A:A12 | PTA-2377 |
| ES 211 | M00073638D:D10 | PTA-2377 |
| ES 211 | M00073639A:G08 | PTA-2377 |
| ES 211 | M00073639B:F02 | PTA-2377 |
| ES 211 | M00073634B:C12 | PTA-2377 |
| ES 211 | M00073640B:G08 | PTA-2377 |
| ES 211 | M00073640C:A03 | PTA-2377 |
| ES 211 | M00073640D:A11 | PTA-2377 |
| ES 211 | M00073640D:G07 | PTA-2377 |
| ES 211 | M00073641B:G07 | PTA-2377 |
| ES 211 | M00073641C:E04 | PTA-2377 |
| ES 211 | M00073643B:E11 | PTA-2377 |
| ES 211 | M00073644A:G12 | PTA-2377 |
| ES 211 | M00073646A:C01 | PTA-2377 |
| ES 211 | M00073647B:H07 | PTA-2377 |
| ES 211 | M00073649A:A03 | PTA-2377 |
| ES 211 | M00073649A:G08 | PTA-2377 |
| ES 211 | M00073651C:F06 | PTA-2377 |
| ES 211 | M00073651C:H07 | PTA-2377 |
| ES 211 | M00073652D:B11 | PTA-2377 |
| ES 211 | M00073655B:A04 | PTA-2377 |
| ES 211 | M00073657B:D05 | PTA-2377 |
| ES 211 | M00073659C:D03 | PTA-2377 |
| ES 211 | M00073663A:E02 | PTA-2377 |
| ES 211 | M00073663D:G06 | PTA-2377 |
| ES 211 | M00073664A:E03 | PTA-2377 |
| ES 211 | M00073666B:B01 | PTA-2377 |
| ES 211 | M00073668A:H03 | PTA-2377 |
| ES 211 | M00073668B:A08 | PTA-2377 |
| ES 211 | M00073668D:D10 | PTA-2377 |
| ES 211 | M00073669A:F04 | PTA-2377 |
| ES 211 | M00073669B:E12 | PTA-2377 |
| ES 211 | M00073669D:G10 | PTA-2377 |
| ES 211 | M00073671B:D09 | PTA-2377 |
| ES 211 | M00073687A:D11 | PTA-2377 |
| ES 211 | M00073699C:E02 | PTA-2377 |
| ES 211 | M00073701D:G10 | PTA-2377 |
| ES 211 | M00073672D:B07 | PTA-2377 |
| ES 211 | M00073672D:E09 | PTA-2377 |
| ES 211 | M00073673A:D11 | PTA-2377 |
| ES 211 | M00073673D:H03 | PTA-2377 |
| ES 211 | M00073674D:F10 | PTA-2377 |
| ES 211 | M00073676A:G08 | PTA-2377 |
| ES 211 | M00073676D:H04 | PTA-2377 |
| ES 211 | M00073677B:F01 | PTA-2377 |
| ES 211 | M00073678B:E08 | PTA-2377 |
| ES 211 | M00073678B:H02 | PTA-2377 |
| ES 211 | M00073679A:D06 | PTA-2377 |
| ES 211 | M00073680D:F11 | PTA-2377 |
| ES 211 | M00073681A:F12 | PTA-2377 |
| ES 211 | M00073684B:F10 | PTA-2377 |
| ES 211 | M00073685A:F07 | PTA-2377 |
| ES 211 | M00073688C:A12 | PTA-2377 |
| ES 211 | M00073688D:C11 | PTA-2377 |
| ES 211 | M00073689C:C09 | PTA-2377 |
| ES 211 | M00073690B:G04 | PTA-2377 |
| ES 211 | M00073691A:G02 | PTA-2377 |
| ES 211 | M00073692D:H02 | PTA-2377 |
| ES 211 | M00073695C:D11 | PTA-2377 |
| ES 211 | M00073696C:D11 | PTA-2377 |
| ES 211 | M00073696D:A08 | PTA-2377 |
| ES 211 | M00073697C:F11 | PTA-2377 |
| ES 211 | M00073699B:D02 | PTA-2377 |
| ES 211 | M00073699B:D09 | PTA-2377 |
| ES 211 | M00073700A:C09 | PTA-2377 |
| ES 211 | M00073700B:D12 | PTA-2377 |
| ES 211 | M00073707B:G08 | PTA-2377 |
| ES 211 | M00073708D:E10 | PTA-2377 |
| ES 211 | M00073708D:F03 | PTA-2377 |
| ES 211 | M00073709B:F01 | PTA-2377 |
| ES 211 | M00073709C:A01 | PTA-2377 |
| ES 211 | M00073709C:A02 | PTA-2377 |
| ES 211 | M00073710B:A09 | PTA-2377 |
| ES 211 | M00073710D:G06 | PTA-2377 |
| ES 211 | M00073711C:E12 | PTA-2377 |
| ES 211 | M00073713D:E07 | PTA-2377 |
| ES 211 | M00073715A:F05 | PTA-2377 |
| ES 211 | M00073715B:B06 | PTA-2377 |
| ES 211 | M00073717C:A12 | PTA-2377 |
| ES 211 | M00073718A:F11 | PTA-2377 |
| ES 211 | M00073720D:H11 | PTA-2377 |
| ES 211 | M00073724D:F04 | PTA-2377 |
| ES 211 | M00073732C:B09 | PTA-2377 |
| ES 211 | M00073733A:A05 | PTA-2377 |
| ES 211 | M00073733A:E03 | PTA-2377 |
| ES 211 | M00073735C:E04 | PTA-2377 |
| ES 211 | M00073737A:C12 | PTA-2377 |
| ES 211 | M00073739D:B04 | PTA-2377 |
| ES 211 | M00073740B:F08 | PTA-2377 |
| ES 211 | M00073741A:B01 | PTA-2377 |
| ES 211 | M00073741C:D05 | PTA-2377 |
| ES 211 | M00073743C:F03 | PTA-2377 |
| ES 211 | M00073746A:H03 | PTA-2377 |
| ES 211 | M00073748A:F09 | PTA-2377 |
| ES 211 | M00073748B:A12 | PTA-2377 |
| ES 211 | M00073748B:F07 | PTA-2377 |
| ES 211 | M00073750A:E08 | PTA-2377 |
| ES 211 | M00073750A:H08 | PTA-2377 |
| ES 211 | M00073750B:D05 | PTA-2377 |
| ES 211 | M00073750C:G06 | PTA-2377 |
| ES 211 | M00073751D:A06 | PTA-2377 |
| ES 211 | M00073753B:B05 | PTA-2377 |
| ES 211 | M00073754B:D05 | PTA-2377 |
| ES 211 | M00073754B:H02 | PTA-2377 |
| ES 211 | M00073754C:C01 | PTA-2377 |
| ES 211 | M00073758C:G03 | PTA-2377 |
| ES 211 | M00073760B:B11 | PTA-2377 |
| ES 211 | M00073760D:F04 | PTA-2377 |
| ES 211 | M00073762A:B09 | PTA-2377 |
| ES 211 | M00073762D:C02 | PTA-2377 |
| ES 211 | M00073763A:D06 | PTA-2377 |
| ES 211 | M00073764B:B09 | PTA-2377 |
| ES 211 | M00073764D:A07 | PTA-2377 |
| ES 211 | M00073764D:B12 | PTA-2377 |
| ES 211 | M00073765A:E02 | PTA-2377 |
| ES 211 | M00073765C:B01 | PTA-2377 |
| ES 211 | M00073766A:B07 | PTA-2377 |
| ES 211 | M00073766B:B07 | PTA-2377 |
| ES 211 | M00073766B:C04 | PTA-2377 |
| ES 211 | M00073769D:G10 | PTA-2377 |
| ES 211 | M00073772B:E07 | PTA-2377 |
| ES 211 | M00073773A:F05 | PTA-2377 |
| ES 211 | M00073773A:G04 | PTA-2377 |
| ES 211 | M00073773B:A09 | PTA-2377 |
| ES 211 | M00073774C:G12 | PTA-2377 |
| ES 211 | M00073776C:F11 | PTA-2377 |
| ES 211 | M00073777A:A01 | PTA-2377 |
| ES 211 | M00073777A:H03 | PTA-2377 |
| ES 211 | M00073779B:B11 | PTA-2377 |
| ES 211 | M00073784A:A12 | PTA-2377 |
| ES 211 | M00073785C:A05 | PTA-2377 |
| ES 211 | M00073785D:D01 | PTA-2377 |
| ES 211 | M00073787D:H12 | PTA-2377 |
| ES 211 | M00073788C:A10 | PTA-2377 |
| ES 211 | M00073790C:E07 | PTA-2377 |
| ES 211 | M00073793C:E09 | PTA-2377 |
| ES 211 | M00073795A:F03 | PTA-2377 |
| ES 211 | M00073795B:B05 | PTA-2377 |
| ES 211 | M00073795B:B09 | PTA-2377 |
| ES 211 | M00073796A:C03 | PTA-2377 |
| ES 211 | M00073798A:H03 | PTA-2377 |
| ES 211 | M00073800D:F08 | PTA-2377 |
| ES 211 | M00073801B:A10 | PTA-2377 |
| ES 211 | M00073802D:B11 | PTA-2377 |
| ES 211 | M00073806D:C09 | PTA-2377 |
| ES 211 | M00073809C:E09 | PTA-2377 |
| ES 211 | M00073810C:F05 | PTA-2377 |
| ES 211 | M00073813D:B06 | PTA-2377 |
| ES 211 | M00073814C:B04 | PTA-2377 |
| ES 211 | M00073786D:B03 | PTA-2377 |
| ES 211 | M00073789C:B06 | PTA-2377 |
| ES 211 | M00073790A:A12 | PTA-2377 |
| ES 211 | M00073792B:A03 | PTA-2377 |
| ES 211 | M00073794B:G09 | PTA-2377 |
| ES 211 | M00073794D:G07 | PTA-2377 |
| ES 211 | M00073796A:D08 | PTA-2377 |
| ES 211 | M00073796B:A03 | PTA-2377 |
| ES 211 | M00073799A:A09 | PTA-2377 |
| ES 211 | M00073799A:G02 | PTA-2377 |
| ES 211 | M00073799D:G04 | PTA-2377 |
| ES 211 | M00073803B:B03 | PTA-2377 |
| ES 211 | M00073803B:C06 | PTA-2377 |
| ES 211 | M00073810B:G10 | PTA-2377 |
| ES 211 | M00073810C:A06 | PTA-2377 |
| ES 211 | M00073813A:E06 | PTA-2377 |
| ES 211 | M00073813B:A01 | PTA-2377 |
| ES 211 | M00073815D:E02 | PTA-2377 |
| ES 211 | M00073818A:A06 | PTA-2377 |
| ES 211 | M00073819D:C11 | PTA-2377 |
| ES 211 | M00073821A:B10 | PTA-2377 |
| ES 211 | M00073821B:H03 | PTA-2377 |
| ES 211 | M00073822C:E02 | PTA-2377 |
| ES 211 | M00073824A:C04 | PTA-2377 |
| ES 211 | M00073826B:C01 | PTA-2377 |
| ES 211 | M00073831B:H09 | PTA-2377 |
| ES 211 | M00073832A:A06 | PTA-2377 |
| ES 211 | M00073832A:G01 | PTA-2377 |
| ES 211 | M00073832B:B05 | PTA-2377 |
| ES 212 | M00073834A:H10 | PTA-2378 |
| ES 212 | M00073834D:E07 | PTA-2378 |
| ES 212 | M00073834D:H06 | PTA-2378 |
| ES 212 | M00073836D:E05 | PTA-2378 |
| ES 212 | M00073837B:D12 | PTA-2378 |
| ES 212 | M00073838A:H07 | PTA-2378 |
| ES 212 | M00073838B:F09 | PTA-2378 |
| ES 212 | M00073838B:H06 | PTA-2378 |
| ES 212 | M00073838D:E01 | PTA-2378 |
| ES 212 | M00073839A:D05 | PTA-2378 |
| ES 212 | M00073840D:C08 | PTA-2378 |
| ES 212 | M00073841A:A03 | PTA-2378 |
| ES 212 | M00073845D:F05 | PTA-2378 |
| ES 212 | M00073850A:H09 | PTA-2378 |
| ES 212 | M00073850D:G04 | PTA-2378 |
| ES 212 | M00073851A:C05 | PTA-2378 |
| ES 212 | M00073851A:E04 | PTA-2378 |
| ES 212 | M00073853C:A01 | PTA-2378 |
| ES 212 | M00073854B:B04 | PTA-2378 |
| ES 212 | M00073854C:F08 | PTA-2378 |
| ES 212 | M00073857A:B12 | PTA-2378 |
| ES 212 | M00073859A:C09 | PTA-2378 |
| ES 212 | M00073860B:F12 | PTA-2378 |
| ES 212 | M00073861D:A09 | PTA-2378 |
| ES 212 | M00073861D:D08 | PTA-2378 |
| ES 212 | M00073862B:D11 | PTA-2378 |
| ES 212 | M00073862D:F06 | PTA-2378 |
| ES 212 | M00073863B:G09 | PTA-2378 |
| ES 212 | M00073863C:D04 | PTA-2378 |
| ES 212 | M00073865B:G04 | PTA-2378 |
| ES 212 | M00073866A:G07 | PTA-2378 |
| ES 212 | M00073867B:E01 | PTA-2378 |
| ES 212 | M00073867D:F10 | PTA-2378 |
| ES 212 | M00073871B:C12 | PTA-2378 |
| ES 212 | M00073872C:B09 | PTA-2378 |
| ES 212 | M00073872D:B01 | PTA-2378 |
| ES 212 | M00073872D:E10 | PTA-2378 |
| ES 212 | M00073873C:A06 | PTA-2378 |
| ES 212 | M00073875A:B03 | PTA-2378 |
| ES 212 | M00073875C:G02 | PTA-2378 |
| ES 212 | M00073878C:A03 | PTA-2378 |
| ES 212 | M00073879D:B08 | PTA-2378 |
| ES 212 | M00073880B:B02 | PTA-2378 |
| ES 212 | M00073880B:B09 | PTA-2378 |
| ES 212 | M00073883B:D03 | PTA-2378 |
| ES 212 | M00073883B:H03 | PTA-2378 |
| ES 212 | M00073886C:C12 | PTA-2378 |
| ES 212 | M00073889B:G08 | PTA-2378 |
| ES 212 | M00073891A:A06 | PTA-2378 |
| ES 212 | M00073892A:E02 | PTA-2378 |
| ES 212 | M00073892B:F12 | PTA-2378 |
| ES 212 | M00073893D:A04 | PTA-2378 |
| ES 212 | M00073895C:F02 | PTA-2378 |
| ES 212 | M00073896A:F07 | PTA-2378 |
| ES 212 | M00073899C:E12 | PTA-2378 |
| ES 212 | M00073905B:A03 | PTA-2378 |
| ES 212 | M00073905D:C11 | PTA-2378 |
| ES 212 | M00073907B:B06 | PTA-2378 |
| ES 212 | M00073884D:B06 | PTA-2378 |
| ES 212 | M00073888C:C10 | PTA-2378 |
| ES 212 | M00073891C:A12 | PTA-2378 |
| ES 212 | M00073893B:C08 | PTA-2378 |
| ES 212 | M00073897B:B11 | PTA-2378 |
| ES 212 | M00073899A:C02 | PTA-2378 |
| ES 212 | M00073899A:D06 | PTA-2378 |
| ES 212 | M00073911B:G10 | PTA-2378 |
| ES 212 | M00073912B:C04 | PTA-2378 |
| ES 212 | M00073916A:B07 | PTA-2378 |
| ES 212 | M00073917B:B07 | PTA-2378 |
| ES 212 | M00073918C:B03 | PTA-2378 |
| ES 212 | M00073921B:H12 | PTA-2378 |
| ES 212 | M00073922C:E02 | PTA-2378 |
| ES 212 | M00073923C:A04 | PTA-2378 |
| ES 212 | M00073924B:H03 | PTA-2378 |
| ES 212 | M00073927D:E09 | PTA-2378 |
| ES 212 | M00073931D:E02 | PTA-2378 |
| ES 212 | M00073932D:G05 | PTA-2378 |
| ES 212 | M00073936D:E05 | PTA-2378 |
| ES 212 | M00073938B:D11 | PTA-2378 |
| ES 212 | M00073908C:D09 | PTA-2378 |
| ES 212 | M00073916C:H11 | PTA-2378 |
| ES 212 | M00073918A:F07 | PTA-2378 |
| ES 212 | M00073918A:G12 | PTA-2378 |
| ES 212 | M00073919C:B04 | PTA-2378 |
| ES 212 | M00073920D:F08 | PTA-2378 |
| ES 212 | M00073922D:G04 | PTA-2378 |
| ES 212 | M00073924C:G05 | PTA-2378 |
| ES 212 | M00073927C:B07 | PTA-2378 |
| ES 212 | M00073933B:B12 | PTA-2378 |
| ES 212 | M00073938B:F09 | PTA-2378 |
| ES 212 | M00073941B:A06 | PTA-2378 |
| ES 212 | M00073941D:H09 | PTA-2378 |
| ES 212 | M00073942B:C01 | PTA-2378 |
| ES 212 | M00073942C:E04 | PTA-2378 |
| ES 212 | M00073942D:D09 | PTA-2378 |
| ES 212 | M00073942D:G05 | PTA-2378 |
| ES 212 | M00073944A:E10 | PTA-2378 |
| ES 212 | M00073944A:H05 | PTA-2378 |
| ES 212 | M00073944C:H07 | PTA-2378 |
| ES 212 | M00073944D:A07 | PTA-2378 |
| ES 212 | M00073944D:E12 | PTA-2378 |
| ES 212 | M00073946D:F07 | PTA-2378 |
| ES 212 | M00073947C:B01 | PTA-2378 |
| ES 212 | M00073947C:E09 | PTA-2378 |
| ES 212 | M00073948A:G05 | PTA-2378 |
| ES 212 | M00073949A:C09 | PTA-2378 |
| ES 212 | M00073949D:C11 | PTA-2378 |
| ES 212 | M00073950C:A05 | PTA-2378 |
| ES 212 | M00073950D:H12 | PTA-2378 |
| ES 212 | M00073952A:G04 | PTA-2378 |
| ES 212 | M00073956D:F02 | PTA-2378 |
| ES 212 | M00073960A:B12 | PTA-2378 |
| ES 212 | M00073960B:A09 | PTA-2378 |
| ES 212 | M00073961B:G01 | PTA-2378 |
| ES 212 | M00073962D:E04 | PTA-2378 |
| ES 212 | M00073963A:G08 | PTA-2378 |
| ES 212 | M00073963B:F04 | PTA-2378 |
| ES 212 | M00073964B:H07 | PTA-2378 |
| ES 212 | M00073967A:A10 | PTA-2378 |
| ES 212 | M00073967C:A01 | PTA-2378 |
| ES 212 | M00073968B:B06 | PTA-2378 |
| ES 212 | M00073968D:F11 | PTA-2378 |
| ES 212 | M00073970B:G01 | PTA-2378 |
| ES 212 | M00073977D:B10 | PTA-2378 |
| ES 212 | M00073978D:A02 | PTA-2378 |
| ES 212 | M00073979C:G07 | PTA-2378 |
| ES 212 | M00073981C:F08 | PTA-2378 |
| ES 212 | M00073983B:D03 | PTA-2378 |
| ES 212 | M00073983C:C07 | PTA-2378 |
| ES 212 | M00073984B:D04 | PTA-2378 |
| ES 212 | M00073984B:E01 | PTA-2378 |
| ES 212 | M00073985C:A05 | PTA-2378 |
| ES 212 | M00073987B:A09 | PTA-2378 |
| ES 212 | M00073988B:C08 | PTA-2378 |
| ES 212 | M00073988D:F09 | PTA-2378 |
| ES 212 | M00073993A:A05 | PTA-2378 |
| ES 212 | M00073965D:A12 | PTA-2378 |
| ES 212 | M00073966C:F08 | PTA-2378 |
| ES 212 | M00073968C:C09 | PTA-2378 |
| ES 212 | M00073968C:F02 | PTA-2378 |
| ES 212 | M00073975A:A12 | PTA-2378 |
| ES 212 | M00073979B:B05 | PTA-2378 |
| ES 212 | M00073979C:B01 | PTA-2378 |
| ES 212 | M00073982B:H01 | PTA-2378 |
| ES 212 | M00073986C:D07 | PTA-2378 |
| ES 212 | M00073988C:G08 | PTA-2378 |
| ES 212 | M00074000C:D06 | PTA-2378 |
| ES 212 | M00074003C:H06 | PTA-2378 |
| ES 212 | M00074004A:H01 | PTA-2378 |
| ES 212 | M00074004C:F03 | PTA-2378 |
| ES 212 | M00074006C:B12 | PTA-2378 |
| ES 212 | M00074007B:A02 | PTA-2378 |
| ES 212 | M00074010B:D07 | PTA-2378 |
| ES 212 | M00074011A:F08 | PTA-2378 |
| ES 212 | M00074011D:C05 | PTA-2378 |
| ES 212 | M00074013B:F07 | PTA-2378 |
| ES 212 | M00074013C:C09 | PTA-2378 |
| ES 212 | M00074014A:G03 | PTA-2378 |
| ES 212 | M00074014D:F04 | PTA-2378 |
| ES 212 | M00074015A:C03 | PTA-2378 |
| ES 212 | M00074017B:G10 | PTA-2378 |
| ES 212 | M00074017D:C01 | PTA-2378 |
| ES 212 | M00074019D:H05 | PTA-2378 |
| ES 212 | M00074020B:G11 | PTA-2378 |
| ES 212 | M00074020C:A05 | PTA-2378 |
| ES 212 | M00074020D:G10 | PTA-2378 |
| ES 212 | M00074021C:H07 | PTA-2378 |
| ES 212 | M00074022A:C06 | PTA-2378 |
| ES 212 | M00074024B:G07 | PTA-2378 |
| ES 212 | M00074025A:F06 | PTA-2378 |
| ES 212 | M00074025B:A12 | PTA-2378 |
| ES 212 | M00074026C:H09 | PTA-2378 |
| ES 212 | M00074027D:B03 | PTA-2378 |
| ES 212 | M00074030D:A12 | PTA-2378 |
| ES 212 | M00074032B:H08 | PTA-2378 |
| ES 212 | M00074032C:E02 | PTA-2378 |
| ES 212 | M00074032C:H07 | PTA-2378 |
| ES 212 | M00074036B:C08 | PTA-2378 |
| ES 212 | M00074036D:B05 | PTA-2378 |
| ES 212 | M00074037A:B03 | PTA-2378 |
| ES 212 | M00074038A:G08 | PTA-2378 |
| ES 212 | M00074038C:B08 | PTA-2378 |
| ES 212 | M00074040A:B06 | PTA-2378 |
| ES 212 | M00074043C:A05 | PTA-2378 |
| ES 212 | M00074050B:H07 | PTA-2378 |
| ES 212 | M00074051C:F05 | PTA-2378 |
| ES 212 | M00074052C:E03 | PTA-2378 |
| ES 212 | M00074053C:E05 | PTA-2378 |
| ES 212 | M00074053C:G11 | PTA-2378 |
| ES 212 | M00074053D:D05 | PTA-2378 |
| ES 212 | M00074054C:B04 | PTA-2378 |
| ES 212 | M00074055A:G08 | PTA-2378 |
| ES 213 | M00072942B:E02 | PTA-2379 |
| ES 213 | M00072942D:F07 | PTA-2379 |
| ES 213 | M00072943B:E04 | PTA-2379 |
| ES 213 | M00072944A:C07 | PTA-2379 |
| ES 213 | M00072944A:E06 | PTA-2379 |
| ES 213 | M00072944C:C02 | PTA-2379 |
| ES 213 | M00072944D:C08 | PTA-2379 |
| ES 213 | M00072947B:G04 | PTA-2379 |
| ES 213 | M00072947D:G05 | PTA-2379 |
| ES 213 | M00072950A:A06 | PTA-2379 |
| ES 213 | M00072961A:G04 | PTA-2379 |
| ES 213 | M00072961B:G10 | PTA-2379 |
| ES 213 | M00072961C:B06 | PTA-2379 |
| ES 213 | M00072962A:B05 | PTA-2379 |
| ES 213 | M00072963B:G11 | PTA-2379 |
| ES 213 | M00072967A:G07 | PTA-2379 |
| ES 213 | M00072967B:G06 | PTA-2379 |
| ES 213 | M00072968A:F08 | PTA-2379 |
| ES 213 | M00072968D:A06 | PTA-2379 |
| ES 213 | M00072968D:E05 | PTA-2379 |
| ES 213 | M00072970C:B07 | PTA-2379 |
| ES 213 | M00074057A:B12 | PTA-2379 |
| ES 213 | M00074058A:H02 | PTA-2379 |
| ES 213 | M00074058B:A10 | PTA-2379 |
| ES 213 | M00074059B:G10 | PTA-2379 |
| ES 213 | M00074060D:A10 | PTA-2379 |
| ES 213 | M00074061B:E01 | PTA-2379 |
| ES 213 | M00074063A:B03 | PTA-2379 |
| ES 213 | M00074063A:D09 | PTA-2379 |
| ES 213 | M00074063B:B12 | PTA-2379 |
| ES 213 | M00074069D:C11 | PTA-2379 |
| ES 213 | M00074070D:G05 | PTA-2379 |
| ES 213 | M00074075B:A09 | PTA-2379 |
| ES 213 | M00074075C:H04 | PTA-2379 |
| ES 213 | M00074076B:F04 | PTA-2379 |
| ES 213 | M00074079A:E07 | PTA-2379 |
| ES 213 | M00074084C:E01 | PTA-2379 |
| ES 213 | M00074084D:B04 | PTA-2379 |
| ES 213 | M00074085A:H10 | PTA-2379 |
| ES 213 | M00074085B:E06 | PTA-2379 |
| ES 213 | M00074085D:E08 | PTA-2379 |
| ES 213 | M00074087B:C09 | PTA-2379 |
| ES 213 | M00074087C:G05 | PTA-2379 |
| ES 213 | M00074088B:A03 | PTA-2379 |
| ES 213 | M00074088C:E07 | PTA-2379 |
| ES 213 | M00074089A:B09 | PTA-2379 |
| ES 213 | M00074089D:E03 | PTA-2379 |
| ES 213 | M00074090A:E09 | PTA-2379 |
| ES 213 | M00074093A:A06 | PTA-2379 |
| ES 213 | M00074093B:A03 | PTA-2379 |
| ES 213 | M00074093B:C07 | PTA-2379 |
| ES 213 | M00074094B:F10 | PTA-2379 |
| ES 213 | M00074096D:G12 | PTA-2379 |
| ES 213 | M00074097A:F10 | PTA-2379 |
| ES 213 | M00074097C:B09 | PTA-2379 |
| ES 213 | M00074098C:B09 | PTA-2379 |
| ES 213 | M00074099C:B09 | PTA-2379 |
| ES 213 | M00074100B:E01 | PTA-2379 |
| ES 213 | M00074101D:D07 | PTA-2379 |
| ES 213 | M00074102A:C04 | PTA-2379 |
| ES 213 | M00074105A:D02 | PTA-2379 |
| ES 213 | M00074106C:E03 | PTA-2379 |
| ES 213 | M00074107C:C08 | PTA-2379 |
| ES 213 | M00074111C:B02 | PTA-2379 |
| ES 213 | M00074111C:G11 | PTA-2379 |
| ES 213 | M00074116C:A03 | PTA-2379 |
| ES 213 | M00074120A:A12 | PTA-2379 |
| ES 213 | M00074123B:A03 | PTA-2379 |
| ES 213 | M00074123B:G07 | PTA-2379 |
| ES 213 | M00074130B:F06 | PTA-2379 |
| ES 213 | M00074131A:H09 | PTA-2379 |
| ES 213 | M00074132C:F10 | PTA-2379 |
| ES 213 | M00074135A:G09 | PTA-2379 |
| ES 213 | M00074135C:E09 | PTA-2379 |
| ES 213 | M00074137C:E05 | PTA-2379 |
| ES 213 | M00074138D:A01 | PTA-2379 |
| ES 213 | M00074138D:A08 | PTA-2379 |
| ES 213 | M00074138D:B07 | PTA-2379 |
| ES 213 | M00074142B:C11 | PTA-2379 |
| ES 213 | M00074142D:A10 | PTA-2379 |
| ES 213 | M00074148B:D09 | PTA-2379 |
| ES 213 | M00074108B:C04 | PTA-2379 |
| ES 213 | M00074122A:B02 | PTA-2379 |
| ES 213 | M00074126B:E12 | PTA-2379 |
| ES 213 | M00074128D:C09 | PTA-2379 |
| ES 213 | M00074132A:E11 | PTA-2379 |
| ES 213 | M00074132B:B07 | PTA-2379 |
| ES 213 | M00074134A:G11 | PTA-2379 |
| ES 213 | M00074149A:B10 | PTA-2379 |
| ES 213 | M00074149A:F12 | PTA-2379 |
| ES 213 | M00074153A:E07 | PTA-2379 |
| ES 213 | M00074153D:A05 | PTA-2379 |
| ES 213 | M00074154A:D03 | PTA-2379 |
| ES 213 | M00074155B:G09 | PTA-2379 |
| ES 213 | M00074157C:G08 | PTA-2379 |
| ES 213 | M00074157D:G05 | PTA-2379 |
| ES 213 | M00074158C:F12 | PTA-2379 |
| ES 213 | M00074158C:H10 | PTA-2379 |
| ES 213 | M00074159C:A05 | PTA-2379 |
| ES 213 | M00074160A:D12 | PTA-2379 |
| ES 213 | M00074161C:F04 | PTA-2379 |
| ES 213 | M00074162A:B03 | PTA-2379 |
| ES 213 | M00074165D:A11 | PTA-2379 |
| ES 213 | M00074170A:D09 | PTA-2379 |
| ES 213 | M00074170D:F05 | PTA-2379 |
| ES 213 | M00074172B:D12 | PTA-2379 |
| ES 213 | M00074174A:C02 | PTA-2379 |
| ES 213 | M00074174C:C03 | PTA-2379 |
| ES 213 | M00074175D:E04 | PTA-2379 |
| ES 213 | M00074176A:A06 | PTA-2379 |
| ES 213 | M00074176A:B10 | PTA-2379 |
| ES 213 | M00074177B:H08 | PTA-2379 |
| ES 213 | M00074178B:G07 | PTA-2379 |
| ES 213 | M00074179A:A01 | PTA-2379 |
| ES 213 | M00074179C:B01 | PTA-2379 |
| ES 213 | M00074184D:A04 | PTA-2379 |
| ES 213 | M00074184D:B01 | PTA-2379 |
| ES 213 | M00074190B:F09 | PTA-2379 |
| ES 213 | M00074191C:D08 | PTA-2379 |
| ES 213 | M00074192C:C10 | PTA-2379 |
| ES 213 | M00074195D:B09 | PTA-2379 |
| ES 213 | M00074197C:A12 | PTA-2379 |
| ES 213 | M00074198C:A12 | PTA-2379 |
| ES 213 | M00074198D:D10 | PTA-2379 |
| ES 213 | M00074199A:C10 | PTA-2379 |
| ES 213 | M00074201A:F03 | PTA-2379 |
| ES 213 | M00074201C:E12 | PTA-2379 |
| ES 213 | M00074202A:A05 | PTA-2379 |
| ES 213 | M00074202B:D03 | PTA-2379 |
| ES 213 | M00074203D:F01 | PTA-2379 |
| ES 213 | M00074206A:G02 | PTA-2379 |
| ES 213 | M00074206A:H12 | PTA-2379 |
| ES 213 | M00074206B:F04 | PTA-2379 |
| ES 213 | M00074207D:E07 | PTA-2379 |
| ES 213 | M00074208B:B05 | PTA-2379 |
| ES 213 | M00074208B:F09 | PTA-2379 |
| ES 213 | M00074208D:E08 | PTA-2379 |
| ES 213 | M00074209D:H11 | PTA-2379 |
| ES 213 | M00074210B:G12 | PTA-2379 |
| ES 213 | M00074213A:C06 | PTA-2379 |
| ES 213 | M00074215A:F09 | PTA-2379 |
| ES 213 | M00074216C:C11 | PTA-2379 |
| ES 213 | M00074216D:H03 | PTA-2379 |
| ES 213 | M00074217A:H01 | PTA-2379 |
| ES 213 | M00074217C:B04 | PTA-2379 |
| ES 213 | M00074217C:C09 | PTA-2379 |
| ES 213 | M00074219D:F03 | PTA-2379 |
| ES 213 | M00074221B:F12 | PTA-2379 |
| ES 213 | M00074223B:D12 | PTA-2379 |
| ES 213 | M00074224A:G06 | PTA-2379 |
| ES 213 | M00074225A:H12 | PTA-2379 |
| ES 213 | M00074226C:E06 | PTA-2379 |
| ES 213 | M00074230D:B05 | PTA-2379 |
| ES 213 | M00074231A:D10 | PTA-2379 |
| ES 213 | M00074231D:G11 | PTA-2379 |
| ES 213 | M00074232B:G06 | PTA-2379 |
| ES 213 | M00074234A:C05 | PTA-2379 |
| ES 213 | M00074234A:E07 | PTA-2379 |
| ES 213 | M00074234B:F07 | PTA-2379 |
| ES 213 | M00074234D:F12 | PTA-2379 |
| ES 213 | M00074235C:D06 | PTA-2379 |
| ES 213 | M00074236B:E06 | PTA-2379 |
| ES 213 | M00074236C:E11 | PTA-2379 |
| ES 213 | M00074242D:F09 | PTA-2379 |
| ES 213 | M00074243A:H08 | PTA-2379 |
| ES 213 | M00074243C:B06 | PTA-2379 |
| ES 213 | M00074244C:B11 | PTA-2379 |
| ES 213 | M00074247B:G11 | PTA-2379 |
| ES 213 | M00074247C:E02 | PTA-2379 |
| ES 213 | M00074248C:E12 | PTA-2379 |
| ES 213 | M00074249C:B11 | PTA-2379 |
| ES 213 | M00074249C:H08 | PTA-2379 |
| ES 213 | M00074250D:E06 | PTA-2379 |
| ES 213 | M00074250D:F06 | PTA-2379 |
| ES 213 | M00074251B:F08 | PTA-2379 |
| ES 213 | M00074251C:B06 | PTA-2379 |
| ES 213 | M00074251C:E03 | PTA-2379 |
| ES 213 | M00074251D:E03 | PTA-2379 |
| ES 213 | M00074252C:E02 | PTA-2379 |
| ES 213 | M00074253C:F03 | PTA-2379 |
| ES 213 | M00074255B:A01 | PTA-2379 |
| ES 213 | M00074258A:H12 | PTA-2379 |
| ES 213 | M00074258A:H09 | PTA-2379 |
| ES 213 | M00074259C:G08 | PTA-2379 |
| ES 213 | M00074260B:A11 | PTA-2379 |
| ES 213 | M00074265B:C07 | PTA-2379 |
| ES 213 | M00074266A:D01 | PTA-2379 |
| ES 213 | M00074267A:B04 | PTA-2379 |
| ES 213 | M00074268A:D08 | PTA-2379 |
| ES 213 | M00074268C:G03 | PTA-2379 |
| ES 213 | M00074270B:A01 | PTA-2379 |
| ES 213 | M00074271B:E11 | PTA-2379 |
| ES 214 | M00072971A:E04 | PTA-2380 |
| ES 214 | M00072971A:F11 | PTA-2380 |
| ES 214 | M00072971C:B07 | PTA-2380 |
| ES 214 | M00072972A:C03 | PTA-2380 |
| ES 214 | M00072974A:A11 | PTA-2380 |
| ES 214 | M00072974D:B04 | PTA-2380 |
| ES 214 | M00072975A:D11 | PTA-2380 |
| ES 214 | M00072975A:E02 | PTA-2380 |
| ES 214 | M00072977A:F06 | PTA-2380 |
| ES 214 | M00072977B:C05 | PTA-2380 |
| ES 214 | M00072980B:C05 | PTA-2380 |
| ES 214 | M00072980B:G01 | PTA-2380 |
| ES 214 | M00073001A:F07 | PTA-2380 |
| ES 214 | M00073001B:E07 | PTA-2380 |
| ES 214 | M00073002B:B12 | PTA-2380 |
| ES 214 | M00073002D:B08 | PTA-2380 |
| ES 214 | M00073003A:E06 | PTA-2380 |
| ES 214 | M00073003B:E10 | PTA-2380 |
| ES 214 | M00073003B:H01 | PTA-2380 |
| ES 214 | M00073003C:C05 | PTA-2380 |
| ES 214 | M00073006A:H08 | PTA-2380 |
| ES 214 | M00073006C:D07 | PTA-2380 |
| ES 214 | M00073007D:E05 | PTA-2380 |
| ES 214 | M00073009B:C08 | PTA-2380 |
| ES 214 | M00073009D:A02 | PTA-2380 |
| ES 214 | M00073012A:C11 | PTA-2380 |
| ES 214 | M00073013A:D10 | PTA-2380 |
| ES 214 | M00073013A:F10 | PTA-2380 |
| ES 214 | M00073013C:B10 | PTA-2380 |
| ES 214 | M00073013C:G05 | PTA-2380 |
| ES 214 | M00073014D:F01 | PTA-2380 |
| ES 214 | M00073015A:E12 | PTA-2380 |
| ES 214 | M00073015A:H06 | PTA-2380 |
| ES 214 | M00073015B:A05 | PTA-2380 |
| ES 214 | M00073015C:E10 | PTA-2380 |
| ES 214 | M00073017A:D06 | PTA-2380 |
| ES 214 | M00073017A:F03 | PTA-2380 |
| ES 214 | M00073019A:H12 | PTA-2380 |
| ES 214 | M00073019B:B12 | PTA-2380 |
| ES 214 | M00073020C:F07 | PTA-2380 |
| ES 214 | M00073020D:C06 | PTA-2380 |
| ES 214 | M00073021C:E04 | PTA-2380 |
| ES 214 | M00073021D:C03 | PTA-2380 |
| ES 214 | M00073023A:D10 | PTA-2380 |
| ES 214 | M00073025A:E11 | PTA-2380 |
| ES 214 | M00073026B:F01 | PTA-2380 |
| ES 214 | M00073026D:G04 | PTA-2380 |
| ES 214 | M00073027B:H12 | PTA-2380 |
| ES 214 | M00073030A:G05 | PTA-2380 |
| ES 214 | M00073030B:C02 | PTA-2380 |
| ES 214 | M00073030C:A02 | PTA-2380 |
| ES 214 | M00073036C:H10 | PTA-2380 |
| ES 214 | M00073037A:C06 | PTA-2380 |
| ES 214 | M00073037D:H02 | PTA-2380 |
| ES 214 | M00073038C:C07 | PTA-2380 |
| ES 214 | M00073038D:D12 | PTA-2380 |
| ES 214 | M00073038D:F10 | PTA-2380 |
| ES 214 | M00073039A:D09 | PTA-2380 |
| ES 214 | M00073039C:B10 | PTA-2380 |
| ES 214 | M00073040A:B02 | PTA-2380 |
| ES 214 | M00073040D:F05 | PTA-2380 |
| ES 214 | M00073043B:C10 | PTA-2380 |
| ES 214 | M00073043B:E08 | PTA-2380 |
| ES 214 | M00073043C:F04 | PTA-2380 |
| ES 214 | M00073043D:H09 | PTA-2380 |
| ES 214 | M00073044B:F08 | PTA-2380 |
| ES 214 | M00073044C:C12 | PTA-2380 |
| ES 214 | M00073044C:D08 | PTA-2380 |
| ES 214 | M00073044C:G12 | PTA-2380 |
| ES 214 | M00073044D:F08 | PTA-2380 |
| ES 214 | M00073045B:A03 | PTA-2380 |
| ES 214 | M00073045B:D06 | PTA-2380 |
| ES 214 | M00073045C:E06 | PTA-2380 |
| ES 214 | M00073045C:E07 | PTA-2380 |
| ES 214 | M00073045D:B04 | PTA-2380 |
| ES 214 | M00073046A:A05 | PTA-2380 |
| ES 214 | M00073046A:A06 | PTA-2380 |
| ES 214 | M00073046B:A12 | PTA-2380 |
| ES 214 | M00073046D:F04 | PTA-2380 |
| ES 214 | M00073047B:E10 | PTA-2380 |
| ES 214 | M00073047C:G01 | PTA-2380 |
| ES 214 | M00073048A:H05 | PTA-2380 |
| ES 214 | M00073048C:A11 | PTA-2380 |
| ES 214 | M00073048C:B01 | PTA-2380 |
| ES 214 | M00073048C:E11 | PTA-2380 |
| ES 214 | M00073049A:H04 | PTA-2380 |
| ES 214 | M00073049B:B03 | PTA-2380 |
| ES 214 | M00073049B:B06 | PTA-2380 |
| ES 214 | M00073049C:C09 | PTA-2380 |
| ES 214 | M00073049C:H07 | PTA-2380 |
| ES 214 | M00073050A:D09 | PTA-2380 |
| ES 214 | M00073051A:D07 | PTA-2380 |
| ES 214 | M00073051A:F12 | PTA-2380 |
| ES 214 | M00073051A:F07 | PTA-2380 |
| ES 214 | M00073052B:H12 | PTA-2380 |
| ES 214 | M00074273B:B03 | PTA-2380 |
| ES 214 | M00074275A:B04 | PTA-2380 |
| ES 214 | M00074276A:A12 | PTA-2380 |
| ES 214 | M00074276A:E02 | PTA-2380 |
| ES 214 | M00074278B:D07 | PTA-2380 |
| ES 214 | M00074278D:E07 | PTA-2380 |
| ES 214 | M00074279C:C11 | PTA-2380 |
| ES 214 | M00074280D:H03 | PTA-2380 |
| ES 214 | M00074284B:B03 | PTA-2380 |
| ES 214 | M00074284C:B06 | PTA-2380 |
| ES 214 | M00074284C:E12 | PTA-2380 |
| ES 214 | M00074288A:F11 | PTA-2380 |
| ES 214 | M00074290A:G10 | PTA-2380 |
| ES 214 | M00074290C:B05 | PTA-2380 |
| ES 214 | M00074292D:B04 | PTA-2380 |
| ES 214 | M00074293D:B05 | PTA-2380 |
| ES 214 | M00074293D:H07 | PTA-2380 |
| ES 214 | M00074296C:G09 | PTA-2380 |
| ES 214 | M00074299B:F01 | PTA-2380 |
| ES 214 | M00074302D:G10 | PTA-2380 |
| ES 214 | M00074304B:C09 | PTA-2380 |
| ES 214 | M00074304D:D07 | PTA-2380 |
| ES 214 | M00074306A:B09 | PTA-2380 |
| ES 214 | M00074306B:H01 | PTA-2380 |
| ES 214 | M00074310D:D02 | PTA-2380 |
| ES 214 | M00074314A:C06 | PTA-2380 |
| ES 214 | M00074315B:A03 | PTA-2380 |
| ES 214 | M00074317C:C01 | PTA-2380 |
| ES 214 | M00074319C:H03 | PTA-2380 |
| ES 214 | M00074320C:B07 | PTA-2380 |
| ES 214 | M00074832B:E05 | PTA-2380 |
| ES 214 | M00074835A:H10 | PTA-2380 |
| ES 214 | M00074835B:F12 | PTA-2380 |
| ES 214 | M00074837A:B06 | PTA-2380 |
| ES 214 | M00074837A:E01 | PTA-2380 |
| ES 214 | M00074838B:E11 | PTA-2380 |
| ES 214 | M00074838D:B06 | PTA-2380 |
| ES 214 | M00074843A:C06 | PTA-2380 |
| ES 214 | M00074843A:F11 | PTA-2380 |
| ES 214 | M00074843D:D02 | PTA-2380 |
| ES 214 | M00074844B:B02 | PTA-2380 |
| ES 214 | M00074844D:F09 | PTA-2380 |
| ES 214 | M00074845A:D12 | PTA-2380 |
| ES 214 | M00074845B:F07 | PTA-2380 |
| ES 214 | M00074845D:D07 | PTA-2380 |
| ES 214 | M00074847B:G03 | PTA-2380 |
| ES 214 | M00074847D:E07 | PTA-2380 |
| ES 214 | M00074849C:A04 | PTA-2380 |
| ES 214 | M00074852A:B01 | PTA-2380 |
| ES214 | M00074852B:A02 | PTA-2380 |
| ES 214 | M00074852D:D08 | PTA-2380 |
| ES 214 | M00074853A:D05 | PTA-2380 |
| ES 214 | M00074854A:C11 | PTA-2380 |
| ES 214 | M00074855B:A05 | PTA-2380 |
| ES 214 | M00074857D:B02 | PTA-2380 |
| ES 214 | M00074858B:E05 | PTA-2380 |
| ES 214 | M00074861D:D01 | PTA-2380 |
| ES 214 | M00074863D:F07 | PTA-2380 |
| ES 214 | M00074864C:B09 | PTA-2380 |
| ES 214 | M00074317D:B08 | PTA-2380 |
| ES 214 | M00074320C:A06 | PTA-2380 |
| ES 214 | M00074865A:F05 | PTA-2380 |
| ES 214 | M00074869C:D04 | PTA-2380 |
| ES 214 | M00074871C:G05 | PTA-2380 |
| ES 214 | M00074874A:G07 | PTA-2380 |
| ES 214 | M00074875B:E08 | PTA-2380 |
| ES 214 | M00074879A:A02 | PTA-2380 |
| ES 214 | M00074879C:D02 | PTA-2380 |
| ES 214 | M00074884C:F10 | PTA-2380 |
| ES 214 | M00074887A:F03 | PTA-2380 |
| ES 214 | M00074890A:E03 | PTA-2380 |
| ES 214 | M00074895D:H12 | PTA-2380 |
| ES 214 | M00074898B:B01 | PTA-2380 |
| ES 214 | M00074900C:E10 | PTA-2380 |
| ES 214 | M00074901C:E05 | PTA-2380 |
| ES 214 | M00074903D:C04 | PTA-2380 |
| ES 214 | M00074904A:E11 | PTA-2380 |
| ES 214 | M00074904B:B07 | PTA-2380 |
| ES 214 | M00074905D:A01 | PTA-2380 |
| ES 214 | M00074906B:H12 | PTA-2380 |
| ES 214 | M00074906D:G02 | PTA-2380 |
| ES 214 | M00074912B:A10 | PTA-2380 |
| ES 214 | M00074912D:H08 | PTA-2380 |
| ES 214 | M00074916A:H03 | PTA-2380 |
| ES 214 | M00074919C:A08 | PTA-2380 |
| ES 214 | M00074921C:E05 | PTA-2380 |
| ES 214 | M00074922A:D06 | PTA-2380 |
| ES 214 | M00074927A:D02 | PTA-2380 |
| ES 214 | M00074927B:G08 | PTA-2380 |
| ES 214 | M00074927D:G09 | PTA-2380 |
| ES 214 | M00074929D:D04 | PTA-2380 |
| ES 214 | M00074930C:D11 | PTA-2380 |
| ES 214 | M00074933A:D04 | PTA-2380 |
| ES 214 | M00074935A:C01 | PTA-2380 |
| ES 214 | M00074936B:E10 | PTA-2380 |
| ES 214 | M00074939B:A06 | PTA-2380 |
| ES 214 | M00074940C:H08 | PTA-2380 |
| ES 215 | M00074950A:D01 | PTA-2381 |
| ES 215 | M00074958D:H10 | PTA-2381 |
| ES 215 | M00074966D:E08 | PTA-2381 |
| ES 215 | M00074967B:A11 | PTA-2381 |
| ES 215 | M00074968D:A02 | PTA-2381 |
| ES 215 | M00074974C:E11 | PTA-2381 |
| ES 215 | M00074980D:E07 | PTA-2381 |
| ES 215 | M00074954A:H06 | PTA-2381 |
| ES 215 | M00074954B:E03 | PTA-2381 |
| ES 215 | M00074957D:F11 | PTA-2381 |
| ES 215 | M00074962B:F08 | PTA-2381 |
| ES 215 | M00074968A:D09 | PTA-2381 |
| ES 215 | M00074973A:H03 | PTA-2381 |
| ES 215 | M00072987B:A03 | PTA-2381 |
| ES 215 | M00072997B:H03 | PTA-2381 |
| ES 215 | M00072951C:C11 | PTA-2381 |
| ES 215 | M00072953B:G03 | PTA-2381 |
| ES 215 | M00072982D:B03 | PTA-2381 |
| ES 215 | M00072985A:C12 | PTA-2381 |
| ES 215 | M00072985B:D03 | PTA-2381 |
| ES 215 | M00072986A:C03 | PTA-2381 |
| ES 215 | M00072993B:D06 | PTA-2381 |
| ES 215 | M00072995C:D07 | PTA-2381 |
| ES 215 | M00072995D:C09 | PTA-2381 |
| ES 215 | M00072996B:A10 | PTA-2381 |
| ES 215 | M00072996C:C04 | PTA-2381 |
| ES 215 | M00072997D:F08 | PTA-2381 |
| ES 215 | M00072997D:H06 | PTA-2381 |
| ES 215 | M00074323D:F09 | PTA-2381 |
| ES 215 | M00074333D:A11 | PTA-2381 |
| ES 215 | M00074335A:H08 | PTA-2381 |
| ES 215 | M00074337A:G08 | PTA-2381 |
| ES 215 | M00074340B:D06 | PTA-2381 |
| ES 215 | M00074343C:A03 | PTA-2381 |
| ES 215 | M00074346A:H09 | PTA-2381 |
| ES 215 | M00074347B:F11 | PTA-2381 |
| ES 215 | M00074349A:E08 | PTA-2381 |
| ES 215 | M00074355D:H06 | PTA-2381 |
| ES 215 | M00074361C:B01 | PTA-2381 |
| ES 215 | M00074365A:E09 | PTA-2381 |
| ES 215 | M00074366A:D07 | PTA-2381 |
| ES 215 | M00074366A:H07 | PTA-2381 |
| ES 215 | M00074370D:G09 | PTA-2381 |
| ES 215 | M00074375D:E05 | PTA-2381 |
| ES 215 | M00074382D:F04 | PTA-2381 |
| ES 215 | M00074384D:G07 | PTA-2381 |
| ES 215 | M00074388B:E07 | PTA-2381 |
| ES 215 | M00074392C:D02 | PTA-2381 |
| ES 215 | M00074405B:A04 | PTA-2381 |
| ES 215 | M00074417D:F07 | PTA-2381 |
| ES 215 | M00074392D:D01 | PTA-2381 |
| ES 215 | M00074406B:F10 | PTA-2381 |
| ES 215 | M00074430D:G09 | PTA-2381 |
| ES 215 | M00074395A:B11 | PTA-2381 |
| ES 215 | M00074404B:H01 | PTA-2381 |
| ES 215 | M00074391B:D02 | PTA-2381 |
| ES 215 | M00074390C:E04 | PTA-2381 |
| ES 215 | M00074411B:G07 | PTA-2381 |
| ES 215 | M00074415B:A01 | PTA-2381 |
| ES 215 | M00074453B:H03 | PTA-2381 |
| ES 215 | M00074453C:E09 | PTA-2381 |
| ES 215 | M00074454A:D08 | PTA-2381 |
| ES 215 | M00074461D:E04 | PTA-2381 |
| ES 215 | M00074463B:C03 | PTA-2381 |
| ES 215 | M00074468B:C03 | PTA-2381 |
| ES 215 | M00074473D:H09 | PTA-2381 |
| ES 215 | M00074474B:F02 | PTA-2381 |
| ES 215 | M00074488C:C10 | PTA-2381 |
| ES 215 | M00074488C:C08 | PTA-2381 |
| ES 215 | M00074492A:F11 | PTA-2381 |
| ES 215 | M00074501A:G07 | PTA-2381 |
| ES 215 | M00074502C:B08 | PTA-2381 |
| ES 215 | M00074515A:E02 | PTA-2381 |
| ES 215 | M00074515C:A11 | PTA-2381 |
| ES 215 | M00074516B:H03 | PTA-2381 |
| ES 215 | M00074525A:B05 | PTA-2381 |
| ES 215 | M00074533A:D07 | PTA-2381 |
| ES 215 | M00074539D:A10 | PTA-2381 |
| ES 215 | M00074540B:H07 | PTA-2381 |
| ES 215 | M00074541D:E07 | PTA-2381 |
| ES 215 | M00074549B:A06 | PTA-2381 |
| ES 215 | M00074557A:G08 | PTA-2381 |
| ES 215 | M00074561D:D12 | PTA-2381 |
| ES 215 | M00074566B:A04 | PTA-2381 |
| ES 215 | M00074569D:D04 | PTA-2381 |
| ES 215 | M00074521D:F01 | PTA-2381 |
| ES 215 | M00074549C:H08 | PTA-2381 |
| ES 215 | M00074555A:E10 | PTA-2381 |
| ES 215 | M00074561A:B09 | PTA-2381 |
| ES 215 | M00074565A:D08 | PTA-2381 |
| ES 215 | M00074571D:F02 | PTA-2381 |
| ES 215 | M00074573A:H02 | PTA-2381 |
| ES 215 | M00074577B:B12 | PTA-2381 |
| ES 215 | M00074577C:A05 | PTA-2381 |
| ES 215 | M00074582C:C02 | PTA-2381 |
| ES 215 | M00074582D:B09 | PTA-2381 |
| ES 215 | M00074584D:C01 | PTA-2381 |
| ES 215 | M00074588C:H06 | PTA-2381 |
| ES 215 | M00074589A:E10 | PTA-2381 |
| ES 215 | M00074593A:F05 | PTA-2381 |
| ES 215 | M00074596D:B12 | PTA-2381 |
| ES 215 | M00074606C:G02 | PTA-2381 |
| ES 215 | M00074607D:A12 | PTA-2381 |
| ES 215 | M00074613D:F01 | PTA-2381 |
| ES 215 | M00074614B:D10 | PTA-2381 |
| ES 215 | M00074625A:C12 | PTA-2381 |
| ES 215 | M00074628C:C11 | PTA-2381 |
| ES 215 | M00074628C:D03 | PTA-2381 |
| ES 215 | M00074633A:B09 | PTA-2381 |
| ES 215 | M00074636D:C01 | PTA-2381 |
| ES 215 | M00074637A:C02 | PTA-2381 |
| ES 215 | M00074638D:C12 | PTA-2381 |
| ES 215 | M00074639A:C08 | PTA-2381 |
| ES 215 | M00074640D:F07 | PTA-2381 |
| ES 215 | M00074645C:B07 | PTA-2381 |
| ES 215 | M00074654D:B05 | PTA-2381 |
| ES 215 | M00074662B:A05 | PTA-2381 |
| ES 215 | M00074662D:D01 | PTA-2381 |
| ES 215 | M00074664C:G09 | PTA-2381 |
| ES 215 | M00074668D:D04 | PTA-2381 |
| ES 215 | M00074674D:D02 | PTA-2381 |
| ES 215 | M00074676D:H07 | PTA-2381 |
| ES 215 | M00074681C:G11 | PTA-2381 |
| ES 215 | M00074681D:A02 | PTA-2381 |
| ES 215 | M00074687B:E01 | PTA-2381 |
| ES 215 | M00074699B:C03 | PTA-2381 |
| ES 215 | M00074701D:H09 | PTA-2381 |
| ES 215 | M00074702B:F12 | PTA-2381 |
| ES 215 | M00074702D:H05 | PTA-2381 |
| ES 215 | M00074713B:F02 | PTA-2381 |
| ES 215 | M00074716C:H07 | PTA-2381 |
| ES 215 | M00074723D:C06 | PTA-2381 |
| ES 215 | M00074723D:D05 | PTA-2381 |
| ES 215 | M00074728C:B08 | PTA-2381 |
| ES 215 | M00074730B:A04 | PTA-2381 |
| ES 215 | M00074740B:F06 | PTA-2381 |
| ES 215 | M00074744B:B12 | PTA-2381 |
| ES 215 | M00074748C:G02 | PTA-2381 |
| ES 215 | M00074752A:D08 | PTA-2381 |
| ES 215 | M00074753C:E10 | PTA-2381 |
| ES 215 | M00074755A:B10 | PTA-2381 |
| ES 215 | M00074755A:E07 | PTA-2381 |
| ES 215 | M00074765D:F06 | PTA-2381 |
| ES 215 | M00074766C:F12 | PTA-2381 |
| ES 215 | M00074768C:A05 | PTA-2381 |
| ES 215 | M00074773C:G03 | PTA-2381 |
| ES 215 | M00074774A:D03 | PTA-2381 |
| ES 215 | M00074777A:E01 | PTA-2381 |
| ES 215 | M00074780C:C02 | PTA-2381 |
| ES 215 | M00074782A:E04 | PTA-2381 |
| ES 215 | M00074808B:H02 | PTA-2381 |
| ES 215 | M00074996C:D07 | PTA-2381 |
| ES 215 | M00074981C:C09 | PTA-2381 |
| ES 215 | M00075000A:D06 | PTA-2381 |
| ES 215 | M00074805A:C12 | PTA-2381 |
| ES 215 | M00074981D:A03 | PTA-2381 |
| ES 215 | M00074794C:H02 | PTA-2381 |
| ES 215 | M00074801C:E06 | PTA-2381 |
| ES 215 | M00074821B:B03 | PTA-2381 |
| ES 215 | M00074823A:E03 | PTA-2381 |
| ES 215 | M00074800B:H01 | PTA-2381 |
| ES 215 | M00074800D:G09 | PTA-2381 |
| ES 215 | M00074812A:F03 | PTA-2381 |
| ES 215 | M00074825C:E06 | PTA-2381 |
| ES 215 | M00074794A:G10 | PTA-2381 |
| ES 215 | M00075018A:G04 | PTA-2381 |
| ES 215 | M00075020D:B04 | PTA-2381 |
| ES 215 | M00075049A:C09 | PTA-2381 |
| ES 215 | M00075032A:F02 | PTA-2381 |
| ES 215 | M00075029B:E03 | PTA-2381 |
| ES 215 | M00075069C:C01 | PTA-2381 |
| ES 215 | M00075039A:E01 | PTA-2381 |
| ES 215 | M00075024C:G05 | PTA-2381 |
| ES 215 | M00075074D:G11 | PTA-2381 |
| ES 215 | M00075011A:C11 | PTA-2381 |
| ES 215 | M00075061A:B03 | PTA-2381 |
| ES 215 | M00075043B:H05 | PTA-2381 |
| ES 215 | M00075035C:C09 | PTA-2381 |
| ES 215 | M00075045D:H03 | PTA-2381 |
| ES 215 | M00075078C:A07 | PTA-2381 |
| ES 215 | M00075075A:D12 | PTA-2381 |
| ES 215 | M00075077C:F09 | PTA-2381 |
| ES 215 | M00075026A:D11 | PTA-2381 |
| ES 215 | M00075044A:C10 | PTA-2381 |
| ES 215 | M00075075A:E09 | PTA-2381 |
| ES 215 | M00075020C:D12 | PTA-2381 |
| ES 215 | M00075117B:B06 | PTA-2381 |
| ES 215 | M00075114C:G11 | PTA-2381 |
| ES 215 | M00075153C:C11 | PTA-2381 |
| ES 215 | M00075161A:E05 | PTA-2381 |
| ES 215 | M00075126B:A06 | PTA-2381 |
| ES 215 | M00075126D:H07 | PTA-2381 |
| ES 216 | M00075092C:F04 | PTA-2382 |
| ES 216 | M00075110C:B03 | PTA-2382 |
| ES 216 | M00075132C:A03 | PTA-2382 |
| ES 216 | M00075152D:C06 | PTA-2382 |
| ES 216 | M00075125B:C07 | PTA-2382 |
| ES 216 | M00075132C:E07 | PTA-2382 |
| ES 216 | M00075160A:E04 | PTA-2382 |
| ES 216 | M00075149B:A01 | PTA-2382 |
| ES 216 | M00075120C:H04 | PTA-2382 |
| ES 216 | M00075093B:F10 | PTA-2382 |
| ES 216 | M00075102A:D02 | PTA-2382 |
| ES 216 | M00075090D:B07 | PTA-2382 |
| ES 216 | M00075161D:G06 | PTA-2382 |
| ES 216 | M00075165B:D04 | PTA-2382 |
| ES 216 | M00075174D:D06 | PTA-2382 |
| ES 216 | M00075180D:F05 | PTA-2382 |
| ES 216 | M00075181D:G10 | PTA-2382 |
| ES 216 | M00075189C:G05 | PTA-2382 |
| ES 216 | M00075199D:D11 | PTA-2382 |
| ES 216 | M00075201D:A05 | PTA-2382 |
| ES 216 | M00075203A:G06 | PTA-2382 |
| ES 216 | M00075211D:F09 | PTA-2382 |
| ES 216 | M00075221C:E02 | PTA-2382 |
| ES 216 | M00075228D:G09 | PTA-2382 |
| ES 216 | M00075232C:A06 | PTA-2382 |
| ES 216 | M00075232D:C06 | PTA-2382 |
| ES 216 | M00075234C:E06 | PTA-2382 |
| ES 216 | M00075239C:D06 | PTA-2382 |
| ES 216 | M00075242A:G04 | PTA-2382 |
| ES 216 | M00075243D:F04 | PTA-2382 |
| ES 216 | M00075245A:A06 | PTA-2382 |
| ES 216 | M00075249A:B08 | PTA-2382 |
| E5 216 | M00075252B:F10 | PTA-2382 |
| ES 216 | M00075255A:G11 | PTA-2382 |
| ES 216 | M00075259C:G02 | PTA-2382 |
| ES 216 | M00075270D:A02 | PTA-2382 |
| ES 216 | M00075273C:E01 | PTA-2382 |
| ES 216 | M00075274B:F06 | PTA-2382 |
| ES 216 | M00075275B:H07 | PTA-2382 |
| ES 216 | M00075279C:E08 | PTA-2382 |
| ES 216 | M00075283A:F04 | PTA-2382 |
| ES 216 | M00075302B:C07 | PTA-2382 |
| ES 216 | M00075305C:C07 | PTA-2382 |
| ES 216 | M00075309C:A06 | PTA-2382 |
| ES 216 | M00075323B:B12 | PTA-2382 |
| ES 216 | M00075324B:C10 | PTA-2382 |
| ES 216 | M00075324D:E02 | PTA-2382 |
| ES 216 | M00075326C:B01 | PTA-2382 |
| ES 216 | M00075326D:A09 | PTA-2382 |
| ES 216 | M00075329B:E10 | PTA-2382 |
| ES 216 | M00075330D:F11 | PTA-2382 |
| ES 216 | M00075333D:B07 | PTA-2382 |
| ES 216 | M00075333D:D10 | PTA-2382 |
| ES 216 | M00075336B:B04 | PTA-2382 |
| ES 216 | M00075344D:A08 | PTA-2382 |
| ES 216 | M00075347D:D01 | PTA-2382 |
| ES 216 | M00075354A:D11 | PTA-2382 |
| ES 216 | M00075354A:G12 | PTA-2382 |
| ES 216 | M00075354C:B12 | PTA-2382 |
| ES 216 | M00075360D:D04 | PTA-2382 |
| ES 216 | M00075365B:B06 | PTA-2382 |
| ES 216 | M00075384A:B03 | PTA-2382 |
| ES 216 | M00075389B:C06 | PTA-2382 |
| ES 216 | M00075391D:D07 | PTA-2382 |
| ES 216 | M00075402A:F01 | PTA-2382 |
| ES 216 | M00075405B:C07 | PTA-2382 |
| ES 216 | M00075405D:A10 | PTA-2382 |
| ES 216 | M00075365D:B08 | PTA-2382 |
| ES 216 | M00075380D:F06 | PTA-2382 |
| ES 216 | M00075356D:C03 | PTA-2382 |
| ES 216 | M00075352D:F09 | PTA-2382 |
| ES 216 | M00075359D:E09 | PTA-2382 |
| ES 216 | M00075365D:H01 | PTA-2382 |
| ES 216 | M00075373C:B09 | PTA-2382 |
| ES 216 | M00075378B:C07 | PTA-2382 |
| ES 216 | M00075379A:E07 | PTA-2382 |
| ES 216 | M00075383A:B11 | PTA-2382 |
| ES 216 | M00075407A:B05 | PTA-2382 |
| ES 216 | M00075409A:E04 | PTA-2382 |
| ES 216 | M00075409B:G12 | PTA-2382 |
| ES 216 | M00075416C:B02 | PTA-2382 |
| ES 216 | M00075458B:F09 | PTA-2382 |
| ES 216 | M00075464C:A07 | PTA-2382 |
| ES 216 | M00075458C:F01 | PTA-2382 |
| ES 216 | M00075463C:E07 | PTA-2382 |
| ES 216 | M00075464C:C04 | PTA-2382 |
| ES 216 | M00075448B:G11 | PTA-2382 |
| ES 216 | M00075434A:D06 | PTA-2382 |
| ES 216 | M00075457C:A06 | PTA-2382 |
| ES 216 | M00075454C:D06 | PTA-2382 |
| ES 216 | M00075460C:B06 | PTA-2382 |
| ES 216 | M00075459A:C02 | PTA-2382 |
| ES 216 | M00075414A:D10 | PTA-2382 |
| ES 216 | M00075433A:C06 | PTA-2382 |
| ES 216 | M00075505B:A04 | PTA-2382 |
| ES 216 | M00075474D:B07 | PTA-2382 |
| ES 216 | M00075504B:A10 | PTA-2382 |
| ES 216 | M00075473C:E08 | PTA-2382 |
| ES 216 | M00075499A:H02 | PTA-2382 |
| ES 216 | M00075495D:D11 | PTA-2382 |
| ES 216 | M00075496D:G05 | PTA-2382 |
| ES 216 | M00075514A:G12 | PTA-2382 |
| ES 216 | M00075495B:C12 | PTA-2382 |
| ES 216 | M00075497D:H03 | PTA-2382 |
| ES 216 | M00075529A:A02 | PTA-2382 |
| ES 216 | M00075538C:E03 | PTA-2382 |
| ES 216 | M00075544A:C03 | PTA-2382 |
| ES 216 | M00075598B:A09 | PTA-2382 |
| ES 216 | M00075521B:E11 | PTA-2382 |
| ES 216 | M00075597C:G01 | PTA-2382 |
| ES 216 | M00075584D:B05 | PTA-2382 |
| ES 216 | M00075590B:G04 | PTA-2382 |
| ES 216 | M00075603D:D09 | PTA-2382 |
| ES 216 | M00075607B:D05 | PTA-2382 |
| ES 216 | M00075609A:H06 | PTA-2382 |
| ES 216 | M00075613D:F01 | PTA-2382 |
| ES 216 | M00075619C:D08 | PTA-2382 |
| ES 216 | M00075621A:F06 | PTA-2382 |
| ES 216 | M00075639A:D12 | PTA-2382 |

We claim:

1. An isolated polynucleotide comprising a nucleotide sequence which hybridizes under stringent conditions to a sequence selected from the group consisting of SEQ ID NOS: 1-1477.

2. An isolated polynucleotide comprising at least 15 contiguous nucleotides of a nucleotide sequence having at least 90% sequence identity to a sequence selected from the group consisting of: SEQ ID NOS: 1-1477, a degenerate variant of SEQ ID NOS: 1-1477, an antisense of SEQ ID NOS: 1-1477, and a complement of SEQ ID NOS: 1-1477.

3. An isolated polynucleotide comprising at least 15 contiguous nucleotides of a nucleotide sequence selected from the group consisting of: SEQ ID NOS: 1-1477, a degenerate variant of SEQ ID NOS: 1-1477, an antisense of SEQ ID NOS: 1-1477, and a complement of SEQ ID NOS: 1-1477.

4. The isolated polynucleotide of claim 3, wherein the polynucleotide comprises at least 100 contiguous nucleotides of the nucleotide sequence.

5. The isolated polynucleotide of claim 3, wherein the polynucleotide comprises at least 200 contiguous nucleotides of the selected nucleotide sequence.

6. An isolated polynucleotide comprising a nucleotide sequence of at least 90% sequence identity to a sequence selected from the group consisting of: SEQ ID NOS: 1-1477, a degenerate variant of SEQ ID NOS: 1-1477, an antisense of SEQ ID NOS: 1-1477, and a complement of SEQ ID NOS: 1-1477.

7. The isolated polynucleotide of claim 6, wherein the polynucleotide comprises a nucleotide sequence of at least 95% sequence identity to the selected nucleotide sequence.

8. The isolated polynucleotide of claim 6, wherein the polynucleotide comprises a nucleotide sequence that is identical to the selected nucleotide sequence.

9. A polynucleotide comprising a nucleotide sequence of an insert contained in a clone deposited as ATCC Accession No. PTA-2918.

10. An isolated cDNA obtained by the process of amplification using a polynucleotide comprising at least 15 contiguous nucleotides of a nucleotide sequence of a sequence selected from the group consisting of SEQ ID NOS: 1-1477.

11. The isolated cDNA of claim 10, wherein the polynucleotide comprises at least 25 contiguous nucleotides of the selected nucleotide sequence.

12. The isolated cDNA of claim 10, wherein the polynucleotide comprises at least 100 contiguous nucleotides of the selected nucleotide sequence.

13. The isolated cDNA of claims 10, 11, or 12, wherein amplification is by polymerase chain reaction (PCR) amplification.

14. An isolated recombinant host cell containing the polynucleotide according to claims 1, 2, 3, 6, 9, or 10.

15. An isolated vector comprising the polynucleotide according to claims 1, 2, 3, 6, 9, or 10.

16. A method for producing a polypeptide, the method comprising the steps of:
culturing a recombinant host cell containing the polynucleotide according to claims 1, 2, 3, 6, 9, or 10., said culturing being under conditions suitable for the expression of an encoded polypeptide; and
recovering the polypeptide from the host cell culture.

17. An isolated polypeptide encoded by the polynucleotide according to claims 1, 2, 3, 6, 9, or 10.

18. An isolated polypeptide comprising an amino acid sequence selected from the group consisting of SEQ ID NOS: 1478-1568.

19. An antibody that specifically binds the polypeptide of claim 17 or 18.

20. A method of detecting differentially expressed genes correlated with a cancerous state of a mammalian cell, the method comprising the step of:
detecting at least one differentially expressed gene product in a test sample derived from a cell suspected of being cancerous, where the gene product is encoded by a gene comprising an identifying sequence of at least one of SEQ ID NOS: 1-1477;
wherein detection of the differentially expressed gene product is correlated with a cancerous state of the cell from which the test sample was derived.

21. A method of detecting differentially expressed genes correlated with a cancerous state of a mammalian cell, the method comprising the step of:
detecting at least one differentially expressed gene product in a test sample derived from a cell suspected of being cancerous, where the gene product comprises an amino acid sequence selected from the group consisting of SEQ ID NOS: 1478-1568;
wherein detection of the differentially expressed gene product is correlated with a cancerous state of the cell from which the test sample was derived.

22. A library of polynucleotides, wherein at least one of the polynucleotides comprises the sequence information of the polynucleotide according to claims 1, 2, 3, 6, 9, or 10.

23. The library of claim 22, wherein the library is provided on a nucleic acid array.

24. The library of claim 22, wherein the library is provided in a computer-readable format.

25. A method of inhibiting tumor growth by modulating expression of a gene product, the gene product being encoded by a gene identified by a sequence selected from the group consisting of SEQ ID NOS: 1-1477.

26. A method of inhibiting tumor growth by modulating expression of a gene product, the gene product comprising an amino acid sequence selected from the group consisting of SEQ ID NOS: 1478-1568.
